(19) United States
(12) Patent Application Publication
(10) Pub. No.: US 2023/0322785 A1
(43) Pub. Date: Oct. 12, 2023

(54) ADENOSINE A2A AND A2B RECEPTOR DUAL ANTAGONISTS FOR IMMUNO-ONCOLOGY

(71) Applicants: Christopher W. BOYCE, West Point, PA (US); Thomas H. GRAHAM, Boston, MA (US); Andrew J. HOOVER, Boston, MA (US); Xianhai HUANG, Warren, NJ (US); Rognze KUANG, Kenilworth, NJ (US); Jae-Hun KIM, Cranford, NJ (US); Joseph M. KELLY, Parlin, NJ (US); Michael Man-Chu LO, Kenilworth, NJ (US); Jing SU, Kenilworth, NJ (US); Dong XIAO, Rahway, NJ (US); Xiaohong ZHU, Kenilworth, NJ (US); Merck Sharp & Dohme LLC, Rahway, NJ (US)

(72) Inventors: Amjad Ali, Freehold, NJ (US); Christopher W. Boyce, Flemington, NJ (US); Jared N. Cumming, Winchester, MA (US); Duane DeMong, Hanover, MA (US); Thomas H. Graham, Somerville, MA (US); Subrahmanyam Gudipati, Middletown, DE (US); Andrew J. Hoover, Framingham, MA (US); Xianhai Huang, Warren, NJ (US); Rongze Kuang, Green Brook, NJ (US); Jae-Hun Kim, Cranford, NJ (US); Joseph M. Kelly, Parlin, NJ (US); Yeon-Hee Lim, South San Francisco, CA (US); Michael Man-Chu Lo, Bedminster, NJ (US); Jesus Moreno, San Diego, CA (US); Jing Su, Scotch Plains, NJ (US); Heping Wu, Edison, NJ (US); Dong Xiao, Warren, NJ (US); Younong Yu, East Brunswick, NJ (US); Xiaohong Zhu, Edison, NJ (US)

(73) Assignee: Merck Sharp & Dohme LLC, Rahway, NJ (US)

(21) Appl. No.: 18/015,387

(22) PCT Filed: Jul. 21, 2022
(86) PCT No.: PCT/US2021/042702

§ 371 Date: Jan. 10, 2023

Related U.S. Application Data
(60) Provisional application No. 63/056,246, filed on Jul. 24, 2020.

(51) Int. Cl. C07D 487/04; C07K 16/28; A61P 35/00

(52) U.S. Cl. CPC C07D 487/04; A61P 35/00; C07K 16/2896

(57) ABSTRACT

The present invention provides compounds of the structural Formula (I) and pharmaceutically acceptable salts thereof, wherein, are as defined herein, pharmaceutical compositions comprising one or more such compounds (alone and in combination with one or more other therapeutically active agents), and methods for their preparation and use, alone and in combination with other therapeutic agents, as antagonists of A2a and/or A2b receptors, and in the treatment of a variety of diseases, conditions, or disorders that are mediated, at least in part, by the adenosine A2a receptor and/or the adenosine A2b receptor.

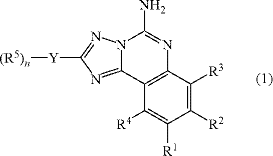

### FIELD OF THE INVENTION

The present invention relates to novel compounds that inhibit at least one of the A2a and A2b adenosine receptors, and pharmaceutically acceptable salts thereof, and compositions comprising such compound(s) and salts, methods for the synthesis of such compounds, and their use in the treatment of a variety of diseases, conditions, or disorders that are mediated, at least in part, by the adenosine A2a receptor and/or the adenosine A2b receptor. Such diseases, conditions, and disorders include but are not limited to cancer and immune-related disorders. The invention further relates to combination therapies, including but not limited to a combination comprising a compound of the invention and a PD-1 antagonist.

### BACKGROUND OF THE INVENTION

Adenosine is a purine nucleoside compound comprised of adenine and ribofuranose, a ribose sugar molecule. Adenosine occurs naturally in mammals and plays important roles in various biochemical processes, including energy transfer (as adenosine triphosphate and adenosine monophosphate) and signal transduction (as cyclic adenosine monophosphate). Adenosine also plays a causative role in processes associated with vasodilation, including cardiac vasodilation. It also acts as a neuromodulator (e.g., it is thought to be involved in promoting sleep). In addition to its involvement in these biochemical processes, adenosine is used as a therapeutic antiarrhythmic agent to treat supraventricular tachycardia and other indications.

The adenosine receptors are a class of purinergic G protein-coupled receptors with adenosine as the endogenous ligand. The four types of adenosine receptors in humans are referred to as A1, A2a, A2b, and A3. Modulation of A1 has been proposed for the management and treatment of neurological disorders, asthma, and heart and renal failure, among others. Modulation of A3 has been proposed for the management and treatment of asthma and chronic obstructive pulmonary diseases, glaucoma, cancer, stroke, and other indications. Modulation of the A2a and A2b receptors are also believed to be of potential therapeutic use.

In the central nervous system, A2a antagonists are believed to exhibit antidepressant properties and to stimulate cognitive functions. A2a receptors are present in high density in the basal ganglia, known to be important in the control of movement. Hence, A2a receptor antagonists are believed to be useful in the treatment of depression and to improve motor impairment due to neurodegenerative diseases such as Parkinson’s disease, senile dementia (as in Alzheimer’s disease), and in various psychoses of organic origin.

In the immune system, adenosine signaling through A2a receptors and A2b receptors, expressed on a variety of immune cells and endothelial cells, has been established as having an important role in protecting tissues during inflammatory responses. In this way (and others), tumors have been shown to evade host responses by inhibiting immune function and promoting tolerance. (See, e.g., Fishman, P., et al., Handb. Exp. Pharmacol. (2009) 193:399-441). Moreover, A2a and A2b cell surface adenosine receptors have been found to be upregulated in various tumor cells. Thus, antagonists of the A2a and/or A2b adenosine receptors represent a new class of promising oncology therapeutics. For example, activation of A2a adenosine receptors results in the inhibition of the immune response to tumors by a variety of cell types, including but not limited to: the inhibition of natural killer cell cytotoxicity, the inhibition of tumor-specific CD4+/CD8+ activity, promoting the generation of LAG-3 and Foxp3+ regulatory T-cells, and mediating the inhibition of regulatory T-cells. Adenosine A2a receptor inhibition has also been shown to increase the efficacy of PD-1 inhibitors through enhanced anti-tumor T cell responses. As each of these immunosuppressive pathways has been identified as a mechanism by which tumors evade host responses, a cancer immunotherapeutic regimen that includes an antagonist of the A2a and/or A2b receptors, alone or together with one or more other therapeutic agents designed to mitigate immune suppression, may result in enhanced tumor immunotherapy. (See, e.g., P. Beavis, et al., Cancer Immunol. Res. DOI: 10.1158/2326-6066. CIR-14-0211, Feb. 11, 2015; Willingham, SB., et al., Cancer Immunol. Res., 6(10), 1136-49; and Leone RD, et al., Cancer Immunol. Immunother., August 2018, Vol. 67, Issue 8, 1271-1284).

Cancer cells release ATP into the tumor microenvironment when treated with chemotherapy and radiation therapy, which is subsequently converted to adenosine. (See Martins, I., et al., Cell Cycle, vol. 8, issue 22, pp. 3723 to 3728.) The adenosine can then bind to A2a receptors and blunt the anti-tumor immune response through mechanisms such as those described above. The administration of A2a receptor antagonists during chemotherapy or radiation therapy has been proposed to lead to the expansion of the tumor-specific T-cells while simultaneously preventing the induction of tumor-specific regulatory T-cells. (Young, A., et al., Cancer Discovery (2014) 4:879-888).

The combination of an A2a receptor antagonist with anti-tumor vaccines is believed to provide at least an additive therapeutic effect in view of their different mechanisms of action. Further, A2a receptor antagonists may be useful in combination with checkpoint blockers. By way of example, the combination of a PD-1 inhibitor and an adenosine A2a receptor inhibitor is thought to mitigate the ability of tumors to inhibit the activity of tumor-specific effector T-cells. (See, e.g., Willingham, SB., et al., Cancer Immunol. Res.; 6(10), 1136-49; Leone, RD., et al., Cancer Immunol. Immunother., August 2018, Vol. 67, Issue 8, pp. 1271-1284; Fishman, P., et al., Handb. Exp. Pharmacol. (2009) 193:399-441; and Sitkovsky, MV., et al., (2014) Cancer Immunol. Res 2:598-605.)

The A2b receptor is a G protein-coupled receptor found in various cell types. A2b receptors require higher concentrations of adenosine for activation than the other adenosine receptor subtypes, including A2a. (Fredholm, BB., et al., Biochem. Pharmacol. (2001) 61:443-448). Conditions which activate A2b have been seen, for example, in tumors where hypoxia is observed. The A2b receptor may thus play an important role in pathophysiological conditions associated with massive adenosine release. While the pathway(s) associated with A2b receptor-mediated inhibition are not well understood, it is believed that the inhibition of A2b receptors (alone or together with A2a receptors) may block pro-tumorigenic functions of adenosine in the tumor microenvironment, including suppression of T-cell function and angiogenesis, and thus expand the types of cancers treatable by the inhibition of these receptors.

A2b receptors are expressed primarily on myeloid cells. The engagement of A2b receptors on myeloid derived suppressor cells (MDSCs) results in their expansion in vitro (Ryzhov, S. et al., J. Immunol. 2011, 187:6120-6129). MDSCs suppress T-cell proliferation and anti-tumor immune responses. Selective inhibitors of A2b receptors and A2b receptor knockouts have been shown to inhibit tumor growth in mouse models by increasing MDSCs in the tumor microenvironment (Iannone, R., et al., Neoplasia Vol. 13 No. 12, (2013) pp. 1400-1409; Ryzhov, S., et al., Neoplasia (2008) 10: 987-995). Thus, A2b receptor inhibition has become an attractive biological target for the treatment of a variety of cancers involving myeloid cells. Examples of cancers that express A2b receptors can be readily obtained through analysis of the publicly available TCGA database. Such cancers include lung, colorectal, head and neck, and cervical cancer, among others, and are discussed in further detail below.

Angiogenesis plays an important role in tumor growth. The angiogenesis process is highly regulated by a variety of factors and is triggered by adenosine under particular circumstances that are associated with hypoxia. The A2b receptor is expressed in human microvascular endothelial cells, where it plays an important role in the regulation of the expression of angiogenic factors such as the vascular endothelial growth factor (VEGF). In certain tumor types, hypoxia has been observed to cause an upregulation of the A2b receptors, suggesting that inhibition of A2b receptors may limit tumor growth by limiting the oxygen supply to the tumor cells. Furthermore, experiments involving adenylate cyclase activation indicate that A2b receptors are the sole adenosine receptor subtype in certain tumor cells, suggesting that A2b receptor antagonists may exhibit effects on particular tumor types. (See, e.g., Feoktistov, I., et al., (2003) Circ. Res. 92:485-492; and P. Fishman, P., et al., Handb. Exp. Pharmacol. (2009) 193:399-441).

In view of their promising and varied therapeutic potential, there remains a need in the art for potent and selective inhibitors of the A2a and/or A2b adenosine receptors, for use alone or in combination with other therapeutic agents. The present invention addresses this and other needs.

### SUMMARY OF THE INVENTION

In one aspect, the present invention provides compounds (hereinafter referred to as compounds of the invention) which, surprisingly and advantageously, have been found to be inhibitors of the adenosine A2a receptor and/or the adenosine A2b receptor. The compounds of the invention have a structure in accordance with the structural Formula (I):

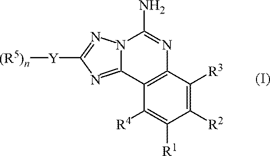

or a pharmaceutically acceptable salt thereof, wherein Y, R¹, R², R³, R⁴, R⁵ and n are as defined below.

In another aspect, the present invention provides pharmaceutical compositions comprising at least one compound of the invention, or a pharmaceutically acceptable salt thereof, in a pharmaceutically acceptable carrier or diluent. Such compositions according to the invention may optionally further include one or more additional therapeutic agents as described herein.

In another aspect, the present invention provides a method for treating or preventing a disease, condition, or disorder that is mediated, at least in part, by the adenosine A2a receptor and/or the adenosine A2b receptor in a subject (e.g., an animal or human) in need thereof, said method comprising administering to the subject a therapeutically effective amount of at least one compound of the invention, or a pharmaceutically acceptable salt thereof, alone or in combination with one or more additional therapeutic agents. These and other aspects and embodiments of the invention are described more fully below.

### DETAILED DESCRIPTION OF THE INVENTION

For each of the following embodiments, any variable not explicitly defined in the embodiment is as defined in Formula (I). In each of the embodiments described herein, each variable is selected independently of the other unless otherwise noted.

In one embodiment, the compounds of the invention have the structural Formula (I):

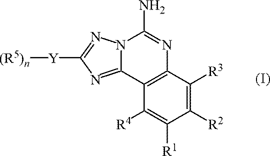

or a pharmaceutically acceptable salt thereof, wherein:
   R¹ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and heterocycloalkyl;
   R² is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and heterocycloalkyl;
   R³ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and heterocycloalkyl;
   R⁴ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and heterocycloalkyl;
   Y is a straight or branched (C₁-C₅)alkyl, wherein one or more —CH₂— groups in Y are independently replaced with a moiety selected from the group consisting of NR⁸, NR⁸C(O), OC(O)NR⁸, a nitrogen-containing heterocycloalkyl and C(O)NR⁸;
   each occurrence of R⁵ is hydrogen, halogen, aryl, (C₃-C₆)cycloalkyl, heterocycloalkyl, heteroaryl, (C₁-C₆)alkylOH, OH, (C₁-C₆)haloalkyl, (C₁-C₆)alkyl, (C₁-C₆)alkynyl, SO₂R⁶, SO(═NH)R⁶, SO(═NCH₃)R⁶ and COO(C₁-C₆)alkyl, wherein the aryl, (C₃-C₆)cycloalkyl, heterocycloalkyl or heteroaryl are optionally substituted with one to three substituents selected from the group consisting of halogen, (C₁-C₆)alkyl, (C₁-C₆)haloalkyl, O(C₁-C₆)alkyl, O(C₁-C₆)haloalkyl, (C₃-C₆)cycloalkyl, O(C₃-C₆)cycloalkyl, (C₁-C₆)haloalkylOH, (C₁-C₆)alkylOH, SO₂N(R⁷)₂, S(halo)₅, haloaryl, halo(C₃-C₆)cycloalkyl, haloheteroaryl, heterocycloalkyl, NR⁷C(O)(C₁-C₆)alkyl, heteroaryl, tert-butoxycarbonyl, or (C₁-C₆)alkylN(R⁷)₂;
   R⁶ is selected from the group consisting of OH, NH₂, (C₁-C₆)alkyl, aryl, (Ci-C₆)haloalkyl, (C₃-C₆)cycloalkyl and haloaryl;
   each occurrence of R⁷ is independently selected from the group consisting of H, (C₁-C₆)alkyl, (C₃-C₆)cycloalkyl, or when two R⁷ substituents are taken together with the nitrogen they are attached, form a heterocycloalkyl;
   R⁸ is independently selected from the group consisting of H, (C₁-C₆)alkyl, (C₃-C₆)cycloalkyl, (C₁-C₆)alkylOH, (C₁-C₆)haloalkyl (C₁-C₆)alkylcycloalkyl, (C₁-C₆)alkylO(C₁-C₆)alkyl, (C₁-C₆)alkylC(O)O(C₁-C₆)alkyl, SO₂(R⁶), CON(R⁷)₂, (C₁-C₆)alkylheterocycloalkyl, or (C₁-C₆)alkenyl;
   n is 1, 2 or 3.

In another embodiment, the compounds of the invention have a structure in accordance with the structural Formula (II):

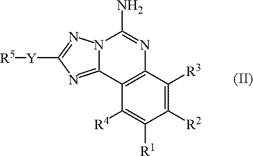

or a pharmaceutically acceptable salt thereof, wherein Y, R¹, R², R³, R⁴ and R⁵ are as defined below.

With regard to the compounds described herein, R¹ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and heterocycloalkyl.

In certain embodiments, R¹ is hydrogen.

In certain embodiments, R¹ is halogen. Suitable halogens include, but are not limited to, a fluorine, a chlorine, a bromine or an iodine. In certain embodiments, R¹ is fluorine.

In certain embodiments, R¹ is (C₁-C₆)alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R¹ is O(C₁-C₆)alkyl. Suitable alkoxys include, but are not limited to, methoxy, ethoxy, n-propoxy, isopropoxy and n-butoxy.

In certain embodiments, R¹ is OH.

In certain embodiments, R¹ is O(C₁-C₆)haloalkyl. Suitable examples of haloalkoxys include, but are not limited to, fluoromethoxys, difluoromethoxy, trifluoromethoxy, 2-fluoroethoxy, 1,2-difluoroethoxy and 2,2-difluoroethoxy.

In certain embodiments, R¹ is (C₁-C₆)haloalkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R¹ is CN.

In certain embodiments, R¹ is (C₃-C₆)cycloalkyl. In certain embodiments, R¹ is a monocyclic cycloalkyl. In other embodiments, R¹ is a bicyclic cycloalkyl. In other embodiments, R¹ is a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R¹ is

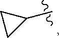

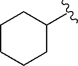

or

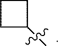

In certain embodiments, R¹ is heterocycloalkyl. In certain embodiments, R¹ is a monocyclic heterocycloalkyl. In other embodiments, R¹ is a bicyclic heterocycloalkyl. In other embodiments, R¹ is a multicyclic heterocycloalkyl. In other embodiments, R¹ is a nitrogen-containing heterocycloalkyl. In other embodiments, R¹ is an oxygen-containing heterocycloalkyl. In other embodiments, R¹ is a sulfur-containing heterocycloalkyl. In certain embodiments the heterocycloalkyl is

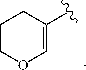

In certain embodiments, R¹ is hydrogen or fluorine.

With regard to the compounds described herein, R² is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and heterocycloalkyl.

In certain embodiments, R² is hydrogen.

In certain embodiments, R² is halogen. Suitable halogens include, but are not limited to, a fluorine, a chlorine, a bromine or an iodine.

In certain embodiments, R² is (C₁-C₆)alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R² is O(C₁-C₆)alkyl. Suitable alkoxys include, but are not limited to, methoxy, ethoxy, n-propoxy, isopropoxy and n-butoxy. In certain embodiments, R² is methoxy.

In certain embodiments, R² is OH.

In certain embodiments, R² is O(C₁-C₆)haloalkyl. Suitable examples of haloalkoxys include, but are not limited to, fluoromethoxys, difluoromethoxy, trifluoromethoxy, 2-fluoroethoxy, 1,2-difluoroethoxy and 2,2-difluoroethoxy.

In certain embodiments, R² is (C₁-C₆)haloalkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R² is CN.

In certain embodiments, R² is (C₃-C₆)cycloalkyl. In certain embodiments, R² is a monocyclic cycloalkyl. In other embodiments, R² is a bicyclic cycloalkyl. In other embodiments, R² is a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R² is

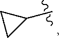

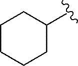

or

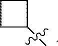

In certain embodiments, R² is heterocycloalkyl. In certain embodiments, R² is a monocyclic heterocycloalkyl. In other embodiments, R² is a bicyclic heterocycloalkyl. In other embodiments, R² is a multicyclic heterocycloalkyl. In other embodiments, R² is a nitrogen-containing heterocycloalkyl. In other embodiments, R² is an oxygen-containing heterocycloalkyl. In other embodiments, R² is a sulfur-containing heterocycloalkyl. In certain embodiments the heterocycloalkyl is

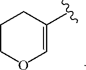

In certain embodiments, R² is hydrogen or methoxy.

With regard to the compounds described herein, R³ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and heterocycloalkyl.

In certain embodiments, R³ is hydrogen.

In certain embodiments, R³ is halogen. Suitable halogens include, but are not limited to, a fluorine, a chlorine, a bromine or an iodine. In certain embodiments, R³ is fluorine.

In certain embodiments, R³ is (C₁-C₆)alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R³ is O(C₁-C₆)alkyl. Suitable alkoxys include, but are not limited to, methoxy, ethoxy, n-propoxy, isopropoxy and n-butoxy. In certain embodiments, R³ is methoxy.

In certain embodiments, R³ is OH.

In certain embodiments, R³ is O(C₁-C₆)haloalkyl. Suitable examples of haloalkoxys include, but are not limited to, fluoromethoxys, difluoromethoxy, trifluoromethoxy, 2-fluoroethoxy, 1,2-difluoroethoxy and 2,2-difluoroethoxy.

In certain embodiments, R³ is (C₁-C₆)haloalkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R³ is CN.

In certain embodiments, R³ is (C₃-C₆)cycloalkyl. In certain embodiments, R³ is a monocyclic cycloalkyl. In other embodiments, R³ is a bicyclic cycloalkyl. In other embodiments, R³ is a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R³ is

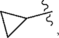

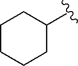

or

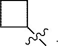

In certain embodiments, R³ is heterocycloalkyl. In certain embodiments, R³ is a monocyclic heterocycloalkyl. In other embodiments, R³ is a bicyclic heterocycloalkyl. In other embodiments, R³ is a multicyclic heterocycloalkyl. In other embodiments, R³ is a nitrogen-containing heterocycloalkyl. In other embodiments, R³ is an oxygen-containing heterocycloalkyl. In other embodiments, R³ is a sulfur-containing heterocycloalkyl. In certain embodiments the heterocycloalkyl is

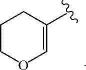

In certain embodiments, R³ is hydrogen, methoxy or fluorine.

With regard to the compounds described herein, R⁴ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and heterocycloalkyl.

In certain embodiments, R⁴ is hydrogen.

In certain embodiments, R⁴ is halogen. Suitable halogens include, but are not limited to, a fluorine, a chlorine, a bromine or an iodine. In certain embodiments, R⁴ is fluorine.

In certain embodiments, R⁴ is (C₁-C₆)alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R⁴ is O(C₁-C₆)alkyl. Suitable alkoxys include, but are not limited to, methoxy, ethoxy, n-propoxy, isopropoxy and n-butoxy. In certain embodiments, R⁴ is methoxy.

In certain embodiments, R⁴ is OH.

In certain embodiments, R⁴ is O(C₁-C₆)haloalkyl. Suitable examples of haloalkoxys include, but are not limited to, fluoromethoxys, difluoromethoxy, trifluoromethoxy, 2-fluoroethoxy, 1,2-difluoroethoxy and 2,2-difluoroethoxy.

In certain embodiments, R⁴ is (C₁-C₆)haloalkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R⁴ is CN.

In certain embodiments, R⁴ is (C₃-C₆)cycloalkyl. In certain embodiments, R⁴ is a monocyclic cycloalkyl. In other embodiments, R⁴ is a bicyclic cycloalkyl. In other embodiments, R⁴ is a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R⁴ is

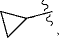

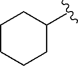

or

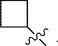

In certain embodiments, R⁴ is heterocycloalkyl. In certain embodiments, R⁴ is a monocyclic heterocycloalkyl. In other embodiments, R⁴ is a bicyclic heterocycloalkyl. In other embodiments, R⁴ is a multicyclic heterocycloalkyl. In other embodiments, R⁴ is a nitrogen-containing heterocycloalkyl. In other embodiments, R⁴ is an oxygen-containing heterocycloalkyl. In other embodiments, R⁴ is a sulfur-containing heterocycloalkyl. In certain embodiments the heterocycloalkyl is

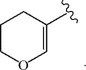

In certain embodiments, R⁴ is hydrogen.

In certain embodiments, R¹, R², R³, R⁴ are not simultaneously hydrogen.

With regard to the compounds described herein, Y is a straight or branched (Ci-Cs)alkyl, wherein one or more —CH₂— groups in Y are independently replaced with a moiety selected from the group consisting of NR⁸, NR⁸C(O), OC(O)NR⁸, a nitrogen-containing heterocycloalkyl and C(O)NR⁸. In certain embodiments, Y is a straight or branched (Ci-Cs)alkyl, wherein one —CH₂—groups in Y are independently replaced with a moiety selected from the group consisting of NR⁸, NR⁸C(O), OC(O)NR⁸, a nitrogen-containing heterocycloalkyl and C(O)NR⁸. In certain embodiments, Y is a straight or branched (Ci-Cs)alkyl, wherein two —CH₂— groups in Y are independently replaced with a moiety selected from the group consisting of NR⁸, NR⁸C(O), OC(O)NR⁸, a nitrogen-containing heterocycloalkyl and C(O)NR⁸. In certain embodiments, Y is a straight or branched (Ci-Cs)alkyl, wherein three —CH₂— groups in Y are independently replaced with a moiety selected from the group consisting of NR⁸, NR⁸C(O), OC(O)NR⁸, a nitrogen-containing heterocycloalkyl and C(O)NR⁸.

In certain embodiments, Y is a straight (Ci-Cs)alkyl, wherein one or more —CH₂— groups in Y are independently replaced with a moiety selected from the group consisting of NR⁸, NR⁸C(O), OC(O)NR⁸, a nitrogen-containing heterocycloalkyl and C(O)NR⁸. In certain embodiments, Y is a straight (Ci-Cs)alkyl, wherein more than one —CH₂— groups in Y are independently replaced with a moiety selected from the group consisting of NR⁸, NR⁸C(O), OC(O)NR⁸, a nitrogen-containing heterocycloalkyl and C(O)NR⁸. In certain embodiments, Y is

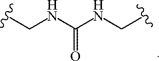

In other embodiments, Y is a branched (Ci-Cs)alkyl, wherein one or more —CH₂— groups in Y are independently replaced with a moiety selected from the group consisting of NR⁸, NR⁸C(O), OC(O)NR⁸, a nitrogen-containing heterocycloalkyl and C(O)NR⁸.

In certain embodiments, Y is a straight (Ci-Cs)alkyl, wherein one or more —CH₂— groups in Y are independently replaced with a moiety selected from the group consisting of NR⁸. In certain embodiments, Y is

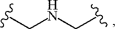

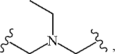

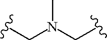

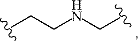

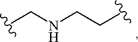

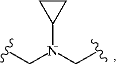

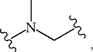

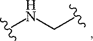

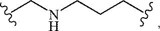

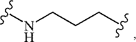

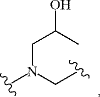

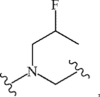

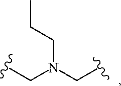

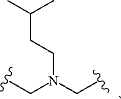

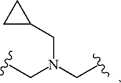

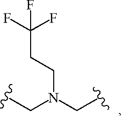

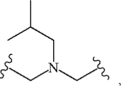

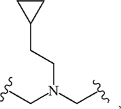

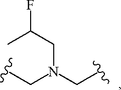

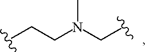

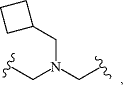

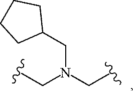

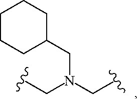

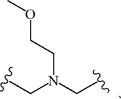

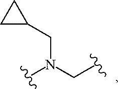

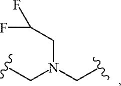

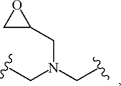

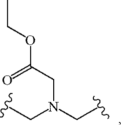

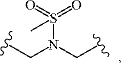

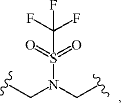

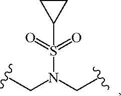

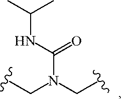

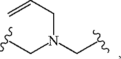

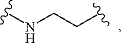

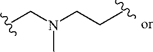

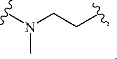

In certain embodiments, Y is a straight (Ci-Cs)alkyl, wherein one or more —CH₂— groups in Y are independently replaced with a moiety selected from the group consisting of NR⁸C(O). In certain embodiments, Y is.

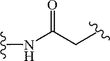

In certain embodiments, Y is a straight (Ci-Cs)alkyl, wherein one or more —CH₂— groups in Y are independently replaced with a moiety selected from the group consisting of OC(O)NR⁸. In certain embodiments, Y is

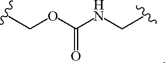

In certain embodiments, Y is a straight (Ci-Cs)alkyl, wherein one or more —CH₂— groups in Y are independently replaced with a moiety selected from the group consisting of a nitrogen-containing heterocycloalkyl. In certain embodiments, Y is

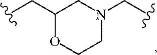

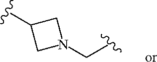

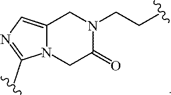

In certain embodiments, Y is a straight (Ci-Cs)alkyl, wherein one or more —CH₂— groups in Y are independently replaced with a moiety selected from the group consisting of C(O)NR⁸. In certain embodiments, Y is

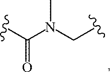

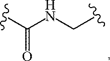

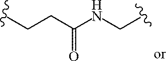

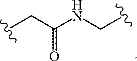

In certain embodiments, Y is a branched (Ci-Cs)alkyl, wherein one or more —CH₂—groups in Y are independently replaced with a moiety selected from the group consisting of NR⁸. In certain embodiments, Y is

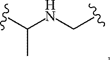

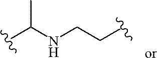

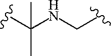

In certain embodiments, Y is a branched (Ci-Cs)alkyl, wherein one or more —CH₂— groups in Y are independently replaced with a moiety selected from the group consisting of NR⁸C(O). In certain embodiments, Y is a branched (Ci-Cs)alkyl, wherein one or more —CH₂— groups in Y are independently replaced with a moiety selected from the group consisting of OC(O)NR⁸. In certain embodiments, Y is a branched (Ci-Cs)alkyl, wherein one or more —CH₂— groups in Y are independently replaced with a moiety selected from the group consisting of a nitrogen-containing heterocycloalkyl. In certain embodiments, Y is a branched (Ci-Cs)alkyl, wherein one or more —CH₂— groups in Y are independently replaced with a moiety selected from the group consisting of C(O)NR⁸.

With regard to the compounds described herein, R⁸ is independently selected from the group consisting of H, (C₁-C₆)alkyl, (C₃-C₆)cycloalkyl, (C₁-C₆)alkylOH, (C₁-C₆)haloalkyl, (Ci-C₆)alkylcycloalkyl, (C₁-C₆)alkylO(C₁-C₆)alkyl, (C₁-C₆)alkylC(O)O(C₁-C₆)alkyl, SO2(C₁-C₆)alkyl, SO₂(C₁-C₆)haloalkyl, CON(R⁷)₂, (C₁-C₆)alkylheterocycloalkyl, SO₂(C₃-C₆)cycloalkyl or (C₁-C₆)alkenyl.

In certain embodiments, R⁸ is hydrogen.

In certain embodiments, R⁸ is (C₁-C₆)alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁸ is methyl, ethyl, n-propyl, isopropyl, n-pentyl and isopentyl.

In certain embodiments, R⁸ is (C₃-C₆)cycloalkyl. In certain embodiments, R⁸ is a monocyclic cycloalkyl. In other embodiments, R⁸ is a bicyclic cycloalkyl. In other embodiments, R⁸ is a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R⁸ is

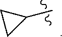

In certain embodiments, R⁸ is (C₁-C₆)alkylOH. Suitable alcohols include, but are not limited to, methanol, ethanol, propanol and butanol. In certain embodiments, R⁸ is

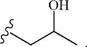

In certain embodiments, R⁸ is (C₁-C₆)haloalkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁸ is

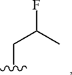

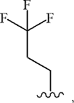

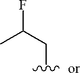

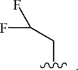

In certain embodiments, R⁸ is (C₁-C₆)alkylcycloalkyl. In certain embodiments, R⁸ is

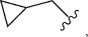

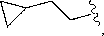

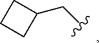

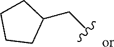

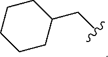

In certain embodiments, R⁸ is (C₁-C₆)alkylO(C₁-C₆)alkyl. In certain embodiments, R⁸ is

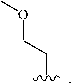

In certain embodiments, R⁸ is (C₁-C₆)alkylC(O)O(C₁-C₆)alkyl. In certain embodiments, R⁸ is

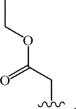

In certain embodiments, R⁸ is SO₂(R⁶). R⁶ is discussed in detail below. In certain embodiments, R⁸ is SO₂(C₁-C₆)alkyl. In certain embodiments, R⁸ is SO₂CH₃. In certain embodiments, R⁸ is SO₂(C₁-C₆)haloalkyl. In certain embodiments, R⁸ is

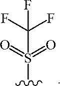

In certain embodiments, R⁸ is SO₂(C₃-C₆)cycloalkyl. In certain embodiments, R⁸ is

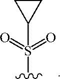

In certain embodiments, R⁸ is CON(R⁷)₂. R⁷ is further defined below. In certain embodiments, R⁸ is

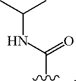

In certain embodiments, R⁸ is (C₁-C₆)alkylheterocycloalkyl. In certain embodiments, R⁸ is

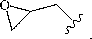

In certain embodiments, R⁸ is (C₁-C₆)alkenyl. In certain embodiments, R⁸ is

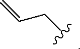

In certain embodiments, Y is

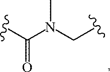

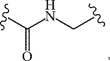

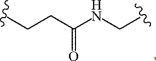

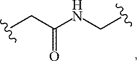

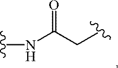

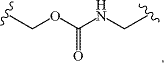

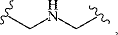

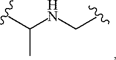

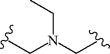

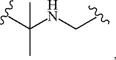

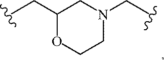

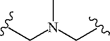

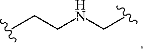

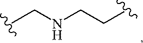

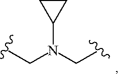

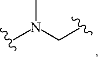

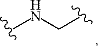

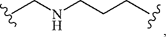

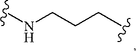

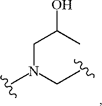

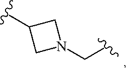

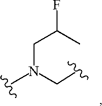

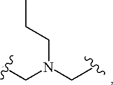

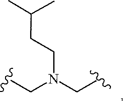

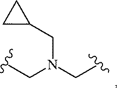

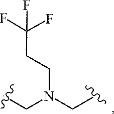

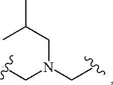

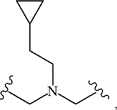

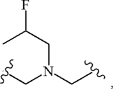

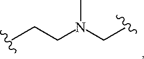

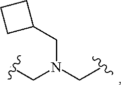

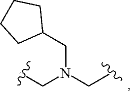

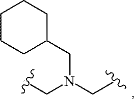

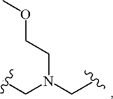

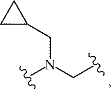

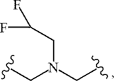

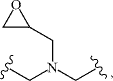

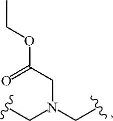

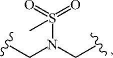

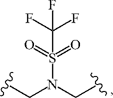

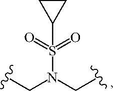

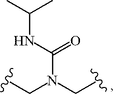

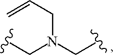

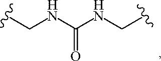

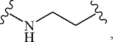

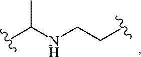

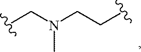

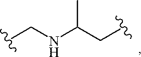

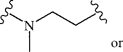

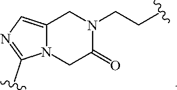

With regard to the compounds described herein, each occurrence of R⁵ is hydrogen, halogen, aryl, (C₃-C₆)cycloalkyl, heterocycloalkyl, heteroaryl, (C₁-C₆)alkylOH, OH, (C₁-C₆)haloalkyl, (C₁-C₆)alkyl, (C₁-C₆)alkynyl, SO₂R⁶, SO(═NH)R⁶, SO(═NCH₃)R⁶ and COO(C₁-C₆)alkyl, wherein the aryl, (C₃-C₆)cycloalkyl, heterocycloalkyl or heteroaryl are optionally substituted with one to three substituents selected from the group consisting of halogen, (C₁-C₆)alkyl, (C₁-C₆)haloalkyl, O(C₁-C₆)alkyl, O(C₁-C₆)haloalkyl, (C₃-C₆)cycloalkyl, O(C₃-C₆)cycloalkyl, (C₁-C₆)haloalkylOH, (C₁-C₆)alkylOH, SO₂N(R⁷)₂, S(halo)₅, haloaryl, halo(C₃-C₆)cycloalkyl, haloheteroaryl, heterocycloalkyl, NR⁷C(O)(C₁-C₆)alkyl, heteroaryl, tert-butoxycarbonyl protecting group (Boc protecting group), or (C₁-C₆)alkylN(R⁷)₂.

In certain embodiments, when R⁵ is attached to Y, wherein Y is a straight or branched (C₁-C₅)alkyl, wherein one or more —CH₂— groups in Y are independently replaced with a moiety selected from the group consisting of NR⁸, NR⁸C(O), OC(O)NR⁸, R⁵ can be attached to a carbon in (C₁-C₅)alkyl, the nitrogen in NR⁸, NR⁸C(O), OC(O)NR⁸, or R⁸. In certain embodiments, R⁵ is attached to (C₁-C₅)alkyl.

In certain embodiments, Y can be substituted with one, two or three R⁵ substutuents. The R5 subtiutuents can be attached to Y at any point, for example

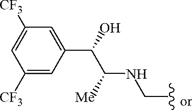

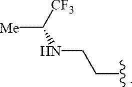

In certain embodiments, R⁵ is attached to NR⁸, NR⁸C(O), OC(O)NR⁸. NR⁸, NR⁸C(O), OC(O)NR⁸. In certain embodiments, R⁵ is attached to R⁸.

In certain embodiments, R⁵ is hydrogen.

In certain embodiments, R⁵ is halogen. Suitable halogens include, but are not limited to, a fluorine, a chlorine, a bromine or an iodine. In certain embodiments, R⁵ is chlorine or fluorine.

In certain embodiments, R⁵ aryl. In certain embodiments, R⁵ is a monocyclic aryl. In other embodiments, R⁵ is a bicyclic aryl. In other embodiments, R⁵ is a multicyclic aryl. Suitable aryls include, but are not limited to, phenyl and naphthyl. In certain embodiments, R⁵ is aryl, wherein the aryl is phenyl. In certain embodiments, R⁵ is aryl, wherein the aryl is naphthyl.

In certain embodiments, the aryl is

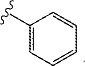

In certain embodiments, R⁵ (C₃-C₆)cycloalkyl. In certain embodiments, R⁵ is a monocyclic cycloalkyl. In other embodiments, R⁵ is a bicyclic cycloalkyl. In other embodiments, R⁵ is a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R⁵ is

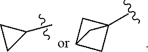

In certain embodiments, R⁵ heterocycloalkyl. In certain embodiments, R⁵ is a nitrogen-containing heterocycloalkyl. In certain embodiments, R⁵ is heterocycloalkyl. In certain embodiments, R⁵ is a sulfur-containing heterocycloalkyl. In certain embodiments, R⁵ is a monocyclic heterocycloalkyl. In other embodiments, R⁵ is a bicyclic heterocycloalkyl. In other embodiments, R⁵ is a multicyclic heterocycloalkyl. In certain embodiments, R⁵ is

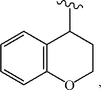

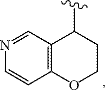

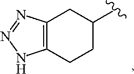

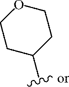

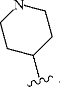

In certain embodiments, R⁵ heteroaryl. In certain embodiments, R⁵ is a nitrogen-containing heteroaryl. In certain embodiments, R⁵ is a monocyclic heteroaryl. In other embodiments, R⁵ is a bicyclic heteroaryl. In other embodiments, R⁵ is a multicyclic heteroaryl. Suitable heteroaryl include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl.

In certain embodiments, R⁵ is heteroaryl, wherein the heteroaryl is

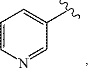

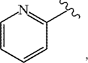

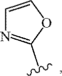

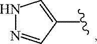

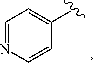

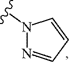

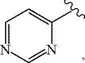

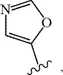

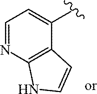

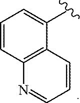

In certain embodiments, R⁵ (C₁-C₆)alkylOH. Suitable alcohols include, but are not limited to, methanol, ethanol, propanol and butanol. In certain embodiments, R⁵ is

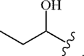

In certain embodiments, R⁵ OH.

In certain embodiments, R⁵ (C₁-C₆)haloalkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁵ is trifluoromethyl,

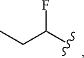

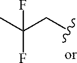

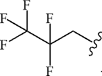

In certain embodiments, R⁵ (C₁-C₆)alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁵ is methyl or ethyl.

In certain embodiments, R⁵ (C₁-C₆)alkynyl.

In certain embodiments, R⁵ is SO₂R⁶, wherein R⁶ is discussed below.

In certain embodiments, R⁵ is SO(═NH)R⁶, wherein R⁶ is discussed below. In certain embodiments, R⁵ is

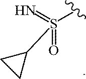

In certain embodiments, R⁵ is SO(═NCH₃)R⁶, wherein R⁶ is discussed below.

In certain embodiments, R⁵ COO(C₁-C₆)alkyl. In certain embodiments, R⁵ is COO(Ci-C₆)alkyl. In certain embodiments, R⁵ is —COOCH₂CH₃.

In certain embodiments, R⁵ is unsubstituted.

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl are optionally substituted with one to three substituents selected from the group consisting of halogen, (C₁-C₆)alkyl, (Ci-C₆)haloalkyl, O(C₁-C₆)alkyl, O(C₁-C₆)haloalkyl, (C₃-C₆)cycloalkyl, O(C₃-C₆)cycloalkyl, (Ci-C₆)haloalkylOH, (C₁-C₆)alkylOH, SO₂N(R⁷)₂, S(halo)₅, haloaryl, halo(C₃-C₆)cycloalkyl, haloheteroaryl, heterocycloalkyl, NR⁷C(O)(C₁-C₆)alkyl, heteroaryl, tert-butoxycarbonyl protecting group (Boc protecting group), or (C₁-C₆)alkylN(R⁷)₂.

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl are substituted with one to three substituents selected from the group consisting of halogen, (C₁-C₆)alkyl, (C₁-C₆)haloalkyl, O(C₁-C₆)alkyl, O(C₁-C₆)haloalkyl, (C₃-C₆)cycloalkyl, O(C₃-C₆)cycloalkyl, (C₁-C₆)haloalkylOH, (C₁-C₆)alkylOH, SO₂N(R⁷)₂, S(halo)₅, haloaryl, halo(C₃-C₆)cycloalkyl, haloheteroaryl, heterocycloalkyl, NR⁷C(O)(C₁-C₆)alkyl, heteroaryl, tert-butoxycarbonyl protecting group (Boc protecting group), or (C₁-C₆)alkylN(R⁷)₂.

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with one substituent selected from the group consisting of halogen, (C₁-C₆)alkyl, (C₁-C₆)haloalkyl, O(C₁-C₆)alkyl, O(C₁-C₆)haloalkyl, (C₃-C₆)cycloalkyl, O(C₃-C₆)cycloalkyl, (C₁-C₆)haloalkylOH, (Ci-C₆)alkylOH, SO₂N(R⁷)₂, S(halo)₅, haloaryl, halo(C₃-C₆)cycloalkyl, haloheteroaryl, heterocycloalkyl, NR⁷C(O)(C₁-C₆)alkyl, heteroaryl, tert-butoxycarbonyl protecting group (Boc protecting group), or (C₁-C₆)alkylN(R⁷)₂.

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with two substituents selected from the group consisting of halogen, (C₁-C₆)alkyl, (C₁-C₆)haloalkyl, O(C₁-C₆)alkyl, O(C₁-C₆)haloalkyl, (C₃-C₆)cycloalkyl, O(C₃-C₆)cycloalkyl, (C₁-C₆)haloalkylOH, (Ci-C₆)alkylOH, SO₂N(R⁷)₂, S(halo)₅, haloaryl, halo(C₃-C₆)cycloalkyl, haloheteroaryl, heterocycloalkyl, NR⁷C(O)(C₁-C₆)alkyl, heteroaryl, tert-butoxycarbonyl protecting group (Boc protecting group), or (C₁-C₆)alkylN(R⁷)₂.

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with three substituents selected from the group consisting of halogen, (C₁-C₆)alkyl, (C₁-C₆)haloalkyl, O(C₁-C₆)alkyl, O(C₁-C₆)haloalkyl, (C₃-C₆)cycloalkyl, O(C₃-C₆)cycloalkyl, (C₁-C₆)haloalkylOH, (C₁-C₆)alkylOH, SO₂N(R⁷)₂, S(halo)₅, haloaryl, halo(C₃-C₆)cycloalkyl, haloheteroaryl, heterocycloalkyl, NR⁷C(O)(C₁-C₆)alkyl, heteroaryl, tert-butoxycarbonyl protecting group (Boc protecting group), or (C₁-C₆)alkylN(R⁷)₂.

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with halogen. Suitable halogens include, but are not limited to, a fluorine, a chlorine, a bromine or an iodine. In certain embodiments, R⁵ is substituted with fluorine, chlorine or bromine.

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with (Ci-C₆)alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁵ is substituted with methyl or tert-butyl.

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with (Ci-C₆)haloalkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁵ is substituted with trifluoromethyl.

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with O(C₁-C₆)alkyl. Suitable alkoxys include, but are not limited to, methoxy, ethoxy, n-propoxy, isopropoxy and n-butoxy. In certain embodiments, R⁵ is substituted with methoxy or isopropoxy.

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with O(C₁-C₆)haloalkyl. In certain embodiments, R⁵ is substituted with

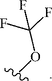

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with (C₃-C₆)cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R⁵ is

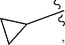

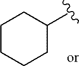

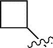

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with halo(C₃-C₆)cycloalkyl. In certain embodiments, R⁵ is substituted with

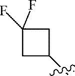

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted O(C₃-C₆)cycloalkyl. In certain embodiments, R⁵ is substituted with

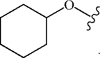

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with (Ci-C₆)haloalkylOH. In certain embodiments, R⁵ is substituted with

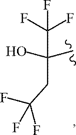

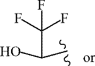

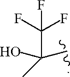

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with (Ci-C₆)alkylOH. In certain embodiments, R⁵ is substituted with

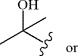

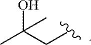

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with SO₂N(R⁷)₂. R⁷ is defined below. In certain embodiments, R⁵ is substituted with

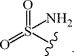

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with S(halo)₅. In certain embodiments, R⁵ is substituted with —S(F)⁵.

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted haloaryl. In certain embodiments, R⁵ is substituted with

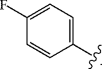

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with haloheteroaryl. In certain embodiments, R⁵ is substituted with

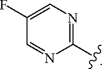

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with heterocycloalkyl. In certain embodiments, R⁵ is substituted with

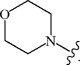

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with NR⁷C(O)(C₁-C₆)alkyl. In certain embodiments, R⁵ is substituted with

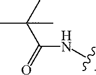

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with heteroaryl. In certain embodiments, R⁵ is substituted with

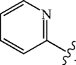

In certain embodiments, wherein R⁵ is heterocycloalkyl or heteroaryl, a nitrogen on the the heterocycloalkyl or heteroaryl is Boc protected.

In certain embodiments, wherein R⁵ is aryl, (C₃-C₆)cycloalkyl, heterocycloalkyl or heteroaryl, the aryl, (C₃-C₆)cycloalkyl, heterocycloalkyl or heteroaryl is substituted with (Ci-C6)alkylN(R⁷)₂. In certain embodiments, R⁵ is substituted with

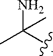

In certain embodiments, R⁵ is aryl, wherein the aryl is phenyl and the phenyl is optionally substituted with one to three substituents selected from the group consisting of halogen, (Ci-C₆)alkyl, (C₁-C₆)haloalkyl, O(C₁-C₆)alkyl, O(C₁-C₆)haloalkyl, (C₃-C₆)cycloalkyl, O(C₃–C₆)cycloalkyl, (C₁-C₆)haloalkylOH, (C₁-C₆)alkylOH, SO₂NH₂, S(F)₅, haloaryl, halo(C₃-C₆)cycloalkyl, haloheteroaryl, heterocycloalkyl, NHC(O)(C₁-C₆)alkyl, heteroaryl, tert-butoxycarbonyl protecting group (Boc protecting group), or (Ci-C₆)alkylNH₂.

In certain embodiments, R⁵ is an unsubstituted or substituted (C₃-C₆)cycloalkyl, wherein the unsubstituted or substituted (C₃-C₆)cycloalkyl is cyclopropyl or

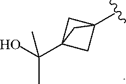

In certain embodiments, R⁵ is an unsubstituted or substituted heteroaryl, wherein the unsubstituted or substituted heteroaryl is

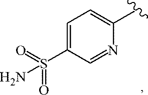

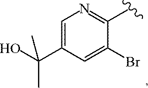

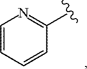

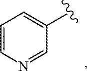

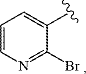

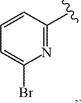

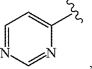

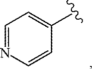

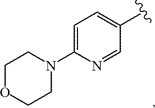

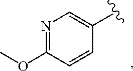

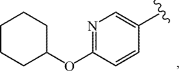

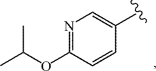

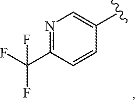

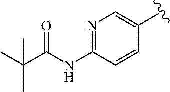

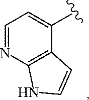

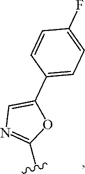

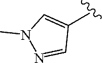

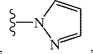

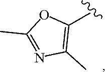

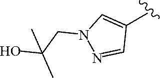

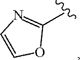

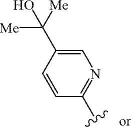

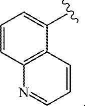

In certain embodiments, R⁵ is an unsubstituted or substituted heterocycloalkyl, wherein the unsubstituted or substituted heterocycloalkyl is

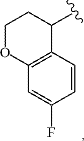

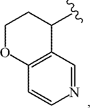

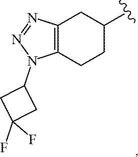

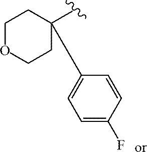

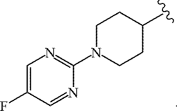

With regard to the compounds described herein, R⁶ is selected from the group consisting of OH, NH₂, (C₁-C₆)alkyl, aryl, (C₁-C₆)haloalkyl, (C₃-C₆)cycloalkyl and haloaryl.

In certain embodiments, R⁶ is OH.

In certain embodiments, R⁶ is NH₂.

In certain embodiments, R⁶ is (C₁-C₆)alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁶ is methyl, cyclopropyl, ethyl, iso-butyl or iso-propyl.

In certain embodiments, R⁶ is aryl. In certain embodiments, R⁶ is a monocyclic aryl. In other embodiments, R⁶ is a bicyclic aryl. In other embodiments, R⁶ is a multicyclic aryl. Suitable aryls include, but are not limited to, phenyl and naphthyl. In certain embodiments, R⁵ is aryl, wherein the aryl is phenyl. In certain embodiments, R⁶ is aryl, wherein the aryl is naphthyl. In certain embodiments, the aryl is

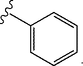

In certain embodiments, R⁶ is (C₁-C₆)haloalkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁶ is trifluoromethyl.

In certain embodiments, R⁶ is (C₃-C₆)cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R⁶ is

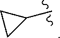

In certain embodiments, R⁶ is haloaryl. In certain embodiments, R⁶ is fluorophenyl.

In certain embodiments, R⁶ is methyl, NH₂, phenyl, cyclopropyl, fluorophenyl, trifluoromethyl, ethyl, iso-butyl or iso-propyl.

With regard to the compounds described herein, each occurrence of R⁷ is independently selected from the group consisting of hydrogen, (C₁-C₆)alkyl, (C₃-C₆)cycloalkyl, or when two R⁷ substituents are taken together with the nitrogen they are attached, form a heterocycloalkyl.

In certain embodiments, R⁷ is hydrogen.

In certain embodiments, R⁷ (C₁-C₆)alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁷ is methyl, cyclopropyl, ethyl, iso-butyl or iso-propyl.

In certain embodiments, R⁷ (C₃-C₆)cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R⁷ is

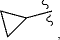

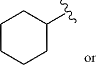

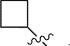

In certain embodiments, when two R⁷ substituents are taken together with the nitrogen they are attached, form a heterocycloalkyl.

In certain embodiments, R⁵ is methyl, ethyl, trifluoromethyl, OH,

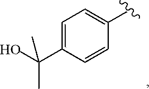

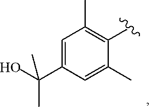

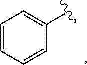

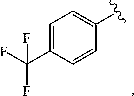

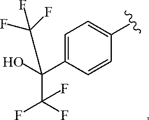

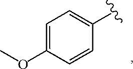

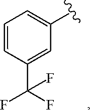

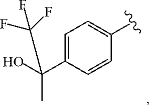

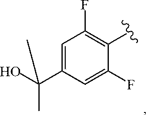

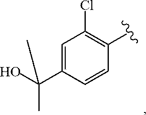

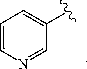

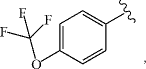

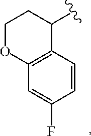

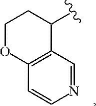

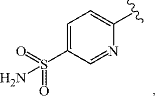

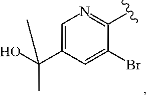

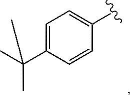

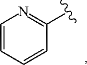

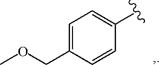

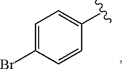

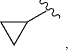

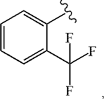

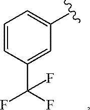

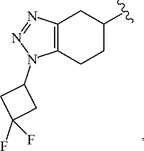

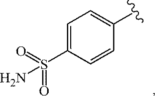

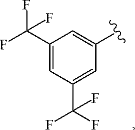

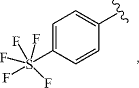

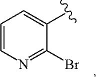

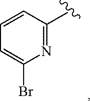

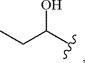

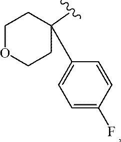

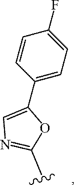

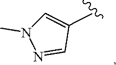

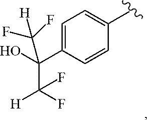

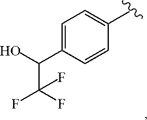

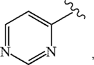

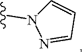

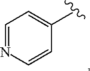

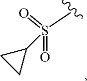

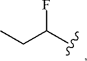

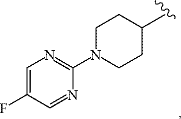

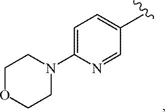

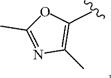

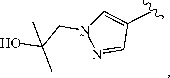

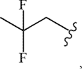

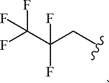

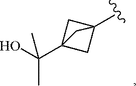

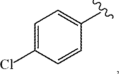

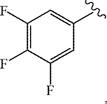

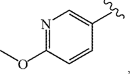

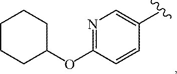

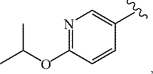

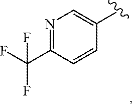

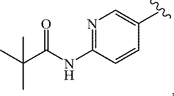

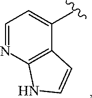

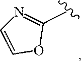

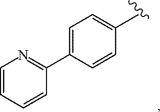

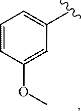

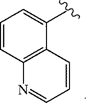

With regard to the compounds described herein, n is 1, 2 or 3. In certain embodiments, n is 1. In certain embodiments, n is 2. In certain embodiments, n is 3.

In certain embodiments, the compounds of the invention comprise the following compounds, and pharmaceutically acceptable salts thereof,

| | |
|---|---|
| 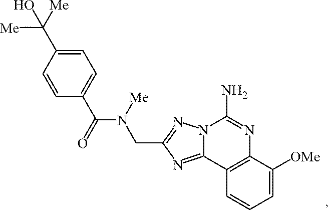 | 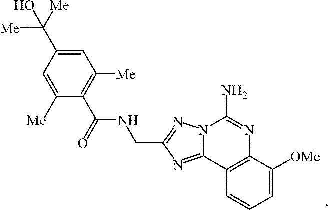 |
| 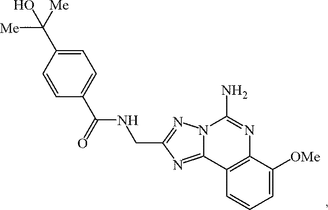 | 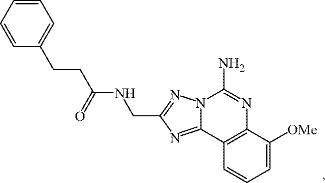 |
| 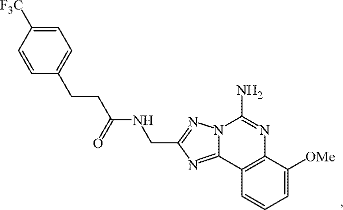 | 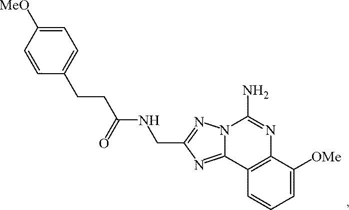 |
| 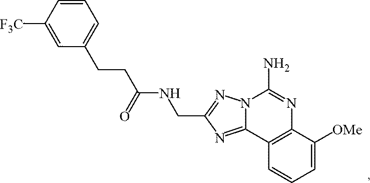 | 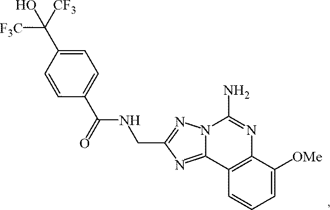 |
| 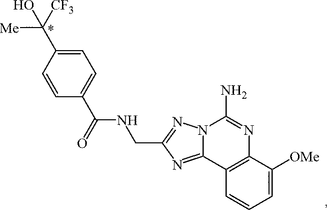 | 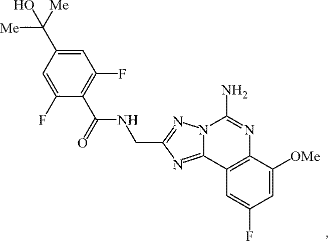 |
| 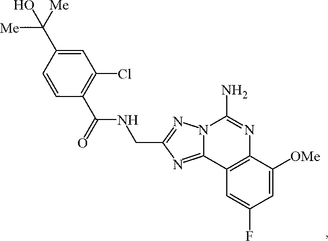 | 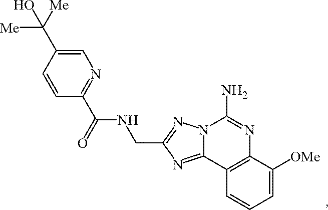 |
| 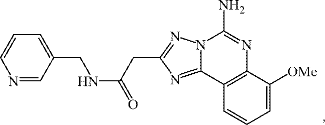 | 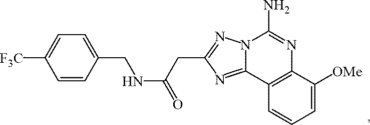 |
| 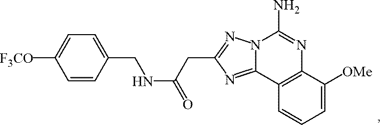 | 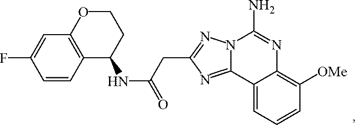 |
| 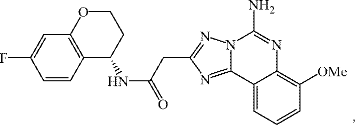 | 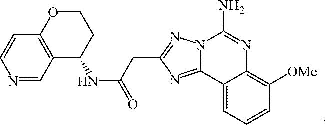 |
| 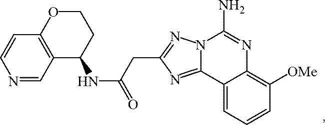 | 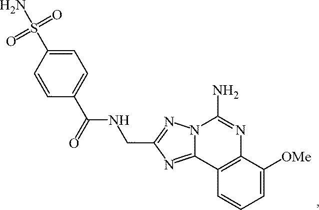 |
| 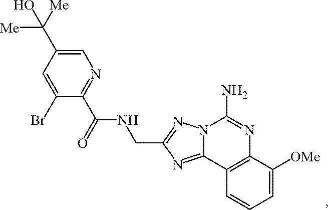 | 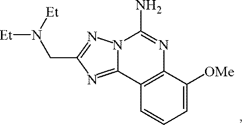 |
| 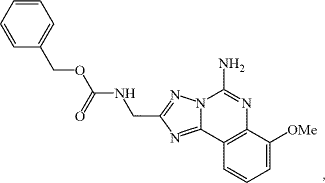 | 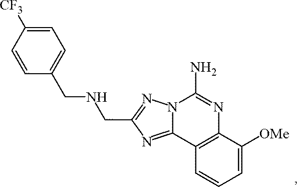 |
| 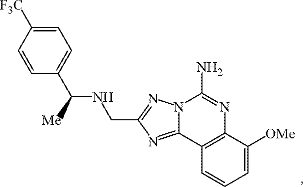 | 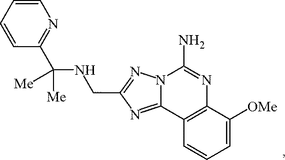 |
| 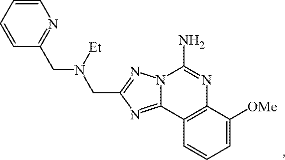 | 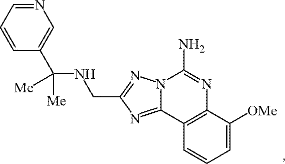 |
| 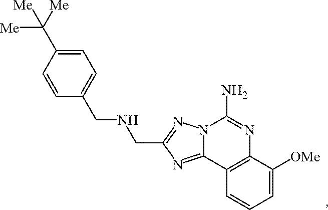 | 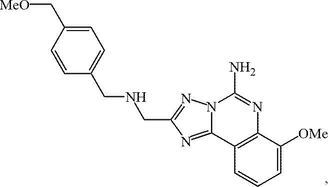 |
| 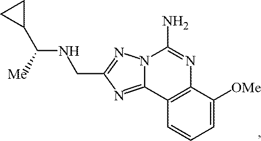 | 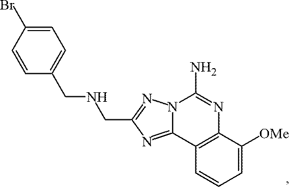 |
| 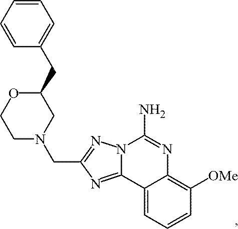 | 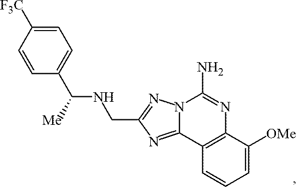 |
| 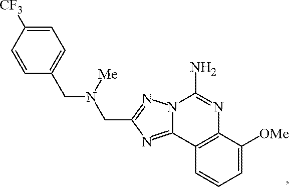 | 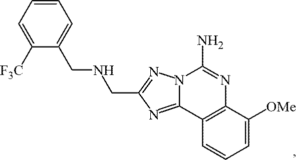 |
| 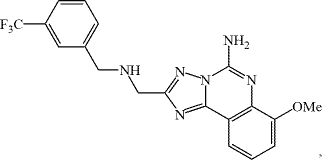 | 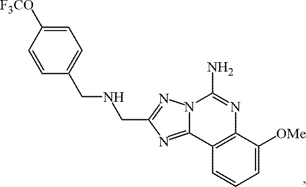 |
| 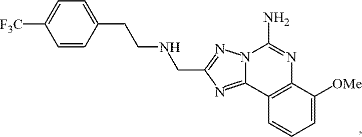 | 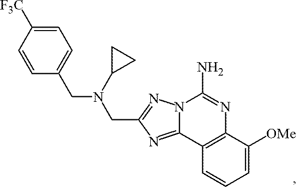 |
| 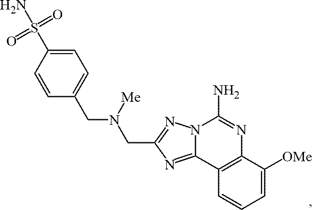 | 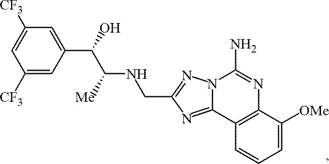 |
| 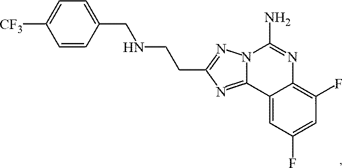 | 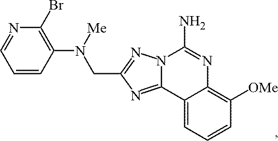 |
| 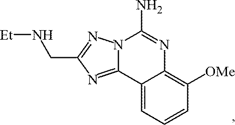 | 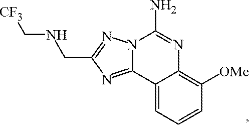 |
| 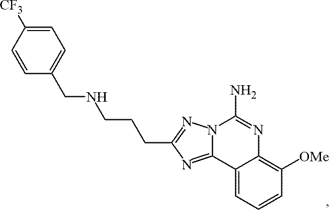 | 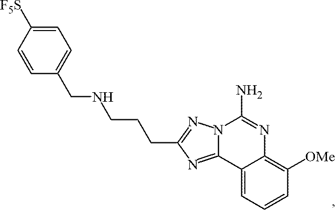 |
| 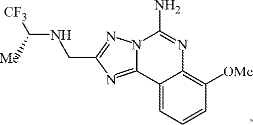 | 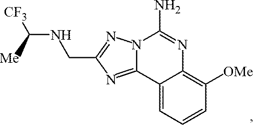 |
| 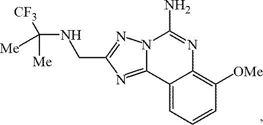 | 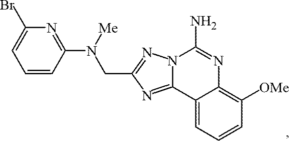 |
| 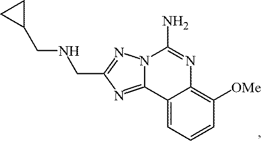 | 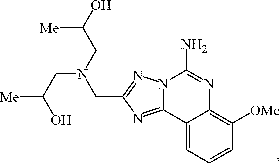 |
| 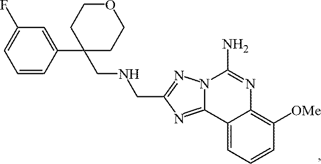 | 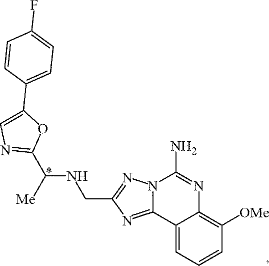 |
| 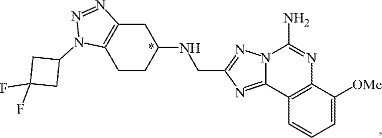 | 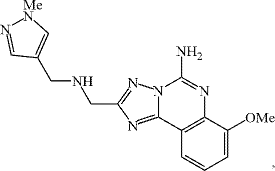 |
| 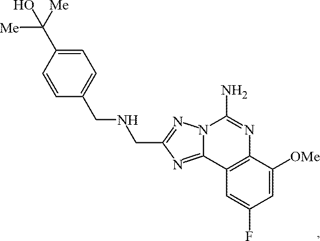 | 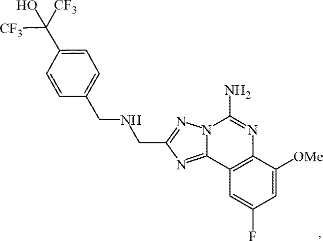 |
| 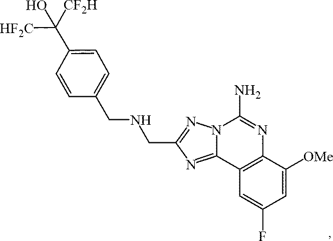 | 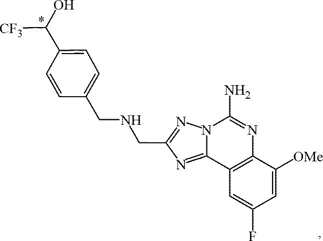 |
| 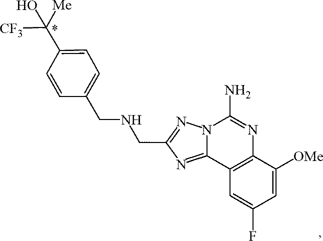 | 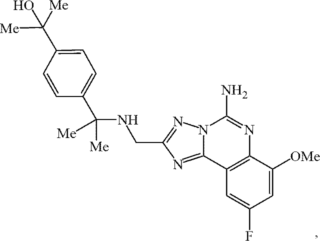 |
| 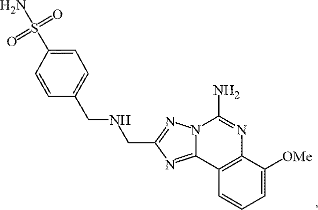 | 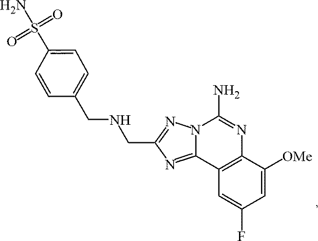 |
| 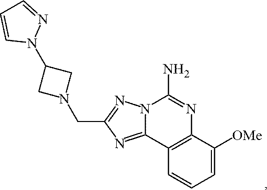 | 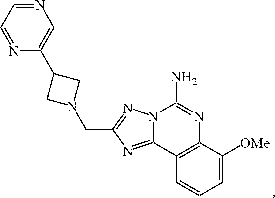 |
| 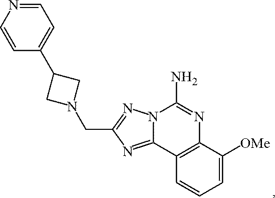 | 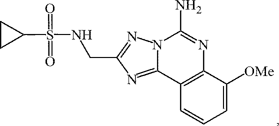 |
| 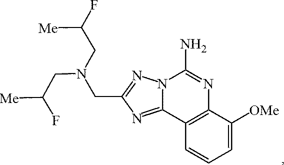 | 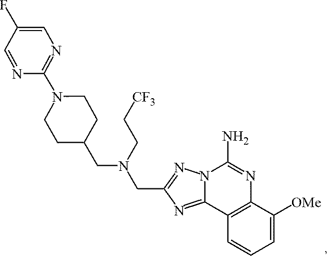 |
| 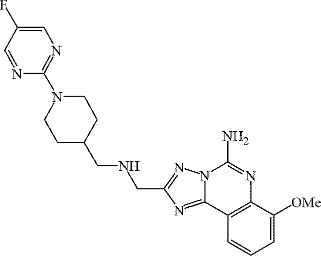 [SMILES: COc1cccc2c1nc(N)n1nc(CNCC3CCN(c4ncc(F)cn4)CC3)nc21] | 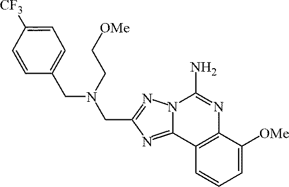 [SMILES: COCCN(Cc1ccc(C(F)(F)F)cc1)Cc1nc2c3cccc(OC)c3nc(N)n2n1] |
| 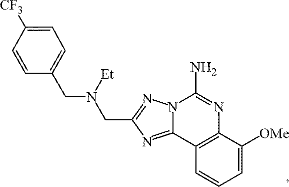 | 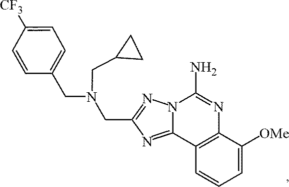 |
| 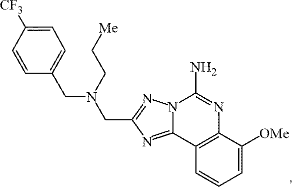 | 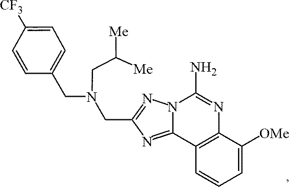 |
| 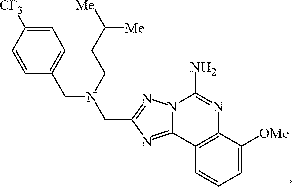 | 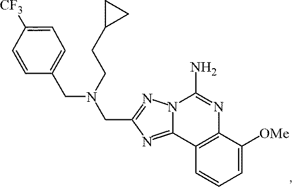 |
| 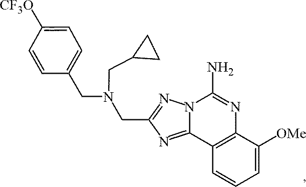 | 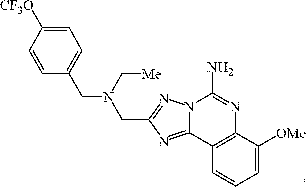 |
| 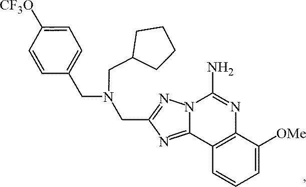 | 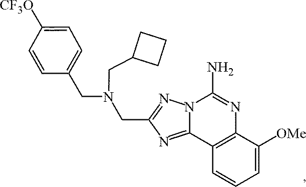 |
| 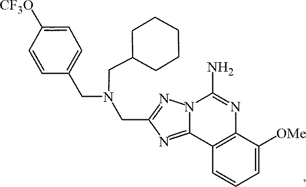 | 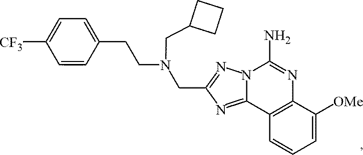 |
| 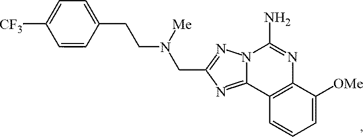 | 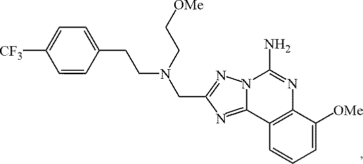 |
| 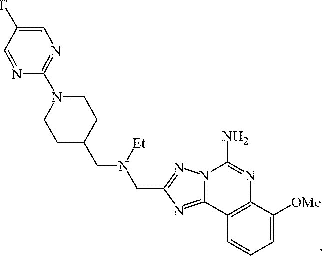 | 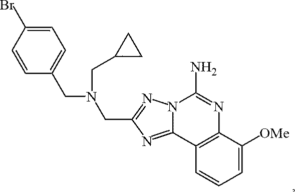 |
| 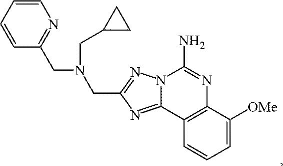 | 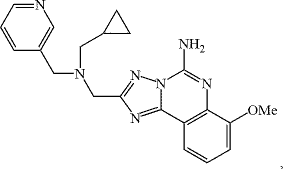 |
| 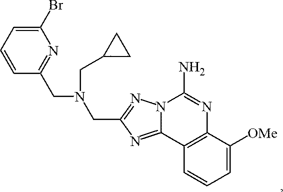 | 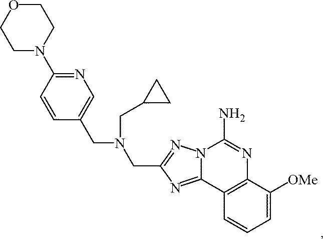 |
| 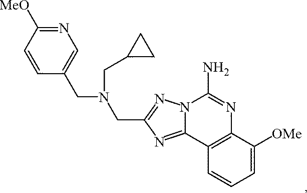 | 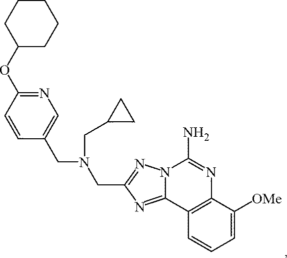 |
| 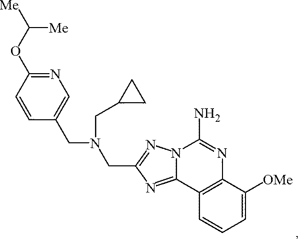 | 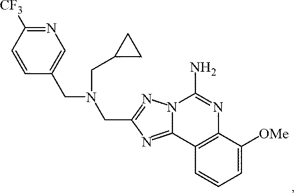 |
| 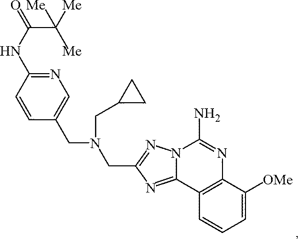 | 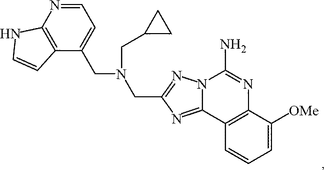 |
| 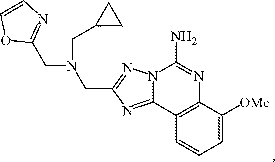 | 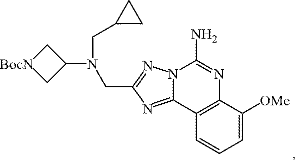 |
| 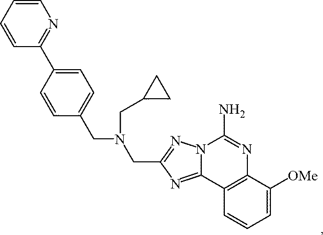 | 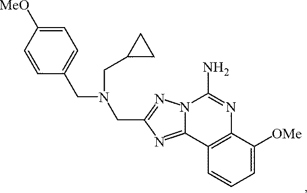 |
| 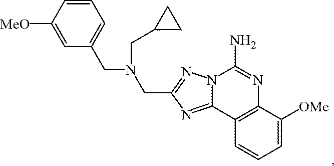 | 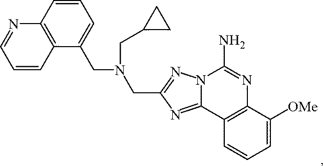 |
| 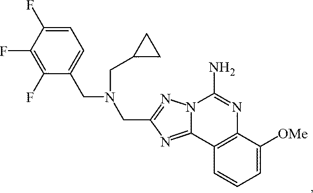 | 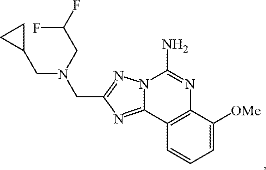 |
| 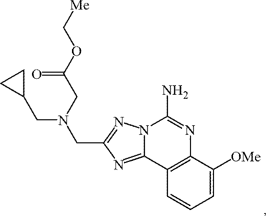 | 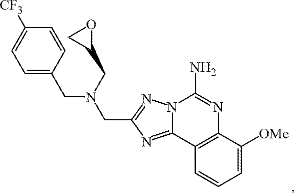 |
| 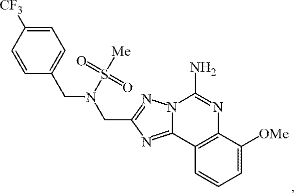 | 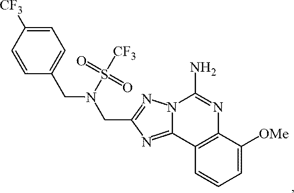 |
| 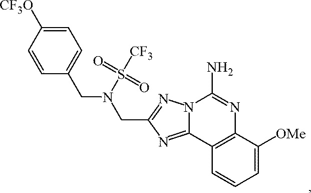 | 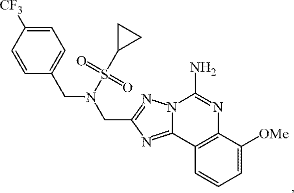 |
| 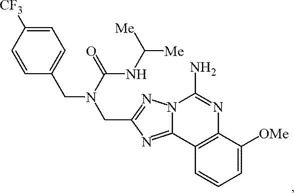 | 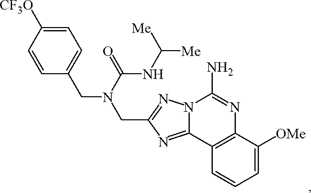 |
| 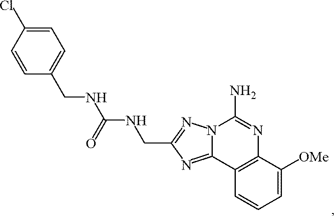 | 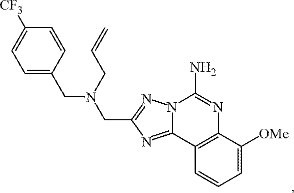 |
| 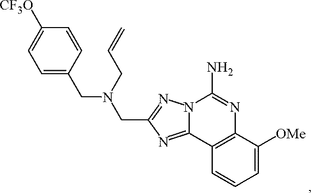 | 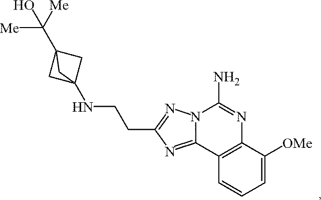 |
| 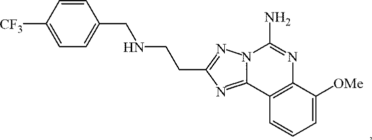 | 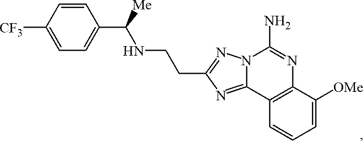 |
| 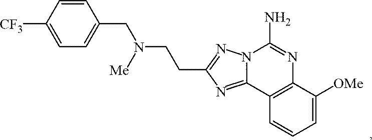 | 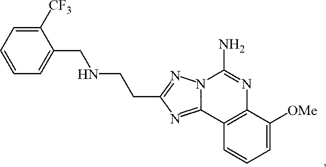 |
| 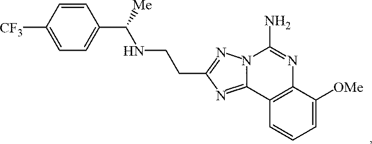 | 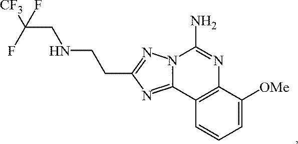 |
| 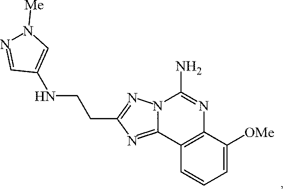 | 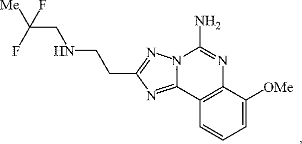 |
| 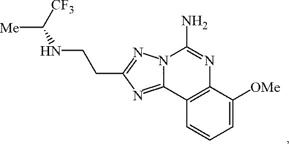 | 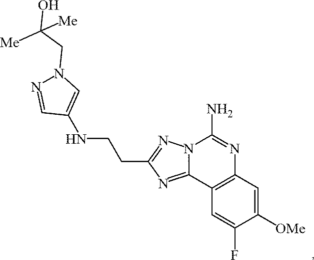 |
| 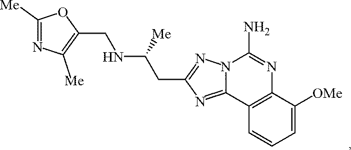 | 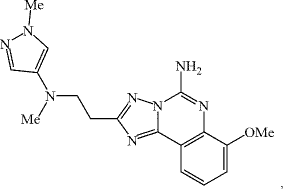 |
| 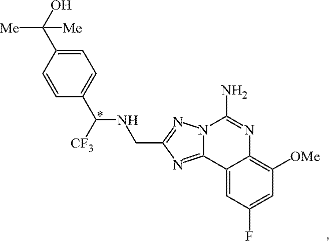 | 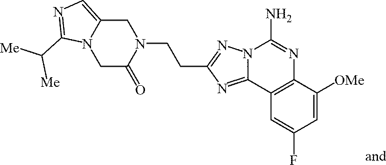 |

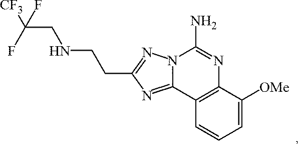

In another embodiment, the compounds of the invention comprise those compounds identified herein as examples, and pharmaceutically acceptable salts thereof.

In another aspect, the present invention provides pharmaceutical compositions comprising a pharmaceutically acceptable carrier and a compound of the invention or a pharmaceutically acceptable salt thereof. Such compositions according to the invention may optionally further include one or more additional therapeutic agents as described herein.

In another aspect, the present invention provides a method for the manufacture of a medicament or a composition which may be useful for treating diseases, conditions, or disorders that are mediated, at least in part, by the adenosine A2a receptor and/or the adenosine A2b receptor, comprising combining a compound of the invention with one or more pharmaceutically acceptable carriers.

In another aspect, the present invention provides a method for treating or preventing a disease, condition, or disorder that is mediated, at least in part, by the adenosine A2a receptor and/or the adenosine A2b receptor in a subject (e.g., an animal or human) in need thereof, said method comprising administering to the subject in need thereof a therapeutically effective amount of at least one compound of the invention, or a pharmaceutically acceptable salt thereof, alone or in combination with one or more additional therapeutic agents. Specific non-limiting examples of such diseases, conditions, and disorders are described herein.

### Oncology

In some embodiments, the disease, condition or disorder is a cancer. Any cancer for which a PD-1 antagonist and/or an A2a and/or A2b inhibitor are thought to be useful by those of ordinary skill in the art are contemplated as cancers treatable by this embodiment, either as a monotherapy or in combination with other therapeutic agents discussed below. Cancers that express high levels of A2a receptors or A2b receptors are among those cancers contemplated as treatable by the compounds of the invention. Examples of cancers that express high levels of A2a and/or A2b receptors may be discerned by those of ordinary skill in the art by reference to the Cancer Genome Atlas (TCGA) database. Non-limiting examples of cancers that express high levels of A2a receptors include cancers of the kidney, breast, lung, and liver. Non-limiting examples of cancers that express high levels of the A2b receptor include lung, colorectal, head & neck cancer, and cervical cancer.

Thus, one embodiment provides a method of treating cancer comprising administering an effective amount of a compound of the invention, or a pharmaceutically acceptable salt thereof, to a subject in need of such treatment, wherein said cancer is a cancer that expresses a high level of A2a receptor. A related embodiment provides a method of treating cancer comprising administering an effective amount of a compound of the invention, or a pharmaceutically acceptable salt thereof, to a subject in need of such treatment, wherein said cancer is selected from kidney (or renal) cancer, breast cancer, lung cancer, and liver cancer.

Another embodiment provides a method of treating cancer comprising administering an effective amount of a compound of the invention, or a pharmaceutically acceptable salt thereof, to a subject in need of such treatment, wherein said cancer is a cancer that expresses a high level of A2b receptor. A related embodiment provides a method of treating cancer comprising administering an effective amount of a compound of the invention, or a pharmaceutically acceptable salt thereof, to a subject in need of such treatment, wherein said cancer is selected from lung cancer, colorectal cancer, head & neck cancer, and cervical cancer.

Additional non-limiting examples of cancers which may be treatable by administration of a compound of the invention (alone or in combination with one or more additional agents described below) include cancers of the prostate (including but not limited to metastatic castration resistant prostate cancer), colon, rectum, pancreas, cervix, stomach, endometrium, brain, liver, bladder, ovary, testis, head, neck, skin (including melanoma and basal carcinoma), mesothelial lining, white blood cell (including lymphoma and leukemia) esophagus, breast, muscle, connective tissue, lung (including but not limited to small cell lung cancer, non-small cell lung cancer, and lung adenocarcinoma), adrenal gland, thyroid, kidney, or bone. Additional cancers treatable by a compound of the invention include glioblastoma, mesothelioma, renal cell carcinoma, gastric carcinoma, sarcoma, choriocarcinoma, cutaneous basocellular carcinoma, and testicular seminoma, and Kaposi’s sarcoma.

### CNS and Neurological Disorders

In other embodiments, the disease, condition or disorder is a central nervous system or a neurological disorder. Non-limiting examples of such diseases, conditions or disorders include movement disorders such as tremors, bradykinesias, gait disorders, dystonias, dyskinesias, tardive dyskinesias, other extrapyramidal syndromes, Parkinson’s disease, and disorders associated with Parkinson’s disease. The compounds of the invention also have the potential, or are believed to have the potential, for use in preventing or reducing the effect of drugs that cause or worsen such movement disorders.

### Infections

In other embodiments, the disease, condition or disorder is an infective disorder. Non-limiting examples of such diseases, conditions or disorders include an acute or chronic viral infection, a bacterial infection, a fungal infection, or a parasitic infection. In one embodiment, the viral infection is human immunodeficiency virus. In another embodiment, the viral infection is cytomegalovirus.

### Immune Disease

In other embodiments, the disease, condition or disorder is an immune-related disease, condition or disorder. Non-limiting examples of immune-related diseases, conditions, or disorders include multiple sclerosis and bacterial infections. (See, e.g., Safarzadeh, E. et al., Inflamm Res 2016 65(7):511-20; and Antonioli, L., et al., Immunol Lett S0165-2478(18)30172-X 2018).

### Additional Indications

Other diseases, conditions, and disorders that have the potential to be treated or prevented, in whole or in part, by the inhibition of the A2a and/or A2b adenosine receptor(s) are also candidate indications for the compounds of the invention and salts thereof. Non-limiting examples of other diseases, conditions or disorders in which a compound of the invention, or a pharmaceutically acceptable salt thereof, may be useful include the treatment of hypersensitivity reaction to a tumor antigen and the amelioration of one or more complications related to bone marrow transplant or to a peripheral blood stem cell transplant. Thus, in another embodiment, the present invention provides a method for treating a subject receiving a bone marrow transplant or a peripheral blood stem cell transplant by administering to said subject a therapeutically effective amount of a compound of the invention, or a pharmaceutically acceptable salt thereof, sufficient to increase the delayed-type hypersensitivity reaction to tumor antigen, to delay the time-to-relapse of post-transplant malignancy, to increase relapse-free survival time post-transplant, and/or to increase long-term post-transplant survival.

### Combination Therapy

In another aspect, the present invention provides methods for the use of a compound of the invention, or a pharmaceutically acceptable salt thereof, (or a pharmaceutically acceptable composition comprising a compound of the invention or pharmaceutically acceptable salt thereof) in combination with one or more additional agents. Such additional agents may have some adenosine A2a and/or A2b receptor activity, or, alternatively, they may function through distinct mechanisms of action. The compounds of the invention may be used in combination with one or more other drugs in the treatment, prevention, suppression or amelioration of diseases or conditions for which the compounds of the invention or the other drugs described herein may have utility, where the combination of the drugs together are safer or more effective than either drug alone. The combination therapy may have an additive or synergistic effect. Such other drug(s) may be administered in an amount commonly used therefore, contemporaneously or sequentially with a compound of the invention or a pharmaceutically acceptable salt thereof. When a compound of the invention is used contemporaneously with one or more other drugs, the pharmaceutical composition may in specific embodiments contain such other drugs and the compound of the invention or its pharmaceutically acceptable salt in separate doses or in unit dosage form. However, the combination therapy may also include therapies in which the compound of the invention or its pharmaceutically acceptable salt and one or more other drugs are administered sequentially, on different or overlapping schedules. It is also contemplated that when used in combination with one or more other active ingredients, the compounds of the invention and the other active ingredients may be used in lower doses than when each is used singly. Accordingly, the pharmaceutical compositions comprising the compounds of the invention include those that contain one or more other active ingredients, in addition to a compound of the invention or a pharmaceutically acceptable salt thereof.

The weight ratio of the compound of the present invention to the second active ingredient may be varied and will depend upon the effective dose of each ingredient. Generally, an effective dose of each will be used. Thus, for example, when a compound of the invention is used in combination with another agent, the weight ratio of the compound of the present invention to the other agent may generally range from about 1000:1 to about 1:1000, in particular embodiments from about 200:1 to about 1:200. Combinations of a compound of the present invention and other active ingredients will generally also be within the aforementioned range, but in each case, an effective dose of each active ingredient should generally be used.

Given the immunosuppressive role of adenosine, the administration of an A2a receptor antagonist, an A2b receptor antagonist, and/or an A2a/A2b receptor dual antagonist according to the invention may enhance the efficacy of immunotherapies such as PD-1 antagonists. Thus, in one embodiment, the additional therapeutic agent comprises an anti-PD-1 antibody. In another embodiment, the additional therapeutic agent is an anti-PD-1 antibody.

As noted above, PD-1 is recognized as having an important role in immune regulation and the maintenance of peripheral tolerance. PD-1 is moderately expressed on naive T-cells, B-cells and NKT-cells and up-regulated by T-cell and B-cell receptor signaling on lymphocytes, monocytes and myeloid cells (Sharpe et al., Nature Immunology (2007); 8:239-245).

Two known ligands for PD-1, PD-L1 (B7-H1) and PD-L2 (B7-DC) are expressed in human cancers arising in various tissues. In large sample sets of, for example, ovarian, renal, colorectal, pancreatic, and liver cancers, and in melanoma, it was shown that PD-L1 expression correlated with poor prognosis and reduced overall survival irrespective of subsequent treatment. (Dong et al., Nat Med. 8(8):793-800 (2002); Yang et al., Invest Ophthamol Vis Sci. 49: 2518-2525 (2008); Ghebeh et al., Neoplasia 8:190-198 (2006); Hamanishi et al., Proc. Natl. Acad. Sci. USA 104: 3360-3365 (2007); Thompson et al., Cancer 5: 206-211 (2006) ; Nomi et al., Clin. Cancer Research 13:2151-2157 (2007); Ohigashi et al., Clin. Cancer Research 11: 2947-2953; Inman et al., Cancer 109: 1499-1505 (2007); Shimauchi et al., Int. J. Cancer 121:2585-2590 (2007); Gao et al., Clin. Cancer Research 15: 971-979 (2009); Nakanishi J., Cancer Immunol Immunother. 56: 1173- 1182 (2007); and Hino et al., Cancer 00: 1-9 (2010)).

Similarly, PD-1 expression on tumor infiltrating lymphocytes was found to mark dysfunctional T-cells in breast cancer and melanoma (Ghebeh et al., BMC Cancer. 2008 8:5714-15 (2008); and Ahmadzadeh et al., Blood 114: 1537-1544 (2009)) and to correlate with poor prognosis in renal cancer (Thompson et al., Clinical Cancer Research 15: 1757-1761(2007)). Thus, it has been proposed that PD-L1 expressing tumor cells interact with PD-1 expressing T-cells to attenuate T-cell activation and to evade immune surveillance, thereby contributing to an impaired immune response against the tumor.

Immune checkpoint therapies targeting the PD-1 axis have resulted in groundbreaking improvements in clinical response in multiple human cancers (Brahmer, et al., N Engl J Med 2012, 366: 2455-65; Garon et al., N Engl J Med 2015, 372: 2018-28; Hamid et al., N Engl J Med 2013, 369: 134-44; Robert et al., Lancet 2014, 384: 1109-17; Robert et al., N Engl J Med 2015, 372: 2521-32; Robert et al., N Engl J Med 2015, 372: 320-30; Topalian et al., N Engl J Med 2012, 366: 2443-54; Topalian et al., J Clin Oncol 2014, 32: 1020-30; and Wolchok et al., N Engl J Med 2013, 369: 122-33).

“PD-1 antagonist” means any chemical compound or biological molecule that blocks binding of PD-L1 expressed on a cancer cell to PD-1 expressed on an immune cell (T-cell, B-cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, medicaments and uses of the present invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP 005009. Human PD-L1 and PD-L2 amino acid sequences can be found in NCBI Locus No.: NP_054862 and NP_079515, respectively.

PD-1 antagonists useful in any of the treatment methods, medicaments and uses of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgGl, IgG2, IgG3 and IgG4 constant regions, and in preferred embodiments, the human constant region is an IgGl or IgG4 constant region. In some embodiments, the antigen binding fragment is selected from the group consisting of Fab, Fab′-SH, F(ab′)2, scFv and Fv fragments. Examples of PD-1 antagonists include, but are not limited to, pembrolizumab (KEYTRUDA®, Merck and Co., Inc., Kenilworth, NJ, USA). “Pembrolizumab” (formerly known as MK-3475, SCH 900475 and lambrolizumab and sometimes referred to as “pembro”) is a humanized IgG4 mAb with the structure described in WHO Drug Information, Vol. 27, No. 2, pages 161-162 (2013). Additional examples of PD-1 antagonists include nivolumab (OPDIVO®, Bristol-Myers Squibb Company, Princeton, NJ, USA), atezolizumab (MPDL3280A; TECENTRIQ®, Genentech, San Francisco, CA, USA), durvalumab (IMFINZIⓇ, Astra Zeneca Pharmaceuticals, LP, Wilmington, DE, and avelumab (BAVENCIO®, Merck KGaA, Darmstadt, Germany and Pfizer, Inc., New York, NY).

Examples of monoclonal antibodies (mAbs) that bind to human PD-1, and useful in the treatment methods, medicaments and uses of the present invention, are described in US7488802, US7521051, US8008449, US8354509, US8168757, WO2004/004771, WO2004/072286, WO2004/056875, and US2011/0271358.

Examples of mAbs that bind to human PD-L1, and useful in the treatment methods, medicaments and uses of the present invention, are described in WO2013/019906, WO2010/077634 Al and US8383796. Specific anti-human PD-Ll mAbs useful as the PD-1 antagonist in the treatment method, medicaments and uses of the present invention include MPDL3280A, BMS-936559, MEDI4736, MSB0010718C and an antibody which comprises the heavy chain and light chain variable regions of SEQ ID NO:24 and SEQ ID NO:21, respectively, of WO2013/019906.

Other PD-1 antagonists useful in any of the treatment methods, medicaments and uses of the present invention include an immunoadhesin that specifically binds to PD-1 or PD- L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesin molecules that specifically bind to PD-1 are described in WO2010/027827 and WO2011/066342. Specific fusion proteins useful as the PD-1 antagonist in the treatment methods, medicaments and uses of the present invention include AMP-224 (also known as B7-DCIg), which is a PD—L2—FC fusion protein that binds to human PD-1.

Thus, one embodiment provides for a method of treating cancer comprising administering an effective amount of a compound of the invention, or a pharmaceutically acceptable salt thereof, in combination with a PD-1 antagonist to a subject in need thereof. In such embodiments, the compounds of the invention, or a pharmaceutically acceptable salt thereof, and PD-1 antagonist are administered concurrently or sequentially.

Specific non-limiting examples of such cancers in accordance with this embodiment include melanoma (including unresectable or metastatic melanoma), head & neck cancer (including recurrent or metastatic head and neck squamous cell cancer (HNSCC)), classical Hodgkin lymphoma (cHL), urothelial carcinoma, gastric cancer, cervical cancer, primary mediastinal large-B-cell lymphoma, microsatellite instability-high (MSI-H) cancer, non-small cell lung cancer, hepatocellular carcinoma, clear cell kidney cancer, colorectal cancer, breast cancer, squamous cell lung cancer, basal carcinoma, sarcoma, bladder cancer, endometrial cancer, pancreatic cancer, liver cancer, gastrointestinal cancer, multiple myeloma, renal cancer, mesothelioma, ovarian cancer, anal cancer, biliary tract cancer, esophageal cancer, and salivary cancer.

In one embodiment, there is provided a method of treating cancer comprising administering an effective amount of a compound of the invention, or a pharmaceutically acceptable salt thereof, to a person in need thereof, in combination with a PD-1 antagonist, wherein said cancer is selected from unresectable or metastatic melanoma, recurrent or metastatic head and neck squamous cell cancer (HNSCC), classical Hodgkin lymphoma (cHL), urothelial carcinoma, gastric cancer, cervical cancer, primary mediastinal large-B-cell lymphoma, microsatellite instability-high (MSI-H) cancer, non-small cell lung cancer, and hepatocellular carcinoma. In one such embodiment, the agent is a PD-1 antagonist. In one such embodiment, the agent is pembrolizumab. In another such embodiment, the agent is nivolumab. In another such embodiment, the agent is atezolizumab.

Pembrolizumab is approved by the U.S. FDA for the treatment of patients with unresectable or metastatic melanoma and for the treatment of certain patients with recurrent or metastatic head and neck squamous cell cancer (HNSCC), classical Hodgkin lymphoma (cHL), urothelial carcinoma, gastric cancer, cervical cancer, primary mediastinal large-B-cell lymphoma, microsatellite instability-high (MSI-H) cancer, non-small cell lung cancer, and hepatocellular carcinoma, as described in the Prescribing Information for KEYTRUDA™ (Merck & Co., Inc., Whitehouse Station, NJ USA; initial U.S. approval 2014, updated November 2018). In another embodiment, there is provided a method of treating cancer comprising administering an effective amount of a compound of the invention, or a pharmaceutically acceptable salt thereof, to a person in need thereof, in combination with pembrolizumab, wherein said cancer is selected from unresectable or metastatic melanoma, recurrent or metastatic head and neck squamous cell cancer (HNSCC), classical Hodgkin lymphoma (cHL), urothelial carcinoma, gastric cancer, cervical cancer, primary mediastinal large-B-cell lymphoma, microsatellite instability-high (MSI-H) cancer, non-small cell lung cancer, and hepatocellular carcinoma.

In another embodiment, there is provided a method of treating cancer comprising administering an effective amount of a compound of the invention, or a pharmaceutically acceptable salt thereof, to a person in need thereof, in combination with a PD-1 antagonist, wherein said cancer is selected from melanoma, non-small cell lung cancer, head and neck squamous cell cancer (HNSCC), Hodgkin lymphoma, primary mediastinal large B-cell lymphoma, urothelial carcinoma, microsatellite instability-high cancer, gastric cancer, Merkel cell carcinoma, hepatocellular carcinoma, esophageal cancer and cervical cancer. In one such embodiment, the agent is a PD-1 antagonist. In one such embodiment, the agent is pembrolizumab. In another such embodiment, the agent is nivolumab. In another such embodiment, the agent is atezolizumab. In another such embodiment, the agent is durvalumab. In another such embodiment, the agent is avelumab.

In another embodiment, there is provided a method of treating cancer comprising administering an effective amount of a compound of the invention, or a pharmaceutically acceptable salt thereof, to a person in need thereof, in combination with a PD-1 antagonist, wherein said cancer is selected from melanoma, non-small cell lung cancer, small cell lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, thyroid cancer, and salivary cancer. In one such embodiment, the agent is a PD-1 antagonist. In one such embodiment, the agent is pembrolizumab. In another such embodiment, the agent is nivolumab. In another such embodiment, the agent is atezolizumab. In another such embodiment, the agent is durvalumab. In another such embodiment, the agent is avelumab.

In one embodiment, there is provided a method of treating unresectable or metastatic melanoma comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating recurrent or metastatic head and neck squamous cell cancer (HNSCC) comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating classical Hodgkin lymphoma (cHL) comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating urothelial carcinoma comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating gastric cancer comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating cervical cancer comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating primary mediastinal large-B-cell lymphoma comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating microsatellite instability-high (MSI-H) cancer comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating non-small cell lung cancer comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating hepatocellular carcinoma comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In another embodiment, the additional therapeutic agent is at least one immunomodulator other than an A2a or A2b receptor inhibitor. Non-limiting examples of immunomodulators include CD40L, B7, B7RP1, anti-CD40, anti-CD38, anti-ICOS, 4-IBB ligand, dendritic cell cancer vaccine, IL2, IL12, ELC/CCL19, SLC/CCL21, MCP-1, IL-4, IL-18, TNF, IL-15, MDC, IFN-a/-13, M-CSF, IL-3, GM-CSF, IL-13, anti-IL-10 and indolamine 2,3-dioxygenase 1 (IDO1) inhibitors.

In another embodiment, the additional therapeutic agent comprises radiation. Such radiation includes localized radiation therapy and total body radiation therapy.

In another embodiment, the additional therapeutic agent is at least one chemotherapeutic agent. Non-limiting examples of chemotherapeutic agents contemplated for use in combination with the compounds of the invention include: pemetrexed, alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nucleoside analogs (e.g., gemcitabine); nitroso ureas such as carmustine, lomustine, and streptozocin; topoisomerase 1 inhibitors (e.g., irinotecan); platinum complexes such as cisplatin, carboplatin and oxaliplatin; bioreductive alkylators such as mitomycin, procarbazine, dacarbazine and altretamine); anthracycline-based therapies (e.g., doxorubicin, daunorubicin, epirubicin and idarubicin); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, estramustine, vinblastine, docetaxol, epothilone derivatives, and paclitaxel); hormonal agents (e.g., estrogens; conjugated estrogens; ethynyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); luteinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide).

In another embodiment, the additional therapeutic agent is at least one signal transduction inhibitor (STI). Non-limiting examples of signal transduction inhibitors include BCR/ABL kinase inhibitors, epidermal growth factor (EGF) receptor inhibitors, HER-2/neu receptor inhibitors, and farnesyl transferase inhibitors (FTIs).

In another embodiment, the additional therapeutic agent is at least one anti-infective agent. Non-limiting examples of anti-infective agents include cytokines, non-limiting examples of which include granulocyte-macrophage colony stimulating factor (GM-CSF) and an flt3 -ligand.

In another embodiment, the present invention provides a method for treating or preventing a viral infection (e.g., a chronic viral infection) including, but not limited to, hepatitis C virus (HCV), human papilloma virus (HPV), cytomegalovirus (CMV), Epstein-Barr virus (EBV), varicella zoster virus, coxsackievirus, and human immunodeficiency virus (HIV).

In another embodiment, the present invention provides a method for the treatment of an infective disorder, said method comprising administering to a subject in need thereof an effective amount of a compound of the invention, or a pharmaceutically acceptable salt thereof, in combination with a vaccine. In some embodiments, the vaccine is an anti-viral vaccine, including, for example, an anti-HTV vaccine. Other antiviral agents contemplated for use include an anti-HIV, anti-HPV, anti HCV, anti HSV agents and the like. In other embodiments, the vaccine is effective against tuberculosis or malaria. In still other embodiments, the vaccine is a tumor vaccine (e.g., a vaccine effective against melanoma); the tumor vaccine may comprise genetically modified tumor cells or a genetically modified cell line, including genetically modified tumor cells or a genetically modified cell line that has been transfected to express granulocyte-macrophage stimulating factor (GM-CSF). In another embodiment, the vaccine includes one or more immunogenic peptides and/or dendritic cells.

In another embodiment, the present invention provides for the treatment of an infection by administering a compound of the invention, or a pharmaceutically acceptable salt thereof, and at least one additional therapeutic agent, wherein a symptom of the infection observed after administering both the compound of the invention (or a pharmaceutically acceptable salt thereof) and the additional therapeutic agent is improved over the same symptom of infection observed after administering either alone. In some embodiments, the symptom of infection observed can be reduction in viral load, increase in CD4+ T cell count, decrease in opportunistic infections, increased survival time, eradication of chronic infection, or a combination thereof.

### DEFINITIONS

As used herein, unless otherwise specified, the following terms have the following meanings.

Unsatisfied valences in the text, schemes, examples, structural formulae, and any Tables herein are assumed to have a hydrogen atom or atoms of sufficient number to satisfy the valences.

When a variable appears more than once in any moiety or in any compound of the invention (e.g., aryl, heterocycloalkyl, N(R)₂), the selection of moieties defining that variable for each occurrence is independent of its definition at every other occurrence unless specified otherwise in the local variable definition.

As used herein, unless otherwise specified, the term “A2a receptor antagonist” (equivalently, A2a antagonist) and/or “A2b receptor antagonist” (equivalently, A2b antagonist) means a compound exhibiting a potency (IC₅₀) of less than about 1 µM with respect to the A2a and/or A2b receptors, respectively, when assayed in accordance with the procedures described herein. Preferred compounds exhibit at least 10-fold selectivity for antagonizing the A2a receptor and/or the A2b receptor over any other adenosine receptor (e.g., A1 or A3).

As described herein, unless otherwise indicated, the use of a compound in treatment means that an amount of the compound, generally presented as a component of a formulation that comprises other excipients, is administered in aliquots of an amount, and at time intervals, which provides and maintains at least a therapeutic serum level of at least one pharmaceutically active form of the compound over the time interval between dose administrations.

The phrase “at least one” used in reference to the number of components comprising a composition, for example, “at least one pharmaceutical excipient” means that one member of the specified group is present in the composition, and more than one may additionally be present. Components of a composition are typically aliquots of isolated pure material added to the composition, where the purity level of the isolated material added into the composition is the normally accepted purity level for a reagent of the type.

Whether used in reference to a substituent on a compound or a component of a pharmaceutical composition the phrase “one or more”, means the same as “at least one”.

“Concurrently” and “contemporaneously” both include in their meaning (1) simultaneously in time (e.g., at the same time); and (2) at different times but within the course of a common treatment schedule.

“Consecutively” means one following the other.

“Sequentially” refers to a series administration of therapeutic agents that awaits a period of efficacy to transpire between administering each additional agent; this is to say that after administration of one component, the next component is administered after an effective time period after the first component; the effective time period is the amount of time given for realization of a benefit from the administration of the first component.

“Effective amount” or “therapeutically effective amount” is meant to describe the provision of an amount of at least one compound of the invention or of a composition comprising at least one compound of the invention which is effective in treating or inhibiting a disease or condition described herein, and thus produce the desired therapeutic, ameliorative, inhibitory or preventative effect. For example, in treating a cancer as described herein with one or more of the compounds of the invention optionally in combination with one or more additional agents, “effective amount” (or “therapeutically effective amount”) means, for example, providing the amount of at least one compound of the invention that results in a therapeutic response in a patient afflicted with the disease, condition, or disorder, including a response suitable to manage, alleviate, ameliorate, or treat the condition or alleviate, ameliorate, reduce, or eradicate one or more symptoms attributed to the condition and/or long-term stabilization of the condition, for example, as may be determined by the analysis of pharmacodynamic markers or clinical evaluation of patients afflicted with the condition.

“Patient” and “subject” includes both human and non-human animals. Non-human animals include those research animals and companion animals such as mice, rats, primates, monkeys, chimpanzees, great apes, dogs, and house cats.

“Prodrug” means compounds that are rapidly transformed, for example, by hydrolysis in blood, in vivo to the parent compound, e.g., conversion of a prodrug of a compound of the invention to a compound of the invention, or to a salt thereof. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference; the scope of this invention includes prodrugs of the novel compounds of this invention.

The term “substituted” means that one or more of the moieties enumerated as substituents (or, where a list of substituents are not specifically enumerated, the substituents specified elsewhere in this application) for the particular type of substrate to which said substituent is appended, provided that such substitution does not exceed the normal valence rules for the atom in the bonding configuration presented in the substrate, and that the substitution ultimately provides a stable compound, which is to say that such substitution does not provide compounds with mutually reactive substituents located geminal or vicinal to each other; and wherein the substitution provides a compound sufficiently robust to survive isolation to a useful degree of purity from a reaction mixture.

Where optional substitution by a moiety is described (e.g. “optionally substituted”) the term means that if substituents are present, one or more of the enumerated (or default) moieties listed as optional substituents for the specified substrate can be present on the substrate in a bonding position normally occupied by the default substituent, for example, a hydrogen atom on an alkyl chain can be substituted by one of the optional substituents, in accordance with the definition of “substituted” presented herein.

“Alkyl” means an aliphatic hydrocarbon group, which may be straight or branched, comprising 1 to 10 carbon atoms. “(C₁-C₆)alkyl” means an aliphatic hydrocarbon group, which may be straight or branched, comprising 1 to 6 carbon atoms. Branched means that one or more lower alkyl groups such as methyl, ethyl or propyl, are attached to a linear alkyl chain. Non-limiting examples of alkyl groups include methyl, ethyl, n-propyl, isopropyl, n-butyl, i-butyl, and t-butyl.

“Haloalkyl” means an alkyl as defined above wherein one or more hydrogen atoms on the alkyl (up to and including each available hydrogen group) is replaced by a halogen atom. As appreciated by those of skill in the art, “halo” or “halogen” as used herein is intended to include chloro (Cl), fluoro (F), bromo (Br) and iodo (I). Chloro (Cl) and fluoro(F) halogens are generally preferred.

“Aryl” means an aromatic monocyclic or multicyclic ring system comprising 6 to 14 carbon atoms, preferably 6 to 10 carbon atoms. The aryl group can be optionally substituted with one or more “ring system substituents” which may be the same or different, and are as defined herein. Non-limiting examples of suitable aryl groups include phenyl and naphthyl. “Monocyclic aryl” means phenyl.

“Heteroaryl” means an aromatic monocyclic or multicyclic ring system comprising 5 to 14 ring atoms, preferably 5 to 10 ring atoms, in which one or more of the ring atoms is an element other than carbon, for example nitrogen, oxygen or sulfur, alone or in combination. Preferred heteroaryls contain 5 to 6 ring atoms. The “heteroaryl” can be optionally substituted by one or more substituents, which may be the same or different, as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl (which alternatively may be referred to as thiophenyl), pyrimidinyl, pyridone (including N-substituted pyridones), isoxazolyl, isothiazolyl, oxazolyl, oxadiazolyl, thiazolyl, thiadiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like. The term “monocyclic heteroaryl” refers to monocyclic versions of heteroaryl as described above and includes 4- to 7-membered monocyclic heteroaryl groups comprising from 1 to 4 ring heteroatoms, said ring heteroatoms being independently selected from the group consisting of N, O, and S, and oxides thereof. The point of attachment to the parent moiety is to any available ring carbon or ring heteroatom. Non-limiting examples of monocyclic heteroaryl moieties include pyridyl, pyrazinyl, furanyl, thienyl, pyrimidinyl, pyridazinyl, pyridinyl, thiazolyl, isothiazolyl, oxazolyl, oxadiazolyl, isoxazolyl, pyrazolyl, furazanyl, pyrrolyl, pyrazolyl, triazolyl, thiadiazolyl (e.g., 1,2,4-thiadiazolyl), imidazolyl, and triazinyl (e.g., 1,2,4-triazinyl), and oxides thereof.

“Cycloalkyl” means a non-aromatic fully or partially saturated monocyclic or multicyclic ring system comprising 3 to 10 carbon atoms, preferably 3 to 6 carbon atoms. The cycloalkyl can be optionally substituted with one or more substituents, which may be the same or different, as described herein. Monocyclic cycloalkyl refers to monocyclic versions of the cycloalkyl moieties described herein. Non-limiting examples of suitable monocyclic cycloalkyls include cyclopropyl, cyclopentyl, cyclohexyl, cycloheptyl and the like. Non-limiting examples of multicyclic cycloalkyls include [1.1.1]-bicyclopentane, 1-decalinyl, norbornyl, adamantyl and the like.

“Heterocycloalkyl” (or “heterocyclyl”) means a non-aromatic saturated or partially saturated monocyclic or multicyclic ring system comprising 3 to 10 ring atoms, preferably 5 to 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocycloalkyl groups contain 4, 5 or 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an -N(Boc), -N(CBz), -N(Tos) group and the like; such protections are also considered part of this invention. The heterocycloalkyl can be optionally substituted by one or more substituents, which may be the same or different, as described herein. The nitrogen or sulfur atom of the heterocycloalkyl can be optionally oxidized to the corresponding N-oxide, S-oxide or S,S-dioxide. Thus, the term “oxide,” when it appears in a definition of a variable in a general structure described herein, refers to the corresponding N-oxide, S-oxide, or S,S-dioxide. “Heterocycloalkyl” also includes rings wherein =O replaces two available hydrogens on the same carbon atom (i.e., heterocyclyl includes rings having a carbonyl group in the ring). Such =O groups may be referred to herein as “oxo.” An example of such a moiety is pyrrolidinone (or pyrrolidone):

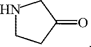

As used herein, the term “monocyclic heterocycloalkyl” refers to monocyclic versions of the heterocycloalkyl moieties described herein and include a 4- to 7-membered monocyclic heterocycloalkyl groups comprising from 1 to 4 ring heteroatoms, said ring heteroatoms being independently selected from the group consisting of N, N-oxide, O, S, S-oxide, S(O), and S(O)₂. The point of attachment to the parent moiety is to any available ring carbon or ring heteroatom. Non-limiting examples of monocyclic heterocycloalkyl groups include piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof. Non-limiting examples of lower alkyl-substituted oxetanyl include the moiety:

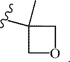

It is noted that in hetero-atom containing ring systems of this invention, there are no hydroxyl groups on carbon atoms adjacent to a N, O or S, and there are no N or S groups on carbon adjacent to another heteroatom.

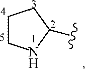

there is no —OH attached directly to carbons marked 2 and 5.

The line

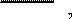

as a bond generally indicates a mixture of, or either of, the possible isomers, e.g., containing (R)- and (S)— stereochemistry. For example:

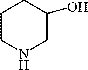

means containing both

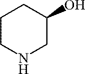

and

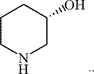

The wavy line

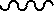

, as used herein, indicates a point of attachment to the rest of the compound. Lines drawn into the ring systems, such as, for example:

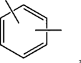

indicate that the indicated line (bond) may be attached to any of the substitutable ring atoms.

“Oxo” is defined as an oxygen atom that is double bonded to a ring carbon in a cycloalkyl, cycloalkenyl, heterocyclyl, heterocycloalkylnyl, or other ring described herein, e.g.,

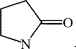

As well known in the art, a bond drawn from a particular atom wherein no moiety is depicted at the terminal end of the bond indicates a methyl group bound through that bond to the atom, unless stated otherwise. For example:

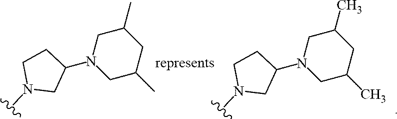

One or more compounds of the invention may also exist as, or optionally be converted to, a solvate. Preparation of solvates is generally known. Thus, for example, M. Caira et al., J. Pharmaceutical Sci., 93(3), 601-611 (2004) describe the preparation of the solvates of the antifungal fluconazole in ethyl acetate as well as from water. Similar preparations of solvates, and hemisolvate, including hydrates (where the solvent is water or aqueous-based) and the like are described by E. C. van Tonder et al., AAPS PharmSciTech., 5(1), article 12 (2004); and A. L. Bingham et al., Chem. Commun., 603-604 (2001). A typical, non-limiting, process involves dissolving the inventive compound in desired amounts of the desired solvent (for example, an organic solvent, an aqueous solvent, water or mixtures of two or more thereof) at a higher than ambient temperature, and cooling the solution, with or without an antisolvent present, at a rate sufficient to form crystals which are then isolated by standard methods. Analytical techniques such as, for example I.R. spectroscopy, show the presence of the solvent (including water) in the crystals as a solvate (or hydrate in the case where water is incorporated into the crystalline form).

The term “purified”, “in purified form” or “in isolated and purified form” for a compound refers to the physical state of said compound after being isolated from a synthetic process or natural source or combination thereof. Thus, the term “purified”, “in purified form” or “in isolated and purified form” for a compound refers to the physical state of said compound after being obtained from a purification process or processes described herein or well known to the skilled artisan, and in sufficient purity to be characterized by standard analytical techniques described herein or well known to the skilled artisan.

This invention also includes the compounds of the invention in isolated and purified form obtained by routine techniques. Polymorphic forms of the compounds of the invention, and of the salts, solvates and prodrugs of the thereof, are intended to be included in the present invention. Certain compounds of the invention may exist in different isomeric forms (e.g., enantiomers, diastereoisomers, atropisomers). The inventive compounds include all isomeric forms thereof, both in pure form and admixtures of two or more, including racemic mixtures.

In similar manner, unless indicated otherwise, presenting a structural representation of any tautomeric form of a compound which exhibits tautomerism is meant to include all such tautomeric forms of the compound. Accordingly, where compounds of the invention, their salts, and solvates and prodrugs thereof, may exist in different tautomeric forms or in equilibrium among such forms, all such forms of the compound are embraced by, and included within the scope of the invention. Examples of such tautomers include, but are not limited to, ketone/enol tautomeric forms, imine-enamine tautomeric forms, and for example heteroaromatic forms such as the following moieties:

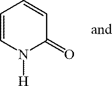

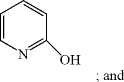

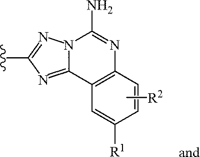

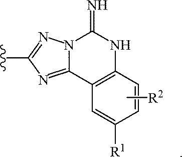

Where a reaction scheme appearing in an example employs a compound having one or more stereocenters, the stereocenters are indicated with an asterisk, as shown below:

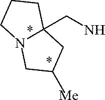

Accordingly, the above depiction consists of the following pairs of isomers: (i) Trans-isomers ((2R,7aS)-2-methylhexahydro-1H-pyrrolizin-7a-yl)methanamine (Compound ABC-1) and ((2S,7aR)-2-methylhexahydro-1H-pyrrolizin-7a-yl)methanamine (Compound ABC-2); and (ii) Cis-isomers ((2R,7aR)-2-methylhexahydro-1H-pyrrolizin-7a-yl)methanamine (Compound ABC-3) and ((2S,7aS)-2-methylhexahydro-1H-pyrrolizin-7a-yl)methanamine (Compound ABC-4).

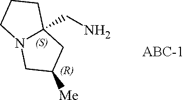 [SMILES: C[C@H]1CN2CCC[C@@]2(CN)C1]

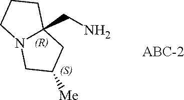 [SMILES: C[C@@H]1CN2CCC[C@]2(CN)C1]

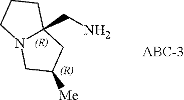 [SMILES: C[C@H]1CN2CCC[C@]2(CN)C1]

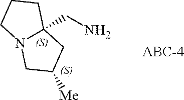 [SMILES: C[C@@H]1CN2CCC[C@@]2(CN)C1]

All stereoisomers of the compounds of the invention (including salts and solvates of the inventive compounds and their prodrugs), such as those which may exist due to asymmetric carbons present in a compound of the invention, and including enantiomeric forms (which may exist even in the absence of asymmetric carbons), rotameric forms, atropisomers, and diastereomeric forms, are contemplated within the scope of this invention. Individual stereoisomers of the compounds of the invention may be isolated in a pure form, for example, substantially free of other isomers, or may be isolated as an admixture of two or more stereoisomers or as a racemate. The chiral centers of the present invention can have the S or R configuration as defined by the IUPAC 1974 Recommendations. The use of the terms “salt”, “solvate” “prodrug” and the like, is intended to equally apply to salts, solvates and prodrugs of isolated enantiomers, stereoisomer pairs or groups, rotamers, tautomers, or racemates of the inventive compounds.

Where diastereomeric mixtures can be separated into their individual diastereomers on the basis of their physical chemical differences by known methods, for example, by chiral chromatography and/or fractional crystallization, simple structural representation of the compound contemplates all diastereomers of the compound. As is known, enantiomers may also be separated by converting the enantiomeric mixture into a diastereomeric mixture by reaction with an appropriate optically active compound (e.g., chiral auxiliary such as a chiral alcohol or Mosher’s acid chloride), separating the diastereomers and converting (e.g., hydrolyzing) the individually isolated diastereomers to the corresponding purified enantiomers.

As the term is employed herein, salts of the inventive compounds, whether acidic salts formed with inorganic and/or organic acids, basic salts formed with inorganic and/or organic bases, salts formed which include zwitterionic character, for example, where a compound contains both a basic moiety, for example, but not limited to, a nitrogen atom, for example, an amine, pyridine or imidazole, and an acidic moiety, for example, but not limited to a carboxylic acid, are included in the scope of the inventive compounds described herein. The formation of pharmaceutically useful salts from basic (or acidic) pharmaceutical compounds are discussed, for example, by S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Int′l. Union of Pure and Applied Chemistry, pp. 330-331. These disclosures are incorporated herein by reference.

The present invention contemplates all available salts, including salts which are generally recognized as safe for use in preparing pharmaceutical formulations and those which may be formed presently within the ordinary skill in the art and are later classified as being “generally recognized as safe” for use in the preparation of pharmaceutical formulations, termed herein as “pharmaceutically acceptable salts”. Examples of pharmaceutically acceptable acid addition salts include, but are not limited to, acetates, including trifluoroacetate salts, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like.

Examples of pharmaceutically acceptable basic salts include, but are not limited to, ammonium salts, alkali metal salts such as sodium, lithium, and potassium salts, alkaline earth metal salts such as calcium and magnesium salts, aluminum salts, zinc salts, salts with organic bases (for example, organic amines) such as benzathines, diethylamine, dicyclohexylamines, hydrabamines (formed with N,N-bis(dehydroabietyl)ethylenediamine), N-methyl-D-glucamines, N-methyl-D-glucamides, t-butyl amines, piperazine, phenylcyclohexyl-amine, choline, tromethamine, and salts with amino acids such as arginine, lysine and the like. Basic nitrogen-containing groups may be converted to an ammonium ion or quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), arylalkyl halides (e.g. benzyl and phenethyl bromides), and others.

All such acid and base salts are intended to be pharmaceutically acceptable salts within the scope of the invention and all acid and base salts are considered equivalent to the free forms of the corresponding compounds for purposes of the scope of the invention.

A functional group in a compound termed “protected” means that the group is in modified form to preclude undesired side reactions at the protected site when the protected compound is subjected to particular reaction conditions aimed at modifying another region of the molecule. Suitable protecting groups are known, for example, as by reference to standard textbooks, for example, T. W. Greene et al., Protective Groups in organic Synthesis (1991), Wiley, New York.

In the compounds of the invention, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of the invention. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds of the invention can be prepared without undue experimentation by conventional techniques well known to those skilled in the art or by processes analogous to those described in the Schemes and Examples herein using appropriate isotopically-enriched reagents and/or intermediates.

The present invention also embraces isotopically-labeled compounds of the present invention which are structurally identical to those recited herein, but for the fact that a statistically significant percentage of one or more atoms in that form of the compound are replaced by an atom having an atomic mass or mass number different from the atomic mass or mass number of the most abundant isotope usually found in nature, thus altering the naturally occurring abundance of that isotope present in a compound of the invention. Examples of isotopes that can be preferentially incorporated into compounds of the invention include isotopes of hydrogen, carbon, nitrogen, oxygen, phosphorus, iodine, fluorine and chlorine, for example, but not limited to: ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, and ³⁶Cl, ¹²³I and ¹²⁵I. It will be appreciated that other isotopes also may be incorporated by known means.

Certain isotopically-labeled compounds of the invention (e.g., those labeled with ³H, ¹¹C and ¹⁴C) are recognized as being particularly useful in compound and/or substrate tissue distribution assays using a variety of known techniques. Tritiated (i.e., ³H) and carbon-14 (i.e., ¹⁴C) isotopes are particularly preferred for their ease of preparation and detection. Further, substitution of a naturally abundant isotope with a heavier isotope, for example, substitution of protium with deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in the reaction Schemes and/or in the Examples herein below, by substituting an appropriate isotopically labeled reagent for a non-isotopically labeled reagent, or by well-known reactions of an appropriately prepared precursor to the compound of the invention which is specifically prepared for such a “labeling” reaction. Such compounds are included also in the present invention.

It is understood that one or more silicon (Si) atoms can be incorporated into the compounds of the instant invention in place of one or more carbon atoms by one of ordinary skill in the art to provide compounds that are chemically stable and that can be readily synthesized by techniques known in the art from readily available starting materials. Carbon and silicon differ in their covalent radius leading to differences in bond distance and the steric arrangement when comparing analogous C-element and Si-element bonds. These differences lead to subtle changes in the size and shape of silicon-containing compounds when compared to carbon. One of ordinary skill in the art would understand that size and shape differences can lead to subtle or dramatic changes in potency, solubility, lack of off-target activity, packaging properties, and so on. (Diass, J. O. et al. Organometallics (2006) 5:1188-1198; Showell, G.A. et al. Bioorganic & Medicinal Chemistry Letters (2006) 16:2555-2558).

The term “composition” is intended to encompass a product comprising the specified ingredients in the specified amounts, and any product which results, directly or indirectly, from combination of the specified ingredients in the specified amounts.

The term “pharmaceutical composition” as used herein encompasses both the bulk composition and individual dosage units comprised of one, or more than one (e.g., two), pharmaceutically active agents such as, for example, a compound of the present invention (optionally together with an additional agent as described herein), along with any pharmaceutically inactive excipients. As will be appreciated by those of ordinary skill in the art, excipients are any constituent which adapts the composition to a particular route of administration or aids the processing of a composition into a dosage form without itself exerting an active pharmaceutical effect. The bulk composition and each individual dosage unit can contain fixed amounts of the aforesaid one, or more than one, pharmaceutically active agents. The bulk composition is material that has not yet been formed into individual dosage units.

It will be appreciated that pharmaceutical formulations of the invention may comprise more than one compound of the invention (or a pharmaceutically acceptable salt thereof), for example, the combination of two or three compounds of the invention, each present in such a composition by adding to the formulation the desired amount of the compound in a pharmaceutically acceptably pure form. It will be appreciated also that in formulating compositions of the invention, a composition may comprise, in addition to one or more of compounds of the invention, one or more other agents which also have pharmacological activity, as described herein.

While formulations of the invention may be employed in bulk form, it will be appreciated that for most applications the inventive formulations will be incorporated into a dosage form suitable for administration to a patient, each dosage form comprising an amount of the selected formulation which contains an effective amount of one or more compounds of the invention. Examples of suitable dosage forms include, but are not limited to, dosage forms adapted for: (i) oral administration, e.g., a liquid, gel, powder, solid or semi-solid pharmaceutical composition which is loaded into a capsule or pressed into a tablet and may comprise additionally one or more coatings which modify its release properties, for example, coatings which impart delayed release or formulations which have extended release properties; (ii) a dosage form adapted for intramuscular administration (IM), for example, an injectable solution or suspension, and which may be adapted to form a depot having extended release properties; (iii) a dosage form adapted for intravenous administration (IV), for example, a solution or suspension, for example, as an IV solution or a concentrate to be injected into a saline IV bag; (iv) a dosage form adapted for administration through tissues of the oral cavity, for example, a rapidly dissolving tablet, a lozenge, a solution, a gel, a sachets or a needle array suitable for providing intramucosal administration; (v) a dosage form adapted for administration via the mucosa of the nasal or upper respiratory cavity, for example a solution, suspension or emulsion formulation for dispersion in the nose or airway; (vi) a dosage form adapted for transdermal administration, for example, a patch, cream or gel; (vii) a dosage form adapted for intradermal administration, for example, a microneedle array; and (viii) a dosage form adapted for delivery via rectal or vaginal mucosa, for example, a suppository.

For preparing pharmaceutical compositions comprising compounds of the invention, generally the compounds of the invention will be combined with one or more pharmaceutically acceptable excipients. These excipients impart to the composition properties which make it easier to handle or process, for example, lubricants or pressing aids in powdered medicaments intended to be tableted, or adapt the formulation to a desired route of administration, for example, excipients which provide a formulation for oral administration, for example, via absorption from the gastrointestinal tract, transdermal or transmucosal administration, for example, via adhesive skin “patch” or buccal administration, or injection, for example, intramuscular or intravenous, routes of administration. These excipients are collectively termed herein “a carrier”. Typically formulations may comprise up to about 95 percent active ingredient, although formulations with greater amounts may be prepared.

Pharmaceutical compositions can be solid, semi-solid or liquid. Solid form preparations can be adapted to a variety of modes of administration, examples of which include, but are not limited to, powders, dispersible granules, mini-tablets, beads, which can be used, for example, for tableting, encapsulation, or direct administration. Liquid form preparations include, but are not limited to, solutions, suspensions and emulsions which for example, but not exclusively, can be employed in the preparation of formulations intended for parenteral injection, for intranasal administration, or for administration to some other mucosal membrane. Formulations prepared for administration to various mucosal membranes may also include additional components adapting them for such administration, for example, viscosity modifiers.

Aerosol preparations, for example, suitable for administration via inhalation or via nasal mucosa, may include solutions and solids in powder form, which may be in combination with a pharmaceutically acceptable propellant, for example, an inert compressed gas, e.g. nitrogen. Also included are solid form preparations which are intended to be converted, shortly before use, to a suspension or a solution, for example, for oral or parenteral administration. Examples of such solid forms include, but are not limited to, freeze dried formulations and liquid formulations adsorbed into a solid absorbent medium.

The compounds of the invention may also be deliverable transdermally or transmucosally, for example, from a liquid, suppository, cream, foam, gel, or rapidly dissolving solid form. It will be appreciated that transdermal compositions can take also the form of creams, lotions, aerosols and/or emulsions and can be provided in a unit dosage form which includes a transdermal patch of any know in the art, for example, a patch which incorporates either a matrix comprising the pharmaceutically active compound or a reservoir which comprises a solid or liquid form of the pharmaceutically active compound.

Examples of pharmaceutically acceptable carriers and methods of manufacture for various compositions mentioned above may be found in A. Gennaro (ed.), Remington: The Science and Practice of Pharmacy, 20^{th} Edition, (2000), Lippincott Williams & Wilkins, Baltimore, MD.

Preferably, the pharmaceutical preparation is in a unit dosage form. In such form, the preparations subdivided into suitably sized unit doses containing appropriate quantities of the active component, e.g., an effective amount to achieve the desired purpose.

The actual dosage employed may be varied depending upon the requirements of the patient and the severity of the condition being treated. Determination of the proper dosage regimen for a particular situation is within the skill in the art. For convenience, the total daily dosage may be divided and administered in portions during the day as required.

In accordance with the present invention, antagonism of adenosine A2a and/or A2b receptors is accomplished by administering to a patient in need of such therapy an effective amount of one or more compounds of the invention, or a pharmaceutically acceptable salt thereof.

In some embodiments it is preferred for the compound to be administered in the form of a pharmaceutical composition comprising the compound of the invention, or a salt thereof, and at least one pharmaceutically acceptable carrier (described herein). It will be appreciated that pharmaceutically formulations of the invention may comprise more than one compound of the invention, or a salt thereof, for example, the combination of two or three compounds of the invention, or, additionally or alternatively, another active agent such as those described herein, each present by adding to the formulation the desired amount of the compound or a salt thereof (or agent, where applicable) which has been isolated in a pharmaceutically acceptably pure form.

As mentioned above, administration of a compound of the invention to effect antagonism of A2a and/or A2b receptors is preferably accomplished by incorporating the compound into a pharmaceutical formulation incorporated into a dosage form, for example, one of the above-described dosage forms comprising an effective amount of at least one compound of the invention (e.g., 1, 2 or 3, or 1 or 2, or 1, and usually 1 compound of the invention), or a pharmaceutically acceptable salt thereof. Methods for determining safe and effective administration of compounds which are pharmaceutically active, for example, a compound of the invention, are known to those skilled in the art, for example, as described in the standard literature, for example, as described in the “Physicians’ Desk Reference” (PDR), e.g., 1996 edition (Medical Economics Company, Montvale, NJ 07645-1742, USA), the Physician’s Desk Reference, 56^{th} Edition, 2002 (published by Medical Economics company, Inc. Montvale, NJ 07645-1742), or the Physician’s Desk Reference, 57^{th} Edition, 2003 (published by Thompson PDR, Montvale, NJ 07645-1742); the disclosures of which is incorporated herein by reference thereto. The amount and frequency of administration of the compounds of the invention and/or the pharmaceutically acceptable salts thereof will be regulated according to the judgment of the attending clinician considering such factors as age, condition and size of the patient as well as severity of the symptoms being treated. Compounds of the invention can be administered at a total daily dosage of up to 1,000 mg, which can be administered in one daily dose or can be divided into multiple doses per 24 hour period, for example, two to four doses per day.

As those of ordinary skill in the art will appreciate, an appropriate dosage level for a compound (or compounds) of the invention will generally be about 0.01 to 500 mg per kg patient body weight per day which can be administered in single or multiple doses. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions may be provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, or may be administered once or twice per day.

Those skilled in the art will appreciate that treatment protocols utilizing at least one compound of the invention can be varied according to the needs of the patient. Thus, compounds of the invention used in the methods of the invention can be administered in variations of the protocols described above. For example, compounds of the invention can be administered discontinuously rather than continuously during a treatment cycle.

In general, in whatever form administered, the dosage form administered will contain an amount of at least one compound of the invention, or a salt thereof, which will provide a therapeutically effective serum level of the compound in some form for a suitable period of time such as at least 2 hours, more preferably at least four hours or longer. In general, as is known in the art, dosages of a pharmaceutical composition providing a therapeutically effective serum level of a compound of the invention can be spaced in time to provide serum level meeting or exceeding the minimum therapeutically effective serum level on a continuous basis throughout the period during which treatment is administered. As will be appreciated the dosage form administered may also be in a form providing an extended release period for the pharmaceutically active compound which will provide a therapeutic serum level for a longer period, necessitating less frequent dosage intervals. As mentioned above, a composition of the invention can incorporate additional pharmaceutically active components or be administered simultaneously, contemporaneously, or sequentially with other pharmaceutically active agents as may be additionally needed or desired in the course of providing treatment. As will be appreciated, the dosage form administered may also be in a form providing an extended release period for the pharmaceutically active compound which will provide a therapeutic serum level for a longer period, necessitating less frequent dosage intervals.

### Preparative Examples

The compounds of the present invention can be prepared readily according to the following schemes and specific examples, or modifications thereof, using readily available starting materials, reagents and conventional synthetic procedures. In these reactions, it is also possible to make use of variants which are themselves known to those of ordinary skill in this art but are not mentioned in detail. The general procedures for making the compounds claimed in this invention can be readily understood and appreciated by one skilled in the art from viewing the following Schemes and descriptions.

Abbreviations used herein have the following meaning:

| | |
|---|---|
| °C | Degrees Celsius |
| µL | Microliter |
| µmol | Micromolar |
| Ac | Acetyl |
| Ac₂O | Acetic anhydride |
| AcOH | Acetic acid |
| aq. | Aqueous |
| B₂Pin₂ | Bis(pinacolato)diboron or 4,4,4′,4′,5,5,5′,5′-Octamethyl-2,2′-bi-1,3,2-dioxaborolane |
| Boc | Tert-butoxycarbonyl |
| Boc₂O | Di-tert-butyl dicarbonate |
| BSA | N,O-Bis(trimethylsilyl)acetamide |
| Celite^{Ⓡ} | Diatomaceous earth |
| COMU | (1-Cyano-2-ethoxy-2-oxoethylidenaminooxy)dimethylamino-morpholino-carbenium hexafluorophosphate |
| DBU | 1,8-Diazabicyclo[5.4.0]undec-7-ene |
| DCE | Dichloroethane |
| DCM | Dichloromethane |
| DDQ | 2,3-Dichloro-5,6-dicyano-1,4-benzoquinone |
| Dioxane | 1,4-dioxane |
| DIPEA | N,N-Diisopropylethylamine |
| DMAP | 4-Dimethylaminopyridine |
| DMF | Dimethylformamide |
| DMP | Dess-Martin periodinane or 1,1,1-Tris(acetyloxy)-1,1-dihydro-1,2-benziodoxol-3-(1H)-one |
| DMSO | Dimethyl Sulfoxide |
| DMSO-d₆ | Deuterated Dimethyl Sulfoxide |
| dppf | Bis(diphenylphosphino)ferrocene |
| ESI | Electrospray Ionization |
| Et₂NH | Diethylamine |
| Et₃N | Triethylamine |
| EtOAc | Ethyl Acetate |
| EtOH | Ethanol |
| G | Grams |
| H | Hour(s) |
| HBPin | Pinacolborane or 4,4,5,5-Tetramethyl-1,3,2-dioxaborolane |
| Hoveyda-Grubbs Catalyst^{Ⓒ} 2^{nd} Generation | 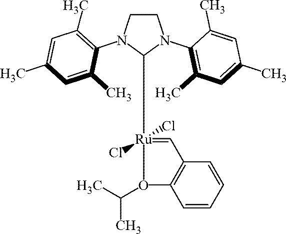 |
| | Dichloro[1,3-bis(2,4,6-trimethylphenyl)-2-imidazolidinylidene](2-isopropoxyphenylmethylene)ruthenium(II) CAS# 301224-40-8 |
| HPLC | High Performance Liquid Chromatography |
| i-PrBr | Isopropyl bromide |
| i-PrOH | Isopropyl alcohol or 2-propanol |
| M | Molar |
| MBTE | Methyl tert-butyl ether |
| MeCN | Acetonitrile |
| MeMgBr | Methylmagnesium bromide |
| MeOD-d₄ | Deuterated Methanol-d4 |
| MeOH | Methanol |
| mg | Milligrams |
| MHz | Megahertz |
| min | Minute(s) |
| mL | Milliliters |
| mmol | Millimoles |
| MP-CNBH₃ | 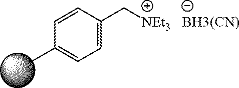 |
| | BiotageⓇ macroporous polymer supported cyanoborohydride Part #: 800405 |
| MS | Mass Spectroscopy |
| Ms | Methanesulfonyl |
| nM | Nanomolar |
| NMM | N-methylmorpholine |
| NMR | Nuclear Magnetic Resonance |
| OAc | Acetate or acetoxy |
| OMe | Methoxide or methoxy |
| OMs | Mesylate or methanesulfonate |
| Oxone® | Potassium peroxymonosulfate triple salt, |
| | 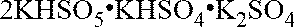 |
| P(t-Bu)₃ Pd G2 | 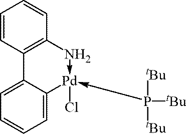 |
| | Chloro[(tri-tert-butylphosphine)-2-(2-aminobiphenyl)] palladium(II) |
| | CAS# 1375325-71-5 |
| Pd/C | Palladium on Carbon |
| Pd(PPh₃)₄ | Tetrakis(triphenylphosphine)palladium(0) |
| Ph | Phenyl |
| PIDA | (Diacetoxyiodo)benzene |
| psi | Pounds per square inch |
| py | Pyridine or pyridyl |
| RockPhos Pd G3 | 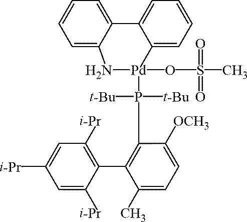 |
| | [(2-Di-tert-butylphosphino-3-methoxy-6-methyl-2′,4′,6′-triisopropyl-1,1′-biphenyl)-2-(2-aminobiphenyl)]palladium(II) methanesulfonate CAS# 2009020-38-4 |
| sat. | Saturated |
| SFC | Supercritical fluid chromatography (CO₂) |
| T3P® | 1-Propanephosphonic anhydride |
| TBAI | Tetra-n-butylammonium iodide |
| TBSCl | Tert-butyldimethylsilyl chloride |
| t-BuXPhos Pd G3 | 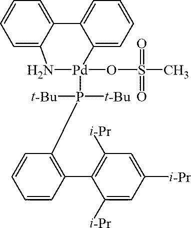 |
| | [(2-Di-tert-butylphosphino-2′,4′,6′-triisopropyl-1,1′-biphenyl)-2-(2′-amino-1,1′-biphenyl)] palladium(II) methanesulfonate CAS# 1447963-75-8 |
| t-BuXPhos | 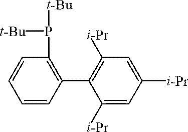 [SMILES: CC(C)c1cc(C(C)C)c(-c2ccccc2P(C(C)(C)C)C(C)(C)C)c(C(C)C)c1] |
| | 2-Di-tert-butylphosphino-2′,4′,6′-triisopropylbiphenyl CAS# 564483-19-8 |
| TFA | Trifluoroacetic acid |
| TFAA | Trifluoroacetic anhydride |
| THF | Tetrahydrofuran |
| TLC | Thin Layer Chromatography |
| TMS | Trimethylsilyl |
| Ts | Toluenesulfonyl |

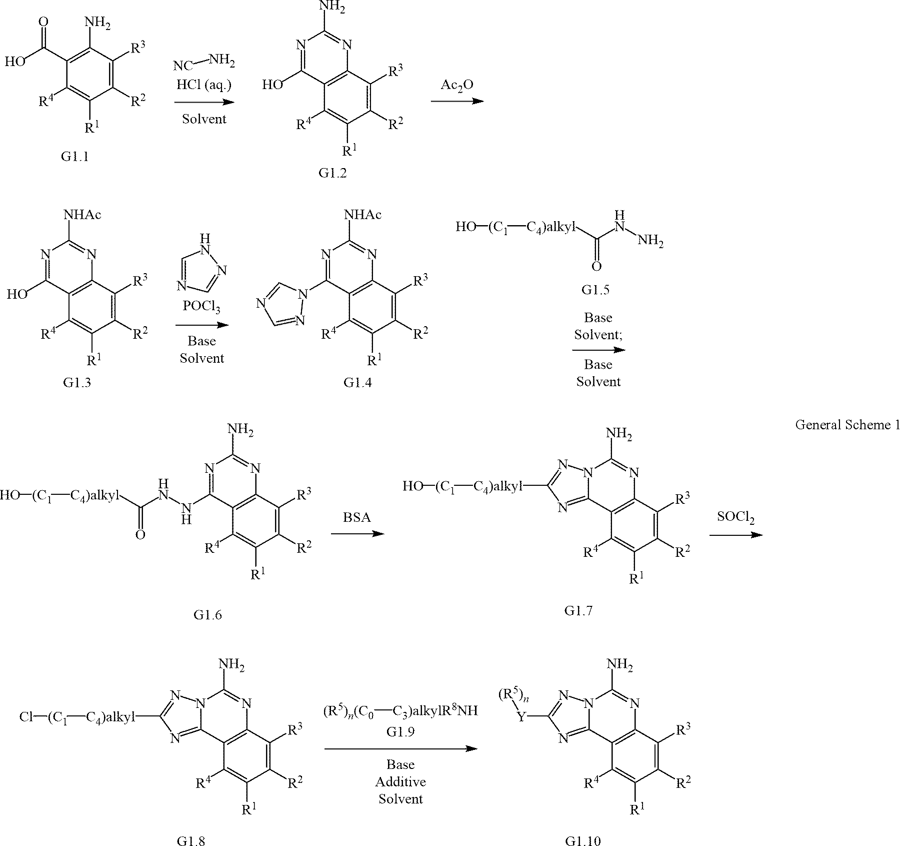

One general strategy for the synthesis of compounds of type G1.10 is via a seven-step procedure shown in General Scheme 1, wherein Y, R¹, R², R³, R⁴, R⁵ and R⁸ are defined in Formula (I). In the first two steps, amino benzoic acids G1.1 can be converted into amino quinazolines G1.3 via treatment with cyanamide in the presence of aqueous HCl in a solvent such as EtOH, followed by subsequent acetylation with Ac₂O. In the third step, intermediates of type G1.3 can be converted into intermediates of type G1.4 through coupling with 1,2,4-triazole, following treatment with POCl₃ in a solvent such as MeCN, and a base such as DIPEA. In the fourth step, intermediates of type G1.4 can be treated with hydrazides G1.5 in a solvent such as THF, and a base such as DIPEA, followed by deprotection with a base such as K₂CO₃ and a solvent such as MeOH to provide products of type G1.6. In the fifth step, intermediates of type G1.6 can undergo a dehydrative cyclization and rearrangement upon heating in neat BSA to form products of type G1.7. In the sixth step, intermediates of type G1.7 can be converted into intermediates of type G1.8 upon heating in neat SOCl₂. In the seventh and final step, intermediates of type G1.8 can be converted into examples of type G1.10 through a displacement reaction with primary or secondary amine nucleophiles G1.9 wherein bases such as DIPEA, Cs₂CO₃, K₂CO₃, and NaH; additives such as NaI, KI, and TBAI; and solvents such as DMF, dioxane, and MeCN can be used. Products of type G1.10 can be purified by trituration, filtering, and washing with an appropriate solvent, preparative TLC, silica gel chromatography, preparative reversed-phase HPLC, and/or chiral SFC. In addition, subsequent manipulations can be performed on G1.10 to provide additional examples.

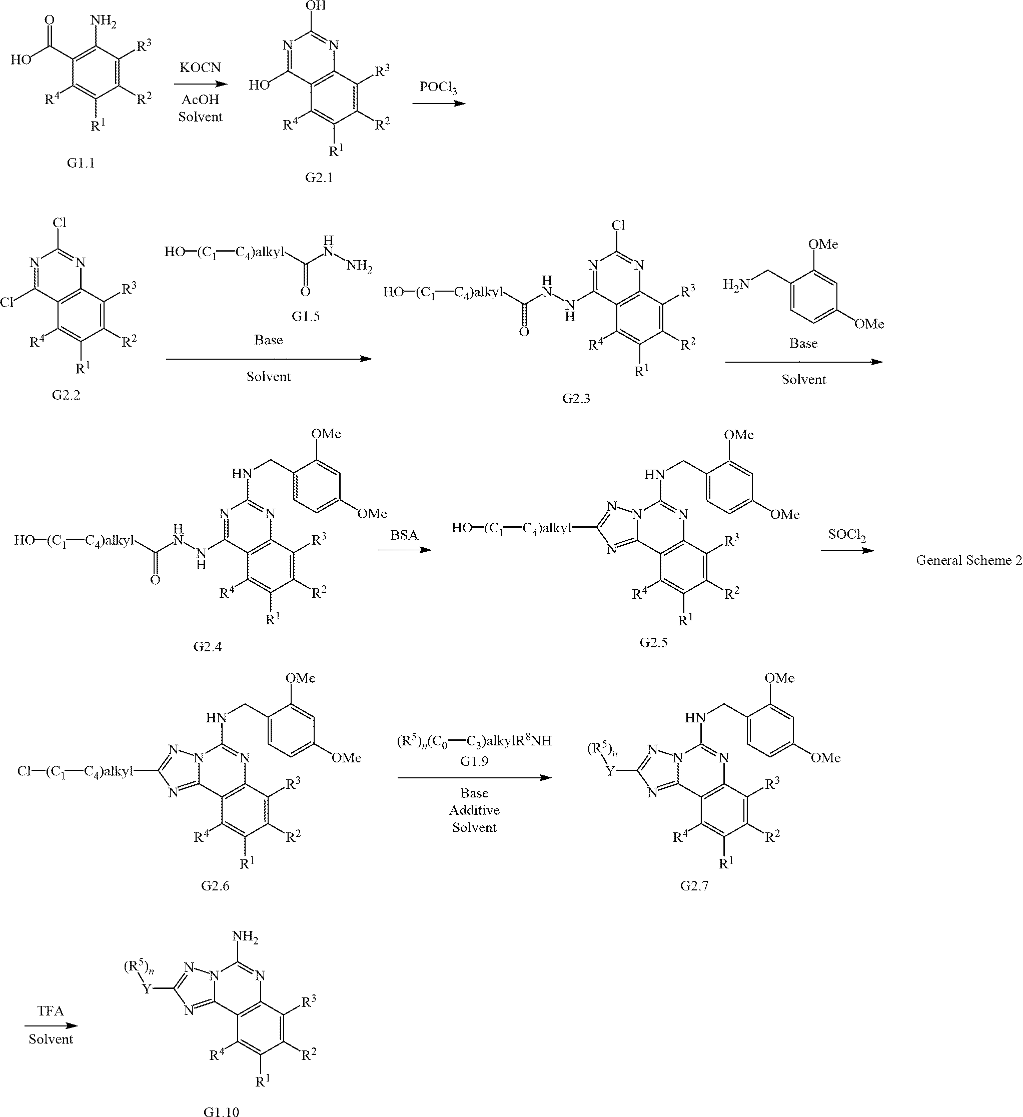

Another general strategy for the synthesis of compounds of type G1.10 is via an eight-step procedure shown in General Scheme 2, wherein Y, R¹, R², R³, R⁴, R⁵ and R⁸ are defined in Formula (I). In the first step, amino benzoic acids G1.1 can be converted into quinazolines G2.1 via treatment with KOCN in the presence of HOAc in a solvent such as water. In the second step, intermediates of type G2.1 can be converted into intermediates of type G2.2, following treatment with POCl₃ in a solvent such as MeCN, and a base such as DIPEA. In the third step, intermediates of type G2.2 can be treated with hydrazides G1.5 in a solvent such as THF, and a base such as DIPEA, to provide products of type G2.3. In the fourth step, 2,4-dimethoxybenzyl amine is added along with a base such as DIPEA in a solvent such as dioxane to generate intermediates of type G2.4. In the fifth step, intermediates of type G2.4 can undergo a dehydrative cyclization and rearrangement upon heating in neat BSA to form products of type G2.5. In the sixth step, intermediates of type G2.5 can be converted into intermediates of type G2.6 upon heating in neat SOCl₂. In the seventh step, intermediates of type G2.6 can be converted into intermediates of type G2.7 through a displacement reaction with primary or secondary amine nucleophiles G1.9 wherein bases such as DIPEA, C_{S2}CO₃, K₂CO₃, and NaH; additives such as NaI, KI, and TBAI; and solvents such as DMF, dioxane, and MeCN can be used. In the eighth and final step, the 2,4-dimethoxybenzyl group of G2.7 is removed via treatment with an acid such as TFA or HCl, either neat or in the presence of a solvent like DCM or water, to provide products of type G1.10. Products of type G1.10 can be purified by trituration, filtering, and washing with an appropriate solvent, preparative TLC, silica gel chromatography, preparative reversed-phase HPLC, and/or chiral SFC. In addition, subsequent manipulations can be performed on G1.10 to provide further elaborated products.

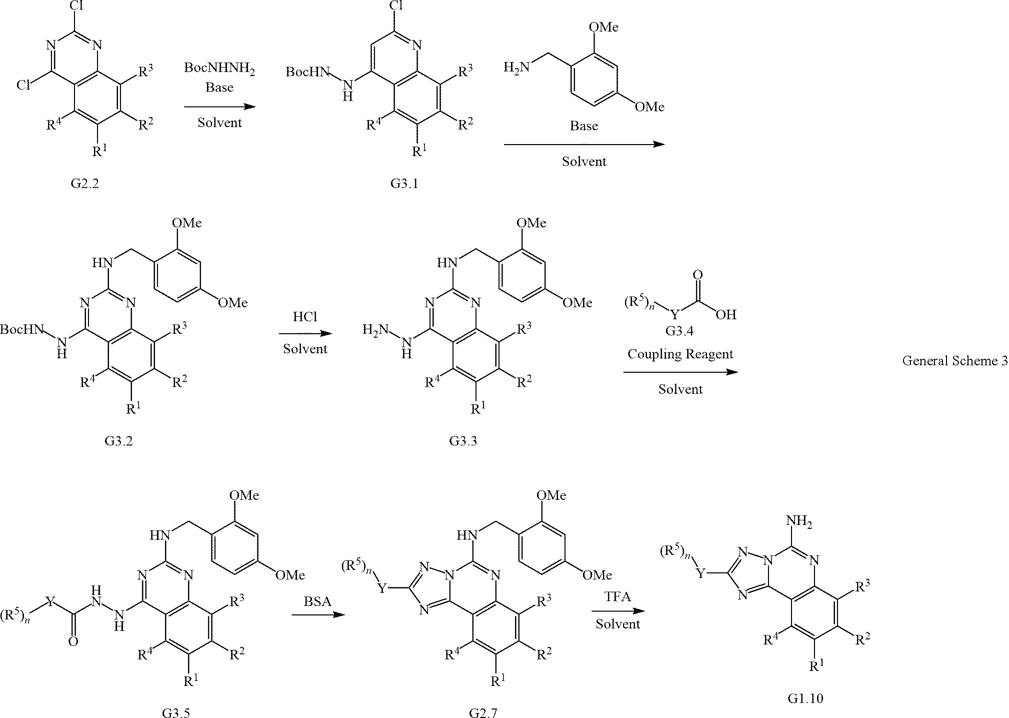

As an extension to the utility of intermediates like G2.2, another general strategy for the synthesis of compounds of type G1.10 is outlined in General Scheme 3, wherein Y, R¹, R², R³, R⁴ and R⁵ are defined in Formula (I). In the first step, analogous to Step 3 in General Scheme 2, intermediates of type G2.2 can be treated with tert-butyl hydrazinecarboxylate in a solvent such as THF, and a base such as DIPEA, to provide products of type G3.1. In the second step, analogous to Step 4 in General Scheme 2, 2,4-dimethoxybenzyl amine is added in along with a base such as DIPEA in a solvent such as dioxane to generate intermediates of type G3.2. In the third step, the Boc group of G3.2 is removed via treatment with dilute HCl, in the presence of a solvent like MeOH, to provide products of type G3.3. In the fourth step, intermediates of type G3.3 can then be combined with acids G3.4 in the presence of a coupling reagent such as T3P® in the presence of a base such as DIPEA, and a solvent such as DCM to produce the coupled products G3.5. In the fifth step, intermediates of type G3.5 can undergo a dehydrative cyclization and rearrangement upon heating in neat BSA to form products of type G2.7, illustrating another route to access these intermediates. In the eighth and final step, the 2,4-dimethoxybenzyl group of G2.7 is removed via treatment with TFA, in the presence of a solvent like DCE, to provide products of type G1.10. Products of type G1.10 can be purified by trituration, filtering, and washing with an appropriate solvent, preparative TLC, silica gel chromatography, preparative reversed-phase HPLC, and/or chiral SFC.

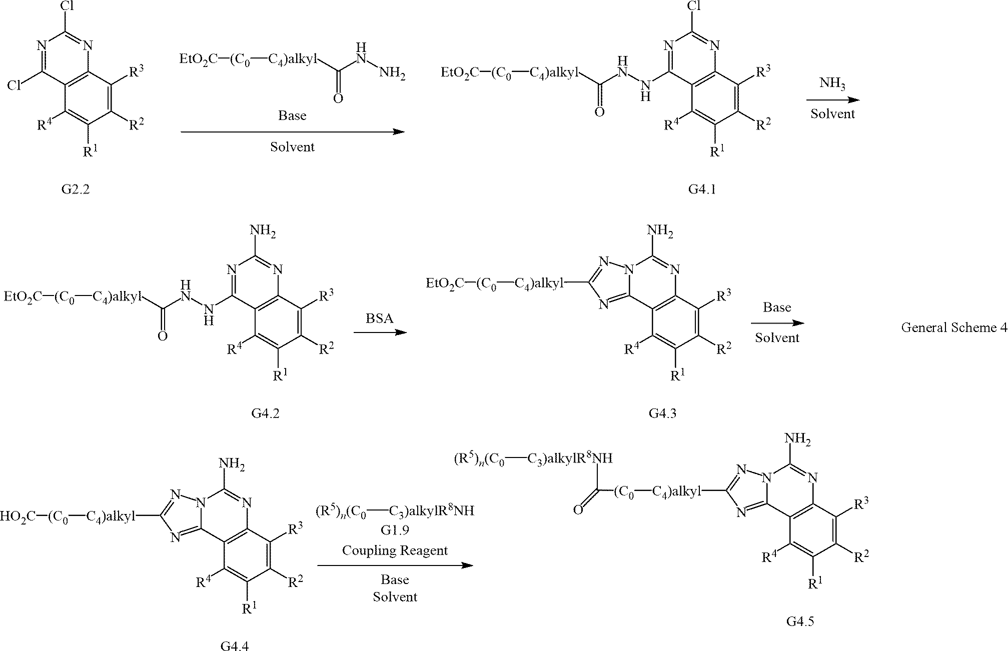

As another extension to the utility of intermediates like G2.2, a general strategy for the synthesis of compounds of type G4.5 is outlined in General Scheme 4, wherein Y, R¹, R², R³, R⁴, and R⁵ are defined in Formula (I). In the first step, analogous to Step 3 in General Scheme 2, intermediates of type G2.2 can be treated with ethyl 3-hydrazinyl-3-oxopropanoate in a solvent such as THF, and a base such as DIPEA, to provide products of type G4.1. In the second step, analogous to Step 4 in General Scheme 2, ammonia is added in a solvent such as i-PrOH to generate intermediates of type G4.2. In the third step, intermediates of type G4.2 can undergo a dehydrative cyclization and rearrangement upon heating in neat BSA to form products of type G4.3. In the fourth step, intermediates of type G4.3 can be treated with LiOH in solvents such as THF and water, to provide products of type G4.4. In the fifth and final step, intermediates of type G4.4 can be combined with amines G1.9 in the presence of a coupling reagent such as T3P® in the presence of a base such as DIPEA, and a solvent such as DMF to provide products of type G4.5. Products of type G4.5 can be purified by trituration, filtering, and washing with an appropriate solvent, preparative TLC, silica gel chromatography, preparative reversed-phase HPLC, and/or chiral SFC.

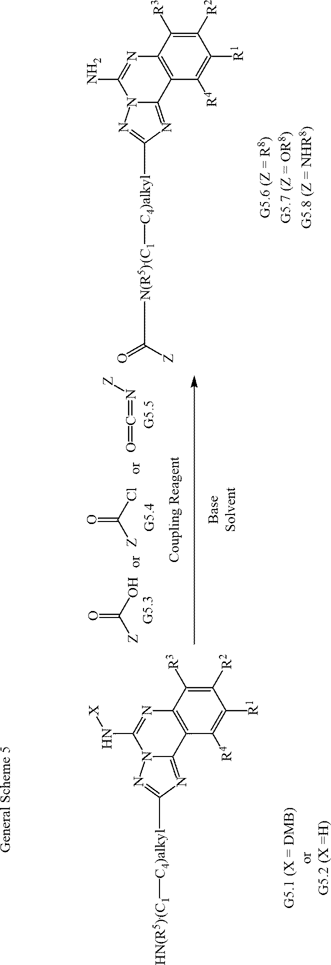

As an extension to the utility of intermediates like G5.1 or G5.2, that can be prepared via methods described in general schemes 1 and 2, one general strategy for the synthesis of compounds of type G5.6-5.8 is shown in General Scheme 5, wherein X is either a hydrogen or dimethoxybenzyl (DMB) group, Z = R⁸, OR⁸, or NHR⁸, and R¹, R², R³, R⁴, R⁵ and R⁸ are defined in Formula (I). In the first step, intermediates of type G5.1 or G5.2 can be treated with either acids G5.3, chloroformates G5.4, or isocyanates G5.5 in the presence of bases such as DIPEA or Et₃N, and solvents such as DMF or DCM to provide the corresponding amide, carbonate, or urea products G5.6, G5.7, and G5.8, respectively. For the synthesis of amides G5.6, coupling reagents such as EDC·HCl and HOBt, HATU, or T3P® are needed to effect the transformation. For products made starting from G5.1 (i.e., when X= DMB), a second transformation is needed to remove the dimethoxybenzyl group. This additional step is analogous to Step 8 of General Scheme 2, wherein the 2,4-dimethoxybenzyl group is removed via treatment with an acid such as TFA or HCl, either neat or in the presence of a solvent like DCM or water. In addition, subsequent manipulations can be done on R⁵, or R⁸ to provide further elaborated products. Products of type G5.6-5.8 can be purified by trituration, filtering, and washing with an appropriate solvent, preparative TLC, silica gel chromatography, preparative reversed-phase HPLC, and/or chiral SFC.

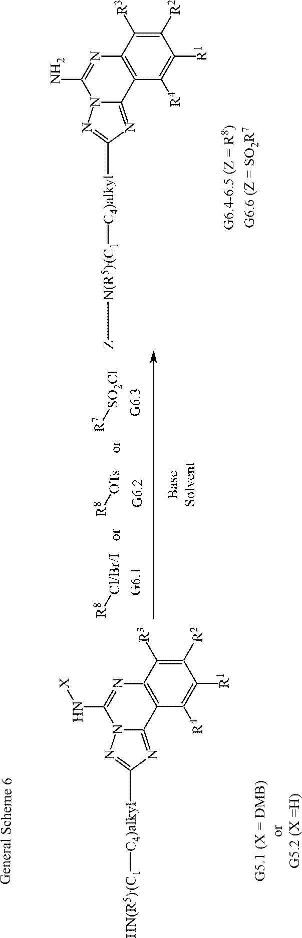

As an extension to the utility of intermediates like G5.1 or G5.2, that can be prepared via methods described in general schemes 1 and 2, one general strategy for the synthesis of compounds of type G6.4-6.6 is shown in General Scheme 6, wherein X is either a hydrogen or dimethoxybenzyl (DMB) group, Z = R⁷ or R⁸, and Y, R¹, R², R³, R⁴, R⁵, R⁷, and R⁸ are defined in Formula (I). In the first step, intermediates of type G5.1 or G5.2 can be treated with either halides G6.1, tosylates G6.2, or sulfonyl chlorides G6.3 in the presence of bases such as K₂CO₃, DIPEA, or Et₃N, and solvents such as DMF, EtOH, or DCM, to provide the corresponding amine, or sulfonamide products G6.4, G6.5, and G6.6, respectively, For products made starting from G2.7 (i.e., when X= DMB), a second transformation is needed to remove the dimethoxybenzyl group. This additional step is analogous to Step 8 of General Scheme 2, wherein the 2,4-dimethoxybenzyl group is removed via treatment with an acid such as TFA either neat or in the presence of a solvent like DCM, or conc. HCl in the presence or absence of water. Products of type G6.4-6.6 can be purified by trituration, filtering, and washing with an appropriate solvent, preparative TLC, silica gel chromatography, preparative reversed-phase HPLC, and/or chiral SFC.

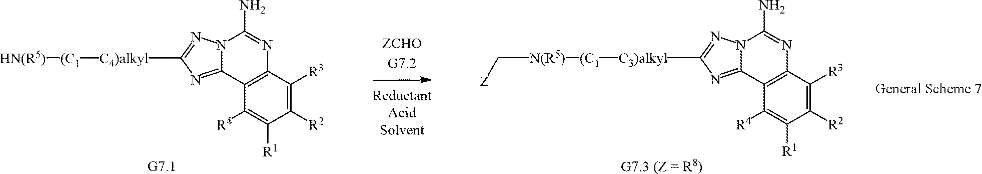

As another extension to the utility of intermediates like G7.1, that can be prepared from the method outlined in General Scheme 1, one general strategy for the synthesis of compounds of type G7.3 is shown in General Scheme 7, wherein Z = R⁸, and Y, R¹, R², R³, R⁴, R⁵, and R⁸ are defined in Formula (I). In the first and only step, intermediates of type G7.1 can be treated with aldehydes G7.2 in the presence of a reductant like NaBH(OAc)₃ or MP-CNBH₃, an acid such as AcOH, and solvents such as MeOH and/or DCM, to afford products of type G7.3. In addition, subsequent manipulations can be done on R⁸ to provide further elaborated products. Products of type G7.3 can be purified by trituration, filtering, and washing with an appropriate solvent, preparative TLC, silica gel chromatography, preparative reversed-phase HPLC, and/or chiral SFC.

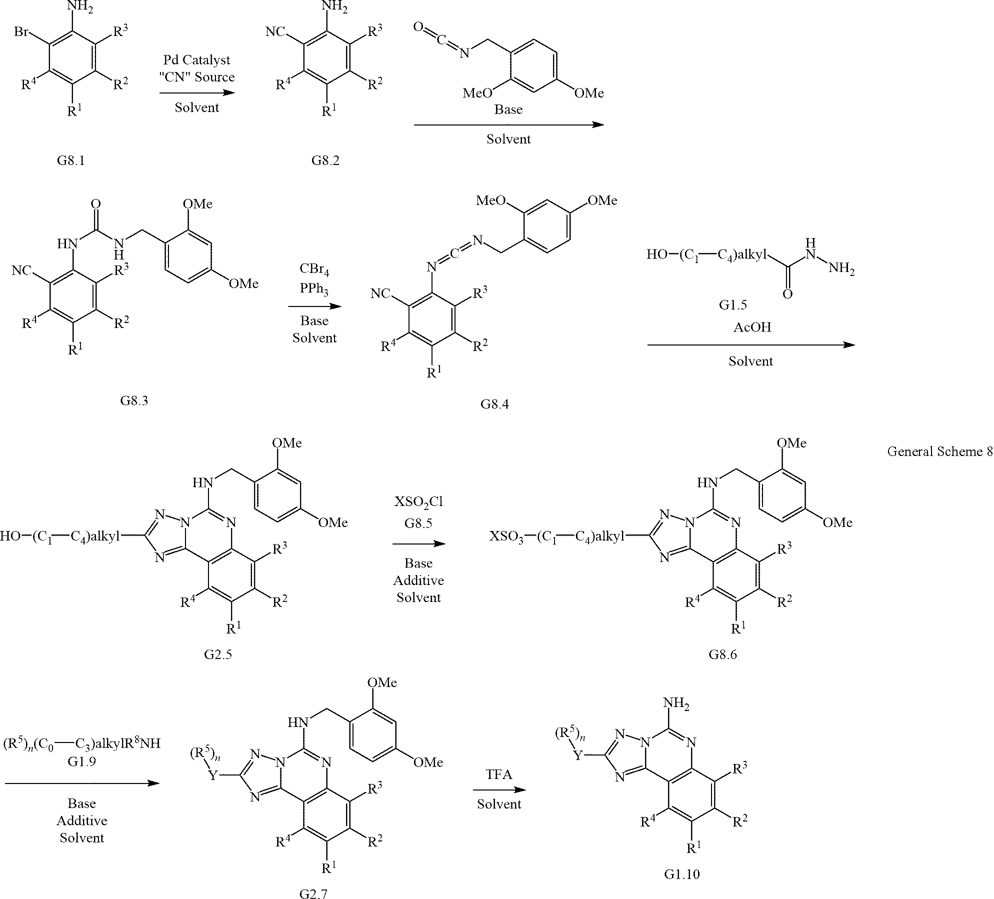

Another general strategy for the synthesis of compounds of type G1.10 is via a seven-step procedure shown in General Scheme 8, wherein X = Me, 4-methylbenzene, or 4-CF₃benzene, and Y, R¹, R², R³, R⁴, R⁵ and R⁸ are defined in Formula (I). In the first step, intermediates of type G8.1 can be converted into intermediates of type G8.2 through a palladium-catalyzed cyanation reaction. The reaction is performed under deoxygenated conditions at the appropriate temperature with palladium catalysts such as Pd(PPh₃)₄, a “CN” source such as Zn(CN)₂, and a solvent such as DMF. In the second step, amino benzonitriles G8.2 can be treated with 1-(isocyanatomethyl)-2,4-dimethoxybenzene in a solvent such as dichloromethane, and a base such as pyridine to form intermediate ureas G8.3. In the third step, ureas G8.3 can be dehydrated to the corresponding carbodiimides G8.4 in the presence of PPh₃, CBr₄, a base such as Et₃N, and a solvent such as DCM. In the fourth step, treatment of carbodiimides G8.4 with a hydrazide of the type G1.5 in the presence of AcOH in a solvent such as DCM, DMF, or dioxane, produces products of the type G2.5, illustrating another route to access these intermediates. In the fifth step, intermediates of type G2.5 can be converted into intermediates of type G8.6 upon treatment with sulfonyl chlorides G8.5 in the presence of a solvent such as THF or DCM, a base such as Et₃N, and an additive such as DMAP. In the sixth step, intermediates of type G8.6 can be treated with amines G1.9 in the presence of bases such as DIPEA or Na₂CO₃, additives such as NaI or KI, and solvents such as DMF or dioxane to provide products of the type G2.7, illustrating another route to access these intermediates. In the seventh and final step, the 2,4-dimethoxybenzyl group of G2.7 is removed via treatment with TFA, in the presence of a solvent like DCE, to provide products of type G1.10. Products of type G1.10 can be purified by trituration, filtering, and washing with an appropriate solvent, preparative TLC, silica gel chromatography, preparative reversed-phase HPLC, and/or chiral SFC.

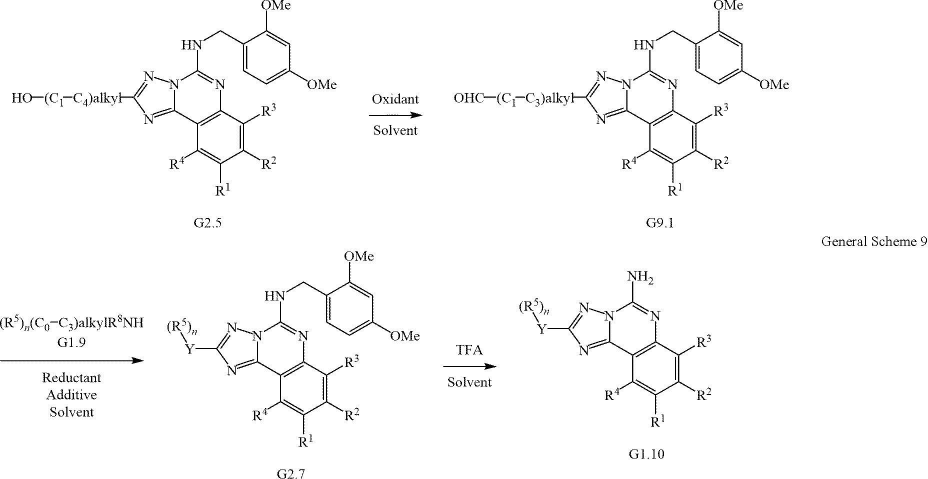

Another general strategy for the synthesis of compounds of type G1.10 is via a three-step procedure shown in General Scheme 9, wherein Y, R¹, R², R³, R⁴ and R⁵ are defined in Formula (I). In the first step, intermediates of type G2.5 can be converted into intermediates of type G9.1 upon treatment with an oxidant such as DMP, and a solvent such as DCM. In the second step, intermediates of type G9.1 can be treated with amines G1.9 in the presence of a reductant like NaCNBH₃, an acid such as AcOH, and solvents such as MeOH and/or DCM, to produce products of type G2.7, illustrating another route to access these intermediates. In the third and final step, the 2,4-dimethoxybenzyl group of G2.7 is removed via treatment with DDQ, in the presence of solvents like DCM and water, to provide products of type G1.10. In addition, subsequent manipulations can be done on R⁵ to provide further elaborated products. Products of type G1.10 can be purified by trituration, filtering, and washing with an appropriate solvent, preparative TLC, silica gel chromatography, preparative reversed-phase HPLC, and/or chiral SFC.

### General Experimental Information

Unless otherwise noted, all reactions were magnetically stirred and performed under an inert atmosphere such as nitrogen or argon.

Unless otherwise noted, diethyl ether used in the experiments described below was Fisher ACS certified material and stabilized with BHT.

Unless otherwise noted, “degassed” refers to a solvent from which oxygen has been removed, generally by bubbling an inert gas such as nitrogen or argon through the solution for 10 to 15 minutes with an outlet needle to normalize pressure.

Unless otherwise noted, “concentrated” means evaporating the solvent from a solution or mixture using a rotary evaporator or vacuum pump.

Unless otherwise noted, flash chromatography was carried out on an ISCO®, Analogix®, or Biotage® automated chromatography system using a commercially available cartridge as the column. Columns were usually filled with silica gel as the stationary phase. Reversed-phase preparative HPLC conditions can be found at the end of the experimental section. Aqueous solutions were concentrated on a Genevac® evaporator or were lyophilized.

Unless otherwise noted, proton nuclear magnetic resonance (¹H NMR) spectra and proton-decoupled carbon nuclear magnetic resonance (¹³C{¹H} NMR) spectra were recorded on 400, 500, or 600 MHz Bruker or Varian NMR spectrometers at ambient temperature. All chemical shifts (δ) were reported in parts per million (ppm). Proton resonances were referenced to residual protium in the NMR solvent, which can include, but is not limited to, CDCl₃, DMSOd₆, and MeOD-d₄. Carbon resonances are referenced to the carbon resonances of the NMR solvent. Data are represented as follows: chemical shift, multiplicity (br = broad, br s = broad singlet, s = singlet, d = doublet, dd = doublet of doublets, ddd = doublet of doublet of doublets, t = triplet, q = quartet, m = multiplet), coupling constants (J) in Hertz (Hz), integration.

### Intermediate A.1, N-(2-(4-bromophenyl)propan-2-yl)-2,2,2-trifluoroacetamide.

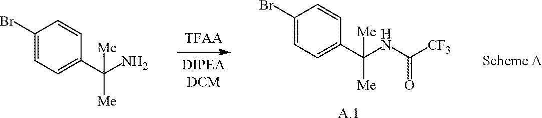

TFAA (0.812 mL, 5.84 mmol) was added dropwise to a suspension of 2-(4-bromophenyl)propan-2-amine (1.00 g, 4.67 mmol) and DIPEA (1.63 mL, 9.34 mmol) in DCM (9.34 mL) over 1 min at room temperature. After 2 h, the reaction was diluted with water (25 mL) and extracted with DCM (3 × 25 mL). The combined organic layers were dried over anhydrous MgSO₄, filtered, and concentrated under reduced pressure. The resulting crude residue was purified by silica gel chromatography (gradient elution: 0-50% [3:1 EtOAc/EtOH]/hexanes) to provide N (2-(4-bromophenyl)propan-2-yl)-2,2,2-trifluoroacetamide, which was used in the subsequent step without further purification. MS (ESI) m/z calc’d for C₁₁H₁₂BrF₃NO [M+H]⁺ 310.0, found 310.0, 312.0.

### Intermediate B.1, 4-(2-hydroxypropan-2-yl)-2,6-dimethylbenzoic Acid.

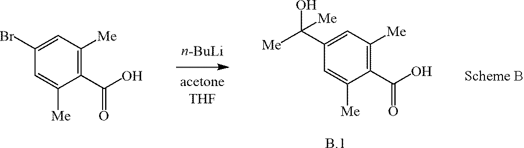

n-butyllithium (1.6 M in hexanes, 3.00 mL, 4.80 mmol) was added dropwise to a solution of 4-bromo-2,6-dimethylbenzoic acid (500 mg, 2.18 mmol) in THF (10.9 mL) over 1 min at -78° C. After 45 min, acetone (0.404 mL, 5.46 mmol) was added at -78° C. The reaction was allowed to warm to room temperature. After 4 h, the reaction was quenched with sat. aq. NH₄Cl (10 mL), diluted with water (10 mL), and extracted with EtOAc (2 × 15 mL). The combined organic layers were dried over anhydrous MgSO₄, filtered, and concentrated under reduced pressure to provide 4-(2-hydroxypropan-2-yl)-2,6-dimethylbenzoic acid, which was used in subsequent steps without further purification. MS (ESI) m/z calc’d for C₁₂H₁₆NaO₃ [M+Na]⁺ 231.1, found 231.2.

The following compound in Table 1 was prepared according to Scheme B starting from intermediate A.1.

TABLE 1

Intermediate Compounds Prepared According to Scheme B

| Entry | Structure | Observed m/z [M + H]⁺ |
|---|---|---|
| | Name | |
| B.2 | 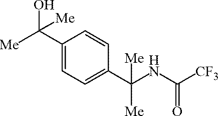 [SMILES: CC(C)(O)c1ccc(C(C)(C)NC(=O)C(F)(F)F)cc1] | 312.1 |
| | 2,2,2-trifluoro-N-(2-(4-(2-hydroxypropan-2-yl)phenyl)propan-2-yl)acetamide | |

### Intermediate C.1, 2-(4-(2-aminopropan-2-yl)phenyl)propan-2-ol.

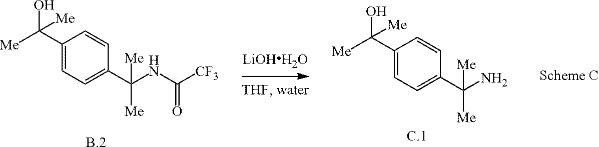

LiOH·H₂O (918 mg, 21.9 mmol) was added to a stirred solution of 2,2,2-trifluoro-N (2-(4-(2-hydroxypropan-2-yl)phenyl)propan-2-yl)acetamide (211 mg, 0.729 mmol) in THF (5.47 mL) and water (1.82 mL). The reaction was stirred vigorously at 65° C. for 48 h. The reaction was then cooled to room temperature, diluted with water (25 mL), and extracted with 3:1 CHCl₃/IPA (2 × 25 mL). The combined organic layers were dried over anhydrous MgSO₄, filtered, and concentrated under reduced pressure to provide 2-(4-(2-aminopropan-2-yl)phenyl)propan-2-ol, which was used in subsequent steps without further purification. MS (ESI) m/z calc’d for C₁₂H₁₈N [M]⁺(—OH fragment) 176.1, found 176.2.

### Intermediate D.1, 3-bromo-5-(2-hydroxypropan-2-yl)picolinic Acid

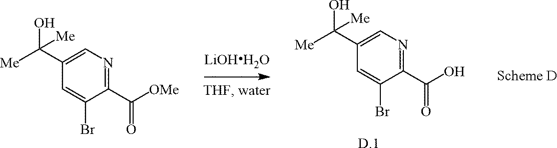

LiOH·H₂O (18.4 mg, 0.440 mmol) was added to a stirred solution of methyl 3-bromo-5-(2-hydroxypropan-2-yl)picolinate (60 mg, 0.220 mmol) in THF (1.65 mL) and water (0.55 mL). The reaction was stirred vigorously at room temperature for 1 h. The reaction was concentrated under reduced pressure to remove the volatiles. The concentrated mixture was cooled to 0° C. and quenched with 6 N aq. HCl (0.110 mL, 0.66 mmol). The reaction was diluted with water (10 mL) and extracted with EtOAc (5 × 15 mL). The combined organic layers were dried over anhydrous MgSO₄, filtered, and concentrated under reduced pressure to provide 3-bromo-5-(2-hydroxypropan-2-yl)picolinic acid, which was used in subsequent steps without further purification. MS (ESI) m/z calc’d for C₉H₁₀BrNO₃ [M+H]⁺ 260.0, found 260.0, 262.0.

### Intermediate E.1, Tert-butyl (3-(2-hydroxypropan-2-yl)bicyclo[1.1.1]pentan-1-yl)carbamate.

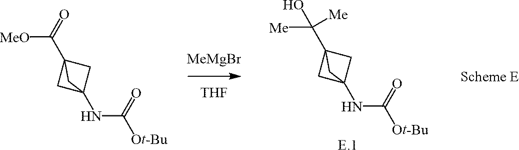

MeMgBr (3.4 M in 2-MeTHF, 1.07 mL, 3.63 mmol) was added dropwise to a stirred solution of methyl 3-((tert-butoxycarbonyl)amino)bicyclo[1.1.1]pentane-1-carboxylate (250 mg, 1.04 mmol) in THF (4.14 mL) over 1 min at 0° C. The reaction was allowed to warm to room temperature. After 4 h, the reaction was quenched with sat. aq. NH₄Cl (5 mL), diluted with water (25 mL), and extracted with EtOAc (3 × 10 mL). The combined organic layers were dried over anhydrous MgSO₄, filtered, and concentrated under reduced pressure to provide tert-butyl (3-(2-hydroxypropan-2-yl)bicyclo[1.1.1]pentan-1-yl)carbamate, which was used in subsequent steps without further purification. MS (ESI) m/z calc’d for C₉H₁₆NO₃ [M+H]⁺ (- t-Bu fragment) 186.1, found 186.1.

### Intermediate F.1, 4-(1,1,1,3,3,3-hexafluoro-2-hydroxypropan-2-yl)benzonitrile.

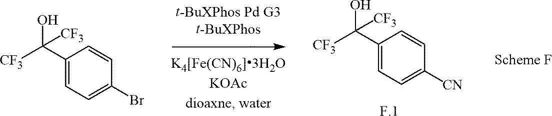

K₄[Fe(CN)₆]·3H₂O (331 mg, 0.90 mmol), KOAc (22 mg, 0.224 mmol), t-BuXPhos (7.6 mg, 0.018 mmol), and t-BuXPhos Pd G3 (14.3 mg, 0.018 mmol) were combined in a reaction vessel. The reaction vessel was sealed and flushed with nitrogen for 5 min, evacuated for 1 min, and backfilled with nitrogen for 1 min. A solution of 2-(4-chlorophenyl)-1,1,1,3,3,3-hexafluoropropan-2-ol (500 mg, 1.795 mmol) in dioxane (4.5 mL) and water (4.5 mL) was then added, and the reaction was degassed by sparging with nitrogen for 15 min, then backfilled with nitrogen for 1 min. The reaction was heated at 100° C. for 1 h in a microwave reactor. The reaction was cooled to room temperature, diluted with brine (25 mL), and extracted with EtOAc (3 × 25 mL). The combined organic layers were dried over anhydrous MgSO₄, filtered, and concentrated under reduced pressure. The resulting crude residue was purified by silica gel chromatography (gradient elution: 0-25% EtOAc/hexanes) to provide 4-(1,1,1,3,3,3-hexafluoro-2-hydroxypropan-2-yl)benzonitrile. MS (ESI) m/z calc’d for C₁₀H₆F₆NO [M+H]⁺ 270.0, found 270.1.

### Intermediate G.1, 4-(2,2,2-trifluoro-1-hydroxyethyl)benzonitrile.

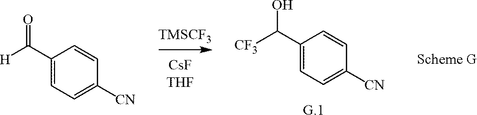

TMSCF₃ (2.0 M in THF, 4.19 mL, 8.39 mmol) was added to a stirred solution of 4-formylbenzonitrile (1.00 g, 7.63 mmol) and CsF (29.0 mg, 0.191 mmol) in THF (76 mL). The reaction was stirred vigorously at room temperature for 4 h. The reaction was then quenched with 6 N aq. HCl (3 mL) and stirred for an additional 16 h. The reaction was diluted with water (50 mL) and extracted with EtOAc (3 × 25 mL). The combined organic layers were dried over anhydrous MgSO₄, filtered, and concentrated under reduced pressure to provide 4-(2,2,2-trifluoro-1-hydroxyethyl)benzonitrile, which was used in the subsequent step without further purification. MS (ESI) m/z calc’d for C₉H₇F₃NO [M+H]⁺ 202.0, found 202.1.

The following compound in Table 2 was prepared according to Scheme G starting from the commercially available ketone.

TABLE 2

Intermediate Compounds Prepared According to Scheme G

| Entry | Structure | Observed m/z [M + H]⁺ |
|---|---|---|
| | Name | |
| G.2 | 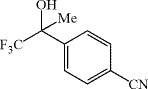 [SMILES: CC(O)(c1ccc(C#N)cc1)C(F)(F)F] | 216.1 |
| | 4-(1,1,1 -trifluoro-2-hydroxypropan-2-yl)benzonitrile | |

### Intermediate H.1, 4-(1,1,3,3-tetrafluoro-2-hydroxypropan-2-yl)benzonitrile.

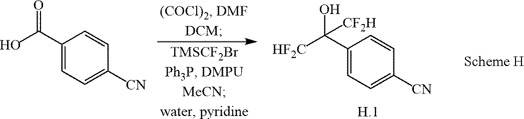

Oxalyl chloride (2 M in DCM, 2.10 mL, 4.20 mmol) was added to a stirred solution of 4-cyanobenzoic acid (441 mg, 3.00 mmol) in DCM (3.00 mL) and DMF (19 µL, 0.245 mmol). The reaction was stirred vigorously at room temperature for 4 h. The reaction was concentrated under reduced pressure and dissolved in MeCN (3.00 mL), at which point, (bromodifluoromethyl) trimethylsilane (1.40 mL, 9.00 mmol), Ph₃P (1.97 g, 7.49 mmol), and DMPU (1.446 mL, 11.99 mmol) were sequentially added. The reaction was stirred vigorously at room temperature for 16 h. Water (3.00 mL) and pyridine (0.970 mL, 12.0 mmol) were added, and the reaction was stirred vigorously at 80° C. for 4 h. The reaction was cooled to room temperature, diluted with water (15 mL), and extracted with MBTE (3 × 15 mL). The combined organic layers were dried over anhydrous MgSO₄, filtered, and concentrated under reduced pressure. The resulting crude residue was purified by silica gel chromatography (gradient elution: 0-75% [3:1 EtOAc/EtOH]/hexanes) to provide 4-(1,1,3,3-tetrafluoro-2-hydroxypropan-2-yl)benzonitrile. MS (ESI) m/z calc’d for C₁₀H₈F₄NO [M+H]⁺ 234.1, found 234.1.

### Intermediates I.1A and 1.1B, Tert-butyl (S)-(4-(2,2,2-trifluoro-1-hydroxyethyl)benzyl)carbamate and Tert-butyl (R)-(4-(2,2,2-trijluoro-1-hydroxyethyl)benzyl)carbamate.

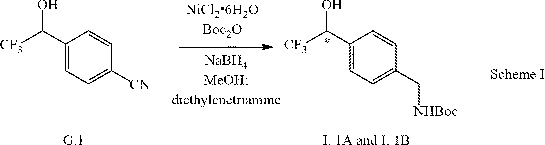

NaBH₄ (2.02 g, 53.4 mmol) was added to a stirred solution of 4-(2,2,2-trifluoro-1-hydroxyethyl)benzonitrile (1.53 g, 7.63 mmol), Boc₂O (3.33 g, 15.3 mmol), and NiCl₂·6H₂O (181 mg, 0.763 mmol) in MeOH (58.7 mL) in 6 equal portions over a period of 30 min at 0° C. The reaction was allowed to warm to room temperature. After 2 h, the reaction was quenched with diethylenetriamine (0.53 mL, 7.63 mmol) and stirred for an additional 30 min. The reaction was concentrated, diluted with sat. aq. NaHCO₃ (25 mL) and extracted with DCM (3 × 25 mL). The combined organic layers were dried over anhydrous MgSO₄, filtered, and concentrated under reduced pressure. The resulting crude residue was purified by silica gel chromatography (gradient elution: 0-50% [3:1 EtOAc/EtOH]/hexanes). The racemic mixture was separated by chiral SFC (Column: AD-H, 21 × 250 mm, 92% MeOH [w/ 1% NH₄OH]/CO₂) to provide Intermediate I.1A (faster eluting) and Intermediate I.1B (slower eluting). Intermediate I.1A (faster eluting): MS (ESI) m/z calc’d for C₁₄H₁₈F₃NNaO₃ [M+Na]⁺ 328.1, found 328.1. Intermediate I.1B (slower eluting): MS (ESI) m/z calc’d for C₁₄H₁₈F₃NNaO₃ [M+Na]⁺ 328.1, found 328.1.

The following compounds in Table 3 were prepared according to Scheme I, starting from the appropriate nitrile intermediates. In addition, any racemic mixtures were not separated by chiral SFC at this time and instead carried forward as a racemic mixture.

TABLE 3

Intermediate Compounds Prepared According to Scheme I

| Entry | Structure | Observed m/z [M + H]⁺ |
|---|---|---|
| | Name | |
| I.2 | 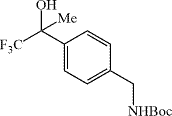 [SMILES: CC(C)(C)OC(=O)NCc1ccc(C(C)(O)C(F)(F)F)cc1] | 342.1 |
| | tert-butyl (4-(1,1,1-trifluoro-2-hydroxypropan-2-yl)benzyl)carbamate | |
| I.3 | 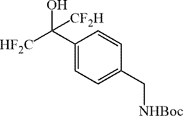 | 360.1 |
| | tert-butyl (4-(1,1,3,3-tetrafluoro-2-hydroxypropan-2-yl)benzyl)carbamate | |
| I.4 | 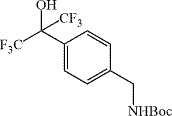 [SMILES: CC(C)(C)OC(=O)NCc1ccc(C(O)(C(F)(F)F)C(F)(F)F)cc1] | 396.1 |
| | tert-butyl (4-(1,1,1,3,3,3-hexafluoro-2-hydroxypropan-2-yl)benzyl)carbamate | |

### Intermediate J.1, 2-(4-(aminomethyl)phenyl)-1,1,3,3-tetrafluoropropan-2-ol hydrochloride.

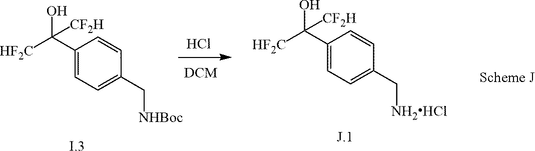

HCl (4 M in dioxane, 1.17 mL, 4.67 mmol) was added to a stirred solution of tert-butyl (4-(1,1,3,3-tetrafluoro-2-hydroxypropan-2-yl)benzyl)carbamate (315 mg, 0.934 mmol) in DCM (3.74 mL). The reaction was stirred vigorously at room temperature for 1 h. The reaction was filtered, and the collected material was washed with diethyl ether (5 × 5 mL). The collected material was further dried under reduced pressure to provide 2-(4-(aminomethyl)phenyl)-1,1,3,3-tetrafluoropropan-2-ol hydrochloride. MS (ESI) m/z calc’d for C₁₀H₁₂F₄NO [M+H]⁺ 238.1, found 238.1.

The following compounds in Table 4 were prepared according to Scheme J starting from the appropriate amine intermediates.

TABLE 4

Intermediate Compounds Prepared According to Scheme J

| Entry | Structure | Observed m/z [M + H]⁺ |
|---|---|---|
| | Name | |
| J.2A | 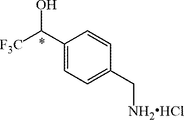 | 206.1 |
| | 1-(4-(aminomethyl)phenyl)-2,2,2-trifluoroethan-1-ol hydrochloride (from Intermediate I.1A) | |
| J.2B | 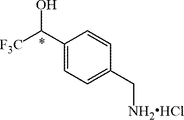 | 206.1 |
| | 1-(4-(aminomethyl)phenyl)-2,2,2-trifluoroethan-1-ol hydrochloride (Intermediate I.1B) | |
| J.3 | 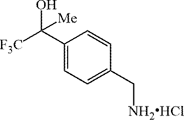 | 220.2 |
| | 2-(4-(aminomethyl)phenyl)-1,1,1-trifluoropropan-2-ol hydrochloride | |
| J.4 | 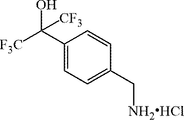 | 274.1 |
| | 2-(4-(aminomethyl)phenyl)-1,1,1,3,3,3-hexafluoropropan-2-ol hydrochloride | |
| J.5 | 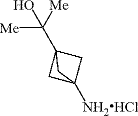 | [M+HCl+Na]⁺ 200.2. |
| | 2-(3-aminobicyclo[1.1.1]pentan-1-yl)propan-2-ol hydrochloride | |

### Intermediate K.5, 1-(3,3-difluorocyclobutyl)-4,5,6,7-tetrahydro-1H-benzo[d][1,2,3]triazol-5-amine.

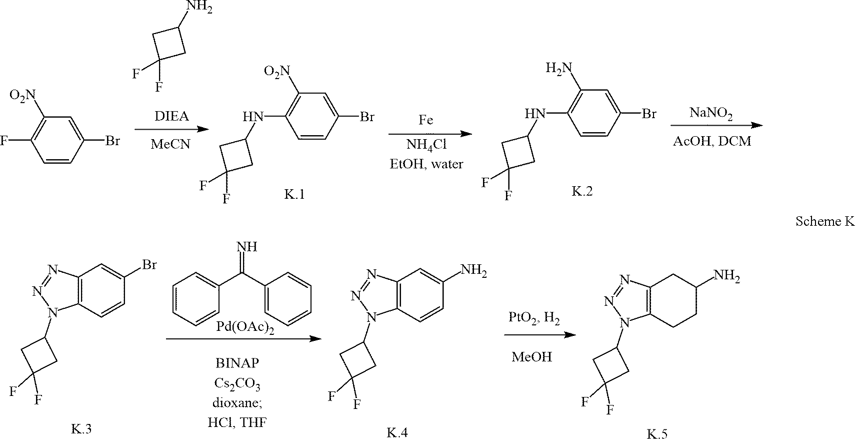

### Step 1 -Intermediate K.1, of 5-bromo-N-(3,3-difluorocyclobutyl)-2-nitroaniline.

3,3-Difluorocyclobutanamine (6.53 g, 45.5 mmol) and DIPEA (12.6 mL, 68.2 mmol) were added to a stirred solution of 4-bromo-2-fluoro-l-nitrobenzene (5.0 g, 22.73 mmol) in MeCN (20 mL) at 80° C. The reaction was stirred vigorously at 80° C. for 10 h. The reaction was cooled to room temperature and concentrated under reduced pressure. The resulting crude residue was purified by silica gel chromatography (gradient elution: 0-30% EtOAc/petroleum ether) to provide of 5-bromo-N-(3,3-difluorocyclobutyl)-2-nitroaniline. MS (ESI) m/z calc’d for C₁₀H₉BrF₂N₂O₂ [M+H]⁺ 309.0, found 309.0.

### Step 2 - Intermediate K.2, 4-bromo-N1-(3,3-difluorocyclobutyl)benzene-1,2-diamine.

Iron (4.44 g, 79.0 mmol) and NH₄Cl (4.25 g, 79.0 mmol) were added to a stirred solution of 5-bromo-N-(3,3-difluorocyclobutyl)-2-nitroaniline (6.10 g, 19.9 mmol) in EtOH (80 mL) and water (80 mL) at 80° C. under an atmosphere of nitrogen. The reaction was heated at 80° C. for 10 h. The reaction was cooled to room temperature, filtered, and concentrated under reduced pressure. The resulting residue was diluted with water (100 mL) and extracted with EtOAc (2 × 100 mL). The combined organic layers were dried over anhydrous Na₂SO₄, filtered, and concentrated under reduced pressure to provide 4-bromo-N¹-(3,3-difluorocyclobutyl)benzene-1,2-diamine, which was used in the subsequent step without further purification. MS (ESI) m/z calc’d for C₁₀H₁₁BrF₂N₂ [M+H]⁺ 279.0, found 279.1.

### Step 3 - Intermediate K.3, 5-bromo-1-(3,3-difluorocyclobutyl)-1H-benzo[d][1,2,3]triazole.

NaNO₂ (2.69 g, 39.0 mmol) was added to a stirred solution of 4-bromo-N¹-(3,3-difluorocyclobutyl)benzene-1,2-diamine (5.40 g, 19.5 mmol) in AcOH (80 mL) and DCM (80 mL) at 0° C. The reaction was stirred vigorously at 0° C. for 1 h. While still at 0° C., the reaction was diluted with water (50 mL) and extracted with DCM (2 × 50 mL). The combined organic layers were dried over anhydrous Na₂SO₄, filtered, and concentrated under reduced pressure. The resulting crude residue was purified by silica gel chromatography (gradient elution: 0-50% EtOAc/petroleum ether) to provide 5-bromo-1-(3,3-difluorocyclobutyl)-1H-benzo[d] [1,2,3]triazole. MS (ESI) m/z calc’d for C₁₀H₈BrF₂N₃ [M+H]⁺ 279.9, found 288.0.

### Step 4 - Intermediate K.4, 1-(3,3-difluorocyclobutyl)-1H-benzo[d][1,2,3]triazol-5-amine.

C_{S2}CO₃ (10.2 g, 31.2 mmol), diphenylmethanimine (4.25 g, 23.4 mmol), BINAP (0.973 g, 1.52 mmol) and Pd(OAc)₂ (0.351 g, 1.56 mmol) was added to a stirred solution of 5-bromo-1-(3,3-difluorocyclobutyl)-1H-benzo[d][1,2,3]triazole (4.5 g, 15.6 mmol) in dioxane (60 mL) under a nitrogen atmosphere. The reaction was heated to 90° C. for 12 h. The reaction was cooled to room temperature, diluted with water (50 mL), and extracted with EtOAc (2 × 50 mL). The combined organic layers were dried over anhydrous Na₂SO₄, filtered, and concentrated under reduced pressure. The resulting crude residue was purified by silica gel chromatography (gradient elution: 0-80% EtOAc/petroleum ether) to provide 1-(3,3-difluorocyclobutyl)-1H-benzo[d|[1,2,3]triazol-5-amine. MS (ESI) m/z calc’d for C₁₀H₁₀F₂N₄ [M+H]⁺ 225.1, found 225.0.

### Step 5 - Intermediate K.5, 1-(3,3-difluorocyclobutyl)-4,5,6,7-tetrahydro-1H-benzo[d][1,2,3]triazol-5-amine.

PtO₂ (2.53 g, 11.15 mmol) was added to a stirred solution of 1-(3,3-difluorocyclobutyl)-1H-benzo[d|[1,2,3]triazol-5-amine (2.5 g, 11.2 mmol) in MeOH (100 mL) under an atmosphere of argon. The reaction was stirred vigorously at 50° C. for 12 h under an atmosphere of H₂ at 50 psi. The reaction was cooled to room temperature, filtered, and concentrated under reduced pressure. The resulting crude residue was purified via reverse phase HPLC [Method A] to provide 1-(3,3-difluorocyclobutyl)-4,5,6,7-tetrahydro-1H-benzo[d][1,2,3]triazol-5-amine. MS (ESI) m/z calc’d for C₁₀H₁₄F₂N₄ [M+H]⁺ 229.1, found 228.9.

### Intermediate L.4, Methyl 2-(5-formyl-2-isopropyl-1H-imidazol-1-yl)acetate.

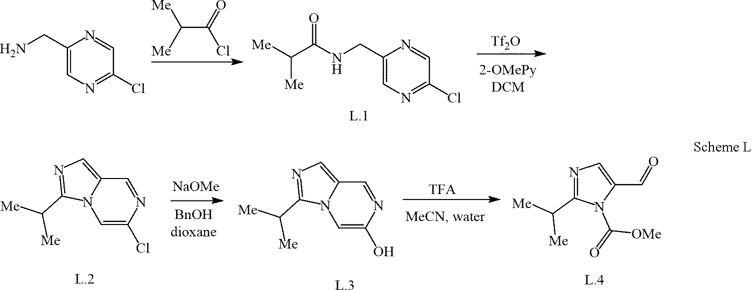

### Step 1 - Intermediate L.1, N-((5-chloropyrazin-2-yl)methyl)isobutyramide.

Et₃N (11.9 mL, 86.0 mmol) and isobutyl chloride (2.95 mL, 28.6 mmol) were added to a stirred solution of (5-chloropyrazin-2-yl)methanamine (4.10 g, 28.6 mmol) in DCM (50 mL). The reaction was stirred vigorously at room temperature for 2 h. The reaction was diluted with water (50 mL) and extracted with DCM (2 × 50 mL). The combined organic layers were dried over anhydrous Na₂SO₄, filtered, and concentrated under reduced pressure. The resulting crude residue was purified by silica gel chromatography (gradient elution: 0-80% EtOAc/petroleum ether) to provide N-((5-chloropyrazin-2-yl)methyl)isobutyramide. MS (ESI) m/z calc’d for C₉H₁₂ClN₃O [M+H]⁺ 214.1, found 214.1.

### Step 2 - Intermediate L.2, 6-chloro-3-isopropylimidazo[1,5-a]pyrazine.

Tf₂O (6.61 mL, 39.3 mmol) was added to a stirred suspension of N-((5-chloropyrazin-2-yl)methyl)isobutyramide (4.2 g, 19.7 mmol) and 2-OMePy (4.29 g, 39.3 mmol) in DCM (20 mL) at 0° C. The reaction was warmed to room temperature. After 2 h, the reaction was quenched with sat. aq. NaHCO₃ (5 mL) and extracted with DCM (2 × 30 mL). The combined organic layers were concentrated under reduced pressure. The resulting crude residue was purified by silica gel chromatography (gradient elution: 0-65% EtOAc/petroleum ether) to provide 6-chloro-3-isopropylimidazo[1,5-α]pyrazine. MS (ESI) m/z calc’d for C₉H₁₀ClN₃ [M+H]⁺ 196.1, found 196.1.

### Step 3 - Intermediate L.3, 3-isopropyl-6-methoxyimidazo[1,5-a]pyrazine.

BnOH (4.15 g, 38.3 mmol) and NaOMe (1.24 g, 23.0 mmol) was added to a stirred solution of 6-chloro-3-isopropylimidazo[1,5-a]pyrazine (1.5 g, 7.67 mmol) in dioxane (40 mL). The reaction was heated to 100° C. for 1 h in a microwave reactor. The reaction was cooled to room temperature and concentrated under reduced pressure. The resulting crude residue was purified by silica gel chromatography (gradient elution: 0-50% EtOAc/petroleum ether) to provide 3-isopropyl-6-methoxyimidazo[1,5-α]pyrazine. MS (ESI) m/z calc’d for C₁₆H₁₇N₃O₃ [M+H]⁺ 268.1, found 268.1.

### Step 4 - Intermediate 1.4, Methyl 2-(5-formyl-2-isopropyl-1H-imidazol-1-yl)acetate.

TFA (40 µL, 0.523 mmol) was added to a stirred solution of 3-isopropyl-6-methoxyimidazo[1,5-α]pyrazine (100 mg, 0.523 mmol) in MeCN (2 mL) and water (2 mL). The reaction was stirred vigorously at room temperature for 1 h. The reaction was quenched with sat. aq. NaHCO₃ (5 mL) and extracted with EtOAc (2 × 5 mL). The combined organic layers were dried over anhydrous Na₂SO₄, filtered, and concentrated under reduced pressure to provide methyl 2-(5-formyl-2-isopropyl-1H-imidazol-1-yl)acetate, which was used in the subsequent step without further purification. ¹H NMR (400 MHz, CDC13) δ 9.61 (s, 1 H), 7.73 (s, 1 H), 3,76 (s, 3 H), 2.98-2.82 (m, 1 H), 1.36-1.28 (m, 6 H).

### Intermediate M.5, N-(2,4-dimethoxybenzyl)-4-hydrazinyl-8-methoxyquinazolin-2-amine.

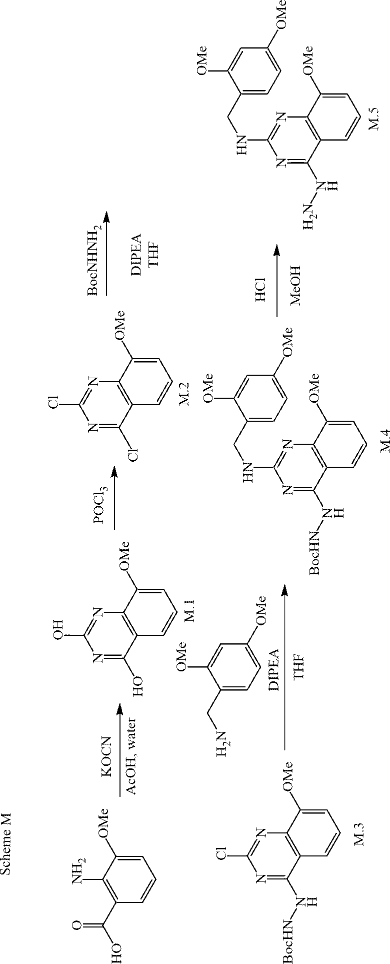

### Step 1 - Intermediate M.1, of 8-methoxyquinazoline-2,4-diol

A solution of KOCN (175 g, 2.15 mol) in water (700 mL) was added to a suspension of 2-amino-3-methoxybenzoic acid (150 g, 0.9 mol) in water (3000 mL) and AcOH (55 mL, 0.96 mol) at 55° C. The reaction was stirred vigorously at 55° C. for 16 h. The reaction was then cooled to room temperature and stirred for an additional 3 h. The reaction was then cooled to 0° C. and acidified to pH 5-6 with conc. HCl. The resulting solid was filtered, washed with water, and dried under reduced pressure to provide 8-methoxyquinazoline-2,4-diol, which was used in the subsequent step without further purification.

### Step 2 - Intermediate M.2, 2,4-dichloro-8-methoxyquinazoline.

POCl₃ (315 mL, 3.38 mol) was added to 8-methoxyquinazoline-2,4-diol (50 g, 0.26 mol). The reaction was heated at 105° C. for 16 h. The reaction was cooled to room temperature and concentrated under reduced pressure. The reaction mixture was diluted with toluene and concentrated under reduced pressure. This process was repeated 3 times. The resulting residue was diluted with EtOAc and washed with sat. aq. NaHCO₃. The combined organic layers were washed with brine, dried over anhydrous MgSO₄, filtered, and concentrated under reduced pressure to provide 2,4-dichloro-8-methoxyquinazoline, which was used in the subsequent step without further purification.

### Step 3 - Intermediate M.3, Tert-butyl 2-(2-chloro-8-methoxyquinazolin-4-yl)hydrazine-1-carboxylate.

DIPEA (9.2 mL, 0.052 mol) and tert-butyl hydrazinecarboxylate (5.8 g, 0.044 mol) were added to a stirred solution of 2,4-dichloro-8-methoxyquinazoline (10 g, 0.044 mol) in THF (200 mL). The reaction was stirred vigorously at 65° C. for 16 h. The reaction was cooled to room temperature and concentrated under reduced pressure to provide tert-butyl 2-(2-chloro-8-methoxyquinazolin-4-yl)hydrazine-1-carboxylate, which was used in the subsequent step without further purification.

### Step 4 - Intermediate M.4, Tert-butyl 2-(2-((2,4-dimethoxybenzyl)amino)-8-methoxyquinazolin-4-yl)hydrazine-1-carboxylate.

DIPEA (19 mL, 0.34 mol) and (2,4-dimethoxyphenyl)methanamine (9.46 g, 0.057 mol) were added to a suspension of tert-butyl 2-(2-chloro-8-methoxyquinazolin-4-yl)hydrazine-1-carboxylate in dioxane (100 mL). The reaction was heated at 100° C. for 16 h. The reaction was cooled to room temperature, concentrated under reduced pressure, and diluted with water. The resulting mixture stirred vigorously at room temperature for 30 min. The reaction was filtered, washed with dioxane and hexanes, and dried under reduced pressure to provide tert-butyl 2-(2-((2,4-dimethoxybenzyl)amino)-8-methoxyquinazolin-4-yl)hydrazine-1-carboxylate, which was used in the subsequent step without further purification.

### Step 5 - Intermediate M.5, N-(2,4-dimethoxybenzyl)-4-hydrazinyl-8-methoxyquinazolin-2-amine.

HCl (2.5 M in MeOH, 1L, 2.5 mol) was added to tert-butyl 2-(2-((2,4-dimethoxybenzyl)amino)-8-methoxyquinazolin-4-yl)hydrazine-1-carboxylate (60 g, 0.132 mol). The reaction was stirred vigorously at room temperature for 16 h. The reaction was diluted with triethanolamine (1.5 L), and the resulting solid was filtered, washed with triethanolamine, and dried under reduced pressure to provide N-(2,4-dimethoxybenzyl)-4-hydrazinyl-8-methoxyquinazolin-2-amine, which was used in subsequent steps without further purification. ¹H NMR (300 MHz, DMSO-d₆): δ 11.43 (s, 1H), 8.27 (s, 1H), 7.80-7.78 (d, 1H), 7.39-7.25 (m, 3H), 6.61 (d, 1H), 6.50-6.47 (d, 1H), 4.69-4.67 (d, 2H), 3.96 (s,3H), 3.86 (s, 3H), 3.76 (s, 3H).

### Intermediate N.4, 2-(5-amino-7-methoxy-[1,2,4]triazolo[1,5-c]quinazolin-2-yl)acetic acid.

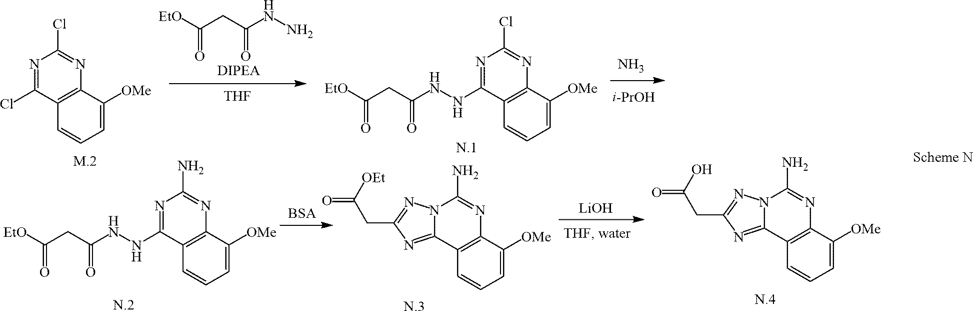

### Step 1- Intermediate N.1, Ethyl 3-(2-(2-chloro-8-methoxyquinazolin-4-yl)hydrazinyl)-3-oxopropanoate.

Ethyl 3-hydrazinyl-3-oxopropanoate (2.01 g, 13.75 mmol) and DIPEA (6.86 ml, 39.3 mmol) were added to a stirred suspension of 2,4-dichloro-8-methoxyquinazoline (3.0 g, 13.10 mmol) in THF (30 mL). The reaction was stirred vigorously at 55° C. for 16 h. The reaction was cooled to room temperature and concentrated under reduced pressure. The concentrated residue was diluted in water and extracted with DCM (3x). The combined organic layers were concentrated under reduced pressure to provide ethyl 3-(2-(2-chloro-8-methoxyquinazolin-4-yl)hydrazinyl)-3-oxopropanoate, which was used in the subsequent step without further purification.

### Step 2 - Intermediate N.2, Ethyl 3-(2-(2-amino-8-methoxyquinazolin-4-yl)hydrazinyl)-3-oxopropanoate.

NH₃ (2 M in i-PrOH, 90 mL) was added to ethyl 3-(2-(2-chloro-8-methoxyquinazolin-4-yl)hydrazinyl)-3-oxopropanoate (2.1 g, 6.20 mmol). The reaction was heated at 105° C. for 16 h. The reaction was cooled to room temperature and concentrated under reduced pressure to afford ethyl 3-(2-(2-amino-8-methoxyquinazolin-4-yl)hydrazinyl)-3-oxopropanoate, which was used in the subsequent step without further purification.

### Step 3 - Intermediate N.3, Ethyl 2-(5-amino-7-methoxy-[1,2,4]triazolo[1,5-c]quinazolin-2-yl)acetate.

BSA (20.9 mL, 85 mmol) was added to ethyl 3-(2-(2-amino-8-methoxyquinazolin-4-yl)hydrazinyl)-3-oxopropanoate (2.72 g, 8.54 mmol). The reaction was stirred vigorously at 130° C. for 16 h. The reaction was cooled to room temperature and concentrated under reduced pressure. The resulting crude residue was purified by silica gel chromatography (gradient elution: 50% EtOAc/hexanes-10% MeOH/DCM) to provide, ethyl 2-(5-amino-7-methoxy-[1,2,4]triazolo[1,5-c]quinazolin-2-yl)acetate.

### Step 4 - Intermediate N.4, 2-(5-amino-7-methoxy-[1,2,4]triazolo[1,5-c]quinazolin-2-yl)acetic Acid,

LiOH was added to a stirred suspension of ethyl 2-(5-amino-7-methoxy-[1,2,4]triazolo[1,5-c]quinazolin-2-yl)acetate from the previous step in THF (20 mL) and water (4 mL). The reaction was stirred vigorously at room temperature for 16 h. The reaction was concentrated under reduced pressure to 2-(5-amino-7-methoxy-[1,2,4]triazolo[1,5-c]quinazolin-2-yl)acetic acid, which was used in subsequent steps without further purification. MS (ESI) m/z calc’d for C₁₂H₁₂N₅O₃ [M+H]⁺ 274.1, found 274.1.

The following compounds in Table 5 were prepared according to Steps 1-3 in Scheme N starting from Intermediate M.2, the appropriate hydrazide for Step 1, and the appropriate amine in Step 2. For Step 2, (2,4-dimethoxyphenyl)methanamine was used as the amine, dioxane was used instead of i-PrOH, and the reaction was run in the presence of DIPEA.

TABLE 5

Intermediate Compounds Prepared According to Scheme N

| Entry | Structure | Observed m/z [M + H]⁺ |
|---|---|---|
| | Name | |
| N.5 | 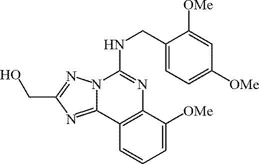 [SMILES: COc1ccc(CNc2nc3c(OC)cccc3c3nc(CO)nn23)c(OC)c1] | 396 |
| | (5-((2,4-dimethoxybenzyl)amino)-7-methoxy-[1,2,4]triazolo[1,5-c]quinazolin-2-yl)methanol | |
| N.6 | 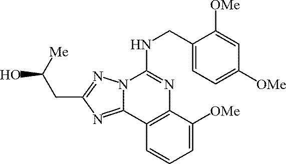 [SMILES: COc1ccc(CNc2nc3c(OC)cccc3c3nc(C[C@H](C)O)nn23)c(OC)c1] | 424.3 |
| | (S)-1-(5-((2,4-dimethoxybenzyl)amino)-7-methoxy-[1,2,4]triazolo[1,5-c]quinazolin-2-yl)propan-2-ol | |

### Intermediate O.5, (5-amino-7-methoxy-[1,2,4]triazolo[1,5-c]quinazolin-2-yl)methanol

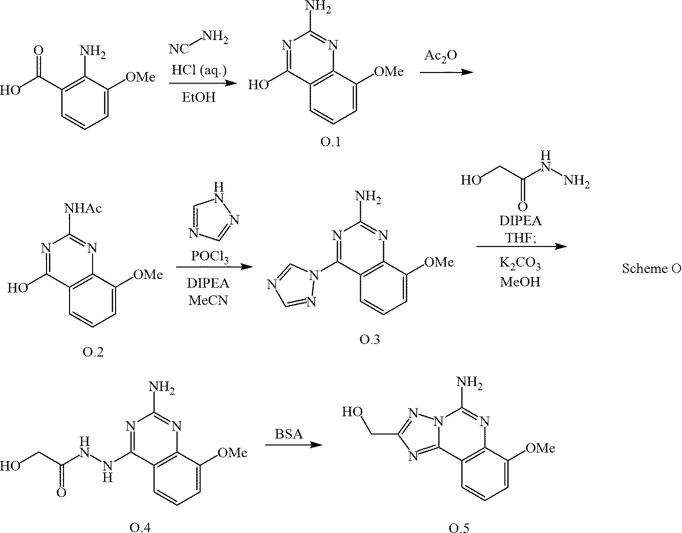

### Step 1 - Intermediate O.1, of 2-amino-8-methoxyquinazolin-4-ol.

Cyanamide (18.9 g, 449 mmol) and HCl (37% aq., 12 mL, 299 mmol) were added to a suspension of 2-amino-3,5-difluorobenzoic acid (50.0 g, 299 mmol) in EtOH (400 mL). The reaction was stirred vigorously at 80° C. for 16 h. The reaction was cooled to room temperature and the resulting solids were filtered and washed with cold EtOH to provide 2-amino-8-methoxyquinazolin-4-ol, which was used in the subsequent step without further purification.

### Step 2 - Intermediate O.2, N-(4-hydroxy-8-methoxyquinazolin-2-yl)acetamide

Ac₂O (60 mL, 52.3 mmol) was added to 2-amino-8-methoxyquinazolin-4-ol (10 g, 52.3 mmol). The reaction was heated at 130° C. for 40 min to provide N-(4-hydroxy-8-methoxyquinazolin-2-yl)acetamide, which was used in the subsequent step without further purification.

### Step 3 - Intermediate O.3, 8-methoxy-4-(1H-1,2,4-triazol-1-yl)quinazolin-2-amine.

POCl₃ (8.22 mL, 90 mmol) was added dropwise to a stirred solution of N-(4-hydroxy-8-methoxyquinazolin-2-yl)acetamide (7.0 g, 30.0 mmol), 1,2,4-triazole (20.7 g, 300 mmol), and DIPEA (15.3 mL, 90 mmol) in MeCN (300 mL). The reaction was stirred vigorously at room temperature for 16 h. The resulting solids were filtered and washed with EtOH and diethyl ether to provide 8-methoxy-4-(1H-1,2,4-triazol-1-yl)quinazolin-2-amine, which was used in the subsequent step without further purification.

### Step 4 - Intermediate O.4, N′-(2-aniino-8-methoxyquinazolin-4-yl)-2-hydroxyacetohydrazide.

2-Hydroxyacetohydrazide (1.39 g, 15.5 mmol) and DIPEA (1.71 g, 17.9 mmol) were added to a stirred suspension of 8-methoxy-4-(1H-1,2,4-triazol-1-yl)quinazolin-2-amine (4.0 g, 14.1 mmol) in THF (300 mL). The reaction was stirred vigorously at 60° C. for 48 h. The reaction was cooled to room temperature and concentrated under reduced pressure. The concentrated residue was dissolved in MeOH (200 mL) and water (100 mL). K₂CO₃ was added, and the reaction was stirred vigorously at 65° C. for 2 h. The reaction was cooled to room temperature and concentrated under reduced pressure. The resulting solids were filtered, washed with cold water and DCM/hexanes, and dried under reduced pressure to provide N′-(2-amino-8-methoxyquinazolin-4-yl)-2-hydroxyacetohydrazide, which was used in the subsequent step without further purification. MS (ESI) m/z calc’d for C₁₁H₁₄N₅O₃ [M+H]⁺ 264, found 264.

### Step 5 - Intermediate O.5, (5-amino-7-methoxy-[1,2,4]triazolo[1,5-c]quinazolin-2-yl)methanoL

BSA (100 mL) was added to N′-(2-amino-8-methoxyquinazolin-4-yl)-2-hydroxyacetohydrazide (3.5 g, 13.3 mmol) The reaction was heated to 120° C. for 3 h. The reaction was cooled to room temperature, concentrated under reduced pressure, and diluted with MeOH. The resulting solids were suspended in MeOH, cooled, filtered, washed with MeOH, and dried under reduced pressure to provide (5-amino-7-methoxy-[1,2,4]triazolo[1,5-c]quinazolin-2-yl)methanol, which was used in subsequent steps without further purification. MS (ESI) m/z calc’d for C₁₁H₁₂N₅O₂ [M+H]⁺ 246, found 246.

The following compounds in Table 6 were prepared according to Scheme O starting from the appropriate carboxylic acid in Step 1, or the appropriate hydrazide in Step 4. For 0.8, Step 2, and thus the second part (i.e. one-pot deprotection) in Step 4, was omitted.

TABLE 6

Intermediate Compounds Prepared According to Scheme O

| Entry | Structure | Observed m/z [M + H]⁺ |
|---|---|---|
| | Name | |
| O.6 | 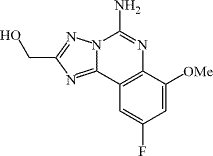 [SMILES: COc1cc(F)cc2c1nc(N)n1nc(CO)nc21] | 264.1 |
| | (5-amino-9-fluoro-7-methoxy-[1,2,4]triazolo[1,5- c]quinazolin-2-yl)methanol | |
| O.7 | 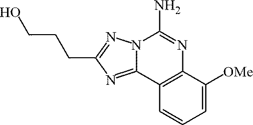 [SMILES: COc1cccc2c1nc(N)n1nc(CCCO)nc21] | 274.2 |
| | 3-(5-amino-7-methoxy-[1,2,4]triazolo[1,5-c]quinazolin-2-yl)propan-1-ol | |
| O.8 | 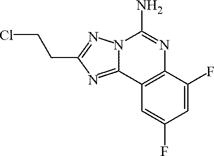 [SMILES: Nc1nc2c(F)cc(F)cc2c2nc(CCCl)nn12] | 274.2 |
| | 2-(2-chloroethyl)-7,9-difluoro-[1,2,4]triazolo[1,5-c]quinazolin-5-amine | |

### Intermediate P.1, 2-(chloromethyl)-7-methoxy-[1,2,4]triazolo[1,5-c]quinazolin-5-amine.

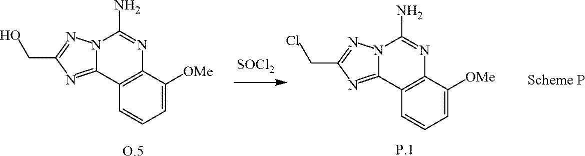

SOCl₂ (10 mL) was added to (5-amino-7-methoxy-[1,2,4]triazolo[1,5-c]quinazolin-2-yl)methanol (2.8 g, 11.4 mmol). The reaction was stirred vigorously at 65° C. for 45 min. The reaction was then cooled to room temperature and concentrated under reduced pressure. The resulting residue was suspended in DCM. The resulting solids were filtered and dried under reduced pressure to provide 2-(chloromethyl)-7-methoxy-[1,2,4]triazolo[1,5-c]quinazolin-5-amine, which was used in subsequent steps without further purification. MS (ESI) m/z calc’d for C₁₁H₁₁ClN₅O [M+H]⁺ 264, found 264.

The following compounds in Table 7 were prepared according to Scheme P starting from the appropriate alcohol intermediates.

TABLE 7

Intermediate Compounds Prepared According to Scheme P

| Entry | Structure | Observed m/z [M + H]⁺ |
|---|---|---|
| | Name | |
| P.2 | 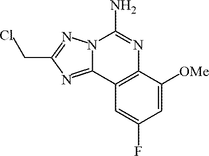 [SMILES: COc1cc(F)cc2c1nc(N)n1nc(CCl)nc21] | 282.1 |
| | 2-(chloromethyl)-9-fluoro-7-methoxy-[1,2,4]triazolo[1,5-c]quinazolin-5-amine | |
| P.3 | 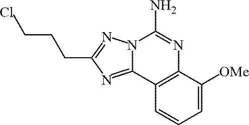 [SMILES: COc1cccc2c1nc(N)n1nc(CCCCl)nc21] | 292.1 |
| | 2-(3-chloropropyl)-7-methoxy-[1,2,4]triazolo[1,5-c]quinazolin-5-amine | |
| P.4 | 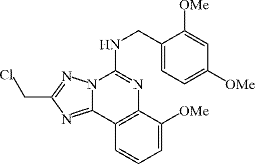 [SMILES: COc1ccc(CNc2nc3c(OC)cccc3c3nc(CCl)nn23)c(OC)c1] | 414.2 |
| | 2-(chloromethyl)-N-(2,4-dimethoxybenzyl)-7-methoxy-[1,2,4]triazolo[1,5-c]quinazolin-5-amine | |

### Intermediate Q.2, 2-(aminomethyl)-7-methoxy-[1,2,4]triazolo[1,5-c]quinazolin-5-amine.

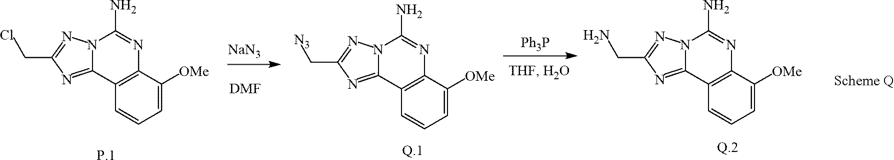

### Step 1- Intermediate Q.1, 2-(azidomethyl)-7-methoxy-[1,2,4]triazolo[1,5-c]quinazolin-5-amine.

NaN₃ (78 mg, 1.20 mmol) was added to a stirred solution of 2-(chloromethyl)-7-methoxy-[1,2,4]triazolo[1,5-c]quinazolin-5-amine (210 mg, 0.80 mmol) in DMF (3.2 mL). The reaction was stirred vigorously at room temperature for 4 h. The reaction was diluted with water (50 mL) and extracted with EtOAc (3 × 15 mL). The combined organic layers were dried over anhydrous MgSO₄, filtered, and concentrated under reduced pressure to provide 2-(azidomethyl)-7-methoxy-[1,2,4]triazolo[1,5-c]quinazolin-5-amine, which was used in the subsequent step without further purification. MS (ESI) m/z calc’d for C₁₁H₁₁N₈O [M+H]⁺ 271.1, found 271.1.

### Step 2 - Intermediate Q.2, 2-(aminomethyl)-7-methoxy-[1,2,4]triazolo[1,5-c]quinazolin-5-amine.

Solid-supported Ph₃P (2.05 mmol/g, 417 mg, 1.60 mmol) was added to a stirred solution of 2-(azidomethyl)-7-methoxy-[1,2,4]triazolo[1,5-c]quinazolin-5-amine (215 mg, 0.800 mmol) in THF (14.5 mL) and water (1.45 mL). The reaction was stirred vigorously at 60° C. for 1.5 h. The reaction was cooled to room temperature, diluted with water (10 mL) and EtOAc (10 mL), filtered through Celite®, and concentrated under reduced pressure to remove the volatiles. The concentrated mixture was extracted with 3:1 CHCl₃/IPA (6 × 25 mL). The combined organic layers were dried over anhydrous MgSO₄, filtered, and concentrated under reduced pressure to provide 2-(amimomethyl)-7-methoxy-[1,2,4]triazolo[1,5-c]quinazolin-5-amine, which was used in subsequent steps without further purification. MS (ESI) m/z calc’d for C₁₁H₁₃N₆O [M+H]⁺ 245.1, found 245.1.

### Intermediate R.1, N-(2,4-dimethoxybenzyl)-7-methoxy-2-((methylamino)methyl)-[1,2,4]triazolo[1,5-c]quinazolin-5-amine.

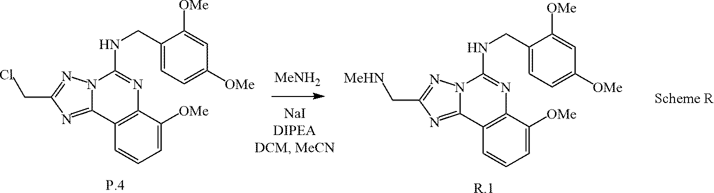

A solution of MeNH₂ (2.0 M in THF, 1.21 mL, 2.42 mmol) was added to a suspension of 2-(chloromethyl)-N-(2,4-dimethoxybenzyl)-7-methoxy-[1,2,4]tnazolo[1,5-c]quinazolin-5-amine (500 mg, 1.21 mmol), NaI (362 mg, 2.42 mmol), DIPEA (0.633 mL, 3.62 mmol) in DCM (12.0 mL) and MeCN (4.0 mL). The reaction was stirred vigorously at 50° C. for 24 h. The reaction was cooled to room temperature, diluted with water (25 mL), and extracted with DCM (3 × 25 mL). The combined organic layers were dried over anhydrous MgSO₄, filtered, and concentrated under reduced pressure. The resulting crude residue was purified by silica gel chromatography (gradient elution: 0-20% MeOH/DCM) to provide N-(2,4-dimethoxybenzyl)-7-methoxy-2-((methylamino)methyl)-[1,2,4]triazolo[1,5-c]quinazolin-5-amine. MS (ESI) m/z calc’d for C₂₁H₂₅N₆O₃ [M+H]⁺ 409.2, found 409.2.

The following compounds in Table 8 were prepared according to Scheme R starting from Intermediate P.4 and the appropriate amine. Intermediate R.2 was prepared in the absence of NaI and dioxane was used instead of DCM and MeCN. Intermediate R.3 was prepared in the absence of NaI and dioxane and water was used instead of DCM and MeCN.

TABLE 8

Intermediate Compounds Prepared According to Scheme R

| Entry | Structure | Observed m/z [M + Na]⁺ |
|---|---|---|
| | Name | |
| R.2 | 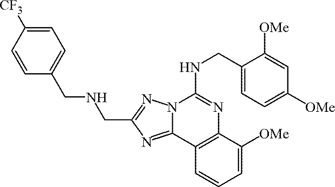 | 607.2 |
| | N-(2,4-dimethoxybenzyl)-7-methoxy-2-(((4-(trifluoromethyl)benzyl)amino)methyl)-[1,2,4]triazolo[1,5-c]quinazolin-5-amine | |
| R.3 | 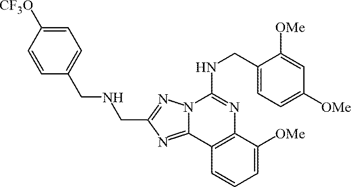 | 569.2 |
| | N-(2,4-dimethoxybenzyl)-7-methoxy-2-(((4-(trifluoromethoxy)benzyl)amino)methyl)-[1,2,4]triazolo[1,5-c]quinazolin-5-amine | |

### Intermediate 5.3, 2-((((2,4-dimethoxybenzyl)imino)methylene)amino)-5-fluoro-4-methoxybenzonitrile.

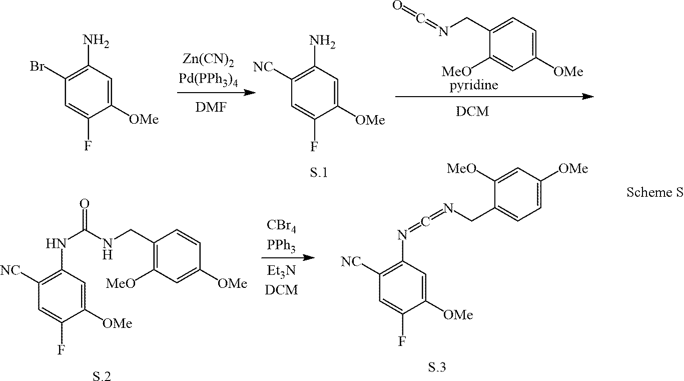

### Step 1 - Intermediate 5.1, 2-amino-5-fluoro-4-methoxybenzonitrile.

Zn(CN)₂ (327 g, 2.78 mol) and Pd(PPh₃)₄ (90.0 g, 0.0778 mol) were added to a stirred solution of 2-bromo-4-fluoro-5-methoxyaniline (300 g, 1.36 mol) in DMF (2.1 L). The mixture was degassed under vacuum and purged with nitrogen. The reaction was stirred vigorously at 130° C. for 1 h. The reaction was cooled to room temperature, diluted with ice water (4 L) and extracted with EtOAc (3 L, 2 L, 1 L). The combined organic layers were washed with brine (2 L, 1.5 L), dried over anhydrous Na₂SO₄, filtered, and concentrated under reduced pressure. The resulting crude residue was purified by silica gel chromatography (gradient elution: 0-50% EtOAc/petroleum ether) to provide 2-amino-5-fluoro-4-methoxybenzonitrile. MS (ESI) m/z calc’d for C₈H₈FN₂O [M+H]⁺ 167, found 167.

### Step 2 - Intermediate 5.2, 1-(2-cyano-4-fluoro-5-methoxyphenyl)-3-(2,4-dimethoxybenzyl)urea.

1-(Isocyanatomethyl)-2,4-dimethoxybenzene (1425 mg, 7.380 mmol) was added to a stirred solution of 2-amino-5-fluoro-4-methoxybenzonitrile (817 mg, 4.92 mmol) and pyridine (1 mL) in DCM (6 mL). The reaction was stirred vigorously at 40° C. for 16 h. The reaction was cooled to room temperature, and the resulting solids were filtered and washed with MeOH (3 × 3 mL), to provide 1-(2-cyano-4-fluoro-5-methoxyphenyl)-3-(2,4-dimethoxybenzyl)urea, which was used in the subsequent step without further purification.

### Step 3 - Intermediate 5.3, 2-((((2,4-dimethoxybenzyl)imino)methylene)amino)-5-fluoro-4-methoxybenzonitrile.

A solution of CBr₄ (2.14 g, 6.44 mmol) in DCM (5 mL) was added to a stirred solution of 1-(2-cyano-4-fluoro-5-methoxyphenyl)-3-(2,4-dimethoxybenzyl)urea (1.16 g, 3.22 mmol), PPh₃ (1.69 g, 6.44 mmol), and Et₃N (1.80 ml, 12.9 mmol) in DCM (25 mL) dropwise at 0° C. The reaction was stirred vigorously at 0° C. for 30 min. The reaction was concentrated, and the resulting residue was purified by silica gel chromatography (gradient elution: 0-70% EtOAc/hexanes) to provide 2-((((2,4-dimethoxybenzyl)imino)methylene)amino)-5-fluoro-4-methoxybenzonitrile. MS (ESI) m/z calc’d for Cl₈H₁₆FN₃NaO₃ [M+Na]⁺ 364, found 364.

The following compounds in Table 9 were prepared according to Scheme S starting from the appropriate commercially available starting materials.

TABLE 9

Intermediate Compounds Prepared According to Scheme S

| Entry | Structure | Observed m/z [M + Na]⁺ |
|---|---|---|
| | Name | |
| S.4 | 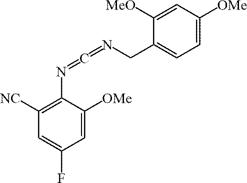 [SMILES: COc1ccc(CN=C=Nc2c(C#N)cc(F)cc2OC)c(OC)c1] | 364 |
| | 2-((((2,4-dimethoxybenzyl)imino)methylene)amino)-5-fluoro-3-methoxybenzonitrile | |

### Intermediate T.1, 2-(5-((2,4-dimethoxybenzyl)amino)-9-fluoro-8-methoxy-[1,2,4]triazolo[1,5-c]quinazolin-2-yl)ethanol.

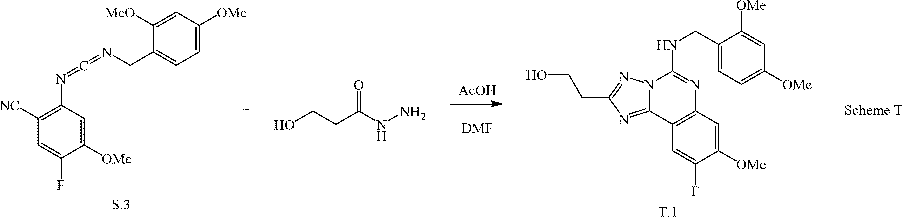

2-((((2,4-dimethoxybenzyl)imino)methylene)amino)-5-fluoro-4-methoxybenzonitrile (3.28 g, 9.61 mmol) and AcOH (0.275 mL, 4.80 mmol) were added to a stirred solution of 3-hydroxypropanehydrazide (1.00 g, 9.61 mmol) in DMF (40 mL). The reaction was stirred vigorously at 40° C. for 15 h. The reaction was cooled to room temperature and concentrated under reduced pressure to remove the volatiles. The concentrated mixture was purified by silica gel chromatography (gradient elution: 0-70% EtOAc/petroleum ether) to provide 2-(5-((2,4-dimethoxybenzyl)amino)-9-fluoro-8-methoxy-[1,2,4]triazolo[1,5-c]quinazolin-2-yl)ethanol. MS (ESI) m/z calc’d for C₂₁H₂₂FN₅O₄ [M+H]⁺ 428.2, found 428.2.

The following compounds in Table 10 were prepared according to Scheme T starting from the appropriate benzonitrile intermediates and hydrazines.

TABLE 10

Intermediate Compounds Prepared According to Scheme T

| Entry | Structure | Observed m/z [M + H]⁺ |
|---|---|---|
| | Name | |
| T.2 | 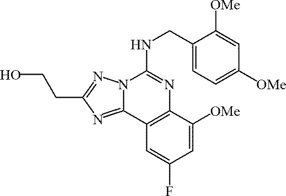 [SMILES: COc1ccc(CNc2nc3c(OC)cc(F)cc3c3nc(CCO)nn23)c(OC)c1] | 428.2 |
| | 2-(5-((2,4-dimethoxybenzyl)amino)-9-fluoro-7-methoxy-[1,2,4]triazolo[1,5-c]quinazolin-2-yl)ethan-1-ol | |
| T.3 | 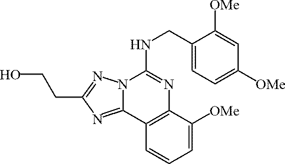 [SMILES: COc1ccc(CNc2nc3c(OC)cccc3c3nc(CCO)nn23)c(OC)c1] | 410.1 |
| | 2-(5-((2,4-dimethoxybenzyl)amino)-7-methoxy-[1,2,4]triazolo[1,5-c]quinazolin-2-yl)ethan-1-ol | |
| T.4 | 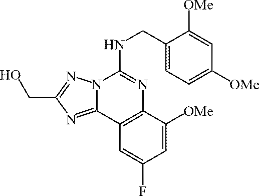 [SMILES: COc1ccc(CNc2nc3c(OC)cc(F)cc3c3nc(CO)nn23)c(OC)c1] | 414.1 |
| | (5-((2,4-dimethoxybenzyl)amino)-9-fluoro-7-methoxy-[1,2,4]triazolo[1,5-c]quinazolin-2-yl)methanol | |
| T.5 | 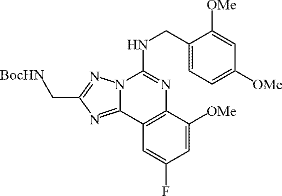 [SMILES: COc1ccc(CNc2nc3c(OC)cc(F)cc3c3nc(CNC(=O)OC(C)(C)C)nn23)c(OC)c1] | 513.6 |
| | tert-butyl((5-((2,4-dimethoxybenzyl)amino)-9-fluoro-7-methoxy-[1,2,4]triazolo[1,5-c]quinazolin-2-yl)methyl)carbamate | |

### Intermediate U.1, 5-((2,4-dimethoxybenzyl)amino)-9-fluoro-7-methoxy[1,2,4]triazolo[1,5-c]quinazoline-2-carbaldehyde.

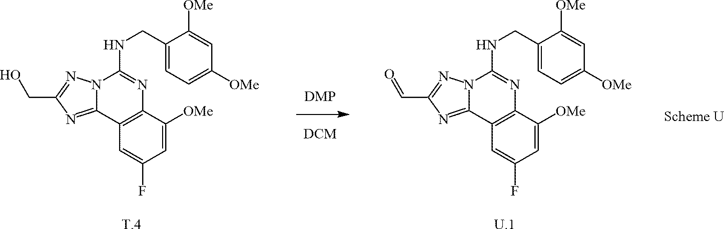

DMP (693 mg, 1.63 mmol) was added to a stirred solution of (5-((2,4-dimethoxybenzyl)amino)-9-fluoro-7-methoxy-[1,2,4]triazolo[1,5-c]quinazolin-2-yl)methanol (450 mg, 1.09 mmol) in DCM (10 mL) at 0° C. The reaction was stirred vigorously at room temperature for 2 h. The reaction was filtered and concentrated under reduced pressure. The resulting crude residue was purified by silica gel chromatography (gradient: 0-60% EtOAc/petroleum ether) to provide 5-((2,4-dimethoxybenzyl)amino)-9-fluoro-7-methoxy-[1,2,4]triazolo[1,S-c]quinazoline-2-carbaldehyde. MS (ESI) m/z calc’d for C₂₀H₁₉FN₅O₄ [M+H]⁺ 412.1, found 412.1.

### Intermediate V.1, 2-(5-((2,4-dimethoxybenzyl)amino)-7-methoxy-[1,2,4]triazolo[1,5-c]quinazolin-2-yl)ethyl methanesulfonate.

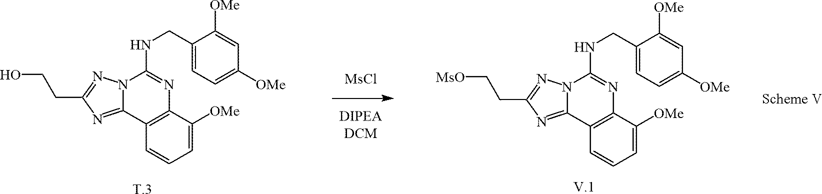

Et₃N (3.06 mL, 22.0 mmol) and MsCl (0.856 mL, 11.0 mmol) was added to a stirred solution of 2-(5-((2,4-dimethoxybenzyl)amino)-7-methoxy-[1,2,4]triazolo[1,5-c]quinazolin-2-yl)ethan-1-ol (3.0 g, 7.33 mmol) in DCM (73 mL) at 0° C. The reaction was stirred vigorously at room temperature for 2 h. The reaction was diluted with sat. aq. NaHCO₃ and extracted with DCM. The combined organic layers were dried over anhydrous MgSO₄, filtered, and concentrated under reduced pressure. The resulting crude residue was purified by silica gel chromatography (gradient: EtOAc/hexanes) to provide 2-(5-((2,4-dimethoxybenzyl)amino)-7-methoxy-[1,2,4]triazolo[1,5-c]quinazolin-2-yl)ethyl methanesulfonate. MS (ESI) m/z calc’d for C₂₂H₂₆N₅O₆S [M+H]⁺ 488, found 488.

### Intermediate W.1, 2-(5-((2,4-dimethoxybenzyl)amino)-7-methoxy-[1,2,4]triazolo[1,5-c]quinazolin-2-yl)ethyl 4-methylbenzenesulfonate.

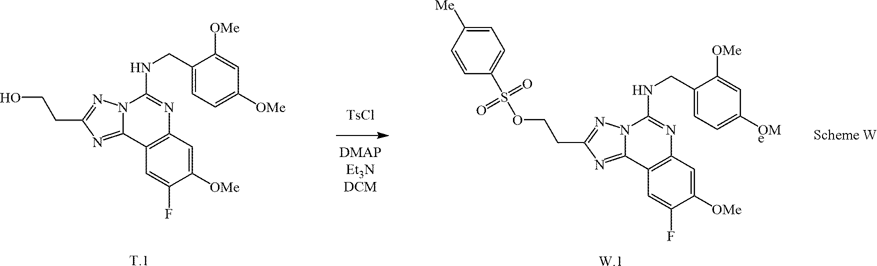

TsCl (126 mg, 0.659 mmol) was added to a stirred solution of Et₃N (0.15 mL, 1.1 mmol), DMAP (81 mg, 0.66 mmol) and 2-(5-((2,4-dimethoxybenzyl)amino)-7-methoxy-[1,2,4]triazolo[1,5-c]quinazolin-2-yl)ethanol (180 mg, 0.440 mmol) in DCM (2 mL) at 0° C. The reaction was warmed to room temperature. After 8 h, the reaction was diluted with brine (10 mL) and extracted with DCM (10 mL). The combined organic layers were dried over anhydrous Na₂SO₄, filtered, and concentrated under reduced pressure. The resulting crude residue was purified by silica gel chromatography (gradient elution: 0-50% EtOAc/petroleum ether) to provide 2-(5-((2,4-dimethoxybenzyl)amino)-7-methoxy-[1,2,4]triazolo[1,5-c]quinazolin-2-yl)ethyl 4-methylbenzenesulfonate, which was used in the subsequent step without further purification. MS (ESI) m/z calc’d for C₂₈H₂₉N₅O₆S [M+H]⁺ 564.2, found 564.3.

### Intermediate X.1, 2-(5-((3,4-dimethylbenzyl)amino)-9-fluoro-8-methoxy-[1,2,4] triazolo[1,5-c]quinazolin-2-yl)ethyl 4-(trifluoromethyl)benzenesulfonate.

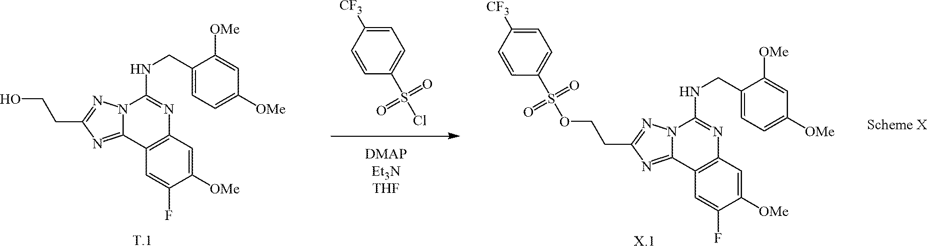

DMAP (13.43 mg, 0.110 mmol), Et₃N (167 mg, 1.649 mmol) and 4-(trifluoromethyl)benzene-1-sulfonyl chloride (202 mg, 0.825 mmol) were added to a stirred solution of 2-(5-((2,4-dimethoxybenzyl)amino)-9-fluoro-8-methoxy-[1,2,4]triazolo[1,5-c]quinazolin-2-yl)ethanol (235 mg, 0.550 mmol) in DCM (15 mL). The reaction was stirred vigorously at room temperature for 15 h. The reaction was filtered, diluted with water (10 mL), and extracted with EtOAc (3 × 15 mL). The combined organic layers were dried over anhydrous Na₂SO₄, filtered, and concentrated under reduced pressure. The resulting crude residue was purified by silica gel chromatography (gradient elution: 0-70% EtOAc/petroleum ether) to provide 2-(5-((3,4-dimethylbenzyl)amino)-9-fluoro-8-methoxyy-[1,2,4]triazolo[1,5-c]quinazolin-2-yl)ethyl 4-(trifluoromethyl)benzenesulfonate. MS (ESI) m/z calc’d for C₂₈H₂₅F₄N₅O₆S [M+H]⁺ 636.2, found 636.2.

The following compounds in Table 11 were prepared according to Scheme X starting from the appropriate alcohol intermediates.

TABLE 11

Intermediate Compounds Prepared According to Scheme X

| Entry | Structure | Observed m/z [M + H]⁺ |
|---|---|---|
| | Name | |
| X.2 | 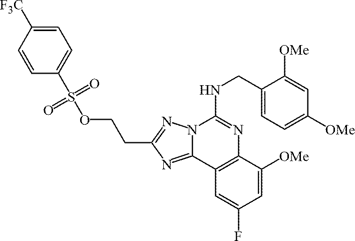 [SMILES: COc1ccc(CNc2nc3c(OC)cc(F)cc3c3nc(CCOS(=O)(=O)c4ccc(C(F)(F)F)cc4)nn23)c(OC)c1] | 636.2 |
| | 2-(5-((2,4-dimethoxybenzyl)amino)-9-fluoro-7-methoxy-[1,2,4]triazolo[1,5-c]quinazolin-2-yl)ethyl 4-(trifluoromethyl)benzenesulfonate | |
| X.3 | 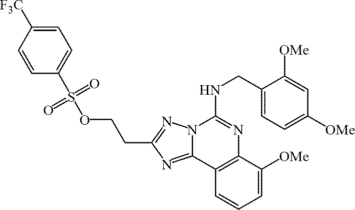 [SMILES: COc1ccc(CNc2nc3c(OC)cccc3c3nc(CCOS(=O)(=O)c4ccc(C(F)(F)F)cc4)nn23)c(OC)c1] | 618.1 |
| | 2-(5-((2,4-dimethoxybenzyl)amino)-7-methoxy-[1,2,4]triazolo[1,5-c]quinazolin-2-yl)ethyl 4-(trifluoromethyl)benzenesulfonate | |
| X.4 | 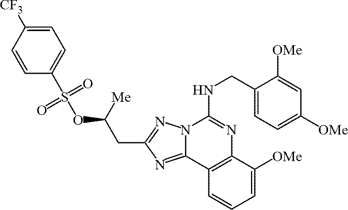 [SMILES: COc1ccc(CNc2nc3c(OC)cccc3c3nc(C[C@H](C)OS(=O)(=O)c4ccc(C(F)(F)F)cc4)nn23)c(OC)c1] | 632.3 |
| | (S)-1-(5-((2,4-dimethoxybenzyl)amino)-7-methoxy-[1,2,4]triazolo[1,5-c]quinazolin-2-yl)propan-2-yl 4-(trifluoromethyl)benzenesulfonate | |

### Intermediate Y.2, 2-(2-aminoethyl)-N-(2,4-dimethoxybenzyl)-9-fluoro-7-methoxy-[1, 2, 4]triazolo[l, 5-c]quinazolin-5-amine.

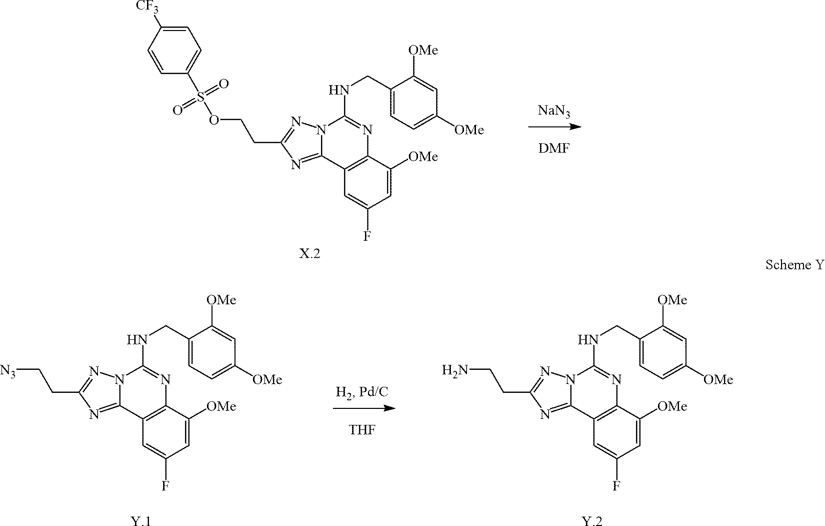

### Step 1 - Intermediate Y.1, 2-(2-azidoethyl)-N-(2,4-dimethoxybenzyl)-9-fluoro-7-methoxy-[1,2,4]triazolo[1,5-c]quinazolin-5-amine.

NaN₃ (20 mg, 0.308 mmol) was added to a stirred solution of 2-(5-((2,4-dimethoxybenzyl)amino)-9-fluoro-7-methoxy-[1,2,4]triazolo[1,5-c]quinazolin-2-yl)ethyl 4-(trifluoromethyl)benzenesulfonate (100 mg, 0.157 mmol) in DMF (2 mL). The reaction was stirred vigorously at 65° C. for 10 h. The reaction was cooled to room temperature, diluted with water (5 mL) and extracted with EtOAc (3 × 5 mL). The combined organic layers were dried over anhydrous Na₂SO₄, filtered, and concentrated under reduced pressure to provide 2-(2-azidoethyl)-7V-(2,4-dimethoxybenzyl)-9-fluoro-7-methoxy-[l,2,4]triazolo[l,5-c]quinazolin-5-amine, which was used in the subsequent step without further purification. MS (ESI) m/z calc’d for C₂₁H₂₁FN₈O₃ [M+H]⁺ 453.1, found 453.1.

### Step 2 - Intermediate Y.2, 2-(2-aminoethyl)-N-(2,4-dimethoxybenzyl)-9-fluoro-7-methoxy-[1,2,4]triazolo[1,5-c]quinazolin-5-amine.

Pd/C (10%, 16.5 mg, 0.015 mmol) was added to a stirred solution of 2-(2-azidoethyl)-N-(2,4-dimethoxybenzyl)-9-fluoro-7-methoxy-[1,2,4]triazolo [1,5-c]quinazolin-5-amine (70 mg, 0.155 mmol) in THF (5 mL). The reaction was stirred vigorously at room temperature for 2 h under an atmosphere of H₂ at 15 psi. The reaction was filtered and concentrated under reduced pressure to provide 2-(2-aminoethyl)-N-(2,4-dimethoxybenzyl)-9-fluoro-7-methoxy-[1,2,4]triazolo[1,5-c]quinazolin-5-amine, which was used in subsequent steps without further purification. MS (ESI) m/z calc’d for C₂₁H₂₃FN₆O₃ [M+H]⁺ 427.1, found 427.3.

### Intermediate Z.1 2-(aminomethyl)-N-(2,4-dimethoxybenzyl)-9-fluoro-7-methoxy-[1,2,4]triazolo[1,5-c]quinazolin-5-amine.

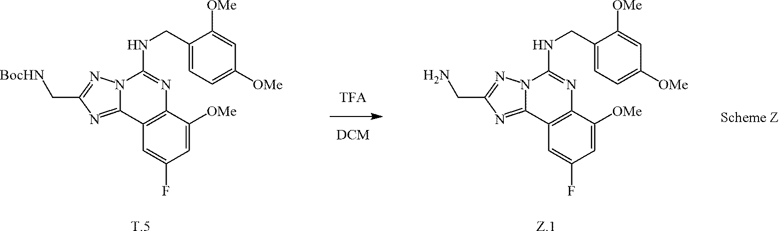

TFA (0.4 mL) was added to a stirred solution of tert-butyl((5-((2,4-dimethoxybenzyl)amino)-9-fluoro-7-methoxy-[1,2,4]triazolo[1,5-c]quinazolin-2-yl)methyl)carbamate (340 mg, 0.663 mmol) in DCM (4 mL) at 0° C. The reaction was stirred vigorously at 0° C. for 30 min. The reaction was quenched with 1 N aq. NaOH and extracted with DCM (3 × 20 mL). The combined organic layers were dried over anhydrous Na₂SO₄, filtered, and concentrated under reduced pressure. The resulting crude residue was purified by silica gel chromatography (gradient elution: 0-10% MeOH/DCM) to provide 2-(aminomethyl)-N-(2,4-dimethoxybenzyl)-9-fluoro-7-methoxy-[1,2,4]triazolo[1,5-c]quinazolin-5-amine. MS (ESI) m/z calc’d for C₂₀H₂₁FN₆O₃ [M+H]⁺ 413.1, found 413.3.

### EXAMPLES

### Example 1.2, N-((5-amino-7-methoxy-[1,2,-4]triazolo[1,5-c]quinazolin-2-yl)methyl)-4-(2-hydroxypropan-2-yl)-2,6-dimethylbenzamide.

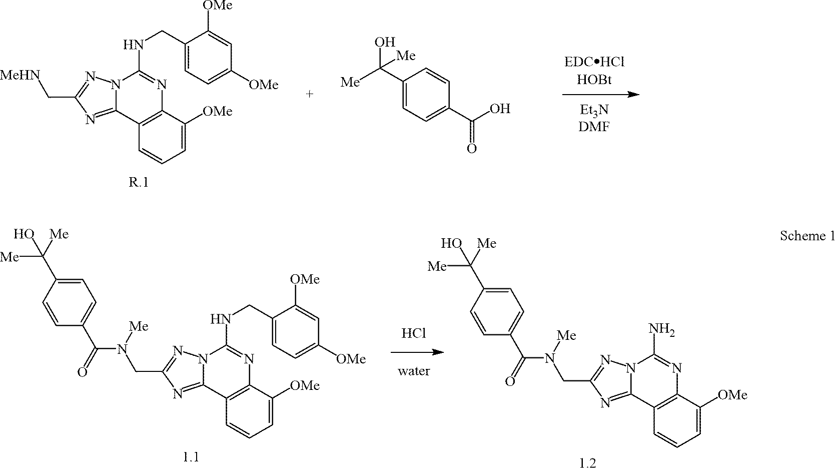

### Step 1- Intermediate 1.1,N-((5-((2,4-dimethoxybenzyl)amino)-7-methoxy-[1,2,4]triazolo[1,5-c]quinazolin-2-yl)methyl)-4-(2-hydroxypropan-2-yl)-N-methylbenzamide.

N-(2,4-dimethoxybenzyl)-7-methoxy-2-((methylamino)methyl)-[1,2,4]triazolo[1,5-c]quinazolin-5-amine (65 mg, 0.159 mmol) was added to a stirred solution of 4-(2-hydroxypropan-2-yl)benzoic acid (31.5 mg, 0.175 mmol), HOBt (26.8 mg, 0.175 mmol), EDC•HCl (33.6 mg, 0.175 mmol), and Et₃N (48.8 µL, 0.350 mmol) in DMF (1.59 mL). The reaction was stirred vigorously at room temperature for 2 h. The reaction was concentrated under reduced pressure to provide N-((5-((2,4-dimethoxybenzyl)amino)-7-methoxy-[1,2,4]triazolo[1,5-c]quinazolin-2-yl)methyl)-4-(2-hydroxypropan-2-yl)-N-methylbenzamide, which was used in the subsequent step without purification.

### Step 2- Example 1.2, N-((5-amino-7-methoxy-[-1,2,4]triazolo[-1,5-c]quinazolin-2-yl)methyl)-4-(2-hydroxypropan-2-yl)-N-methylbenzamide.

HCl (37% aq., 0.33 mL, 3.98 mmol) and water (0.33 mL) was added to N-((5-((2,4-dimethoxybenzyl)amino)-7-methoxy-[1,2,4]triazolo[1,5-c]quinazolin-2-yl)methyl)-4-(2-hydroxypropan-2-yl)-N-methylbenzamide from the previous step. The reaction was stirred vigorously at 60° C. for 1 h. The reaction was cooled to room temperature, quenched with a solution ofNaOH (191 mg, 4.77 mmol) in water (2 mL), and extracted with 3:1 EtOAc/EtOH (2 × 10 mL). The combined organic layers were dried over anhydrous MgSO₄, filtered, and concentrated under reduced pressure. The resulting crude residue was purified by reversed-phase HPLC [Method A] to provideN-((5-amino-7-methoxy-[1,2,4]triazolo[1,5-c]quinazolin-2-yl)methyl)-4-(2-hydroxypropan-2-yl)-N-methylbenzamide (Example 1.2). MS (ESI) m/z calc’d for C₂₂H₂₅N₆O₃ [M+H]⁺ 421.2, found 421.3. ¹H NMR (23 of 24 observed, 500 MHz, DMSO-d₆) δ 7.97 (br s, 2H), 7.78 (dd, J= 7.9, 1.0 Hz, 1H), 7.56 (br s, 3H), 7.47 (br s, 1H), 7.34 (t, J= 7.9 Hz, 1H), 7.27 (d, J= 8.0 Hz, 1H), 4.99 (s, 1H), 4.74 (s, 1H), 3.93 (s, 3H), 3.08 (app d, J= 32.8 Hz, 3H), 1.44 (d, J= 9.1 Hz, 6H). A_{2A} IC₅₀ 0.3 nM (A).

The following example in Table 12 was prepared according to Step 1 of Scheme 1 and General Scheme 5, using Intermediates Q.2 and B.1. Asterisk (*) indicates that A_{2B} data is not available.

TABLE 12

Examples Prepared According to Scheme 1

| Entry | Structure | Observed m/z [M + H]⁺ | A2a IC₅₀ (nM) |
|---|---|---|---|
| | Name | | A2b IC₅₀ (nM) |
| 1.3 | 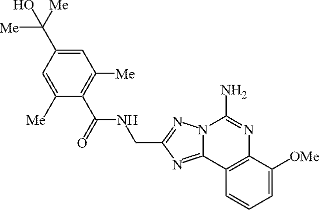 [SMILES: COc1cccc2c1nc(N)n1nc(CNC(=O)c3c(C)cc(C(C)(C)O)cc3C)nc21] | 434.2 | 10.2 (A) |
| | N-((5-amino-7-methoxy-[1,2,4]triazolo[1,5-c]quinazolin-2-yl)methyl)-4-(2-hydroxypropan-2-yl)-2,6-dimethylbenzamide | | * |

### Example 2.1, N-((5-amino-7-methoxy-[1,2,4]triazolo[1,5-c]quinazolin-2-yl)methyl)-4-(2-hydroxypropan-2-yl)benzamide.

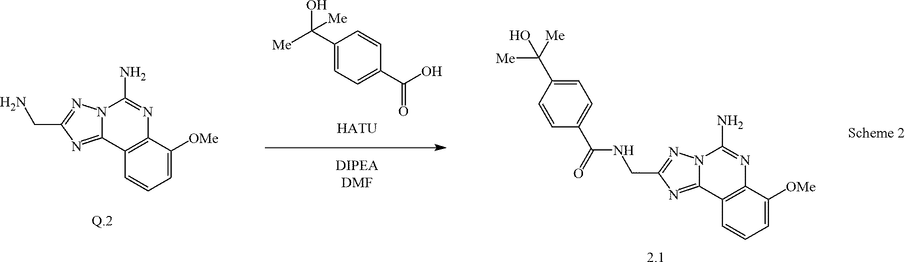

DIPEA (52.3 µL, 0.30 mmol) and HATU (76 mg, 0.20 mmol) were sequentially added to a suspension of 2-(aminomethyl)-7-methoxy-[1,2,4]triazolo[1,5-c]quinazolin-5-amine (26.8 mg, 0.11 mmol) and 4-(2-hydroxypropan-2-yl)benzoic acid (18 mg, 0.10 mmol) in DCM (1 mL). The reaction was stirred vigorously at room temperature for 16 h. The reaction was concentrated under reduced pressure, and the resulting crude residue was purified by reversed-phase HPLC [Method B] to provide N-((5-amino-7-methoxy-[1,2,4]triazolo[1,5-c]quinazolin-2-yl)methyl)-4-(2-hydroxypropan-2-yl)benzamide (Example 2.1). MS (ESI) m/z calc’d for C₂₁H₂₃N₆O₃ [M+H]⁺ 407.2, found 407.1. ¹H NMR (500 MHz, DMSO-d₆) δ 7 9.12 (t, J= 5.7 Hz, 1H), 7.88 (m, 4H), 7.73 (dd, J= 7.9, 1.1 Hz, 1H), 7.57 (d, J= 8.4 Hz, 2H), 7.30 (t, J = 7.9 Hz, 1H), 7.23 (d, J= 8.0 Hz, 1H), 5.13 (s, 1H), 4.77 (d, J= 5.6 Hz, 2H), 3.91 (s, 3H), 1.44 (s, 6H). A_{2A} IC₅₀ 0.6 nM (A).

The following examples in Table 13 were prepared according to Scheme 2 and General Schemes 5, using the appropriate acid and amine intermediates. Example 2.7 was further resolved by chiral SFC. SFC conditions for the resolution of Examples 2.7A and 2.7B are provided, following the table. Asterisk (*) indicates that A_{2B} data is not available.

TABLE 13

Examples Prepared According to Scheme 2

| Entry | Structure | Observed m/z [M + H]⁺ | A2a IC₅₀ (nM) |
|---|---|---|---|
| | Name | | A2b IC₅₀ (nM) |
| 2.2 | 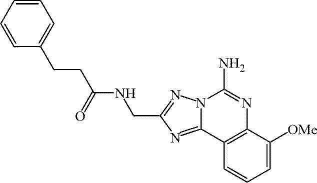 [SMILES: COc1cccc2c1nc(N)n1nc(CNC(=O)CCc3ccccc3)nc21] | 377.3 | 12.7 (B) |
| | N-((5-amino-7-methoxy-[1,2,4]triazolo[1,5-c]quinazolin-2-yl)methyl)-3-phenylpropanamide | | ∗ |
| 2.3 | 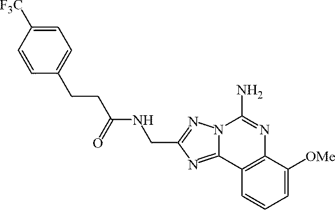 [SMILES: COc1cccc2c1nc(N)n1nc(CNC(=O)CCc3ccc(C(F)(F)F)cc3)nc21] | 445.2 | 4.5 (B) |
| | N-((5-amino-7-methoxy-[1,2,4]triazolo[1,5-c]quinazolin-2-yl)methyl)-3-(4-(trifluoromethyl)phenyl)propenamide | | ∗ |
| 2.4 | 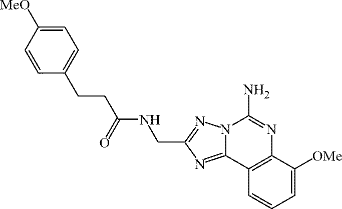 [SMILES: COc1ccc(CCC(=O)NCc2nc3c4cccc(OC)c4nc(N)n3n2)cc1] | 407.3 | 7.5 (B) |
| | N-((5-amino-7-methoxy-[1,2,4]triazolo[1,5-c]quinazolin-2-yl)methyl)-3-(4-methoxyphenyl)propenamide | | ∗ |
| 2.5 | 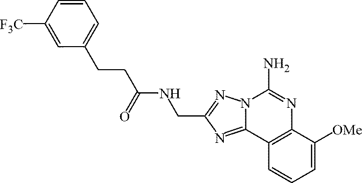 [SMILES: COc1cccc2c1nc(N)n1nc(CNC(=O)CCc3cccc(C(F)(F)F)c3)nc21] | 445.2 | 25.8 (B) |
| | N-((5-amino-7-methoxy-[1,2,4]triazolo[1,5-c]quinazolin-2-yl)methyl)-3-(3-(trifluoromethyl)phenyl)propenamide | | ∗ |
| 2.6 | 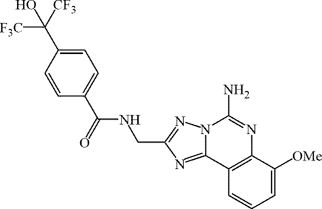 [SMILES: COc1cccc2c1nc(N)n1nc(CNC(=O)c3ccc(C(O)(C(F)(F)F)C(F)(F)F)cc3)nc21] | 515.1 | 0.3 (A) |
| | N-((5-amino-7-methoxy-[1,2,4]triazolo[1,5-c]quinazolin-2-yl)methyl)-4-(1,1,1,3,3,3-hexafluoro-2-hydroxypropan-2-yl)benzamide | | ∗ |
| 2.7A (faster eluting) | 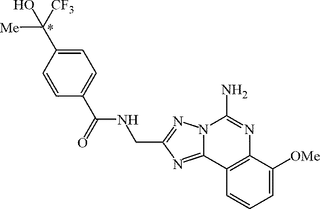 | 461.2 | 0.3 (A) |
| | (S or R)-N-((5-amino-7-methoxy-[1,2,4]triazolo[1,5-c]quinazolin-2-yl)methyl)-4-(1,1,1-trifluoro-2-hydroxypropan-2-yl)benzamide | | ∗ |
| 2.7B (slower eluting) | 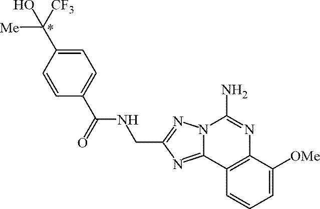 | 461.1 | 0.5 (A) |
| | (R or S)-N-((5-amino-7-methoxy-[1,2,4]triazolo[1,5-c]quinazolin-2-yl)methyl)-4-(1,1,1-trifluoro-2-hydroxypropan-2-yl)benzamide | | ∗ |

### Examples 2.7A and 2.7B

Examples 2.7A and 2.7B were resolved by chiral SFC (Column: EP, 21 × 250 mm, 75% MeOH [w/ 0.1% NH₄OH]/CO₂).

### Example 3.4, N-((5-amino-9-fluoro-7-methoxy-[1,2,4]triazolo[1,5-c]quinazolin-2-yl)methyl)-2,6-difluoro-4-(2-hydroxypropan-2-yl)benzamide.

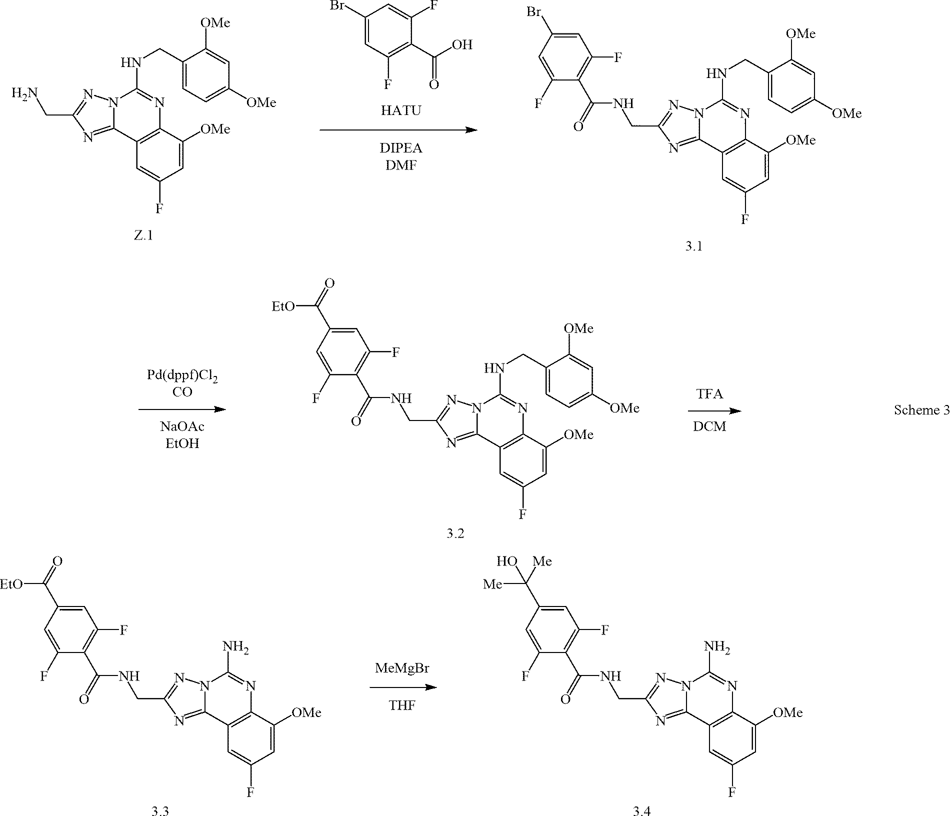

### Step 1 - Intermediate 3.1, 4-bromo-N-((5-((2,4-dimethoxybenzyl)amino)-9-fluoro-7-methoxy-[1,2,4]triazolo[1,5-c]quinazolin-2-yl)methyl)-2,6-difluorobenzamide.

2-(aminomethyl)-N (2,4-dimethoxybenzyl)-9-fluoro-7-methoxy-[1,2,4]triazolo[1,5-c]quinazolin-5-amine (100 mg, 0.242 mmol) was added to a stirred solution of 4-bromo-2,6-difluorobenzoic acid (86 mg, 0.364 mmol), HATU (138 mg, 0.364 mmol), and DIEA (42 µL, 0.242 mmol) in DMF (2 mL). The reaction was stirred vigorously at room temperature for 2 h. The reaction was filtered to provide 4-bromo-N-((5-((2,4-dimethoxybenzyl)amino)-9-fluoro-7-methoxy-[1,2,4]triazolo[1,5-c]quinazolin-2-yl)methyl)-2,6-difluorobenzamide, which was used in the subsequent step without purification. MS (ESI) m/z calc’d for C₂₇H₂₃BrF₃NeO₄ [M+H]⁺ 633.4, found 633.0.

### Step 2 - Intermediate 3.2, Ethyl 4-(((5-((2,4-dimethoxybenzyl)amino)-9-fluoro-7-methoxy-[1,2,4]triazolo[1,5-c]quinazolin-2-yl)methyl)carbamoyl)-3,5-difluorobenzoate.

PdCl₂(dppf) (8.69 mg, 0.012 mmol) and NaOAc (32.5 mg, 0.396 mmol) were added to a stirred solution of 4-bromo-A-((5-((2,4-dimethoxybenzyl)amino)-9-fluoro-7-methoxy-[1,2,4]triazolo[1,5-c]quinazolin-2-yl)methyl)-2,6-difluorobenzamide (50 mg, 0.079 mmol) in EtOH (5 mL). The reaction was stirred vigorously at 80° C. under an atmosphere of CO at 50 psi for 10 h. The reaction was cooled to room temperature and filtered to provide ethyl 4-(((5-((2,4-dimethoxybenzyl)amino)-9-fluoro-7-methoxy-[1,2,4]triazolo[1,5-c]quinazolin-2-yl)methyl)carbamoyl)-3,5-difluorobenzoate, which was used in the subsequent step without purification. MS (ESI) m/z calc’d for C₃₀H₂₈F₃N₆O₆ [M+H]⁺ 625.5, found 625.1.

### Step 3 - Intermediate 3.3, Ethyl 4-(((5-amino-9-fluoro-7-methoxy-[1,2,4]triazolo[1,5-c]quinazolin-2-yl)methyl)carbamoyl)-3,5-difluorobenzoate.

TFA (1 mL) was added to a stirred solution of ethyl 4-(((5-((2,4-dimethoxybenzyl)amino)-9-fluoro-7-methoxy-[1,2,4]triazolo[1,5-c] quinazolin-2-yl)methyl)carbamoyl)-3,5-difluorobenzoate (30 mg) in DCM (1 mL). The reaction was stirred vigorously at room temperature for 12 h. The reaction was quenched with sat. aq. NaHCO₃ to pH ~7-8 and extracted with DCM (3 × 30 mL). The combined organic layers were washed with brine (30 mL), dried over anhydrous Na₂SO₄, filtered, and concentrated under reduced pressure to provide ethyl 4-(((5-amino-9-fluoro-7-methoxy-[1,2,4]triazolo[1,5-c]quinazolin-2-yl)methyl)carbamoyl)-3,5-difluorobenzoate, which was used in the subsequent step without further purification. MS (ESI) m/z calc’d for C₂₁H₁₈F₃N₆O₄ [M+H]⁺ 475.3, found 475.1.

### Step 4 - Example 3A,N-((5-amino-9-fluoro-7-methoxy-[1,2,4]triazolo[1,5-c]quinazolin-2-yl)methyl)-2,6-difluoro-4-(2-hydroxypropan-2-yl)benzamide.

MeMgBr (84 µL, 0.253 mmol) was added dropwise to a solution of ethyl 4-(((5-amino-9-fluoro-7-methoxy-[1,2,4]triazolo[1,5-c]quinazolin-2-yl)methyl)carbamoyl)-3,5-difluorobenzoate (20 mg, 0.042 mmol) in THF (2 mL). The reaction was stirred vigorously at room temperature for 1 h. The reaction was quenched with sat. aq. NH₄Cl (10 mL) and extracted with EtOAc (3 × 30 mL). The combined organic layers were washed with brine (30 mL), dried over anhydrous Na₂SO₄, filtered, and concentrated under reduced pressure. The resulting crude residue was purified by reversed-phase HPLC [Method B] to provide N-((5-amino-9-fluoro-7-methoxy-[1,2,4]triazolo[1,5-c]quinazolin-2-yl)methyl)-2,6-difluoro-4-(2-hydroxypropan-2-yl)benzamide (Example 3.4). MS (ESI) m/z calc’d for C₂₁H₂₀F₃N₆O₃ [M+H]⁺ 461.4, found 461.1. ¹H NMR (15 of 19 observed, 400 MHz, MeOD-d₄) δ 7.51 (dd, J= 8.4, 2.5 Hz, 1H), 7.22-7.07 (m, 3H), 4.60 (br s, 2H), 4.02 (s, 3H), 1.50 (s, 6H). A_{2A} IC₅₀ 11.1 nM (A).

The following examples in Table 14 were prepared according to Steps 1, 3 and 4 of Scheme 3 and General Scheme 5, using Intermediate Z.1 and the appropriate acid. Asterisk (*) indicates that A_{2B} data is not available.

TABLE 14

Examples Prepared According to Scheme 3

| Entry | Structure | Observed m/z [M + H]⁺ | A2a IC₅₀ (nM) |
|---|---|---|---|
| | Name | | A2b IC₅₀ (nM) |
| 3.5 | 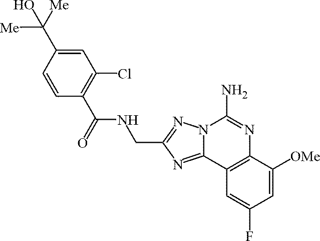 [SMILES: COc1cc(F)cc2c1nc(N)n1nc(CNC(=O)c3ccc(C(C)(C)O)cc3Cl)nc21] | 459.1 | 2.5 (A) |
| | N-((5-amino-9-fluoro-7-methoxy-[1,2,4]triazolo[1,5-c]quinazolin-2-y1)methyl)-2-chloro-4-(2-hydroxypropan-2-yl)benzamide | | ∗ |

### Example 4.2, N-((5-amino-7-methoxy-[-1,2,4]triazolo[-1,5-c]quinazolin-2-yl)methyl)-5-(2-hydroxypropan-2-yl)picolinamide.

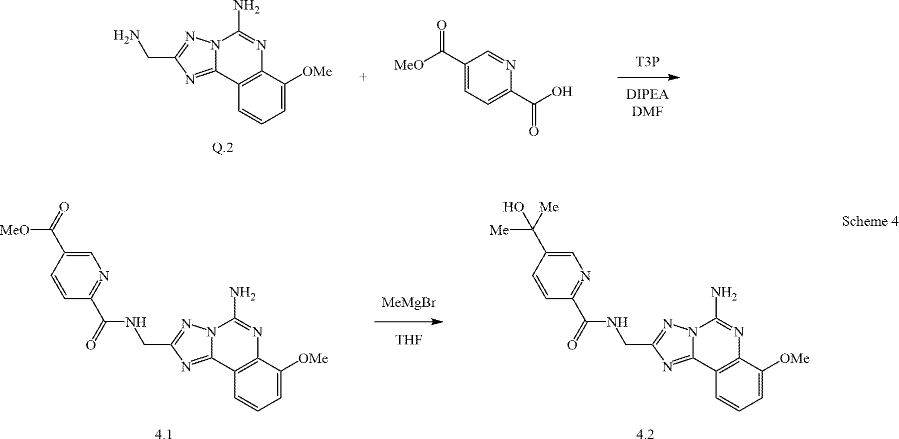

### Step 1 - Intermediate 4.1, Methyl 6-(((5-amino-7-methoxy-[1,2,4]triazolo[1,5-c]quinazolin-2-yl)methyl)carbamoyl)nicotinate

A solution of 5-(methoxycarbonyl)picolinic acid (43.6 mg, 0.241 mmol) in DMF (2.00 mL), DIPEA (0.11 mL, 0.602 mmol), and T3P^{®} (50% weight in EtOAc, 0.24 mL, 0.401 mmol) were sequentially added to 2-(aminomethyl)-7-methoxy-[1,2,4]triazolo[1,5-c]quinazolin-5-amine (49 mg, 0.201 mmol). The reaction was stirred vigorously at room temperature for 1 h. The reaction was concentrated under reduced pressure. The resulting crude residue was purified by silica gel chromatography (gradient elution: 0-100% [3:1 EtOAc/EtOH]/hexanes) to provide 6-(((5-amino-7-methoxy-[1,2,4]triazolo[1,5-c]quinazolin-2-yl)methyl)carbamoyl)nicotinate. MS (ESI) m/z calc’d for C₁₉H₁₈N₇O₄ [M+H]⁺ 408.1, found 408.1.

### Step 2 - Example 4.2, N-((5-amino-7-methoxy-[1,2,4]triazolo[1,5-c]quinazolin-2-yl)methyl)-5-(2-hydroxypropan-2-yl)picolinamide.

MeMgBr (3.4 M in 2-MeTHF, 0.254 mL, 0.864 mmol) was added dropwise to a solution of 6-(((5-amino-7-methoxy-[1,2,4]triazolo[1,5-c]quinazolin-2-yl)methyl)carbamoyl)nicotinate (22 mg, 0.054 mmol) in THF (2.16 mL) at -78° C. The reaction was stirred vigorously at -78° C. for 7 h. The reaction was warmed to room temperature and was quenched with 1 N HCl (5 mL). The mixture was diluted with water (10 mL) and extracted with EtOAc (3 × 10 mL). The combined organic layers were dried over anhydrous MgSO₄, filtered, and concentrated under reduced pressure. The resulting crude residue was purified by reversed-phase HPLC [Method B] to provide N-((5-amino-7-methoxy-[1,2,4]triazolo[1,5-c]quinazolin-2-yl)methyl)-5-(2-hydroxypropan-2-yl)picolinamide (Example 4.2). MS (ESI) m/z calc’d for C₂₀H₂₂N₇O₃ [M+H]⁺ 408.2, found 408.1. ¹H NMR (500 MHz, DMSO-d₆) δ 9.30 (t, J= 6.0 Hz, 1H), 8.79 (d, J= 2.0 Hz, 1H), 8.05 (d, J= 2.2 Hz, 1H), 8.01 (d, J= 8.1 Hz, 1H), 7.84 (br s, 2H), 7.72 (dd, J= 7.9, 1.0 Hz, 1H), 7.30 (t, J= 7.9 Hz, 1H), 7.23 (d, J= 7.2 Hz, 1H), 5.39 (s, 1H), 4.82 (d, J= 6.0 Hz, 2H), 3.91 (s, 3H), 1.50 (s, 6H). A_{2A} IC₅₀ 1.4 nM (A).

The following examples in Table 15 were prepared according to Step 1 of Scheme 4 and General Scheme 4-5, using the appropriate acid and amine intermediates. Asterisk (*) indicates that A_{2B} data is not available.

TABLE 15

Examples Prepared According to Scheme 4

| Entry | Structure | Observed m/z [M + H]⁺ | A2a IC₅₀ (nM) |
|---|---|---|---|
| | Name | | A2b IC₅₀ (nM) |
| 4.3 | 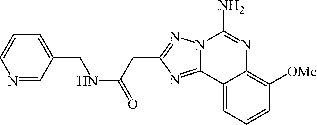 [SMILES: COc1cccc2c1nc(N)n1nc(CC(=O)NCc3cccnc3)nc21] | 363.0 | 44.3 (B) |
| | 2-(5-amino-7-methoxy-[1,2,4]triazolo[1,5-c]quinazolin-2-yl)-N-(pyridin-3-ylmethyl)acetamide | | ∗ |
| 4.4 | 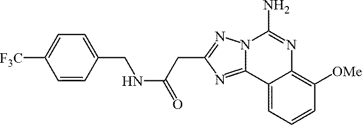 [SMILES: COc1cccc2c1nc(N)n1nc(CC(=O)NCc3ccc(C(F)(F)F)cc3)nc21] | 431.4 | 9.4 (B) |
| | 2-(5-amino-7-methoxy-[1,2,4]triazolo[1,5-c]quinazolin-2-y1)-N-(4-(trifluoromethyl)benzyl)acetamide | | ∗ |
| 4.5 | 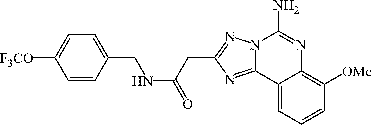 [SMILES: COc1cccc2c1nc(N)n1nc(CC(=O)NCc3ccc(OC(F)(F)F)cc3)nc21] | 447.2 | 5.8 (B) |
| | 2-(5-amino-7-methoxy-[1,2,4]triazolo[1,5-c]quinazolin-2-yl)-N-(4-(trifluoromethoxy)benzyl)acetamide | | * |
| 4.6 | 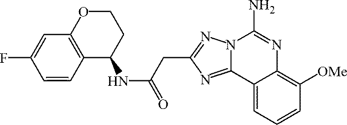 [SMILES: COc1cccc2c1nc(N)n1nc(CC(=O)N[C@@H]3CCOc4cc(F)ccc43)nc21] | 423.3 | 2.0 (B) |
| | (R)-2-(5-amino-7-methoxy-[1,2,4]triazolo[1,5-c]quinazolin-2-yl)-N-(7-fluorochroman-4-yl)acetamide | | ∗ |
| 4.7 | 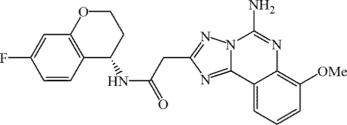 [SMILES: COc1cccc2c1nc(N)n1nc(CC(=O)N[C@H]3CCOc4cc(F)ccc43)nc21] | 423.3 | 6.7 (B) |
| | (S)-2-(5-amino-7-methoxy-[1,2,4]triazolo[1,5-c]quinazolin-2-yl)-N-(7-fluorochroman-4-yl)acetamide | | ∗ |
| 4.8 | 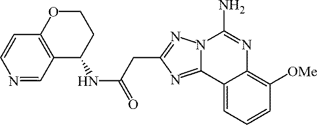 [SMILES: COc1cccc2c1nc(N)n1nc(CC(=O)N[C@H]3CCOc4ccncc43)nc21] | 363.0 | 111 (B) |
| | (S)-2-(5-amino-7-methoxy-[1,2,4]triazolo[1,5-c]quinazolin-2-yl)-N-(3,4-dihydro-2H-pyrano[3,2-c]pyridin-4-yl)acetamide | | ∗ |
| 4.9 | 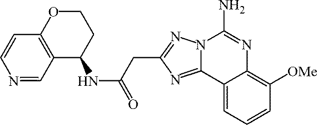 [SMILES: COc1cccc2c1nc(N)n1nc(CC(=O)N[C@@H]3CCOc4ccncc43)nc21] | 363.0 | 5.8 (B) |
| 4.10 | 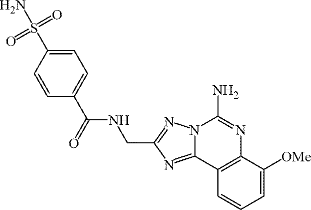 [SMILES: COc1cccc2c1nc(N)n1nc(CNC(=O)c3ccc(S(N)(=O)=O)cc3)nc21] | 428.1 | 636 (A) |
| | N-((5-amino-7-methoxy-[1,2,4]triazolo[1,5-c]quinazolin-2-y1)methyl)-4-sulfamoylbenzamide | | ∗ |
| 4.11 | 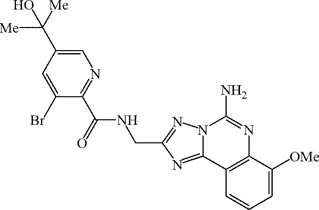 [SMILES: COc1cccc2c1nc(N)n1nc(CNC(=O)c3ncc(C(C)(C)O)cc3Br)nc21] | 486, 488.0 | 1.5 (A) |
| | N-((5-amino-7-methoxy-[1,2,4]triazolo[1,5-c]quinazolin-2-yl)methyl)-3-bromo-5-(2-hydroxypropan-2-yl)picolinamide | | ∗ |

### Example 5.3,2-((diethylamino)methyl)-7methoxy-[1,2,4]triazolo[1,5-c]quinazolin-5-amine.

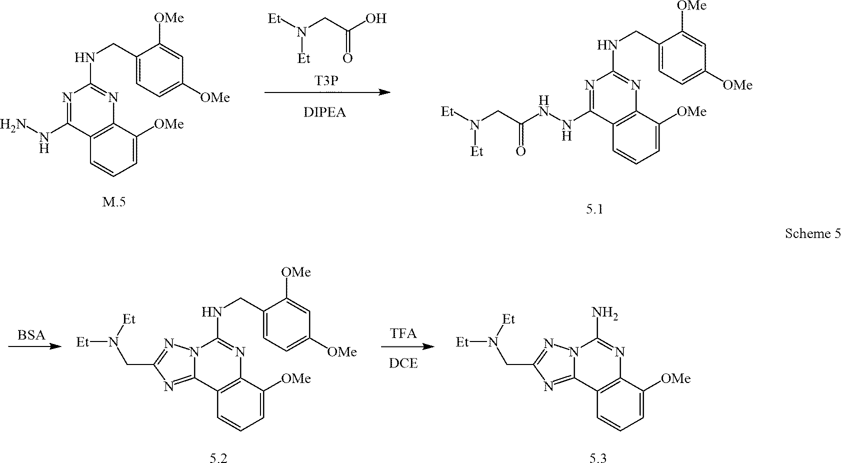

### Step 1 - Intermediate 5.1, 2-(diethylamino)-N′-(2-((2,4-dimethoxybenzyl)amino)-8-methoxyquinazolin-4-yl)acetohydrazide.

T3P® (50% weight in EtOAc, 93 mg, 0.146 mmol) was added to a stirred solution N-(2,4-dimethoxybenzyl)-4-hydrazinyl-8-methoxyquinazolin-2-amine (40 mg, 0.113 mmol), diethylglycine (18 mg, 0.135 mmol), DIPEA (59 µL) in DCM (1 mL). The reaction was stirred vigorously at room temperature for 48 h. The reaction was quenched with sat. aq. NaHCO₃ (1 mL). The combined organic layers were concentrated under reduced pressure to provide 2-(diethylamino)-N′-(2-((2,4-dimethoxybenzyl)amino)-8-methoxyquinazolin-4-yl)acetohydrazide, which was used in the subsequent step without further purification.

### Step 2 - Intermediate 5.2, 2-((diethylamino)methyl)-N-(2,4-dimethoxybenzyl)-7-methoxy-[-1,2,4]triazolo[-1,5-c]quinazolin-5-amine.

BSA (1 mL, 4.09 mmol) was added to 2-(diethylamino)-7V′-(2-((2,4-dimethoxybenzyl)amino)-8-methoxyquinazolin-4-yl)acetohydrazide from the previous step. The reaction was stirred vigorously at 130° C. for 22 h. The reaction was cooled to room temperature and concentrated under reduced pressure. The reaction mixture was diluted with MeOH (0.5 mL) and concentrated under reduced pressure to provide 2-((diethylamino)methyl)-N-(2,4-dimethoxybenzyl)-7-methoxy-[1,2,4]triazolo[1,5-c]quinazolin-5-amine, which was used in the subsequent step without further purification.

### Step 3 - Example 5.3, 2-((diethylamino)methyl)-7-methoxy-[1,2,4]triazolo[-1,5-c]quinazolin-5-amine.

TFA (1 mL) was added to a stirred solution of 2-((diethylamino)methyl)-7V-(2,4-dimethoxybenzyl)-7-methoxy-[1,2,4]triazolo[1,5-c]quinazolin-5-amine from the previous step in DCE (1 mL). The reaction was stirred vigorously at room temperature for 48 h. The reaction was concentrated under reduced pressure. The resulting crude residue was purified by reversed-phase HPLC to provide 2-((diethylamino)methyl)-7-methoxy-[1,2,4]triazolo[1,5-c]quinazolin-5-amine (Example 5.3). MS (ESI) m/z calc’d for C₁₅H₂₁N₆O [M+H]⁺ 301.2, found 301.2. A_{2A} IC₅₀ 24.4 nM (B).

### Example 6.1, Benzyl ((5-amino-7-methoxy-[-1,2,4]triazolo[1,5-c]quinazolin-2-yl)methyl)carbamate.

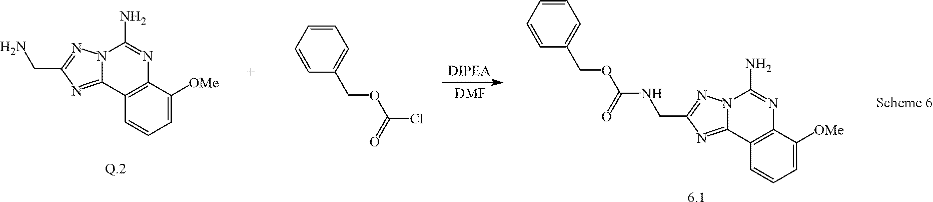

Benzyl chloroformate (18 µL, 0.123 mmol) and DIPEA (56 µL, 0.328 mmol) were sequentially added to a stirred solution of 2-(aminomethyl)-7-methoxy-[1,2,4]triazolo[1,5-c]quinazolin-5-amine (20.0 mg, 0.082 mmol) in DMF (2.00 mL). The reaction was stirred vigorously at room temperature for 16 h. The resulting crude residue was purified by reversed-phase HPLC [Method A] to provide benzyl ((5-amino-7-methoxy-[1,2,4]triazolo[1,5-c]quinazolin-2-yl)methyl)carbamate (Example 6.1). MS (ESI) m/z calc’d for C₁₉H₁₉N₆O₃ [M+H]⁺ 379.2, found 379.1. A_{2A} IC₅₀ 5.9 nM (B).

### Example 7.1, 7-methoxy-2-(((4-(trifluoromethyl)benzyl)amino)methyl)-[1,2,4]triazolo[1,5-c]quinazolin-5-amine.

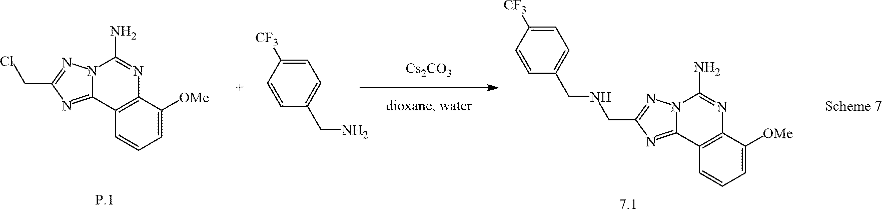

A mixture of 2-(chloromethyl)-7-methoxy-[1,2,4]triazolo[1,5-c]quinazolin-5-amine (120 mg, 0.400 mmol), (4-(trifluoromethyl)phenyl)methanamine (200 mg, 1.142 mmol), and C_{S2}CO₃ (220 mg, 0.675 mmol) in dioxane (5 mL) and water (0.5 mL) was stirred vigorously at 60° C. for 16 h. The reaction was cooled to room temperature and concentrated under reduced pressure. The concentrated residue was diluted with water (30 mL) and extracted with DCM (30 mL). The combined organic layers were dried over anhydrous Na₂SO₄, filtered, and concentrated under reduced pressure. The resulting crude residue was diluted in DCM (10 mL) and triturated with diethyl ether (40 mL). The mixture was filtered and washed with diethyl ether (10 mL) to provide 7-methoxy-2-(((4-(trifluoromethyl)benzyl)amino)methyl)-[1,2,4]triazolo[1,5-c]quinazolin-5-amine (Example 7.1). MS (ESI) m/z calc’d for C₁₉H₁₈F₃N₆O [M+H]⁺ 403.2, found 403.2. A_{2A} IC₅₀ 8.9 nM (B).

The following examples in Table 16 were prepared according to Scheme 7 and General Scheme 1, using Intermediate P.1 and the appropriate amine. Compounds were generally purified by trituration, filtering, and washing with an appropriate solvent, preparative TLC, or reversed-phase prep-HPLC. Asterisk (*) indicates that A_{2B} data is not available.

TABLE 16

Examples Prepared According to Scheme 7

| Entry | Structure | Observed m/z [M + H]⁺ | A2a IC₅₀ (nM) |
|---|---|---|---|
| | Name | | A2b IC₅₀ (nM) |
| 7.2 | 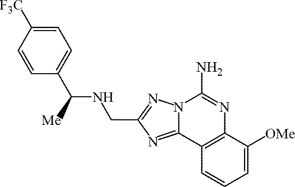 [SMILES: COc1cccc2c1nc(N)n1nc(CN[C@@H](C)c3ccc(C(F)(F)F)cc3)nc21] | 417.2 | 7.2 (B) |
| | (S)-7-methoxy-2-(((1-(4-(trifluoromethyl)phenyl)ethyl)amino)methyl)-[1,2,4]triazolo[1,5-c]quinazolin-5-amine | | ∗ |
| 7.3 | 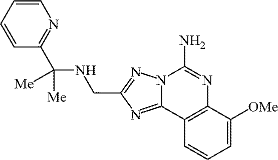 [SMILES: COc1cccc2c1nc(N)n1nc(CNC(C)(C)c3ccccn3)nc21] | 364.2 | 18.5 (B) |
| | 7-methoxy-2-(((2-(pyridin-2-yl)propan-2-yl)amino)methyl)-[1,2,4]triazolo[1,5-c]quinazolin-5-amine | | ∗ |
| 7.4 | 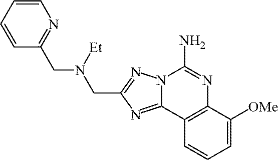 [SMILES: CCN(Cc1ccccn1)Cc1nc2c3cccc(OC)c3nc(N)n2n1] | 364.2 | 31.4 (B) |
| | 2-((ethyl(pyridin-2-ylmethyl)amino)methyl)-7-methoxy-[1,2,4]triazolo[1,5-c]quinazolin-5-amine | | ∗ |
| 7.5 | 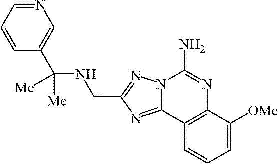 [SMILES: COc1cccc2c1nc(N)n1nc(CNC(C)(C)c3cccnc3)nc21] | 364.2 | 20.5 (B) |
| | 7-methoxy-2-(((2-(pyridin-3-yl)propan-2-yl)amino)methyl)-[1,2,4]triazolo[1,5-c]quinazolin-5-amine | | ∗ |
| 7.6 | 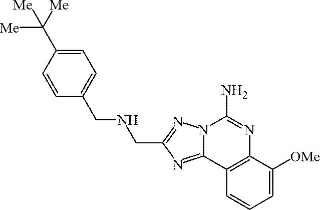 [SMILES: COc1cccc2c1nc(N)n1nc(CNCc3ccc(C(C)(C)C)cc3)nc21] | 391.0 | 4.3 (B) |
| | 2-(((4-(tert-butyl)benzyl)amino)methyl)-7-methoxy-[1,2,4]triazolo[1,5-c]quinazolin-5-amine | | * |
| 7.7 | 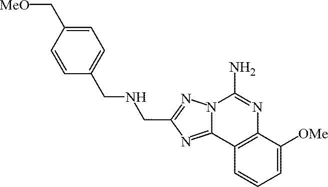 [SMILES: COCc1ccc(CNCc2nc3c4cccc(OC)c4nc(N)n3n2)cc1] | 379.0 | 2.2 (B) |
| | 7-methoxy-2-(((4-(methoxymethyl)benzyl)amino)methyl)-[1,2,4]triazol[1,5-c]quinazolin-5-amine | | ∗ |
| 7.8 | 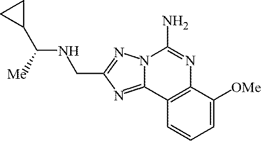 [SMILES: COc1cccc2c1nc(N)n1nc(CN[C@H](C)C3CC3)nc21] | 313.2 | 12.0 (B) |
| | (R)-2-(((1-cyclopropylethyl)amino)methyl)-7-methoxy-[1,2,4]triazolo[1,5-c]quinazolin-5-amine | | ∗ |
| 7.9 | 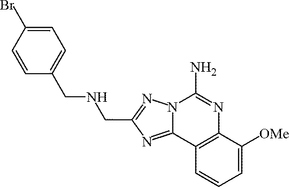 [SMILES: COc1cccc2c1nc(N)n1nc(CNCc3ccc(Br)cc3)nc21] | 413.0, 415.0 | 5.4 (B) |
| | 2-(((4-bromobenzyl)amino)methyl)-7-methoxy-[1,2,4]triazolo[1,5-c]quinazolin-5-amine | | ∗ |

### Example 8.1, 7-methoxy-2-(((4-(trifluoromethyl)benzyl)amino)methyl)-[1,2,4]triazolo[1,5-c]quinazolin-5-amine.

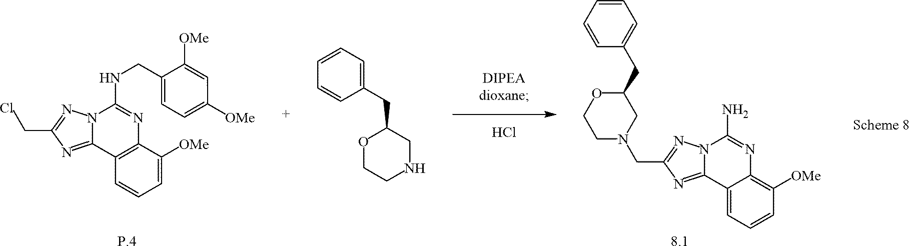

Dioxane (0.30 mL) and DIPEA (0.021 mL, 0.12 mmol) were sequentially added to a mixture of 2-(chloromethyl)-N-(2,4-dimethoxybenzyl)-7-methoxy-[1,2,4]triazolo[1,5-c]quinazolin-5-amine (25 mg, 0.060 mmol) and (S)-2-benzylmorpholine (11.8 mg, 0.067 mmol). The reaction was stirred vigorously at 110° C. 3 h. The reaction was cooled to room temperature. HCl (37% aq., 0.18 mL) was added, and the reaction was stirred vigorously at 35° C. for 60 h. The reaction as cooled to room temperature and diluted with DMSO (3 mL) and NH₄OH (28-30 wt%, 0.125 mL). The resulting mixture was filtered and purified by reverse-phase HPLC [Method A] to provide (S)-2-((2-benzylmorpholino)methyl)-7-methoxy-[1,2,4]triazolo[1,5-c]quinazolin-5-amine (Example 8.1). MS (ESI) m/z calc’d for C₂₂H₂₅N₆O₂ [M+H]⁺ 405.2, found 405.2. ¹H NMR (500 MHz, DMSO-d₆): δ 7.97 (br s, 2 H), 7.75 (d, J = 7.9 Hz, 1 H), 7.41 - 7.34 (m, 1H), 7.33 - 7.20 (m, 6 H), 4.71 (s, 2 H), 4.03 (d, J= 12.5 Hz, 1 H), 3.98 - 3.88 (m, 4 H), 3.79 - 3.52 (m, 3 H), 3.32 - 3.19 (m, 1 H), 3.13 - 3.00 (m, 1 H), 2.85 - 2.71 (m, 2 H). A_{2A} IC₅₀ 6.0 nM (A).

The following examples in Table 17 were prepared according to Scheme 8 and General Scheme 1, using Intermediate P.1 and the appropriate amine (i.e., the addition of HCl was omitted). Compounds were generally purified by column chromatography, preparative TLC, or trituration, filtering, and washing with an appropriate solvent. Asterisk (*) indicates that A_{2B} data is not available.

TABLE 17

Examples Prepared According to Scheme 8

| Entry | Structure | Observed m/z [M + H]⁺ | A2a IC₅₀ (nM) |
|---|---|---|---|
| | Name | | A2b IC₅₀ (nM) |
| 8.2 | 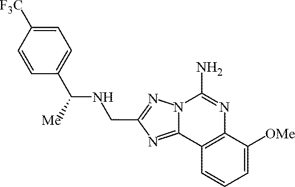 [SMILES: COc1cccc2c1nc(N)n1nc(CN[C@H](C)c3ccc(C(F)(F)F)cc3)nc21] | 417.0 | 22.4 (B) |
| | (R)-7-methoxy-2-(((1-(4-(trifluoromethyl)phenyl)ethyl)amino)methyl)-[1,2,4]triazolo[1,5-c]quinazolin-5-amine | | ∗ |
| 8.3 | 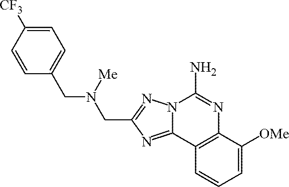 [SMILES: COc1cccc2c1nc(N)n1nc(CN(C)Cc3ccc(C(F)(F)F)cc3)nc21] | 417.0 | 6.4 (B) |
| | 7-methoxy-2-((methyl(4-(trifluoromethyl)benzyl)amino)methyl)-[1,2,4]triazolo[1,5-c]quinazolin-5-amine | | ∗ |
| 8.4 | 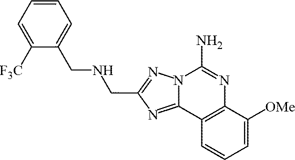 [SMILES: COc1cccc2c1nc(N)n1nc(CNCc3ccccc3C(F)(F)F)nc21] | 403.0 | 3.2 (B) |
| | 7-methoxy-2-(((2-(trifluoromethyl)benzyl)amino)methyl)-[1,2,4]triazolo[1,5-c]quinazolin-5-amine | | ∗ |
| 8.5 | 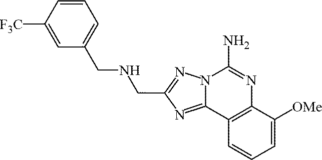 [SMILES: COc1cccc2c1nc(N)n1nc(CNCc3cccc(C(F)(F)F)c3)nc21] | 403.2 | 11.2 (B) |
| | 7-methoxy-2-(((3-(trifluoromethyl)benzyl)amino)methyl)-[1,2,4]triazolo[1,5-c]quinazolin-5-amine | | ∗ |
| 8.6 | 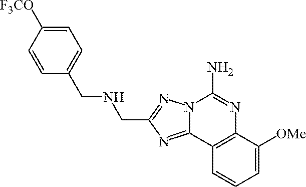 [SMILES: COc1cccc2c1nc(N)n1nc(CNCc3ccc(OC(F)(F)F)cc3)nc21] | 419.2 | 8.9 (B) |
| | 7-methoxy-2-(((4-(trifluoromethoxy)benzyl)amino)methyl)-[1,2,4]triazolo[1,5-c]quinazolin-5-amine | | ∗ |
| 8.7 | 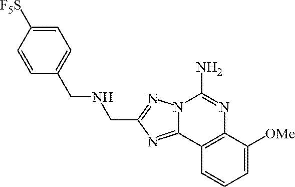 | 461.0 | 6.4 (B) |
| | 7-methoxy-2-(((4-(pentafluoro-λ⁶- sulfanyl)benzyl)amino)methyl)-[1,2,4]triazolo[1,5-c]quinazolin-5-amine | | ∗ |
| 8.8 | 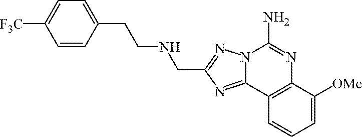 [SMILES: COc1cccc2c1nc(N)n1nc(CNCCc3ccc(C(F)(F)F)cc3)nc21] | 417.2 | 17.3 (B) |
| | 7-methoxy-2-(((4-(trifluoromethoxy)phenethyl)amino)methyl)-[1,2,4]triazolo[1,5-c]quinazolin-5-amine | | * |
| 8.9 | 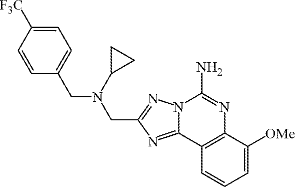 [SMILES: COc1cccc2c1nc(N)n1nc(CN(Cc3ccc(C(F)(F)F)cc3)C3CC3)nc21] | 443.2 | 2.5 (B) |
| | 2-((cyclopropyl(4-(trifluoromethyl)benzyl)amino)methyl)-7-methoxy-[1,2,4]triazolo[1,5-c]quinazolin-5-amine | | ∗ |

### Example 9.2, 4-((((5-amino-7-methoxy-[1,2,4]triazolo[1,5-c]quinazolin-2-yl)methyl)(methyl)amino)methyl)benzenesulfonamide.

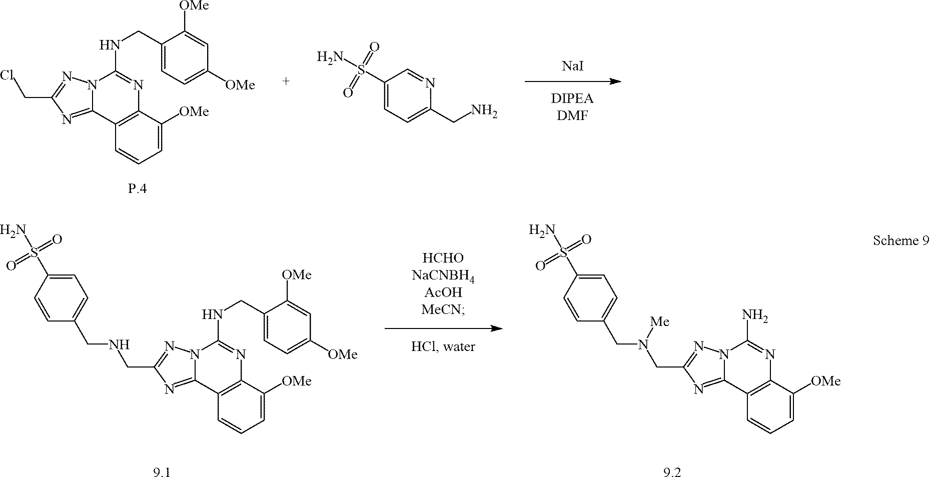

### Step 1 - Intermediate 9.1, 4-((((5-((2,4-dimethoxybenzyl)amino)-7-methoxy-[1,2,4]triazolo[1,5-c]quinazolin-2-yl)methyl)(methyl)amino)methyl)benzenesulfonamide.

DMF (3.00 mL) and DIPEA (0.16 mL, 0.90 mmol) were sequentially added to a mixture of 2-(chloromethyl)-N-(2,4-dimethoxybenzyl)-7-methoxy-[1,2,4]triazolo[1,5-c]quinazolin-5-amine (124 mg, 0.30 mmol), 4-(aminomethyl)benzenesulfonamide (58.6 mg, 0.315 mmol), and NaI (90 mg, 0.60 mmol). The reaction was stirred vigorously at 60° C. for 2 h. The reaction was cooled to room temperature and concentrated under reduced pressure. The resulting crude residue was purified by silica gel chromatography (gradient elution: 0-100% [3:1 EtOAc/EtOH]/hexanes) to provide 4-((((5-((2,4-dimethoxybenzyl)amino)-7-methoxy-[1,2,4]triazolo[1,5-c]quinazolin-2-yl)methyl)(methyl)amino)methyl)benzenesulfonamide. MS (ESI) m/z calc’d for C₂₇H₃₀N₇O₅S [M+H]⁺ 564.2, found 564.2.

### Step 2 - Example 9.2, 4-((((5-amino-7-methoxy-[1,2,4]triazolo[1,5-c]quinazolin-2-yl)methyl)(methyl)amino)methyl)benzenesulfonamide.

HCHO (37% in water, 34 µL, 0.464 mmol) and AcOH (54 µL, 0.926 mmol) were added to a stirred solution of 4-((((5-((2,4-dimethoxybenzyl)amino)-7-methoxy-[1,2,4]triazolo[1,5-c]quinazolin-2-yl)methyl)(methyl)amino)methyl)benzenesulfonamide (87 mg, 0.154 mmol) in MeCN at 0° C. The reaction was stirred vigorously at room temperature for 5 h. The reaction was diluted with sat. aq. NaHCO₃ (10 mL) and extracted with EtOAc (3 × 10 mL). The combined organic layers were dried over anhydrous MgSO₄, filtered, and concentrated under reduced pressure. The resulting crude residue was suspended in HCl (37% aq., 0.32 mL, 3.86 mmol) and water (0.3 mL). The reaction was stirred vigorously at 60° C. for 1 h. The reaction was cooled to room temperature. The reaction was diluted with a solution of NaOH (185 mg) in water (5 mL) and extracted with 3:1 CHCl₃/i-PrOH (2 × 10 mL) and 3:1 EtOAc/EtOH (2 × 10 mL). The combined organic layers were dried over anhydrous MgSO₄, filtered, and concentrated under reduced pressure. The resulting crude residue was purified by reversed-phase HPLC [Method B] to provide 4-((((5-amino-7-methoxy-[1,2,4]triazolo[1,5-c]quinazolin-2-yl)methyl)(methyl)amino)methyl)benzenesulfonamide (Example 9.2). MS (ESI) m/z calc’d for C₁₉H₂₂N₇O₃S [M+H]⁺ 428.1, found 428.1. ¹H NMR (500 MHz, DMSO-d₆) δ 7.86 (br s, 2H), 7.81 (d, J= 8.3 Hz, 2H), 7.78 (dd, J= 8.0, 1.2 Hz, 1H), 7.60 (d, J= 8.3 Hz, 2H), 7.32 (t, J= 3.9 Hz, 3H), 7.24 (d, J= 8.0 Hz, 1H), 3.92 (s, 3H), 3.88 (s, 2H), 3.76 (s, 2H), 2.28 (s, 3H). A_{2A} IC₅₀ 2.5 nM (A). Example 10.1, (1S,2R)-2-(((5-amino-7-methoxy-[1,2,4]triazolo[1,-5-c]quinazolin-2-yl)methyl)amino)-1-(3, 5-bis(trifluoromethyl)phenyl)propan-1-ol.

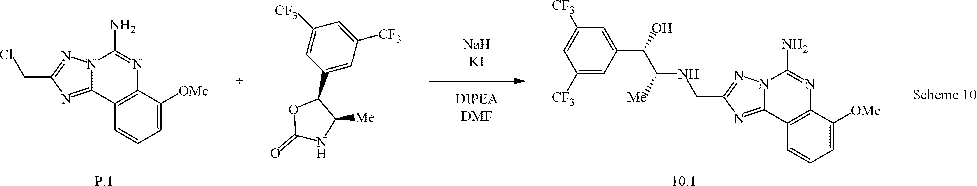

NaH (60% in mineral oil, 25.2 mg, 1.00 mmol) was added to a stirred solution of (4R,5S)-5-(3,5-bis(trifluoromethyl)phenyl)-4-methyloxazolidin-2-one (209 mg, 0.666 mmol) in DMF (3.33 mL) at 0° C. The reaction was stirred vigorously at 0° C. for 45 min. A solution of 2-(chloromethyl)-7-methoxy-[1,2,4]triazolo[1,5-c]quinazolin-5-amine (100 mg, 0.333 mmol), KI (55.3 mg, 0.333 mmol), and DIPEA (87 µL, 0.500 mmol) in DMF (3.33 mL) was added at 0° C. The reaction was stirred vigorously at 80° C. for 3 h. The reaction was cooled to room temperature and quenched with sat. aq. NH₄Cl. The resulting precipitate was filtered, redissolved in 10% MeOH/DCM, and concentrated under reduced pressure. The resulting crude residue was purified by preparative TLC to provide (1S,2R)-2-(((5-amino-7-methoxy-[1,2,4]triazolo[1,5-c]quinazolin-2-yl)methyl)amino)-1-(3,5-bis(trifluoromethyl)phenyl)propan-1-ol (Example 10.1) as a major by-product of the reaction. MS (ESI) m/z calc’d for C₂₂H₂₁F₆N₆O₂ [M+H]⁺ 515.2, found 515.2. A_{2A} IC₅₀ 40.2 nM (B).

The following examples in Table 18 were prepared according to Scheme 10 and General Scheme 1, using the appropriate chloride and amine intermediates, in the absence of NaH. Compounds were generally purified by reversed-phase prep-HPLC or silica gel chromatography. Example 10.15 was further resolved by chiral SFC. SFC conditions for the resolution involved in Example 10.15 are provided, following the table. Asterisk (*) indicates that A_{2B} data is not available.

TABLE 18

Examples Prepared According to Scheme 10

| Entry | Structure | Observed m/z [M + H]⁺ | A2a IC₅₀ (nM) |
|---|---|---|---|
| | Name | | A2b IC₅₀ (nM) |
| 10.2 | 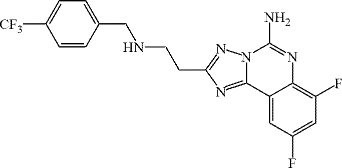 [SMILES: Nc1nc2c(F)cc(F)cc2c2nc(CCNCc3ccc(C(F)(F)F)cc3)nn12] | 423.0 | 110 (B) |
| | 7,9-difluoro-2-(2-((4-(trifluoromethyl)benzyl)amino)ethyl)-[1,2,4]triazolo[1,5-c]quinazolin-5-amine | | ∗ |
| 10.3 | 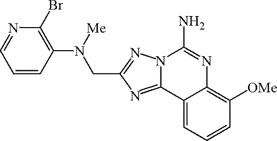 [SMILES: COc1cccc2c1nc(N)n1nc(CN(C)c3cccnc3Br)nc21] | 414.1, 416.1 | 41.6 (B) |
| | 2-(((2-bromopyridin-3-yl)(methyl)amino)methyl)-7-methoxy-[1,2,4]triazolo[1,5-c]quinazolin-5-amine | | ∗ |
| 10.4 | 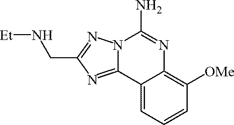 [SMILES: CCNCc1nc2c3cccc(OC)c3nc(N)n2n1] | 273.2 | 46.0 (B) |
| | 2-((ethylamino)methyl)-7-methoxy-[1,2,4]triazolo[1,5-c]quinazolin-5-amine | | ∗ |
| 10.5 | 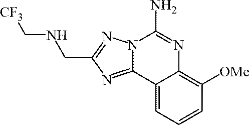 [SMILES: COc1cccc2c1nc(N)n1nc(CNCC(F)(F)F)nc21] | 327.1 | 5.9 (B) |
| | 7-methoxy-2-(((2,2,2-trifluoroethyl)amino)methyl)-[1,2,4]triazolo[1,5-c]quinazolin-5-amine | | ∗ |
| 10.6 | 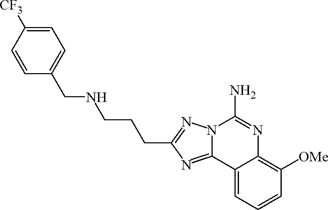 [SMILES: COc1cccc2c1nc(N)n1nc(CCCNCc3ccc(C(F)(F)F)cc3)nc21] | 431.2 | 8.7 (B) |
| | 7-methoxy-2-(3-((4-(trifluoromethyl)benzyl)amino)propyl)-[1,2,4]triazolo[1,5-c]quinazolin-5-amine | | ∗ |
| 10.7 | 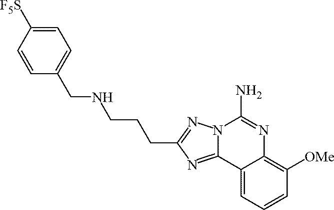 | 489.2 | 45.2 (B) |
| | 7-methoxy-2-(3-((4-(pentafluoro-λ⁶- sulfaneyl)benzyl)amino)propyl)-[1,2,4]triazolo[1,5-c]quinazolin-5-amine | | ∗ |
| 10.8 | 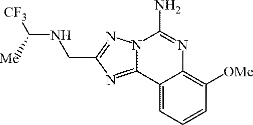 [SMILES: COc1cccc2c1nc(N)n1nc(CN[C@H](C)C(F)(F)F)nc21] | 341.1 | 3.9 (B) |
| | (R)-7-methoxy-2-(((1,1,1-trifluoropropan-2-yl)amino)methyl)-[1,2,4]triazolo[1,5-c]quinazolin-5-amine | | ∗ |
| 10.9 | 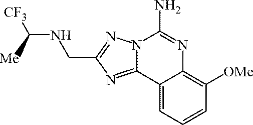 [SMILES: COc1cccc2c1nc(N)n1nc(CN[C@@H](C)C(F)(F)F)nc21] | 341.2 | 5.9 (B) |
| 10.10 | 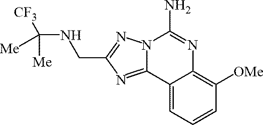 [SMILES: COc1cccc2c1nc(N)n1nc(CNC(C)(C)C(F)(F)F)nc21] | 355.2 | 4.8 (B) |
| | 7-methoxy-2-(((1,1,1-trifluoro-2-methylpropan-2-yl)amino)methyl)-[1,2,4]triazolo[1,5-c]quinazolin-5-amine | | ∗ |
| 10.11 | 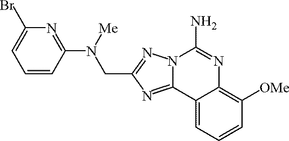 [SMILES: COc1cccc2c1nc(N)n1nc(CN(C)c3cccc(Br)n3)nc21] | 414.1, 416.1 | 2.2 (B) |
| | 2-(((6-bromopyridin-2-yl)(methyl)amino)methyl)-7-methoxy-[1,2,4]triazolo [1,5 -c]quinazolin-5-amine | | ∗ |
| 10.12 | 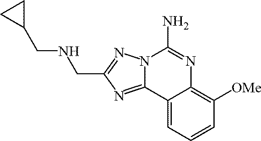 [SMILES: COc1cccc2c1nc(N)n1nc(CNCC3CC3)nc21] | 299.1 | 27.3 (B) |
| | 2-(((cyclopropylmethyl)amino)methyl)-7-methoxy-[1,2,4]triazolo[1,5-c]quinazolin-5-amine | | ∗ |
| 10.13 | 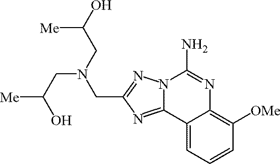 [SMILES: COc1cccc2c1nc(N)n1nc(CN(CC(C)O)CC(C)O)nc21] | 361.2 | 32.2 (B) |
| | 1,1-(((5-amino-7-methoxy-[1,2,4]triazolo[1,5-c]quinazolin-2-yl)methyl)azanediyl)bis(propan-2-ol) | | * |
| 10.14 | 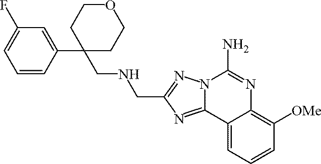 [SMILES: COc1cccc2c1nc(N)n1nc(CNCC3(c4cccc(F)c4)CCOCC3)nc21] | 437.2 | 6.4 (B) |
| | 2-((((4-(3-fluorophenyl)tetrahydro-2H-pyran-4-yl)methyl)amino)methyl)-7-methoxy-[1,2,4]triazolo[1,5-c]quinazolin-5-amine | | ∗ |
| 10.15 (Slower eluting) | 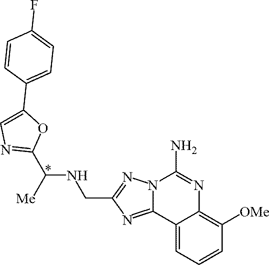 | 434.2 | 44.3 (B) |
| | (S or R)-2-(((1-(5-(4-fluorophenyl)oxazol-2-yl)ethyl)amino)methyl)-7-methoxy-[1,2,4]triazolo[1,5-c]quinazolin-5-amine | | ∗ |

### Example 10.15 (Slower Eluting)

Example 10.15 was the slower eluting compound from the resolution of the corresponding racemic mixture by chiral SFC (Column: OJ, 30 × 250 mm, 50% MeOH [w/ 0.2% Et₂NH]/CO₂).

### Examples 11.2A and 11.2B, (S or R)-2-(((1-(3,3-difluorocyclobutyl)-4,5,6,7-tetrahydro-1H-benzo[d][1,2,3]triazol-5-yl)amino)methyl)-7-methoxy-[1,2,4]triazolo[1,5-c]quinazolin-5-amine and (R or S)-2-(((l-(3,3-difluorocyclobutyl)-4,5,6,7-tetrahydro-1H-benzol[d][1,2,3]triazol-5-yl)amino)methyl)-7-methoxy[1,2,4]triazolo[1,5-c]quinazolin-5-amine.

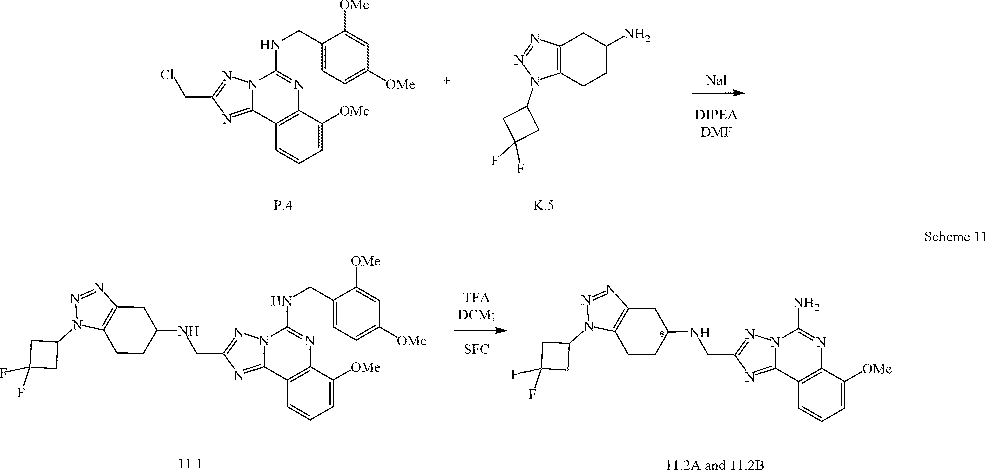

### Step 1- Intermediate 11.1, 2-(((1-(3,3-difluorocyclobutyl)-4,5,6,7-tetrahydro-1H-benzo[d][1,2,3]triazol-5-yl)amino)methyl)-N-(2,4-dimethoxybenzyl)-7-methoxy-[1,2,4]triazolo[1,5-c]quinazolin-5-amine.

DIEA (0.169 mL, 0.967 mmol), NaI (72.4 mg, 0.483 mmol) and 2-(chloromethyl)-N-(2,4-dimethoxybenzyl)-7-methoxy-[1,2,4]triazolo[1,5-c]quinazolin-5-amine (100 mg, 0.242 mmol) were sequentially added to a stirred solution of 1-(3,3-difluorocyclobutyl)-4,5,6,7-tetrahydro-1H-benzo[d][1,2,3]triazol-5-amine (55.1 mg, 0.242 mmol) in DMF (5 mL). The reaction was stirred vigorously at 60° C. for 12 h. The reaction was cooled to room temperature, diluted with water (5 mL) and extracted with EtOAc (2 × 5 mL). The combined organic layers were dried over anhydrous Na₂SO₄, filtered, and concentrated under reduced pressure. The resulting crude residue was purified by preparative TLC (10% MeOH/DCM) to provide 2-(((1-(3,3-difluorocyclobutyl)-4,5,6,7-tetrahydro-1H-benzo[d][1,2,3]triazol-5-yl)amino)methyl)-N-(2,4-dimethoxybenzyl)-7-methoxy-[1,2,4]triazolo[1,5-c]quinazolin-5-amine, which was used in the subsequent step without purification.

### Step 2 ― Examples 11.2A and 11.2B, (S or R)-2-(((1-(3,3-difluorocyclobutyl)-4,5,6,7-tetrahydro-1H-benzo[d][1,2,3]triazol-5-yl)amino)methyl)-7-methoxy-[1,2,4]triazolo[1,5-c]quinazolin-5-amine and (R or S)-2-(((1-(3,3-difluorocyclobutyl)-4,5,6,7-tetrahydro-1H-benzo[d][1,2,3]triazol-5-yl)amino)methyl)-7-methoxy-[1,2,4]triazolo[1,5-c]quinazolin-5-amine.

TFA (10.8 µL, 0.140 mmol) was added to a stirred solution of 2-(((1-(3,3-difluorocyclobutyl)-4,5,6,7-tetrahydro-1H-benzo[d][1,2,3]triazol-5-yl)amino)methyl)-N(2,4-dimethoxybenzyl)-7-methoxy-[1,2,4]triazolo[1,5-c]quinazolin-5-amine (85 mg, 0.140 mmol) in DCM (2 mL). The reaction was stirred vigorously at 40° C. for 12 h. The reaction was cooled to room temperature and concentrated under reduced pressure. The resulting crude residue was purified by reversed-phase HPLC [Method A]. The racemic mixture was separated by chiral SFC (Column: OD-H, 30 × 250 mm, 35% MeOH [w/ 0.1% NH₄OH]/CO₂) to provide Example 11.2A (faster eluting) and Example 11.2B (slower eluting).

Fasting eluting (Example 11.2A): MS (ESI) m/z: calc’d for C₂₁H₂₄F₂N₉O [M+H]⁺: 456.1, found 456.2. ¹H NMR (500 MHz, CDCl₃) δ 7.93 (br d, J= 7.0 Hz, 1H), 7.39 (br t, J= 7.9 Hz, 1H), 7.17 (br d, J= 8.5 Hz, 1H), 5.93 (br s, 2H), 4.63 (br s, 1H), 4.24 (s, 2H), 4.06 (s, 3H), 3.53 - 3.31 (m, 2H), 3.17 (br d, J = 12.8 Hz, 4H), 2.79 - 2.67 (m, 2H), 2.60 (br s, 1H), 2.12 (br s, 1H), 2.05 - 1.86 (m, 2H). A_{2A} IC₅₀ 0.6 nM (A).

Slower eluting (Example 11.2B): MS (ESI) m/z: calc’d for C₂₁H₂₄F₂N₉O [M+H]⁺: 456.1, found 456.2. ¹H NMR (500 MHz, CDCl₃) δ 7.87 (d, J = 7.9 Hz, 1H), 7.40 (t, J = 7.9 Hz, 1H), 7.30 (d, J= 7.6 Hz, 1H), 4.23 (s, 2H), 4.11 - 4.02 (m, 3H), 3.29 - 3.10 (m, 6H), 2.78 - 2.76 (m, 1H), 2.74 - 2.60 (m, 2H), 2.24 - 2.16 (m, 1H), 2.11 - 1.87 (m, 2H). A_{2A} IC₅₀ 3.4 nM (A).

Examples 11.3-11.11 in Table 19 were prepared according to Step 1 of Scheme 11 and General Scheme 1, using the appropriate chloride and amine intermediates. For Examples 11.12-11.14, TBAI was used instead of NaI, and MeCN was used instead of DMF. Example 11.8 was further resolved by chiral SFC. SFC conditions for the resolution involved in the synthesis of Examples 11.8A and 11.8B are provided, following the table. Asterisk (*) indicates that A_{2B} data is not available.

TABLE 19

Examples Prepared According to Scheme 11

| Entry | Structure | Observed m/z [M + H]⁺ | A2a IC₅₀ (nM) |
|---|---|---|---|
| | Name | | A2b IC₅₀ (nM) |
| 11.3 | 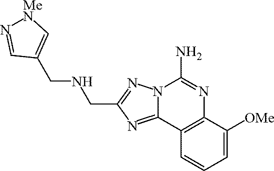 [SMILES: COc1cccc2c1nc(N)n1nc(CNCc3cnn(C)c3)nc21] | 339.1 | 0.1 (A) |
| | 7-methoxy-2-((((1-methyl-1H-pyrazol-4-yl)methyl)amino)methyl)-[1,2,4]triazolo[1,5-c]quinazolin-5-amine | | 1043 (B) |
| 11.4 | 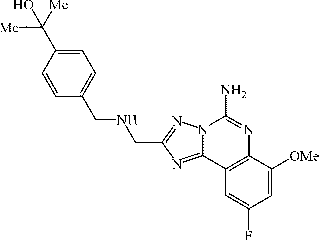 [SMILES: COc1cc(F)cc2c1nc(N)n1nc(CNCc3ccc(C(C)(C)O)cc3)nc21] | 411.2 | 0.1 (A) |
| | 2-(4-((((5-amino-9-fluoro-7-methoxy-[1,2,4]triazolo[1,5-c]quinazolin-2-yl)methyl)amino)methyl)phenyl)propan-2-ol | | 2.9 (B) |
| 11.5 | 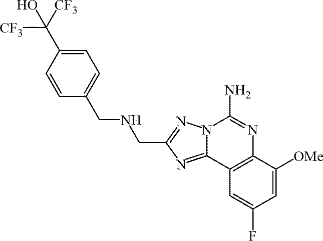 [SMILES: COc1cc(F)cc2c1nc(N)n1nc(CNCc3ccc(C(O)(C(F)(F)F)C(F)(F)F)cc3)nc21] | 519.1 | 0.2 (A) |
| | 2-(4-((((5-amino-9-fluoro-7-methoxy-[1,2,4]triazolo[1,5-c]quinazolin-2-yl)methyl)amino)methyl)phenyl)-1,1,1,3,3,3-hexafluoropropan-2-ol | | 1.0 (A) |
| 11.6 | 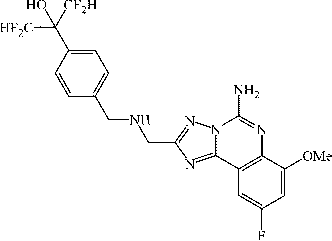 | 519.1 | 0.3 (A) |
| | 2-(4-((((5-amino-9-fluoro-7-methoxy-[1,2,4]triazolo[1,5-c]quinazolin-2-yl)methyl)amino)methyl)phenyl)-1,1,3,3-tetrafluoropropan-2-ol | | 3.7 (A) |
| 11.7A (from faster eluting intermediate J.2A) | 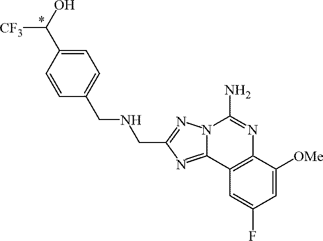 | 451.1 | 0.5 (A) |
| | (S or R)-1-(4-((((5-amino-9-fluoro-7-methoxy-[1,2,4]triazolo[1,5-c]quinazolin-2-yl)methyl)amino)methyl)phenyl)-2,2,2-trifluoroethan-1-ol | | 21.2 (A) |
| 11.7B (from slower eluting intermediate J.2B) | 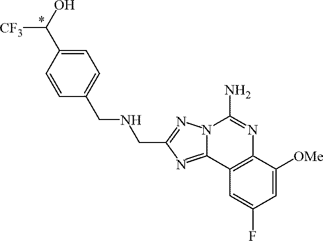 | 451.1 | 0.5 (A) |
| | (R or S)-1-(4-((((5-amino-9-fluoro-7-methoxy-[1,2,4]triazolo[1,5-c]quinazolin-2-yl)methyl)amino)methyl)phenyl)-2,2,2-trifluoroethan-1-ol | | 14.7 (A) |
| 11.8A (faster eluting) | 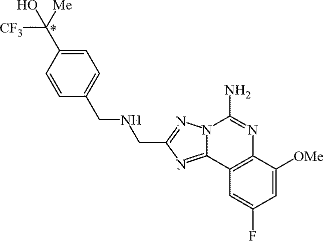 | 465.1 | 0.8 (A) |
| | (S or R)-2-(4-((((5-amino-9-fluoro-7-methoxy-[1,2,4]triazolo[1,5-c]quinazolin-2-yl)methyl)amino)methyl)phenyl)-1,1,1-trifluoropropan-2-ol | | 11.3 (A) |
| 11.8B (slower eluting) | 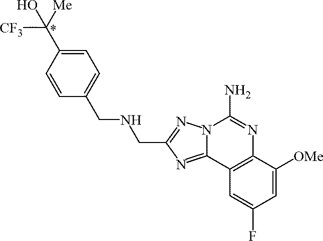 | 465.1 | 0.4 (A) |
| | (R or S)-2-(4-((((5-amino-9-fluoro-7-methoxy-[1,2,4]triazolo[1,5-c]quinazolin-2-yl)methyl)amino)methyl)phenyl)-1,1,1-trifluoropropan-2-ol | | 2.9 (A) |
| 11.9 | 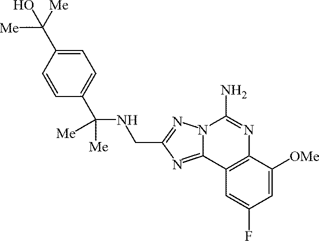 [SMILES: COc1cc(F)cc2c1nc(N)n1nc(CNC(C)(C)c3ccc(C(C)(C)O)cc3)nc21] | 461.2 [M+ Na]⁺ | 1.1 (A) |
| | 2-(4-(2-(((5-amino-9-fluoro-7-methoxy-[1,2,4]triazolo[1,5-c]quinazolin-2-yl)methyl)amino)propan-2-yl)phenyl)propan-2-ol | | ∗ |
| 11.10 | 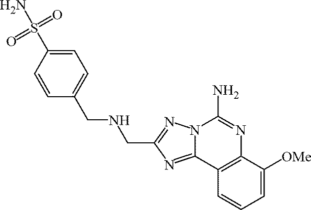 [SMILES: COc1cccc2c1nc(N)n1nc(CNCc3ccc(S(N)(=O)=O)cc3)nc21] | 414.1 | 1.4 (A) |
| | 4-((((5-amino-7-methoxy-[1,2,4]triazolo[1,5-c]quinazolin-2-yl)methyl)amino)methyl)benzenesulfonamide | | ∗ |
| 11.11 | 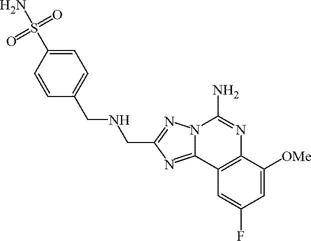 [SMILES: COc1cc(F)cc2c1nc(N)n1nc(CNCc3ccc(S(N)(=O)=O)cc3)nc21] | 454.1 [M+ Na]⁺ | 2.1 (A) |
| | 4-((((5-amino-9-fluoro-7-methoxy-[1,2,4]triazolo[1,5-c]quinazolin-2-yl)methyl)amino)methyl)benzenesulfonamide | | 15.6 (A) |
| 11.12 | 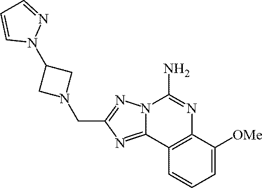 [SMILES: COc1cccc2c1nc(N)n1nc(CN3CC(n4cccn4)C3)nc21] | 351.2 | 3.7 (A) |
| | 2-((3-(1H-pyrazol-1-yl)azetidin-1-yl)methyl)-7-methoxy-[1,2,4]triazolo[1,5-c]quinazolin-5-amine | | ∗ |
| 11.13 | 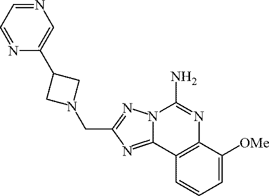 [SMILES: COc1cccc2c1nc(N)n1nc(CN3CC(c4cnccn4)C3)nc21] | 363.2 | 21.5 (A) |
| | 7-methoxy-2-((3-(pyrimidin-4-yl)azetidin-1-yl)methyl)-[1,2,4]triazolo[1,5-c]quinazolin-5-amine | | ∗ |
| 11.14 | 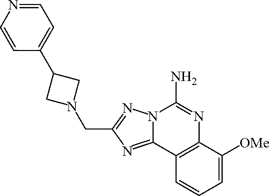 [SMILES: COc1cccc2c1nc(N)n1nc(CN3CC(c4ccncc4)C3)nc21] | 362.2 | 9.1 (A) |
| | 7-methoxy-2-((3-(pyridin-4-yl)azetidin-1-yl)methyl)-[1,2,4]triazolo[1,5-c]quinazolin-5-amine | | ∗ |

### Examples 11.8A and 11.8B

Examples 11.8A and 11.8B were resolved by chiral SFC (Column: Lux-3, 21 × 250 mm, 80% MeOH [w/ 0.1% NH₄OH]/CO₂).

### Example 12.2, N-((5-amino-7-methoxy-[1,2,4]triazolo[1,5-c]quinazolin-2-yl)methyl)cyclopropanesulfonamide.

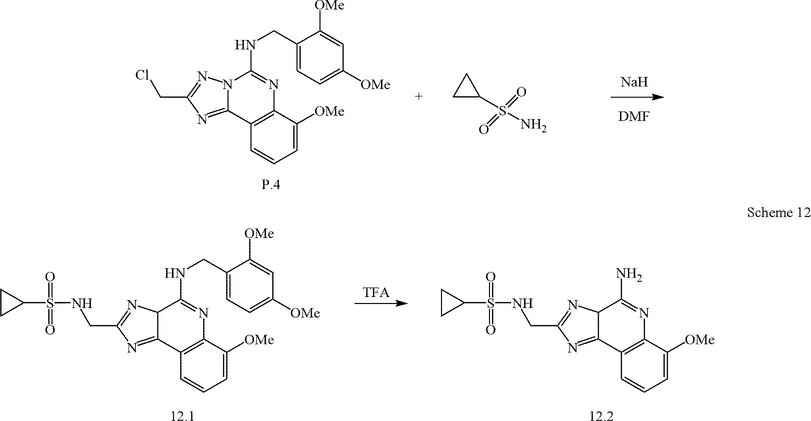

### Step 1- Intermediate 12.1, N-((5-((2,4-dimethoxybenzyl)amino)-7-methoxy-[1,2,4]triazolo[1,5-c]quinazolin-2-yl)methyl)cyclopropanesulfonamide.

NaH (60% in mineral oil, 48.3 mg, 1.21 mmol) was added to a stirred solution of cyclopropanesulfonamide (146 mg, 1.21 mmol) in DMF (5 mL). The reaction was stirred vigorously at room temperature for 30 min, at which point, a solution of 2-(chloromethyl)-N-(2,4-dimethoxybenzyl)-7-methoxy-[1,2,4]triazolo[1,5-c]quinazolin-5-amine (250 mg, 0.604 mmol) in DMF (3 mL) was added. The reaction was stirred vigorously at room temperature for 16 h. The reaction was diluted with 1 N aq. HCl (5 mL) and extracted with EtOAc (2 × 10 mL). The combined organic layers were washed with brine (5 mL), and concentrated under reduced pressure to provide N-((5-((2,4-dimethoxybenzyl)amino)-7-methoxy-[1,2,4]triazolo[1,5-c]quinazolin-2-yl)methyl)cyclopropanesulfonamide, which was used in the subsequent step without further purification. MS (ESI) m/z calc’d for C₂₃H₂₇N₆O₅S [M+H]⁺ 499.5, found 499.2.

### Step 2- Example 12.2, N-((5-amino-7-methoxy-[1,2,4]triazolo[1,5-c]quinazolin-2-yl)methyl)cyclopropanesulfonamide.

TFA (1 mL) was added to N-((5-((2,4-dimethoxybenzyl)amino)-7-methoxy-[1,2,4]triazolo[1,5-c]quinazolin-2-yl)methyl)cyclopropanesulfonamide (300 mg, 0.602 mmol). The reaction was stirred vigorously at 50° C. for 3 h. The reaction was cooled to room temperature and concentrated under reduced pressure. The concentrated residue was quenched with sat. aq. NaHCO₃ and extracted with DCM (10 mL). The organic layer was concentrated under reduced pressure and purified by silica gel chromatography (gradient elution: 15-25% MeOH/DCM N-((5-amino-7-methoxy-[1,2,4]triazolo[1,5-c]quinazolin-2-yl)methyl)cyclopropanesulfonamide (Example 12.2). MS (ESI) m/z calc’d for C₁₄H₁₆N₆O₃S [M+H]⁺ 349.3, found 349.1. ¹H NMR (500 MHz, DMSO-d₆) δ 7.87 (t, J = 6.1 Hz, 2H), 7.76 (dd, J = 8.0, 1.2 Hz, 1H), 7.33 (t, J = 7.9 Hz, 1H), 7.24 (d, 1H), 4.48 (d, J= 6.1 Hz, 2H), 3.92 (s, 3H), 2.71 - 2.59 (m, 1H), 1.02 - 0.77 (m, 4H). A_{2A} IC₅₀ 0.6 nM (A), A_{2B} IC₅₀ 116 nM (A).

### Example 13.1, 2-((bis(2-fluoropropyl)amino)methyl)-7-methoxy-[1,2,4]triazolo[1,5-c]quinazolin-5-amine.

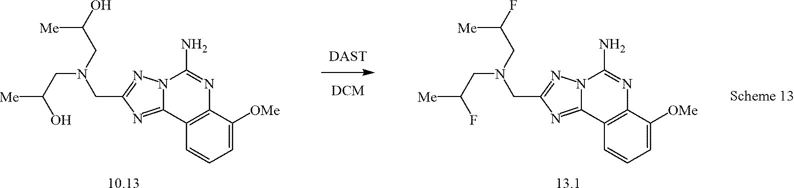

DAST (88 µL, 0.666 mmol) was added to a stirred solution of 1,1′-(((5-amino-7-methoxy-[1,2,4]triazolo[1,5-c]quinazolin-2-yl)methyl)azanediyl)bis(propan-2-ol) (40 mg, 0.111 mmol) in DCM (2 mL) at 0° C. The reaction was stirred vigorously at 0° C. for 2 h, then warmed to room temperature and stirred for an additional 16 h. The reaction was quenched with water/MeOH (0.5 mL) and DMF (2 mL) and concentrated under reduced pressure. The resulting crude residue was purified by reversed-phase HPLC [Method A], followed by preparative TLC to provide 2-((bis(2-fluoropropyl)amino)methyl)-7-methoxy-[1,2,4]triazolo[1,5-c]quinazolin-5-amine (Example 13.1). MS (ESI) m/z calc’d for C₁₇H₂₃F₂N₆O [M+H]⁺ 365.2 found 365.2. A_{2A} IC₅₀ 3.3 nM (B).

### Example 14.4, 2-((((1-(5 Fluoropyrimidin-2-yl)piperidin-4-yl)methyl)(3,3,3-trifluoropropyl)amino)methyl)-7-methoxy-[1,2,4]triazolo[1,5-c]quinazolin-5-amine.

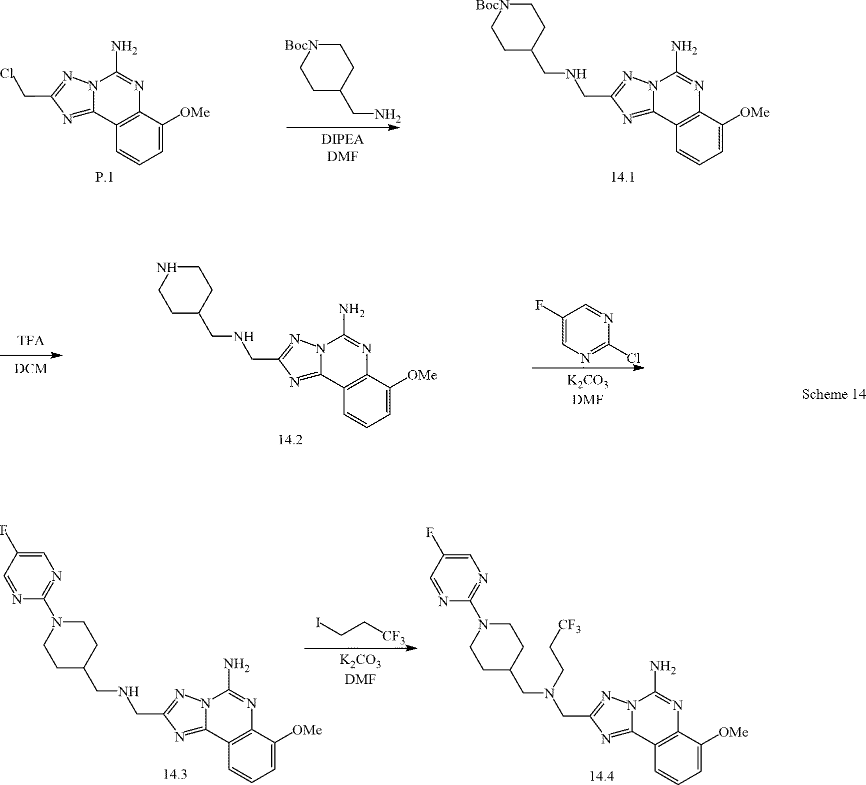

### Step 1- Intermediate 14.1, Tert-butyl 4-((((5-amino-7-methoxy-[-1,2,4]triazolo[1,5-c]quinazolin-2-yl)methyl)amino)methyl)piperidine-1-carboxylate.

DIPEA (99 µL, 0.569 mmol) was added to a stirred mixture of 2-(chloromethyl)-7-methoxy-[1,2,4]triazolo[1,5-c]quinazolin-5-amine (50 mg, 0.19 mmol) and tert-butyl 4-(aminomethyl)piperidine-1-carboxylate (61 mg, 0.284 mmol) in DMF (2 mL). The reaction was stirred vigorously at 80° C. for 16 h. The reaction was cooled to room temperature and concentrated under reduced pressure. The resulting crude residue was purified by preparative TLC (15% MeOH/DCM) to provide tert-butyl 4-((((5-amino-7-methoxy-[1,2,4]triazolo[1,5-c]quinazolin-2-yl)methyl)amino)methyl)piperidine-1-carboxylate. MS (ESI) m/z calc’d for C₂₂H₃₂N₇O₃ [M+H]⁺ 442.3, found 442.2.

### Step 2 - Intermediate 14.2, 7-methoxy-2-(((piperidin-4-ylmethyl)amino)methyl)-[1,2,4]triazolo[1,5-c]quinazolin-5-amine.

TFA (1 mL) was added to a stirred solution of tert-butyl 4-((((5-amino-7-methoxy-[1,2,4]triazolo[1,5-c]quinazolin-2-yl)methyl)amino)methyl)piperidine-1-carboxylate in DCM (1 mL). The reaction was stirred vigorously at room temperature for 3 h. The reaction was concentrated under reduced pressure. The resulting crude residue was purified by preparative TLC (15% MeOH[7 N in NH₃]/DCM) to provide 7-methoxy-2-(((piperidin-4-ylmethyl)amino)methyl)-[1,2,4]triazolo[1,5-c]quinazolin-5-amine. MS (ESI) m/z calc’d for C₁₇H₂₄N₇O [M+H]⁺ 342.2, found 342.2.

### Step 3 - Example 14.3, 2-((((1-(5-fluoropyrimidin-2-yl)piperidin-4-yl)methyl)amino)methyl)-7-methoxy-[1,2,4]triazolo[1,5-c]quinazolin-5-amine.

A mixture of 7-methoxy-2-(((piperidin-4-ylmethyl)amino)methyl)-[1,2,4]triazolo[1,5-c]quinazolin-5-amine (30 mg, 0.0.088 mmol), 2-chloro-5-fluoro-pyrimidine (23.3 mg, 0.176 mmol), and K₂CO₃ (36.4 mg, 0.264 mmol) in DMF (5 mL) was stirred vigorously at 80° C. for 3 h. The reaction was cooled to room temperature and concentrated under reduced pressure The resulting crude residue was purified by preparative TLC (15% MeOH/DCM) to provide 2-((((1-(5-fluoropyrimidin-2-yl)piperidin-4-yl)methyl)amino)methyl)-7-methoxy-[1,2,4]triazolo[1,5-c]quinazolin-5-amine (Example 14.3). MS (ESI) m/z calc’d for C₂₁H₂₅FN₉O [M+H]⁺ 438.2, found 438.2. A_{2A} IC₅₀ 10.0 nM (B).

### Step 4 - Example 14.4, 2-((((1-(5 Fluoropyrimidin-2-yl)piperidin-4-yl)methyl)(3,3,3-trifluoropropyl)amino)methyl)-7-methoxy-[1,2,4]triazolo[1,5-c]quinazolin-5-amine.

DIPEA (33.4 µL, 0.192 mmol) was added to a mixture of 2-((((1-(5-fluoropyrimidin-2-yl)piperidin-4-yl)methyl)amino)methyl)-7-methoxy-[1,2,4]triazolo[1,5-c]quinazolin-5-amine (28 mg, 0.064 mmol) and 1,1,1-trifluoro-3-iodopropane (15 µL, 0.128 mmol) in DMF (1 mL). The reaction was stirred vigorously at 80° C. for 16 h. The reaction was cooled to room temperature and concentrated under reduced pressure. The resulting crude residue was purified by preparative TLC (100% EtOAc) to provide 2-((((1-(5-fluoropyrimidin-2-yl)piperidin-4-yl)methyl)(3,3,3-trifluoropropyl)amino)methyl)-7-methoxy-[1,2,4]triazolo[1,5-c]quinazolin-5-amine (Example 14.4). MS (ESI) m/z calc’d for C₂₂H₃₂N₇O₃ [M+H]⁺ 534.3, found 534.3. A_{2A} IC₅₀ 38.2 nM (B).

### Example 15.1, 2-((ethyl(4-(trifluoromethyl)benzyl)amino)methyl)-7-methoxy-[1,2,4]triazolo[1,5-c]quinazolin-5-amine.

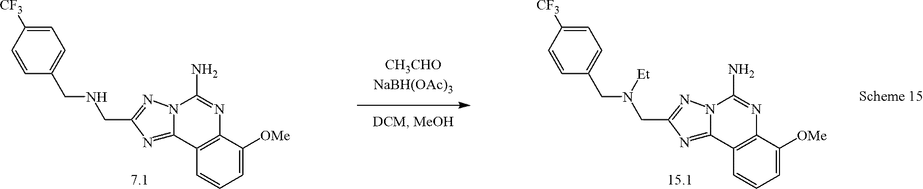

A solution of CH₃CHO (60 mg, 1.362 mmol) in DCM (1 mL) was added to a stirred solution of 7-methoxy-2-(((4-(trifluoromethyl)benzyl)amino)methyl)-[1,2,4]triazolo[1,5-c]quinazolin-5-amine (75 mg, 0.186 mmol) and NaBH(OAc)₃ (70 mg, 0.330 mmol) in DCM (9 mL) and MeOH (1 mL). The reaction was stirred vigorously at room temperature for 1 h. The reaction was diluted with water (10 mL) and extracted with DCM (100 mL). The combined organic layers were dried over anhydrous Na₂SO₄, filtered, and concentrated under reduced pressure. The resulting crude residue was purified by preparative TLC (100% EtOAc). The resulting solid was diluted in diethyl ether (5 mL) and triturated in hexanes (30 mL). The resulting solid was filtered, washed with hexanes (10 mL), and dried under reduced pressure to provide 2-((ethyl(4-(trifluoromethyl)benzyl)amino)methyl)-7-methoxy-[1,2,4]triazolo[1,5-c]quinazolin-5-amine (Example 15.1). MS (ESI) m/z calc’d for C₂₁H₂₂F₃N₆O [M+H]⁺ 431.2 found 430.9. A_{2A} IC₅₀ 3.2 nM (B).

The following examples in Table 20 were prepared according to Scheme 15 and General Scheme 7, using the appropriate amine and aldehyde intermediates. Compounds were generally purified by trituration, filtering, and washing with an appropriate solvent, preparative TLC, silica gel chromatography, or reversed-phase prep-HPLC. Asterisk (*) indicates that A_{2B} data is not available.

TABLE 20

Examples Prepared According to Scheme 15

| Entry | Structure | Observed m/z [M + H]⁺ | A2a IC₅₀ (nM) |
|---|---|---|---|
| | Name | | A2b IC₅₀ (nM) |
| 15.2 | 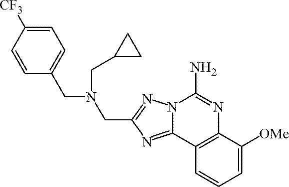 [SMILES: COc1cccc2c1nc(N)n1nc(CN(Cc3ccc(C(F)(F)F)cc3)CC3CC3)nc21] | 456.9 | 3.0 (B) |
| | 2-(((cyclopropylmethyl)(4-(trifluoromethyl)benzyl)amino)methyl)-7-methoxy-[1,2,4]triazolo[1,5-c]quinazolin-5-amine | | ∗ |
| 15.3 | 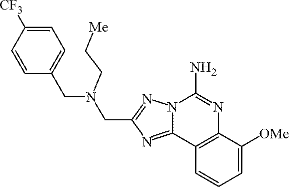 [SMILES: CCCN(Cc1ccc(C(F)(F)F)cc1)Cc1nc2c3cccc(OC)c3nc(N)n2n1] | 444.9 | 2.3 (B) |
| | 7-methoxy-2-((propyl(4-(trifluoromethyl)benzyl)amino)methyl)-[1,2,4]triazolo[1,5-c]quinazolin-5-amine | | ∗ |
| 15.4 | 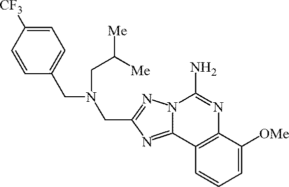 [SMILES: COc1cccc2c1nc(N)n1nc(CN(Cc3ccc(C(F)(F)F)cc3)CC(C)C)nc21] | 459.4 | 4.2 (B) |
| | 2-((isobutyl(4-(trifluoromethyl)benzyl)amino)methyl)-7-methoxy-[1,2,4]triazolo[1,5-c]quinazolin-5-amine | | ∗ |
| 15.5 | 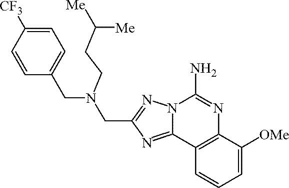 [SMILES: COc1cccc2c1nc(N)n1nc(CN(CCC(C)C)Cc3ccc(C(F)(F)F)cc3)nc21] | 473.2 | 101 (B) |
| | 2-((isopentyl(4-(trifluoromethyl)benzyl)amino)methyl)-7-methoxy-[1,2,4]triazolo[1,5-c]quinazolin-5-amine | | ∗ |
| 15.6 | 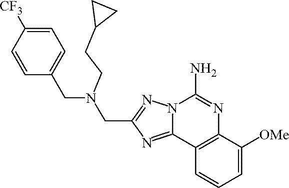 [SMILES: COc1cccc2c1nc(N)n1nc(CN(CCC3CC3)Cc3ccc(C(F)(F)F)cc3)nc21] | 471.2 | 7.1 (B) |
| | 2-(((2-cyclopropylethyl)(4-(trifluoromethyl)benzyl)amino)methyl)-7-methoxy-[1,2,4]triazolo[1,5-c]quinazolin-5-amine | | ∗ |
| 15.7 | 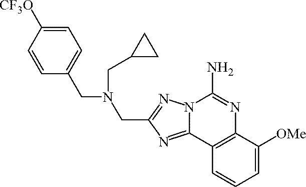 | 473.2 | 3.9 (B) |
| | 2-(((cyclopropylmethyl)(4-(trifluoromethoxy)benzyl)amino)methyl)-7-methoxy-[1,2,4]triazolo[1,5-c]quinazolin-5-amine | | ∗ |
| 15.8 | 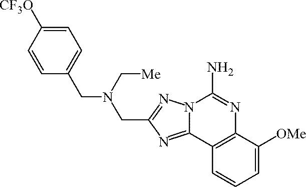 | 447.2 | 3.2 (B) |
| | 2-((ethyl(4-(trifluoromethoxy)benzyl)amino)methyl)-7-methoxy-[1,2,4]triazolo[1,5-c]quinazolin-5-amine | | ∗ |
| 15.9 | 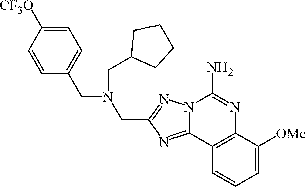 | 501.2 | 123 (B) |
| | 2-(((cyclopentylmethyl)(4-(trifluoromethoxy)benzyl)amino)methyl)-7-methoxy-[1,2,4]triazolo[1,5-c]quinazolin-5-amine | | ∗ |
| 15.10 | 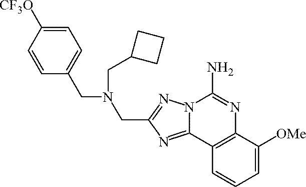 | 487.0 | 8.5 (B) |
| | 2-(((cyclobutylmethyl)(4-(trifluoromethoxy)benzyl)amino)methyl)-7-methoxy-[1,2,4]triazolo[1,5-c]quinazolin-5-amine | | ∗ |
| 15.11 | 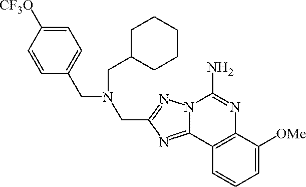 | 515.0 | 123 (B) |
| | 2-(((cyclohexylmethyl)(4-(trifluoromethoxy)benzyl)amino)methyl)-7-methoxy-[1,2,4]triazolo[1,5-c]quinazolin-5-amine | | ∗ |
| 15.12 | 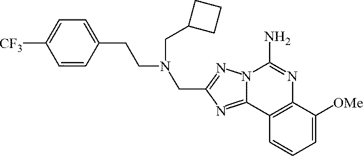 [SMILES: COc1cccc2c1nc(N)n1nc(CN(CCc3ccc(C(F)(F)F)cc3)CC3CCC3)nc21] | 485.2 | 23.6 (B) |
| | 2-(((cyclobutylmethyl)(4-(trifluoromethyl)phenethyl)amino)methyl)-7-methoxy-[1,2,4]triazolo[1,5-c]quinazolin-5-amine | | ∗ |
| 15.13 | 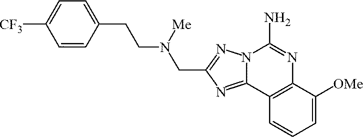 [SMILES: COc1cccc2c1nc(N)n1nc(CN(C)CCc3ccc(C(F)(F)F)cc3)nc21] | 431.0 | 7.9 (B) |
| | 7-methoxy-2-((methyl(4-(trifluoromethyl)phenethyl)amino)methyl)-[1,2,4]triazolo[1,5-c]quinazolin-5-amine | | ∗ |
| 15.14 | 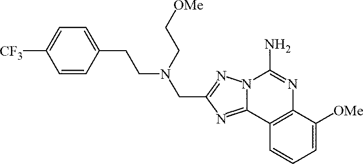 [SMILES: COCCN(CCc1ccc(C(F)(F)F)cc1)Cc1nc2c3cccc(OC)c3nc(N)n2n1] | 475.2 | 16.5 (B) |
| | 7-methoxy-2-(((2-methoxyethyl)(4-(trifluoromethyl)phenethyl)amino)methyl)-[1,2,4]triazolo[1,5-c]quinazolin-5-amine | | ∗ |
| 15.15 | 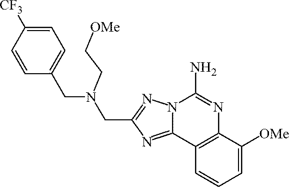 [SMILES: COCCN(Cc1ccc(C(F)(F)F)cc1)Cc1nc2c3cccc(OC)c3nc(N)n2n1] | 461.3 | 6.6 (B) |
| | 7-methoxy-2-(((2-methoxyethyl)(4-(trifluoromethyl)benzyl)amino)methyl)-[1,2,4]triazolo[1,5-c]quinazolin-5-amine | | ∗ |
| 15.16 | 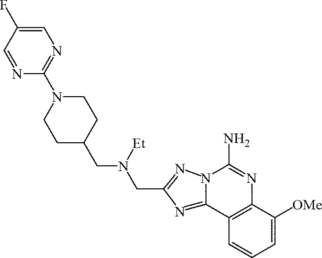 [SMILES: CCN(Cc1nc2c3cccc(OC)c3nc(N)n2n1)CC1CCN(c2ncc(F)cn2)CC1] | 466.3 | 31.0 (B) |
| | 2-((ethyl((1-(5-fluoropyrimidin-2-yl)piperidin-4-yl)methyl)amino)methyl)-7-methoxy-[1,2,4]triazolo[1,5-c]quinazolin-5-amine | | ∗ |
| 15.17 | 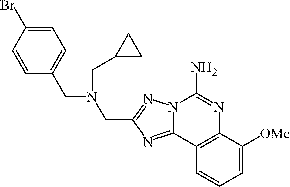 [SMILES: COc1cccc2c1nc(N)n1nc(CN(Cc3ccc(Br)cc3)CC3CC3)nc21] | 467.2, 469.2 | 3.2 (B) |
| | 2-(((4-bromobenzyl)(cyclopropylmethyl)amino)methyl)- | | ∗ |
| | 7-methoxy-[1,2,4]triazolo[1,5-c]quinazolin-5-amine | | |

### Example 16.1,2-(((cyclopropylmethyl)(pyridin-2-ylmethyl)amino)methyl)-7-methoxy-[1,2,4]triazolo[1,5-c]quinazolin-5-amine.

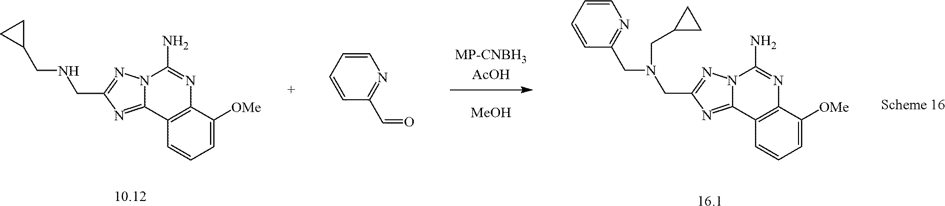

AcOH (0.15 mL), picolinaldehyde (35.9 mg, 0.335 mmol), and MP-CNBH₃ (1.23 mmol/g, 410 mg) were sequentially added to a stirred solution of 2-(((cyclopropylmethyl)amino)methyl)-7-methoxy-[1,2,4]triazolo[1,5-c]quinazolin-5-amine (50 mg, 0.168 mmol) in MeOH (1.5 mL). The reaction was stirred vigorously at room temperature for 1 h. The reaction was then filtered and concentrated under reduced pressure. The resulting crude residue was purified by reversed-phase prep-HPLC [Method A] to provide 2-(((cyclopropylmethyl)(pyridin-2-ylmethyl)amino)methyl)-7-methoxy-[1,2,4]triazolo[1,5-c]quinazolin-5-amine (Example 16.1). MS (ESI) m/z calc’d for C₂₁H₂₄N₇O [M+H]⁺ 390.2 found 390.3. A_{2A} IC₅₀ 12.8 nM (B).

The following examples in Table 21 were prepared according to Scheme 16 and General Scheme 7, using Intermediate 10.12 and the appropriate carbonyl. Asterisk (*) indicates that A_{2B} data is not available.

TABLE 21

Examples Prepared According to Scheme 16

| Entry | Structure | Observed m/z [M + H]⁺ | A2a IC₅₀ (nM) |
|---|---|---|---|
| | Name | | A2b IC₅₀ (nM) |
| 16.2 | 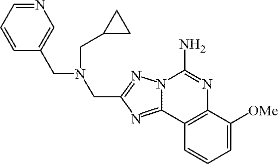 [SMILES: COc1cccc2c1nc(N)n1nc(CN(Cc3cccnc3)CC3CC3)nc21] | 390.2 | 5.3 (B) |
| | 2-(((cyclopropylmethyl)(pyridin-3-ylmethyl)amino)methyl)-7-methoxy-[1,2,4]triazolo[1,5-c]quinazolin-5-amine | | ∗ |
| 16.3 | 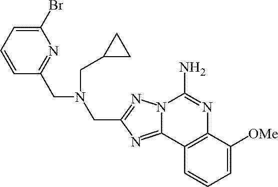 [SMILES: COc1cccc2c1nc(N)n1nc(CN(Cc3cccc(Br)n3)CC3CC3)nc21] | 468.2, 470.2 | 14.6 (B) |
| | 2-((((6-bromopyridin-2-yl)methyl)(cyclopropylmethyl)amino)methyl)-7-methoxy-[1,2,4]triazolo[1,5-c]quinazolin-5-amine | | ∗ |
| 16.4 | 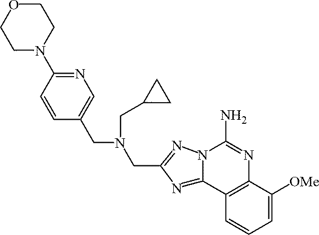 [SMILES: COc1cccc2c1nc(N)n1nc(CN(Cc3ccc(N4CCOCC4)nc3)CC3CC3)nc21] | 475.2 | 10.9 (B) |
| | 2-(((cyclopropylmethyl)((6-morpholinopyridin-3-yl)methyl)amino)methyl)-7-methoxy-[1,2,4]triazolo[1,5-c]quinazolin-5-amine | | ∗ |
| 16.5 | 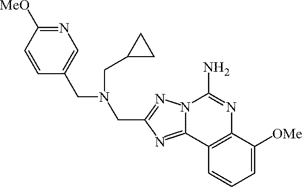 [SMILES: COc1ccc(CN(Cc2nc3c4cccc(OC)c4nc(N)n3n2)CC2CC2)cn1] | 420.6 | 9.2 (B) |
| | 2-(((cyclopropylmethyl)((6-methoxypyridin-3-yl)methyl)amino)methyl)-7-methoxy-[1,2,4]triazolo[1,5-c]quinazolin-5-amine | | ∗ |
| 16.6 | 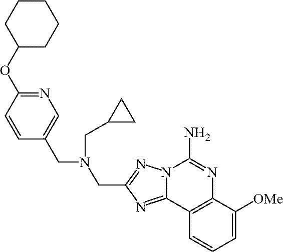 [SMILES: COc1cccc2c1nc(N)n1nc(CN(Cc3ccc(OC4CCCCC4)nc3)CC3CC3)nc21] | 488.5 | 6.6 (B) |
| | 2-((((6-(cyclohexyloxy)pyridin-3-yl)methyl)(cyclopropylmethyl)amino)methyl)-7-methoxy-[1,2,4]triazolo[1,5-c]quinazolin-5-amine | | ∗ |
| 16.7 | 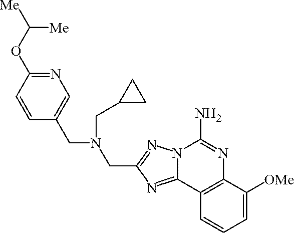 [SMILES: COc1cccc2c1nc(N)n1nc(CN(Cc3ccc(OC(C)C)nc3)CC3CC3)nc21] | 448.3 | 13.5 (B) |
| | 2-(((cyclopropylmethyl)((6-isopropoxypyridin-3-yl)methyl)amino)methyl)-7-methoxy-[1,2,4]triazolo[1,5-c]quinazolin-5-amine | | ∗ |
| 16.8 | 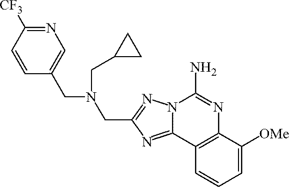 [SMILES: COc1cccc2c1nc(N)n1nc(CN(Cc3ccc(C(F)(F)F)nc3)CC3CC3)nc21] | 458.4 | 24.1 (B) |
| | 2-(((cyclopropylmethyl)((6-(trifluoromethyl)pyridin-3-yl)methyl)amino)methyl)-7-methoxy-[1,2,4]triazolo[1,5-c]quinazolin-5-amine | | ∗ |
| 16.9 | 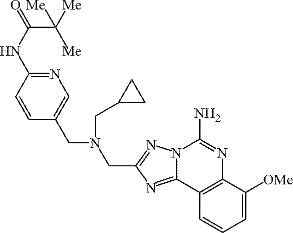 [SMILES: COc1cccc2c1nc(N)n1nc(CN(Cc3ccc(NC(=O)C(C)(C)C)nc3)CC3CC3)nc21] | 489.3 | 5.2 (B) |
| | tert-butyl (5-((((5-amino-7-methoxy-[1,2,4]triazolo[1,5-c]quinazolin-2-yl)methyl)(cyclopropylmethyl)amino)methyl)pyridin-2-yl)carbamate | | ∗ |
| 16.10 | 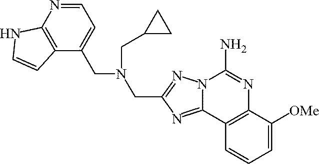 [SMILES: COc1cccc2c1nc(N)n1nc(CN(Cc3ccnc4[nH]ccc34)CC3CC3)nc21] | 429.5 | 2.3 (B) |
| | 2-((((1H-pyrrolo[2,3-b]pyridin-4-yl)methyl)(cyclopropylmethyl)amino)methyl)-7-methoxy-[1,2,4]triazolo[1,5-c]quinazolin-5-amine | | ∗ |
| 16.11 | 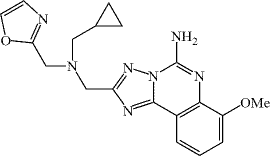 [SMILES: COc1cccc2c1nc(N)n1nc(CN(Cc3ncco3)CC3CC3)nc21] | 380.2 | 9.4 (B) |
| | 2-(((cyclopropylmethyl)(oxazol-2-ylmethyl)amino)methyl)-7-methoxy-[1,2,4]triazolo[1,5-c]quinazolin-5-amine | | ∗ |
| 16.12 | 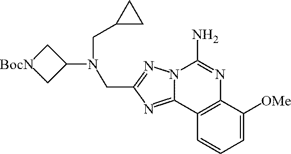 [SMILES: COc1cccc2c1nc(N)n1nc(CN(CC3CC3)C3CN(C(=O)OC(C)(C)C)C3)nc21] | 454.3 | 5.7 (B) |
| | tert-butyl 3-(((5-amino-7-methoxy-[1,2,4]triazolo[1,5-c]quinazolin-2-yl)methyl)(cyclopropylmethyl)amino)azetidine-1-carboxylate | | ∗ |
| 16.13 | 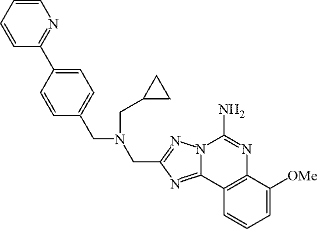 [SMILES: COc1cccc2c1nc(N)n1nc(CN(Cc3ccc(-c4ccccn4)cc3)CC3CC3)nc21] | 466.2 | 2.6 (B) |
| | 2-(((cyclopropylmethyl)(4-(pyridin-2-yl)benzyl)amino)methyl)-7-methoxy-[1,2,4]triazolo[1,5-c]quinazolin-5-amine | | ∗ |
| 16.14 | 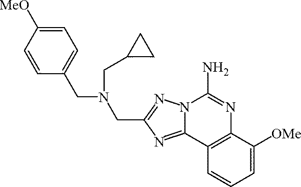 [SMILES: COc1ccc(CN(Cc2nc3c4cccc(OC)c4nc(N)n3n2)CC2CC2)cc1] | 419.1 | 7.2 (B) |
| | 2-(((cyclopropylmethyl)(4-methoxybenzyl)amino)methyl)-7-methoxy-[1,2,4]triazolo[1,5-c]quinazolin-5-amine | | ∗ |
| 16.15 | 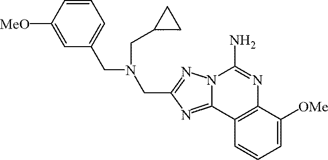 [SMILES: COc1cccc(CN(Cc2nc3c4cccc(OC)c4nc(N)n3n2)CC2CC2)c1] | 419.5 | 3.5 (B) |
| | 2-(((cyclopropylmethyl)(3-methoxybenzyl)amino)methyl)-7-methoxy-[1,2,4]triazolo[1,5-c]quinazolin-5-amine | | ∗ |
| 16.16 | 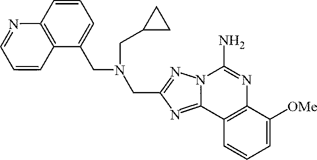 [SMILES: COc1cccc2c1nc(N)n1nc(CN(Cc3cccc4ncccc34)CC3CC3)nc21] | 440.2 | 3.5 (B) |
| | 2-(((cyclopropylmethyl)(quinolin-5-ylmethyl)amino)methyl)-7-methoxy-[1,2,4]triazolo[1,5-c]quinazolin-5-amine | | ∗ |
| 16.17 | 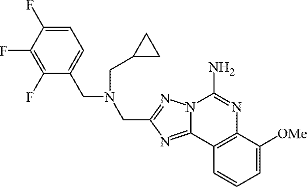 [SMILES: COc1cccc2c1nc(N)n1nc(CN(Cc3ccc(F)c(F)c3F)CC3CC3)nc21] | 443.4 | 4.8 (B) |
| | 2-(((cyclopropylmethyl)(2,3,4-trifluorobenzyl)amino)methyl)-7-methoxy-[1,2,4]triazolo[1,5-c]quinazolin-5-amine | | ∗ |

### Example 17.1,2-(((cyclopropylmethyl)(2,2-difluoroethyl)amino)methyl)-7-methoxy-[1,2,4]triazolo[1,5-c]quinazolin-5-amine.

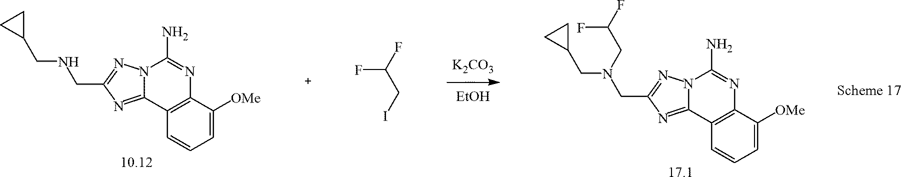

A mixture of 2-(((cyclopropylmethyl)amino)methyl)-7-methoxy-[1,2,4]triazolo[1,5-c]quinazolin-5-amine (60 mg, 0.201 mmol), 1,1-difluoro-2-iodoethane (193 mg, 1.01 mmol), and K₂CO₃ (111 mg, 0.804 mmol) in EtOH (2.5 mL) was heated to 95° C. for 16 h in a microwave reactor. The reaction was cooled to room temperature and concentrated under reduced pressure. The resulting crude residue was purified by reversed-phase HPLC [Method A] to provide 2-(((cyclopropylmethyl)(2,2-difluoroethyl)amino)methyl)-7-methoxy-[1,2,4]triazolo[1,5-c]quinazolin-5-amine (Example 17.1). MS (ESI) m/z calc’d for C₁₇H₂₁F₂N₆O [M+H]⁺ 363.2 found 363.1. A_{2A} IC₅₀ 2.5 nM (B).

The following example in Table 22 was prepared according to Scheme 17 and General Scheme 6, using Intermediate 10.12 and the appropriate halide. Asterisk (*) indicates that A_{2B} data is not available.

TABLE 22

Examples Prepared According to Scheme 17

| Entry | Structure | Observed m/z [M + H]⁺ | A2a IC₅₀ (nM) |
|---|---|---|---|
| | Name | | A2b IC₅₀ (nM) |
| 17.2 | 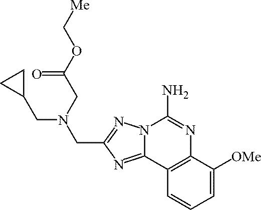 [SMILES: CCOC(=O)CN(Cc1nc2c3cccc(OC)c3nc(N)n2n1)CC1CC1] | 385.2 | 13.0 (B) |
| | ethyl N-((5-amino-7-methoxy-[1,2,4]triazolo[1,5-c]quinazolin-2-yl)methyl)-N-(cyclopropylmethyl)glycinate | | ∗ |

### Example 18.1, (R)-7-methoxy-2-(((oxiran-2-ylmethyl)(4-(trifluoromethyl)benzyl)amino)methyl)-[1,2,4]triazolo[1,5-c]quinazolin-5-amine.

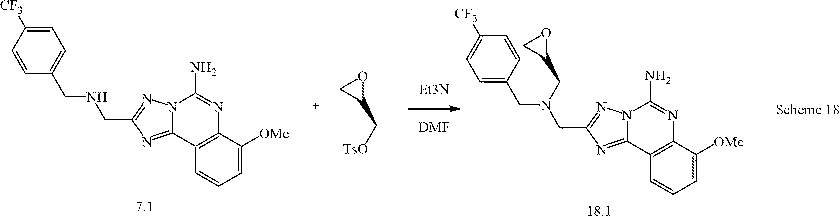

(S)-Oxiran-2-ylmethyl 4-methylbenzenesulfonate (70 mg, 0.307 mmol) was added to a stirred solution of 7-methoxy-2-(((4-(trifluoromethyl)benzyl)amino)methyl)-[1,2,4]triazolo[1,5-c]quinazolin-5-amine (30 mg, 0.075 mmol) and Et₃N (0.2 mL, 1.435 mmol) in DMF (2 mL). The reaction was stirred vigorously at 80° C. for 48 h. The reaction was cooled to room temperature, diluted with water (20 mL), and extracted with EtOAc (50 mL). The combined organic layers were dried over anhydrous Na₂SO₄, filtered, and concentrated under reduced pressure. The resulting crude residue was purified by preparative TLC (80% EtOAc/hexanes) to provide (R)-7-methoxy-2-(((oxiran-2-ylmethyl)(4-(trifluoromethyl)benzyl)amino)methyl)-[1,2,4]triazolo[1,5-c]quinazolin-5-amine (Example 18.1). MS (ESI) m/z calc’d for C₂₂H₁₂F₃N₆O₂ [M+H]⁺ 459.2 found 458.9 A_{2A} IC₅₀ 20.2 nM (B).

### Example 19.2, N-((5-amino-7-methoxy-[1,2,4]triazolo[1,5-c]quinazolin-2-yl)methyl)-N-(4-(trifluoromethyl)benzyl)methanesulfonamide.

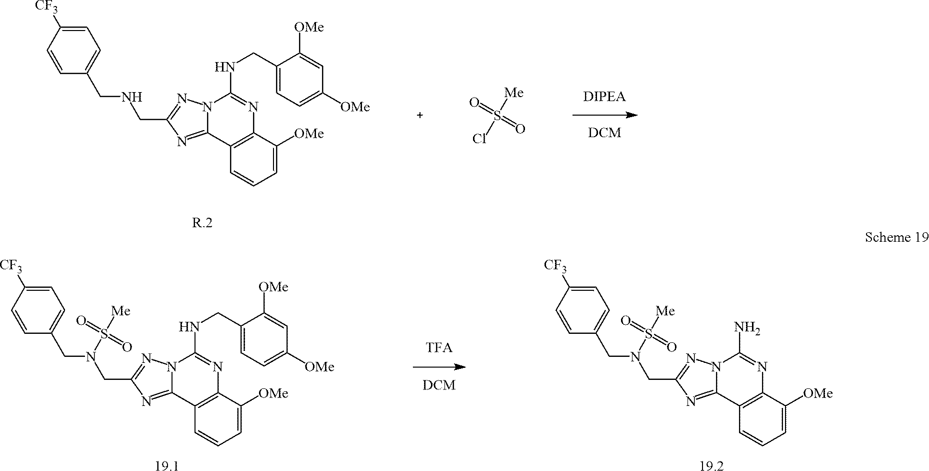

### Step 1- Intermediate 19.1, N-((5-((2,4-dimethoxybenzyl)amino)-7-methoxy-[1,2,4]triazolo[1,5-c]quinazolin-2-yl)methyl)-N-(4-(trifluoromethyl)benzyl)methanesulfonamide.

DIPEA (0.4 mL, 2.30 mmol) was added to a stirred solution of N-(2,4-dimethoxybenzyl)-7-methoxy-2-(((4-(trifluoromethyl)benzyl)amino)methyl)-[1,2,4]triazolo[1,5-c]quinazolin-5-amine (100 mg, 0.180 mmol) and methanesulfonyl chloride (100 mg, 0.873 mmol) in DCM (5 mL). The reaction was stirred vigorously at room temperature for 16 h. The reaction was diluted with water (20 mL) and extracted with DCM (50 mL). The combined organic layers were dried over anhydrous Na₂SO₄, filtered, and concentrated under reduced pressure. The resulting crude residue was purified by silica gel chromatography (gradient elution: 0-50% EtOAc/hexanes) to provide N-((5-((2,4-dimethoxybenzyl)amino)-7-methoxy-[1,2,4]triazolo[1,5-c]quinazolin-2-yl)methyl)-N-(4-(trifluoromethyl)benzyl)methanesulfonamide. MS (ESI) m/z calc’d for C₂₉H₃₀F₃N₆O₅S [M+H]⁺ 631.2, found 630.8.

### Step 2- Example 19.2, N-((5-amino-7-methoxy-[1,2,4]triazolo[1,5-clquinazolin-2-yl)methyl)-N-(4-(trifluoromethyl)benzyl)methanesulfonamide.

TFA (2 mL) was added to a stirred solution of N-((5-((2,4-dimethoxybenzyl)amino)-7-methoxy-[1,2,4]triazolo[1,5-c]quinazolin-2-yl)methyl)-N-(4-(trifluoromethyl)benzyl)methanesulfonamide (70 mg, 0.111 mmol) in DCM (3 mL). The reaction was stirred vigorously at 40° C. for 16 h. The reaction was cooled to room temperature, diluted with water (20 mL) and 1N aq. NaOH (2 mL), and extracted with DCM (50 mL). The combined organic layers were dried over anhydrous Na₂SO₄, filtered, and concentrated under reduced pressure. The resulting crude residue was purified by preparative TLC (5% MeOH/DCM) to provide N-((5-amino-7-methoxy-[1,2,4]triazolo[1,5-c]quinazolin-2-yl)methyl)-N-(4-(trifluoromethyl)benzyl)methanesulfonamide (Example 19.2). MS (ESI) m/z calc’d for C₂₀H₂₀F₃N₆O₃S [M+H]⁺ 481.1, found 481.2. A_{2A} IC₅₀ 7.5 nM (B).

The following examples in Table 23 were prepared according to Scheme 19 and General Scheme 6, using the appropriate amine intermediate and sulfonyl chloride. Asterisk (^{∗}) indicates that A_{2B} data is not available.

TABLE 23

Examples Prepared According to Scheme 19

| Entry | Structure | Observed m/z [M + H]⁺ | A2a IC₅₀ (nM) |
|---|---|---|---|
| | Name | | A2b IC₅₀ (nM) |
| 19.3 | 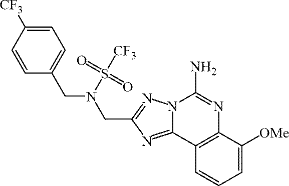 [SMILES: COc1cccc2c1nc(N)n1nc(CN(Cc3ccc(C(F)(F)F)cc3)S(=O)(=O)C(F)(F)F)nc21] | 535.2 | 115 (B) |
| | N-((5-amino-7-methoxy-[1,2,4]triazolo[1,5-c]quinazolin-2-yl)methyl)-1,1,1-trifluoro-N-(4-(trifluoromethyl)benzyl)methanesulfonamide | | ∗ |
| 19.4 | 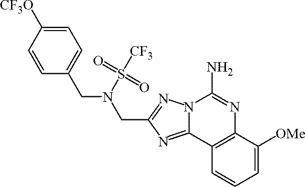 | 551.2 | 50.4 (B) |
| | N-((5-amino-7-methoxy-[1,2,4]triazolo[1,5-c]quinazolin-2-yl)methyl)-1,1,1-trifluoro-N-(4-(trifluoromethoxy)benzyl)methanesulfonamide | | ∗ |
| 19.5 | 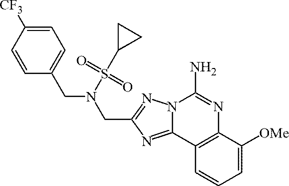 [SMILES: COc1cccc2c1nc(N)n1nc(CN(Cc3ccc(C(F)(F)F)cc3)S(=O)(=O)C3CC3)nc21] | 507.2 | 17.6 (B) |
| | N-((5-amino-7-methoxy-[1,2,4]triazolo[1,5-c]quinazolin-2-yl)methyl)-N-(4-(trifluoromethyl)benzyl)cyclopropanesulfonamide | | ∗ |

### Example 20.2, 1-((5-amino-7-methoxy-[1,2,4]triazolo[1,5-c]quinazolin-2-yl)methyl)-3-isopropyl-1-(4-(trifluoromethyl)benzyl)urea.

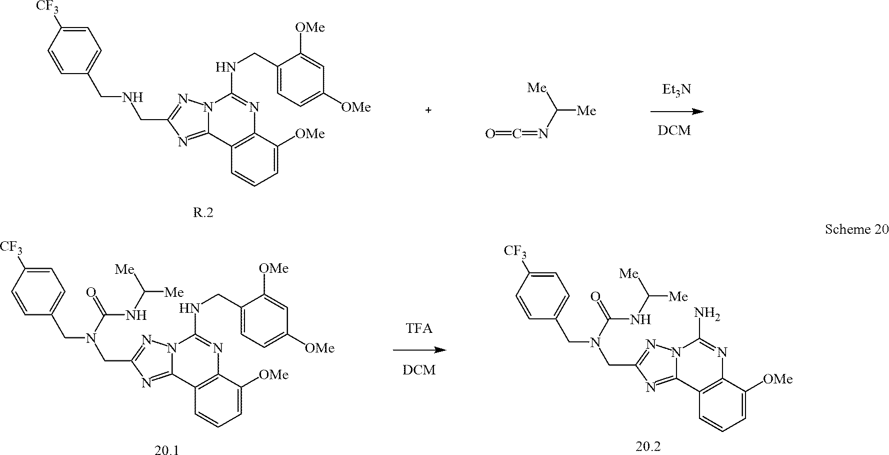

### Step 1- Intermediate 20.1, 1-((5-((2,4-dimethoxybenzyl)amino)-7-methoxy-[1,2,4]triazolo[1,5-c]quinazolin-2-yl)methyl)-3-isopropyl-1-(4-(trifluoromethyl)benzyl)urea.

Et₃N (0.2 mL, 1.435 mmol) was added to a stirred solution of N-(2,4-dimethoxybenzyl)-7-methoxy-2-(((4-(trifluoromethyl)benzyl)amino)methyl)-[1,2,4]triazolo[1,5-c]quinazolin-5-amine (150 mg, 0.271 mmol) and isopropyl isocyanate (60 mg, 0.705 mmol) in DCM. The reaction was stirred vigorously at room temperature for 16 h. The reaction was diluted with water (20 mL) and extracted with DCM (50 mL). The combined organic layers were dried over anhydrous Na₂SO₄, filtered, and concentrated under reduced pressure. The resulting crude residue was purified by preparative TLC (50% EtOAc/hexanes) to provide 1-((5-((2,4-dimethoxybenzyl)amino)-7-methoxy-[1,2,4]triazolo[1,5-c]quinazolin-2-yl)methyl)-3-isopropyl-1-(4-(trifluoromethyl)benzyl)urea. MS (ESI) m/z calc’d for C₃₂H₃₅F₃N₇O₄ [M+H]⁺ 638.3, found 638.4.

### Step 2 - Example 20.2, 1-((5-amino-7-methoxy-[1,2,4]triazolo[1,5-c]quinazolin-2-yl)methyl)-3-isopropyl-1-(4-(trifluoromethyl)benzyl)urea.

TFA (2 mL) was added to a stirred solution of 1-((5-((2,4-dimethoxybenzyl)amino)-7-methoxy-[1,2,4]triazolo[1,5-c]quinazolin-2-yl)methyl)-3-isopropyl-1-(4-(trifluoromethyl)benzyl)urea (180 mg, 0.183 mmol) in DCM (3 mL). The reaction was stirred vigorously at 50° C. for 3 h. The reaction was cooled to room temperature and concentrated under reduced pressure. The resulting crude residue was purified by preparative TLC (10% MeOH/DCM) to 1-((5-amino-7-methoxy-[1,2,4]triazolo[1,5-c]quinazolin-2-yl)methyl)-3-isopropyl-1-(4-(trifluoromethyl)benzyl)urea (Example 20.2). MS (ESI) m/z calc’d for C₂₃H₂₅F₃N₇O₂ [M+H]⁺ 488.2, found 488.2. A_{2A} IC₅₀ 50.7 nM (B).

The following examples in Table 24 were prepared according to Scheme 20 and General Scheme 6, using the appropriate amine and isocyanate. For Example 20.4, Step 1 was conducted with DIPEA instead of Et3N, and THF instead of DCM; and Step 2 was omitted as the corresponding amine (i.e. Intermediate P.1) does not have a 2,4-dimethoxybenzylamino group. Asterisk (*) indicates that A_{2B} data is not available.

TABLE 24

Examples Prepared According to Scheme 20

| Entry | Structure | Observed m/z [M + H]⁺ | A2a IC₅₀ (nM) |
|---|---|---|---|
| | Name | | A2b IC₅₀ (nM) |
| 20.3 | 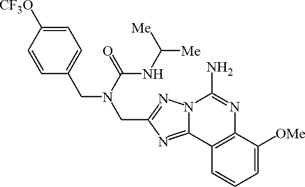 | 535.2 | 22.6 (B) |
| | 1-((5-amino-7-methoxy-[1,2,4]triazolo[1,5-c]quinazolin-2-yl)methyl)-3-isopropyl-1-(4-(trifluoromethoxy)benzyl)urea | | ∗ |
| 20.4 | 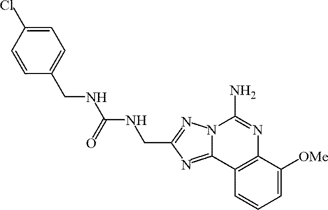 [SMILES: COc1cccc2c1nc(N)n1nc(CNC(=O)NCc3ccc(Cl)cc3)nc21] | 412.2 | 5.7 (B) |
| | 1-((5-amino-7-methoxy-[1,2,4]triazolo[1,5-c]quinazolin-2-yl)methyl)-3-(4-chlorobenzyl)urea | | ∗ |

### Example 21.1, 2-((allyl(4-(trifluoromethyl)benzyl)amino)methyl)-7-methoxy-[1,2,4]triazolo[1,5-c]quinazolin-5-amine.

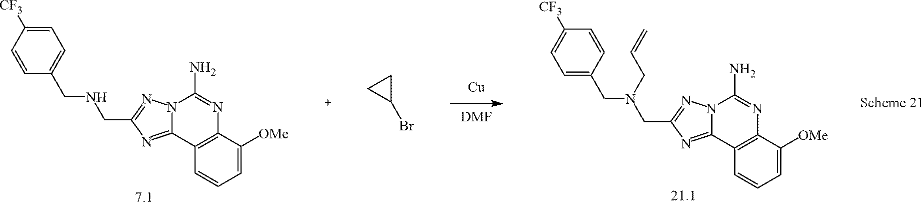

Cyclopropyl bromide (50 mg, 0.413 mmol) was added to a stirred solution of 7-methoxy-2-(((4-(trifluoromethyl)benzyl)amino)methyl)-[1,2,4]triazolo[1,5-c]quinazolin-5-amine (50 mg, 0.124 mmol) and Cu (45 micron powder, 10 mg, 0.157 mmol) in DMF (2 mL). The reaction was heated to 100° C. for 1 h in a microwave reactor. The reaction was cooled to room temperature, diluted with water (30 mL), and extracted with EtOAc (100 mL). The combined organic layers were dried over anhydrous Na₂SO₄, filtered, and concentrated under reduced pressure. The resulting crude residue was purified by preparative TLC (50% EtOAc/hexanes) to provide 2-((allyl(4-(trifluoromethyl)benzyl)amino)methyl)-7-methoxy-[1,2,4]triazolo[1,5-c]quinazolin-5-amine as a major by-product of the reaction (Example 21.1). MS (ESI) m/z calc’d for C₂₂H₂₂F₃N₆O [M+H]⁺ 443.2 found 443.9 A_{2A} IC₅₀ 11.1 nM (B).

The following example in Table 25 was prepared according to Scheme 21 and General Scheme 6, using Example 8.6 and cyclopropyl bromide. Asterisk (*) indicates that A_{2B} data is not available.

TABLE 25

Examples Prepared According to Scheme 21

| Entry | Structure | Observed m/z [M + H]⁺ | A2a IC₅₀ (nM) |
|---|---|---|---|
| | Name | | A2b IC₅₀ (nM) |
| 21.2 | 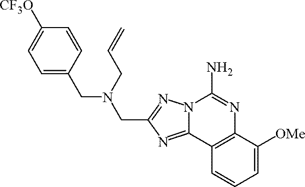 | 459.2 | 3.1 (B) |
| | 2-((allyl(4-(trifluoromethoxy)benzyl)amino)methyl)-7-methoxy-[1,2,4]triazolo[1,5-c]quinazolin-5-amine | | ∗ |

### Example 22.2, 2-(3-((2-(5-amino-7-methoxy-[1,2,4]triazolo[1,5-c]quinazolin-2-yl)ethyl)amino)bicyclo[1.1.1]pentan-1-yl)propan-2-ol.

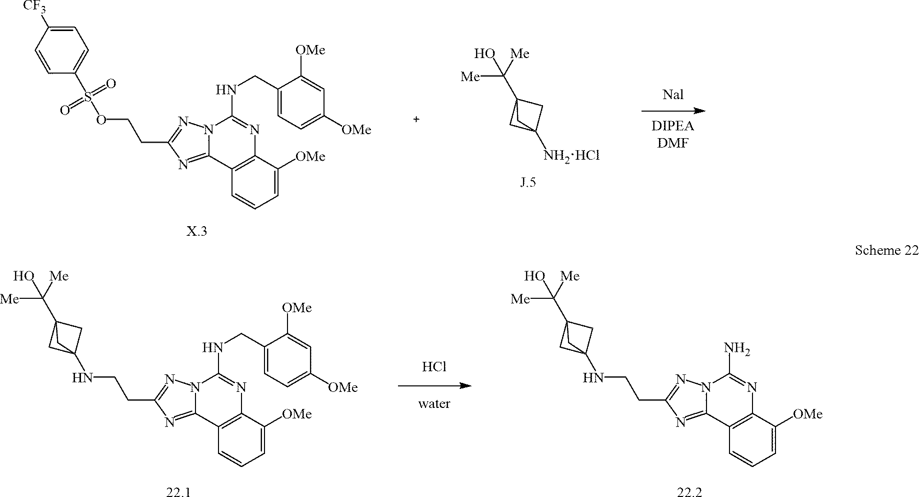

### Step 1 - Intermediate 22.1, 2-(3-((2-(5-((2,4-dimethoxybenzyl)amino)-7-methoxy-[1,2,4]triazolo[1,5-c]quinazolin-2-yl)ethyl)amino)bicyclo[1.1.1]pentan-1-yl)propan-2-ol.

DIPEA (52.3 µL, 0.300 mmol) was added to a suspension of 2-(5-((2,4-dimethoxybenzyl)amino)-7-methoxy-[1,2,4]triazolo[1,5-c]quinazolin-2-yl)ethyl 4-(trifluoromethyl)benzenesulfonate (81 mg, 0.150 mmol), 2-(3-aminobicyclo[1.1.1]pentan-1-yl)propan-2-ol hydrochloride (27.9 mg, 0.157 mmol), and NaI (44.8 mg, 0.300 mmol) in DMF (1.5 mL). The reaction was stirred vigorously at 60° C. for 16 h. The reaction was cooled to room temperature, and concentrated under reduced pressure to provide 2-(3-((2-(5-((2,4-dimethoxybenzyl)amino)-7-methoxy-[1,2,4]triazolo[1,5-c]quinazolin-2-yl)ethyl)amino)bicyclo[1.1.1]pentan-1-yl)propan-2-ol, which was carried crude without further purification. MS (ESI) m/z calc’d for C₂₉H₃₇N₆O₄ [M+H]⁺ 533.3, found 533.3.

### Step 2 - Example 22.2, 2-(3-((2-(5-amino-7-methoxy-[1,2,4]triazolo[1,5-c]quinazolin-2-yl)ethyl)amino)bicyclo[1.1.1]pentan-1-yl)propan-2-ol.

Water (0.75 mL) and HCl (37% aq., 0.31 mL, 3.74 mmol) were sequentially added to 2-(3-((2-(5-((2,4-dimethoxybenzyl)amino)-7-methoxy-[1,2,4]triazolo[1,5-c]quinazolin-2-yl)ethyl)amino)bicyclo[1.1.1]pentan-1-yl)propan-2-ol from the previous step. The reaction was stirred vigorously at 60° C. for 16 h. The reaction was cooled to room temperature and filtered. The resulting filtrate was cooled to 0° C. and quenched with a solution of NaOH (179 mg, 4.49 mmol) in water (5 mL). The resulting mixture was extracted with 3:1 EtOAc/EtOH (3 × 10 mL). The combined organic layers were dried over anhydrous MgSOa, filtered, and concentrated under reduced pressure. The resulting crude residue was purified by reversed-phase HPLC [Method A] to provide 2-(3-((2-(5-amino-7-methoxy-[1,2,4]triazolo[1,5-c]quinazolin-2-yl)ethyl)amino)bicyclo[1.1.1]pentan-1-yl)propan-2-ol (Example 22.2). MS (ESI) m/z calc’d for C₂₀H₂₇N₆O₂ [M+H]⁺ 383.2, found 383.2. A_{2A} IC₅₀ 12.3 nM (A).

The following examples in Table 26 were prepared according to Scheme 22 and General Scheme 8, using Intermediate V.1 and the appropriate amine. Step 1 of Examples 22.3-22.7 was conducted in the absence of NaI. Step 1 of Example 22.8 was conducted using KI instead of NaI. Step 2 of all examples listed in the table was conducted using TFA and DCM as described throughout. Asterisk (^{∗}) indicates that A_{2B} data is not available.

TABLE 26

Examples Prepared According to Scheme 22

| Entry | Structure | Observed m/z [M + H]⁺ | A2a IC₅₀ (nM) |
|---|---|---|---|
| | Name | | A2b IC₅₀ (nM) |
| 22.3 | 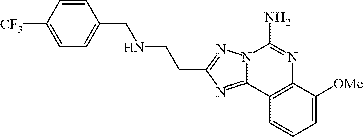 [SMILES: COc1cccc2c1nc(N)n1nc(CCNCc3ccc(C(F)(F)F)cc3)nc21] | 417.2 | 16.7 (B) |
| | 7-methoxy-2-(2-((4-(trifluoromethyl)benzyl)amino)ethyl)-[1,2,4]triazolo[1,5-c]quinazolin-5-amine | | ∗ |
| 22.4 | 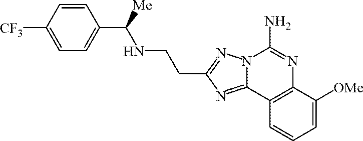 [SMILES: COc1cccc2c1nc(N)n1nc(CCN[C@H](C)c3ccc(C(F)(F)F)cc3)nc21] | 431.2 | 15.9 (B) |
| | (R)-7-methoxy-2-(2-((1-(4-(trifluoromethyl)phenyl)ethyl)amino)ethyl)-[1,2,4]triazolo[1,5-c]quinazolin-5-amine | | ∗ |
| 22.5 | 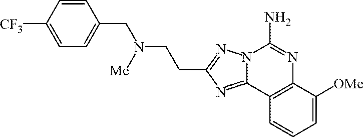 [SMILES: COc1cccc2c1nc(N)n1nc(CCN(C)Cc3ccc(C(F)(F)F)cc3)nc21] | 431.2 | 4.1 (B) |
| | 7-methoxy-2-(2-(methyl(4-(trifluoromethyl)benzyl)amino)ethyl)-[1,2,4]triazolo[1,5-c]quinazolin-5-amine | | ∗ |
| 22.6 | 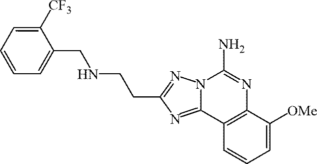 [SMILES: COc1cccc2c1nc(N)n1nc(CCNCc3ccccc3C(F)(F)F)nc21] | 417.2 | 9.7 (B) |
| | 7-methoxy-2-(2-((2-(trifluoromethyl)benzyl)amino)ethyl)-[1,2,4]triazolo[1,5-c]quinazolin-5-amine | | ∗ |
| 22.7 | 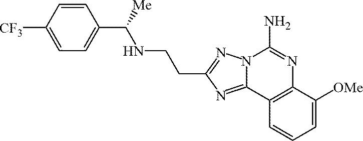 [SMILES: COc1cccc2c1nc(N)n1nc(CCN[C@@H](C)c3ccc(C(F)(F)F)cc3)nc21] | 431.0 | 27.3 (B) |
| | (S)-7-methoxy-2-(2-((1-(4-(trifluoromethyl)phenyl)ethyl)amino)ethyl)-[1,2,4]triazolo[1,5-c]quinazolin-5-amine | | ∗ |
| 22.8 | 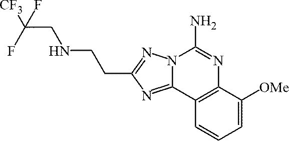 [SMILES: COc1cccc2c1nc(N)n1nc(CCNCC(F)(F)C(F)(F)F)nc21] | 391.2 | 10.9 (B) |
| | 7-methoxy-2-(2-((2,2,3,3,3-pentafluoropropyl)amino)ethyl)-[1,2,4]triazolo[1,5-c] quinazolin-5-amine | | ∗ |

### Example 23.2, 7-methoxy-2-(2-((1-methyl-1H-pyrazol-4-yl)amino)ethyl)-[1,2,4]triazolo[1,5-c]quinazolin-5-amine.

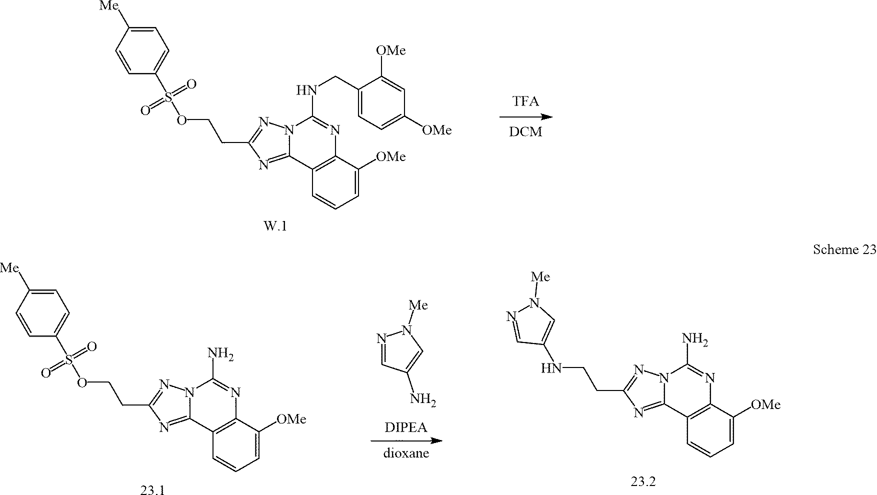

### Step 1 - Intermediate 23.1, 2-(5-amino-9-fluoro-8-methoxy-[1,2,4]triazolo[1,5-c]quinazolin-2-yl)ethyl 4-methylbenzenesulfonate.

TFA (2 mL, 26.0 mmol) was added to 2-(5-((2,4-dimethoxybenzyl)amino)-7-methoxy-[1,2,4]triazolo[1,5-c]quinazolin-2-yl)ethyl 4-methylbenzenesulfonate (140 mg, 0.248 mmol). The reaction was stirred vigorously at 55° C. for 4 h. The reaction was cooled to room temperature and concentrated under reduced pressure to provide 2-(5-amino-9-fluoro-8-methoxy-[1,2,4]triazolo[1,5-c]quinazolin-2-yl)ethyl 4-methylbenzenesulfonate, which was used in subsequent steps without further purification. MS (ESI) m/z calc’d for C₁₉H₁₉N₅O₄S [M+H]⁺ 339.2, found 339.1.

### Step 2 - Example 23.2, 7-methoxy-2-(2-((1-methyl-1H-pyrazol-4-yl)amino)ethyl)-[1,2,4]triazolo[1,5-c]quinazolin-5-amine.

1-Methyl-1H-pyrazol-4-amine (47 mg, 0.484 mmol) was added to a stirred solution of -(5-amino-9-fluoro-8-methoxy-[1,2,4]triazolo[1,5-c]quinazolin-2-yl)ethyl 4-methylbenzenesulfonate (100 mg, 0.24 mmol) and DIPEA (94 mg, 0.726 mmol) in dioxane (2 mL) at 0° C. The reaction was stirred vigorously at 100° C. for 12 h. The reaction cooled to room temperature and concentrated under reduced pressure. The resulting crude residue was purified by reversed-phase HPLC [Method A] to provide 7-methoxy-2-(2-((1-methyl-1H-pyrazol-4-yl)amino)ethyl)-[1,2,4]triazolo[1,5-c]quinazolin-5-amine (Example 23.2). MS (ESI) m/z calc’d for C₁₆H₁₉N₈O [M+H]⁺ 339.2, found 339.1¹H NMR (500 MHz, DMSO-d₆): δ 7.84 - 7.68 (m, 3H), 7.31-7.21 (m, 1H), 7.22-7.16 (m, 1H), 7.10 (s, 1H), 6.95 (s, 1H), 4.46 (t, J= 6.48 Hz, 1H), 3.88 (s, 3H), 3.67 (s, 3H), 3.35 - 3.30 (m, 2H), 3.10 - 3.03 (m, 2H). A_{2A} IC₅₀ 4.2 nM (A).

The following examples in Table 27 were prepared according to Scheme 23 and General Scheme 8, using Intermediate V.1 and the appropriate amine. Step 2 of Examples 23.3-23.4 was conducted using DMF instead of dioxane, and with the addition of KI as described throughout. Asterisk (^{∗}) indicates that A_{2B} data is not available.

TABLE 27

Examples Prepared According to Scheme 23

| Entry | Structure | Observed m/z [M + H]⁺ | A2a IC₅₀ (nM) |
|---|---|---|---|
| | Name | | A2b IC₅₀ (nM) |
| 23.3 | 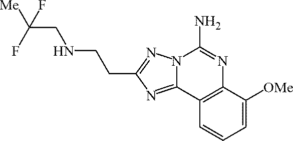 [SMILES: COc1cccc2c1nc(N)n1nc(CCNCC(C)(F)F)nc21] | 337.1 | 12.2 (B) |
| | 2-(2-((2,2-difluoropropyl)amino)ethyl)-7-methoxy-[1,2,4]triazolo[1,5-c]quinazolin-5-amine | | ∗ |
| 23.4 | 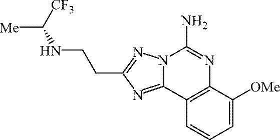 [SMILES: COc1cccc2c1nc(N)n1nc(CCN[C@H](C)C(F)(F)F)nc21] | 355.1 | 5.8 (B) |
| | (R)-7-methoxy-2-(2-((1,1,1-trifluoropropan-2-yl)amino)ethyl)-[1,2,4]triazolo[1,5-c]quinazolin-5-amine | | ∗ |

### Example 24.2,1-(4-((2-(5-amino-9-fluoro-8-methoxy-[1,2,4]triazolo[1,5-c]quinazolin-2-yl)ethyl)amino)-1H-pyrazol-1-yl)-2-methylpropan-2-ol.

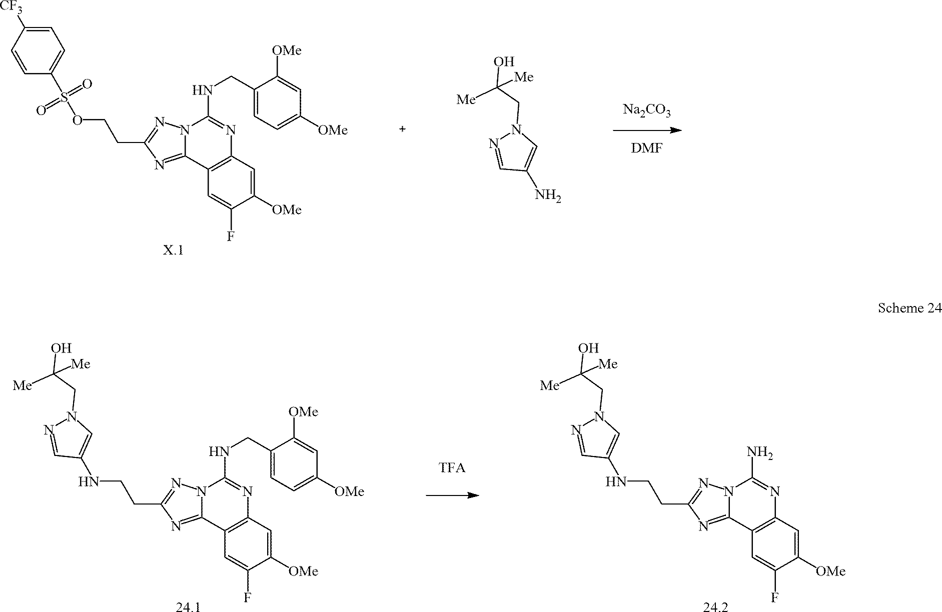

### Step 1 - Intermediate 24.1, 1-(4-((2-(5-((2,4-dimethoxybenzyl)amino)-9-fluoro-8-methoxy-[1,2,4]triazolo[1,5-c]quinazolin-2-yl)ethyl)amino)-1H-pyrazol-1-yl)-2-methylpropan-2-ol.

1-Amino-1H-pyrazol-1-yl)-2-methylpropan-2-ol (22 mg, 0.142 mmol) and NaHCO₃ (19.8 mg, 0.236 mmol) were sequentially added to a stirred solution of 2-(5-((2,4-dimethoxybenzyl)amino)-7-methoxy-[1,2,4]triazolo[1,5-c] quinazolin-2-yl)ethyl 4-(trifluoromethyl)benzenesulfonate (75 mg, 0.118 mmol) in MeCN (1 mL). The reaction was stirred vigorously at 80° C. for 15 h. The reaction was cooled to room temperature and concentrated under reduced pressure. The resulting crude residue was purified by preparative TLC (100% EtOAc) to provide 1-(4-((2-(5-((2,4-dimethoxybenzyl)amino)-9-fluoro-8-methoxy-[1,2,4]triazolo[1,5-c]quinazolin-2-yl)ethyl)amino)-1H-pyrazol-1-yl)-2-methylpropan-2-ol. MS (ESI) m/z calc’d for C₂₈H₃₄FN₈O₄ [M+H]⁺ 565.3, found 565.3.

### Step 2 - Example 24.2, 1-(4-((2-(5-((2,4-dimethoxybenzyl)amino)-9-fluoro-8-methoxy-[1,2,4]triazolo[1,5-c]quinazolin-2-yl)ethyl)amino)-1H-pyrazol-1-yl)-2-methylpropan-2-ol.

TFA (0.30 mL) was added to a stirred solution of 1-(4-((2-(5-((2,4-dimethoxybenzyl)amino)-9-fluoro-8-methoxy-[1,2,4]triazolo[1,5-c]quinazolin-2-yl)ethyl)amino)-1H-pyrazol-1-yl)-2-methylpropan-2-ol (50 mg, 0.089 mmol) in DCM (1 mL). The reaction was stirred vigorously at 40° C. for 3 h, then cooled to room temperature and concentrated under reduced pressure. The resulting crude residue was purified by reversed-phase HPLC [Method A] to provide 1-(4-((2-(5-((2,4-dimethoxybenzyl)amino)-9-fluoro-8-methoxy-[1,2,4]triazolo[1,5-c]quinazolin-2-yl)ethyl)amino)-1H-pyrazol-1-yl)-2-methylpropan-2-ol (Example 24.2). MS (ESI) m/z calc’d for C₁₉H₂₄FN₈O₂ [M+H]⁺ 415.2, found 415.1. ¹H NMR (500 MHz, DMSO-d₆): δ 7.96 (s, 1 H), 7.93 (s, 1 H), 7.42 (s, 2 H), 7.20 (d, J= 7.02 Hz, 1 H), 4.06 (s, 3 H), 4.02 (s, 2 H), 3.66 - 3.63 (m, 2 H), 3.29 (s, 2 H), 2.18 (s, 2 H), 1.18 (s, 6 H). A_{2A} IC₅₀ 26.5 nM (A).

The following examples in Table 28 were prepared according to Scheme 24 and General Scheme 8, using Intermediate X.1 and the appropriate amine. For Example 24.3, the order of operations was reversed. Step 1 of Example 24.4 was Na₂CO₃ instead of NaHCO₃. Asterisk (*) indicates that A_{2B} data is not available.

TABLE 28

Examples Prepared According to Scheme 24

| Entry | Structure | Observed m/z [M + H]⁺ | A2a IC₅₀ (nM) |
|---|---|---|---|
| | Name | | A2b IC₅₀ (nM) |
| 24.3 | 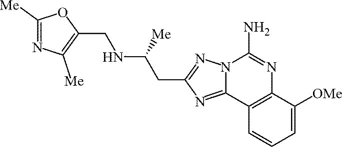 [SMILES: COc1cccc2c1nc(N)n1nc(C[C@@H](C)NCc3oc(C)nc3C)nc21] | 382.2 | 11.1 (B) |
| | (R)-2-(2-(((2,4-dimethyloxazol-5-yl)methyl)amino)propyl)-7-methoxy-[1,2,4]triazolo[1,5-c]quinazolin-5-amine | | ∗ |
| 24.4 | 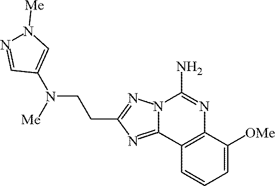 [SMILES: COc1cccc2c1nc(N)n1nc(CCN(C)c3cnn(C)c3)nc21] | 353.1 | 0.9 (A) |
| | 7-methoxy-2-(2-(methyl(1-methyl-1H-pyrazol-4-yl)amino)ethyl)-[1,2,4]triazolo[1,5-c]quinazolin-5-amine | | ∗ |

### Examples 25.3A and 25.3B, (S or R)-2-(4-(1-(((5-amino-9-fluoro-7-methoxy-[1,2,4]triazolo[1,5-c]quinazolin-2-yl)methyl)amino)-2,2,2-trifluoroethyl)phenyl)propan-2-ol and (R or S)-2-(4-(1-(((5-amino-9-fluoro-7-methoxy-[1,2,4]triazolo[1,5-c]quinazolin-2-yl)methyl)amino)-2,2,2-trifluoroethyl)phenyl)propan-2-ol.

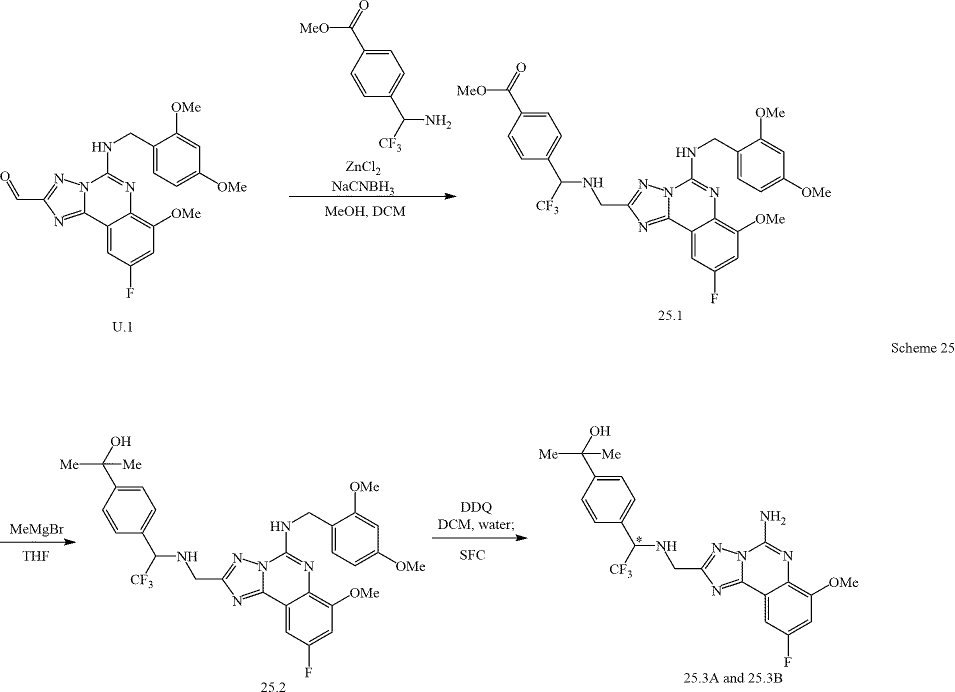

### Step 1 - Intermediate 25.1, Methyl 4-(1-(((5-((2,4-dimethoxybenzyl)amino)-9-fluoro-7-methoxy-[1,2,4]triazolo[1,5-c]quinazolin-2-yl)methyl)amino)-2,2,2-trifluoroethyl)benzoate.

Methyl 4-(1-amino-2,2,2-trifluoroethyl)benzoate (96 mg. 0.413 mmol) and ZnCl₂ (113 mg, 0.826 mmol) were added to a stirred solution of 5-((2,4-dimethoxybenzyl)amino)-9-fluoro-7-methoxy-[1,2,4]triazolo[1,5-c]quinazoline-2-carbaldehyde (229 mg, 1.07 mmol) in MeOH (3 mL) and DCM (2 mL). The reaction was stirred vigorously at room temperature for 1 h, at which point, NaCNBH₃ (78 mg, 1.24 mmol) was added. The reaction was stirred vigorously at 40° C. for 15 h. The reaction was cooled to room temperature, filtered, and concentrated under reduced pressure. The concentrated residue was purified by preparative TLC (gradient elution: 50% EtOAc/petroleum ether) to provide methyl 4-(1-(((5-((2,4-dimethoxybenzyl)amino)-9-fluoro-7-methoxy-[1,2,4]triazolo[1,5-c]quinazolin-2-yl)methyl)amino)-2,2,2-trifluoroethyl)benzoate. MS (ESI) m/z: calc’d for C₃₀H₂₉F₄N₆O₅ [M+H]⁺: 629.2, found 629.2.

### Step 2 - Intermediate 25.2, 2-(4-(1-(((5-((2,4-dimethoxybenzyl)amino)-9-fluoro-7-methoxy-[1,2,4]triazolo[1,5-c]quinazolin-2-yl)methyl)amino)-2,2,2-trifluoroethyl)phenyl)propan-2-ol.

MeMgBr (3 M, 0.223 mL, 0.668 mmol) was added dropwise to a solution provide methyl 4-(1-(((5-((2,4-dimethoxybenzyl)amino)-9-fluoro-7-methoxy-[1,2,4]triazolo[1,5-c]quinazolin-2-yl)methyl)amino)-2,2,2-trifluoroethyl)benzoate in THF (1 mL) at 0° C. The reaction was stirred vigorously at 0° C. for 2 h. The reaction was quenched with sat. aq. NH₄Cl (5 mL), diluted with brine (10 mL), and extracted with EtOAc (3 × 5 mL). The combined organic layers were dried over anhydrous Na₂SO₄, filtered, and concentrated under reduced pressure. The resulting crude residue was purified by preparative TLC (gradient elution: 50% EtOAc/petroleum ether) to provide 2-(4-(1-(((5-((2,4-dimethoxybenzyl)amino)-9-fluoro-7-methoxy-[1,2,4]triazolo[1,5-c]quinazolin-2-yl)methyl)amino)-2,2,2-trifluoroethyl)phenyl)propan-2-ol. MS (ESI) m/z: calc’d for C₃₁H₃₃F₄N₆O₄ [M+Na]+: 629.2, found 629.2.

### Step 3 - Examples 25.3A and 25.3B, (S or R)-2-(4-(1-(((5-amino-9-fluoro-7-methoxy-[1,2,4]triazolo[1,5-c]quinazolin-2-yl)methyl)amino)-2,2,2-trifluoroethyl)phenyl)propan-2-ol and (R or S)-2-(4-(1-(((5-amino-9-fluoro-7-methoxy-[1,2,4]triazolo[1,5-c]quinazolin-2-yl)methyl)amino)-2,2,2-trifluoroethyl)phenyl)propan-2-ol.

DDQ (24.4 mg, 0.107 mmol) was added to a stirred solution of 2-(4-(1-(((5-((2,4-dimethoxybenzyl)amino)-9-fluoro-7-methoxy-[1,2,4]triazolo[1,5-c]quinazolin-2-yl)methyl)amino)-2,2,2-trifluoroethyl)phenyl)propan-2-ol (45 mg, 0.072 mmol) in DCM (2 mL) and water (0.4 mL) at 0° C. The reaction was stirred vigorously at 0° C. for 1 h. The reaction was quenched with sat. aq. Na₂SO₃ (2 mL), and extracted with DCM (3 × 5 mL). The combined organic layers were dried over anhydrous Na₂SO₄, filtered, and concentrated under reduced pressure. The resulting crude residue was purified by preparative TLC (gradient elution: 10% MeOH/DCM). The racemic mixture was separated by chiral SFC (Column: OJ-3, 4.6 × 150 mm, 40% MeOH [w/ 0.05% Et₂NH]/CO₂) to provide Example 25.3A (faster eluting) and Example 25.3B (slower eluting).

Fasting eluting (Example 25.3A): MS (ESI) m/z: calc’d for C₂₂H₂₃F₄N₆O₂ [M+H]⁺: 479.2, found 479.2. ¹H NMR (500 MHz, CDCl₃) δ 7.55 (dd, J = 2.52, 8.2 Hz, 1H), 7.50 (br d, J = 8.2 Hz, 2H), 7.44 - 7.40 (m, 2H), 6.94 (dd, J= 2.3, 10.4 Hz, 1H), 5.88 (br s, 2H), 4.42 - 4.36 (m, 1H), 4.19-4.08 (m, 2H), 4.06 (s, 3H), 1.57 (br s, 6H). A_{2A} IC₅₀ 19.3 nM (A).

Slower eluting (Example 25.3B): MS (ESI) m/z: calc’d for C₂₂H₂₃F₄N₆O₂ [M+H]⁺: 479.2, found 479.2. ¹H NMR (500 MHz, CDCl₃) δ 7.55 (dd, J= 2.59, 8.1 Hz, 1H), 7.51-7.48 (m, 2H), 7.44-7.41 (m, 2H), 6.94 (dd, J= 2.44, 10.2 Hz, 1H), 5.87 (br s, 2H), 4.42-4.36 (m, 1H), 4.17-4.08 (m, 2H), 4.07 (s, 3H), 1.57 (br s, 6H). A_{2A} IC₅₀ 29.2 nM (A).

### Example 26, 7-(2-(5-amino-9-fluoro-7-methoxy-[1,2,4]triazolo[1,5-c]quinazolin-2-yl)ethyl)-3-isopropyl-7,8-dihydroimidazo[1,5-a]pyrazin-6(5H)-one.

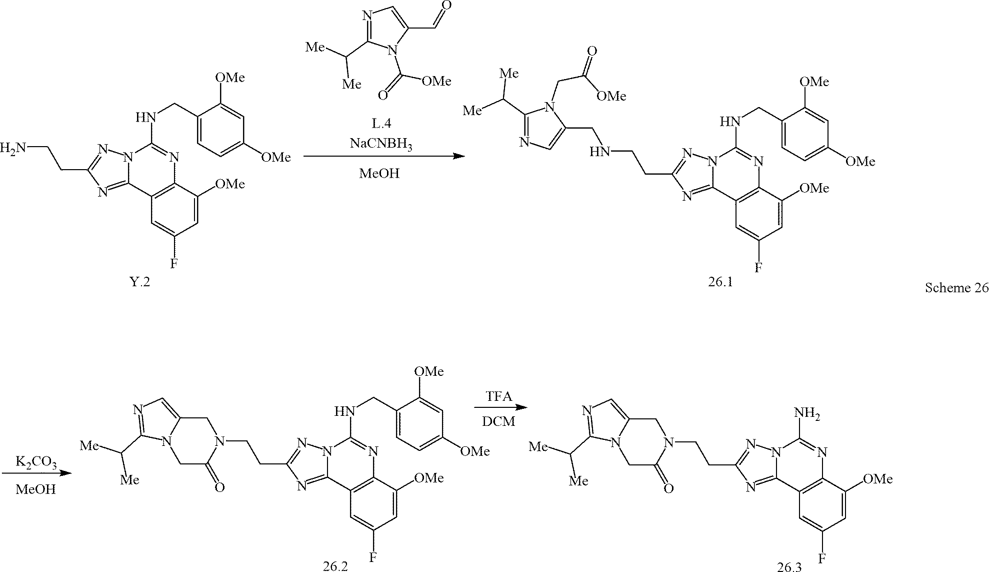

### Step 1 - Intermediate 26.1, Methyl 2-(5-(((2-(5-((2,4-dimethoxybenzyl)amino)-9-fluoro-7-methoxy-[1,2,4]triazolo[1,5-c]quinazolin-2-yl)ethyl)amino)methyl)-2-isopropyl-1H-imidazol-1-yl)acetate.

Methyl 5-formyl-2-isopropyl-1H-imidazole-1-carboxylate (20 mg, 0.095 mmol) and NaCNBH₃ (18 mg, 0.285 mmol) were sequentially added to a stirred solution of 2-(2-aminoethyl)-N-(2,4-dimethoxybenzyl)-9-fluoro-7-methoxy-[1,2,4]triazolo[1,5-c]quinazolin-5-amine (60.9 mg, 0.143 mmol) in MeOH (5 mL). The reaction was stirred vigorously at room temperature for 10 h. The reaction was purified by reversed-phase HPLC [Method A] to provide methyl 2-(5-(((2-(5-((2,4-dimethoxybenzyl)amino)-9-fluoro-7-methoxy-[1,2,4]triazolo[1,5-c]quinazolin-2-yl)ethyl)amino)methyl)-2-isopropyl-1H-imidazol-1-yl)acetate. MS (ESI) m/z calc’d for C₃₁H₃₈FN₈O₅ [M+H]⁺ 621.1, found 621.5.

### Step 2 - Intermediate 26.2, 7-(2-(5-((2,4-dimethoxybenzyl)amino)-9-fluoro-7-methoxy-[1,2,4]triazolo[1,5-c]quinazolin-2-yl)ethyl)-3-isopropyl-7,8-dihydroimidazo[1,5-a]pyrazin-6(5H)-one.

K₂CO₃ (7 mg, 0.051 mmol) was added to a stirred solution of methyl 2-(5-(((2-(5-((2,4-dimethoxybenzyl)amino)-9-fluoro-7-methoxy-[1,2,4]triazolo[1,5-c]quinazolin-2-yl)ethyl)amino)methyl)-2-isopropyl-1H-imidazol-1-yl)acetate (30 mg, 0.017 mmol) in MeOH (2 mL). The reaction was stirred vigorously at 50° C. for 1 h. The reaction was cooled to room temperature and was purified by preparative TLC (2% MeOH/EtOAc) to provide 7-(2-(5-((2,4-dimethoxybenzyl)amino)-9-fluoro-7-methoxy-[1,2,4]triazolo[1,5-c]quinazolin-2-yl)ethyl)-3-isopropyl-7,8-dihydroimidazo[1,5-a]pyrazin-6(5H)-one. MS (ESI) m/z calc’d for C₃₀H₃₄FN₈O₄ [M+H]⁺ 589.1, found 589.2.

### Step 3 - Example 26.3, 7-(2-(5-amino-9-fluoro-7-methoxy-[1,2,4]triazolo[1,5-c]quinazolin-2-yl)ethyl)-3-isopropyl-7,8-dihydroimidazo[1,5-a]pyrazin-6(5H)-one.

TFA (2 mL) was added to a stirred solution of 7-(2-(5-((2,4-dimethoxybenzyl)amino)-9-fluoro-7-methoxy-[1,2,4]triazolo[1,5-c]quinazolin-2-yl)ethyl)-3-isopropyl-7,8-dihydroimidazo[1,5-a]pyrazin-6(5H)-one (7 mg, 0.012 mmol) in DCM. The reaction was stirred vigorously at room temperature for 12 h. The reaction was concentrated under reduced pressure. The resulting crude residue was purified by reversed-phase HPLC [Method A] to provide 7-(2-(5-amino-9-fluoro-7-methoxy-[1,2,4]triazolo[1,5-c]quinazolin-2-yl)ethyl)-3-isopropyl-7,8-dihydroimidazo[1,5-a]pyrazin-6(5H)-one (Example 26.3). MS (ESI) m/z calc’d for C₂₁H₂₄FN₈O₂ [M+H]⁺ 439.1, found 439.2. ¹H NMR (400 MHz, MeOD) δ 7.42 (dd, J = 2.7, 8.2 Hz, 1H), 7.28 (s, 1H), 7.14 (dd, J= 2.7, 10.6 Hz, 1H), 4.84 (s, 2H), 4.73 (s, 2H), 4.06 (t, J= 6.7 Hz, 2H), 4.02 (s, 3H), 3.49 - 3.38 (m, 1H), 3.37 - 3.33 (m, 1H), 1.39 (d, J = 7.0 Hz, 6H). A_{2A} IC₅₀ 6.4 nM (A).

### Biological Assays

The IC₅₀ values reported for each of the compounds of the invention shown in the tables below were measured in accordance with the methods described below. Method (A) describes the procedure used to measure A_{2A} binding affinity using radioligand binding. Method (B) describes the procedure used to measure A_{2A} binding affinity using SPA technology. The method used to measure A_{2B} binding affinity is also described below. The method used to determine the A_{2A} IC₅₀ value reported for each compound in the table is indicated next to the reported value. The A_{2B} IC₅₀ value measured using the A_{2B} binding affinity assay is shown in the table next to the compound under the corresponding A_{2A} value. An asterisk (*) indicates that the IC₅₀ value was not available.

The A_{2A} receptor affinity binding assay measured the amount of binding of a tritiated ligand with high affinity for the A_{2A} adenosine receptor to membranes made from HEK293 or CHO cells recombinantly expressing the human A_{2A} adenosine receptor, in the presence of varying concentrations of a compound of the invention. The data were generated using either filtration binding or a homogenous scintillation proximity assay (SPA). In both assay formats, the tested compounds of the invention were solubilized in 100% DMSO and further diluted in 100% DMSO to generate, typically, a 10-point titration at half-log intervals such that the final assay concentrations did not exceed 10 µM of compound or 1% DMSO.

### Method (A): Measurement of A_{2A} Binding Affinity Using Radioligand Binding

148 µL (5 µg/mL) membranes (Perkin Elmer, Cat. No. RBHA2aM400UA) and 2 µL compounds of the invention to be tested (test compound) were transferred to individual wells of a 96-well polypropylene assay plate and incubated for 15 to 30 min at room temperature. [³H] SCH58261 ((7-(2-phenylethyl)-5-amino-2-(2-furyl)-pyrazolo-[4,3-e]-1,2,4-triazolo[1,5-c]pyrimidine)) was diluted in assay buffer (50 mM Tris pH 7.4, 10 mM MgCl₂, 0.005% Tween20) to a concentration of 4 nM and 50 µL transferred to each well of the assay plate. To define total and non-specific binding, wells containing 1% DMSO and 1 µM ZM241385 (Tocris Bioscience, Cat. No. 1036) respectively, were also included. The assay plate was incubated at room temperature for 60 min with agitation. Using a FilterMate Harvester® (Perkin Elmer), the contents of the assay plate were filtered through a UniFilter-96® PEI coated plate (Perkin Elmer Cat. No. 6005274 or 6005277). Filtering was achieved by aspirating the contents of the assay plate for 5 sec, then washing and aspirating the contents three times with ice-cooled wash buffer (50 mM Tris-HCl pH 7.4, 150 mM NaCl) and allowing the vacuum manifold to dry the plate for 30 sec. The filter plate was incubated for at least 1 h at 55° C. and allowed to dry. The bottom of the filter plate was sealed with backing tape. 40 µL Ultima Gold™ (Perkin Elmer, Cat. No. 6013329) was added to each well of the filter plate and the top of the plate was sealed with TopSeal-A PLUS® clear plate seal (Perkin Elmer, Cat. No. 6050185). The plate was incubated for at least 20 min, and then the amount of radioactivity remaining in each well was determined using a TopCount® (Perkin Elmer) scintillation counter. After normalization to total and non-specific binding, the percent effect at each compound concentration was calculated. The plot of percent effect versus the log of compound concentration was analyzed electronically using a 4-parameter logistic fit based on the Levenberg-Marquardt algorithm to generate IC₅₀ values.

### Method (B): Measurement of A_{2A} Binding Affinity Using SPA

Binding affinity using SPA was conducted as follows. Test compounds (50 nL) were dispensed into individual wells of a 384-well OptiPlate™ well (Perkin Elmer) by Echo® acoustic liquid transfer (Labcyte). 20 µL of 1.25 nM [³H] SCH58261 ((7-(2-phenylethyl)-5-amino-2-(2-furyl)-pyrazolo-[4,3-e]-1,2,4-triazolo[1,5-c]pyrimidine)) in DPBS assay buffer (Dulbecco’s phosphate buffered saline without calcium and magnesium, ThermoFisher Scientific, Cat. No. A1285601) supplemented with 10 mM MgCl₂ was added. A_{2A} receptor-expressing membranes were incubated with 20 µg/mL adenosine deaminase (Roche, Cat. No. 10 102 105 001) for 15 min at room temperature. The receptor-expressing membranes were then combined with wheat germ agglutinin-coated yttrium silicate SPA beads (GE Healthcare, Cat. No. RPNQ0023) in a ratio of 1:1000 (w/w) and incubated for 30 min at room temperature. 30 µL of the membrane/bead mixture (0.25 µg and 25 µg per well respectively) were added to the 384-well OptiPlate™ well. To define total and non-specific binding, wells containing 1% DMSO or 1 µM CGS 15943 (Tocris Bioscience, Cat. No. 1699) respectively were also included in the experiment. The plate was incubated for 1 h at room temperature with agitation. The assay plate was then incubated for an h to allow the beads to settle before data were collected using a TopCount® (Perkin Elmer) scintillation counter. After normalization to total and non-specific binding, the percent effect at each compound concentration was calculated. The plot of percent effect versus the log of compound concentration was analyzed electronically using a 4-parameter logistic fit based on the Levenberg-Marquardt algorithm to generate IC₅₀ values.

### Measurement of A_{2B} Binding Affinity

The reported affinity of the compounds of the invention for the human A_{2B} adenosine receptor was determined experimentally using a radioligand filtration binding assay. This assay measures the amount of binding of a tritiated proprietary A_{2B} receptor antagonist, in the presence and absence of a compound of the invention, to membranes made from HEK293 cells recombinantly expressing the human A_{2B} adenosine receptor (Perkin Elmer, Cat. No. ES-013-C). To perform the assay, compounds of the invention to be tested were first solubilized in 100% DMSO and further diluted in 100% DMSO to generate, typically, a 10-point titration at half-log intervals such that the final assay concentrations did not exceed 10 µM of compound or 1% DMSO. 148 µL (135 µg/mL) membranes and 2 µL test compounds were transferred to individual wells of a 96-well polypropylene assay plate and incubated for 15 to 30 min at room temperature with agitation. Tritiated radioligand was diluted to a concentration of 14 nM in assay buffer (phosphate buffered saline without Magnesium and Calcium, pH 7.4; GE Healthcare Life Sciences, Cat. No. SH30256.01) and then 50 µL of the solution were transferred to each well of the assay plate. To define total and non-specific binding, wells containing 1% DMSO and 20 µM N-ethylcarboxamidoadenosine (Tocris Bioscience, Cat. No. 1691) respectively, were also included. The wells of the assay plate were incubated at room temperature for 60 min with agitation, then filtered using a FilterMate Harvester® (Perkin Elmer) or similar equipment through a UniFilter-96® PEI coated plate (Perkin Elmer Cat. No. 6005274 or 6005277). Filtering was achieved by aspirating the contents of the assay plate for 5 sec, then washing and aspirating the contents three times with ice-cooled wash buffer (assay buffer supplemented with 0.0025% Brij58) and allowing the vacuum manifold to dry the plate for 30 sec. The filter plate was incubated for at least 1 h at 55° C. and allowed to dry. The bottom of the filter plate was then sealed with backing tape. 40 µL Ultima Gold™ (Perkin Elmer, Cat. No. 6013329) was added to each well of the filter plate and the top of the plate was sealed with TopSeal-A PLUS® clear plate seal (Perkin Elmer, Cat. No. 6050185). The plates were then incubated for at least 20 min, and then the amount of radioactivity remaining in each well was determined using a TopCount® (Perkin Elmer) scintillation counter. After normalization to total and non-specific binding, the percent effect at each compound concentration was calculated. The plot of percent effect versus the log of compound concentration was analyzed electronically using a 4-parameter logistic fit based on the Levenberg-Marquardt algorithm to generate IC₅₀ values.

What is claimed is:

1. A compound having a structural Formula (I): 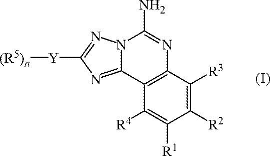
or a pharmaceutically acceptable salt thereof, wherein:
R¹ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and heterocycloalkyl;
R² is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and heterocycloalkyl;
R³ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and heterocycloalkyl;
R⁴ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and heterocycloalkyl;
Y is a straight or branched (Ci-Cs)alkyl, wherein one or more —CH₂— groups in Y are independently replaced with a moiety selected from the group consisting of N(R⁸)₂, NR⁸C(O), OC(O)N(R⁸)₂, a nitrogen-containing heterocycloalkyl and C(O)N(R⁸)₂;
each occurrence of R⁵ is hydrogen, halogen, aryl, (C₃-C₆)cycloalkyl, heterocycloalkyl, heteroaryl, (C₁-C₆)alkylOH, OH, (C₁-C₆)haloalkyl, (C₁-C₆)alkyl, (C₁-C₆)alkynyl, SO₂R⁶, SO(═NH)R⁶, SO(═NCH₃)R⁶ and COO(C₁-C₆)alkyl, wherein the aryl, (C₃-C₆)cycloalkyl, heterocycloalkyl and heteroaryl are optionally substituted with one to three substituents selected from the group consisting of halogen, (C₁-C₆)alkyl, (C₁-C₆)haloalkyl, O(C₁-C₆)alkyl, O(C₁-C₆)haloalkyl, (C₃-C₆)cycloalkyl, O(C₃-C₆)cycloalkyl, (C₁-C₆)haloalkylOH, (C₁-C₆)alkylOH, SO₂N(R⁷)₂, S(halo)₅, haloaryl, halo(C₃-C₆)cycloalkyl, haloheteroaryl, heterocycloalkyl, NR⁷C(O)(C₁-C₆)alkyl, heteroaryl, Boc, and (C₁-C₆)alkylN(R⁷)₂;
R⁶ is selected from the group consisting of OH, NH₂, (C₁-C₆)alkyl, aryl, (C₁-C₆)haloalkyl, (C₃-C₆)cycloalkyl and haloaryl;
each occurrence of R⁷ is independently selected from the group consisting of H, (C₁-C₆)alkyl, and (C₃-C₆)cycloalkyl, wherein when two R⁷ substituents are taken together with the nitrogen they are attached to, a heterocycloalkyl is formed;
each occurrence of R⁸ is independently selected from the group consisting of H, (C₁-C₆)alkyl, (C₃-C₆)cycloalkyl, (C₁-C₆)alkylOH, (C₁-C₆)haloalkyl, (C₁-C₆)alkylcycloalkyl, (C₁-C₆)alkylO(C₁-C₆)alkyl, (C₁-C₆)alkylC(O)O(C₁-C₆)alkyl, SO₂(R⁶), SO₂(C₁-C₆)alkyl, SO₂(C₁-C₆)haloalkyl, CONH(C₁-C₆)alkyl, SO₂(C₃-C₆)cycloalkyl, CON(R⁷)₂, (C₁-C₆)alkylheterocycloalkyl and (C₁₋C₆)alkenyl; and
n is 1, 2 or 3.

2. The compound of claim 1, or a pharmaceutically acceptable salt thereof, wherein:
R¹ is selected from the group consisting of hydrogen, halogen, (C₁-C₆)alkyl and O(C₁-C₆)alkyl;
R² is selected from the group consisting of hydrogen, halogen, (C₁-C₆)alkyl and O(C₁-C₆)alkyl;
R³ is selected from the group consisting of hydrogen, halogen, (C₁-C₆)alkyl and O(C₁-C₆)alkyl;
R⁴ is selected from the group consisting of hydrogen, halogen, (C₁-C₆)alkyl and O(C₁-C₆)alkyl;
R⁵ is hydrogen, halogen, aryl, (C₃-C₆)cycloalkyl, heterocycloalkyl, heteroaryl, (C₁-C₆)alkylOH, OH, (C₁-C₆)haloalkyl, (C₁-C₆)alkyl, (C₁-C₆)alkynyl and SO₂R⁶, wherein the aryl, (C₃-C₆)cycloalkyl, heterocycloalkyl and heteroaryl are optionally substituted with one to three substituents selected from the group consisting of halogen, (C₁-C₆)alkyl, (C₁-C₆)haloalkyl, O(C₁-C₆)alkyl, O(C₁-C₆)haloalkyl, (C₃-C₆)cycloalkyl, O(C₃-C₆)cycloalkyl, (C₁-C₆)haloalkylOH, (C₁-C₆)alkylOH, SO₂NH₂, S(F)₅, haloaryl, halo(C₃-C₆)cycloalkyl, haloheteroaryl, heterocycloalkyl, NHC(O)(C₁-C₆)alkyl, heteroaryl, Boc, and (C₁-C₆)alkylNH₂;
R⁶ is selected from the group consisting of NH₂, (C₁-C₆)alkyl, aryl, (C₁–C₆)haloalkyl, (C₃-C₆)cycloalkyl and haloaryl;
each occurrence of R⁸ is independently selected from the group consisting of H, (C₁-C₆)alkyl, (C₃-C₆)cycloalkyl, (C₁-C₆)alkylOH, (C₁-C₆)haloalkyl, (C₁-C₆)alkylcycloalkyl, (C₁-C₆)alkylO(C₁-C₆)alkyl, (C₁-C₆)alkylC(O)O(C₁-C₆)alkyl, SO₂(C₁-C₆)alkyl, SO₂(C₁-C₆)haloalkyl, CONH(C₁-C₆)alkyl, (C₁-C₆)alkylheterocycloalkyl, SO₂(C₃-C₆)cycloalkyl and (C₁-C₆)alkenyl; and
n is 1, 2 or 3.

3. The compound of claim 1, or a pharmaceutically acceptable salt thereof, wherein R¹ is selected from the group consisting of hydrogen and fluorine.

4. The compound of claim 1, or a pharmaceutically acceptable salt thereof, wherein R² is selected from the group consisting of hydrogen and methoxy.

5. The compound of claim 1, or a pharmaceutically acceptable salt thereof, wherein R³ is selected from the group consisting of hydrogen, fluorine and methoxy.

6. The compound of claim 1, or a pharmaceutically acceptable salt thereof, wherein R⁴ is hydrogen.

7. The compound of any of claim 1, or a pharmaceutically acceptable salt thereof, wherein R¹, R², R³ and R⁴ are not simultaneously hydrogen.

8. The compound of claim 1, or a pharmaceutically acceptable salt thereof, wherein Y is a straight (C₁-C₅)alkyl, wherein one or more —CH₂— groups in Y are independently replaced with N(R⁸)₂, wherein each occurrence of R⁸ is independently selected from the group consisting of H, (C₁-C₆)alkyl, (C₃-C₆)cycloalkyl, (C₁-C₆)alkylOH, (C₁-C₆)haloalkyl (C₁-C₆)alkylcycloalkyl, (C₁-C₆)alkylO(C₁-C₆)alkyl, (C₁-C₆)alkylC(O)O(C₁-C₆)alkyl, SO₂(C₁-C₆)alkyl, SO₂(C₁-C₆)haloalkyl, CONH(C₁-C₆)alkyl, (C₁-C₆)alkylheterocycloalkyl, SO₂(C₃-C₆)cycloalkyl and (C₁-C₆)alkenyl.

9. The compound of claim 1, or a pharmaceutically acceptable salt thereof, wherein Y is a straight (C₁-C₅)alkyl, wherein one or more —CH₂— groups in Y are independently replaced with C(O)N(R⁸)₂, wherein each occurrence of R⁸ is independently selected from the group consisting of H, (C₁-C₆)alkyl, (C₃-C₆)cycloalkyl, (C₁-C₆)alkylOH, (C₁-C₆)haloalkyl (C₁-C₆)alkylcycloalkyl, (C₁-C₆)alkylO(C₁-C₆)alkyl, (C₁-C₆)alkylC(O)O(C₁-C₆)alkyl, SO₂(C₁-C₆)alkyl, SO₂(C₁-C₆)haloalkyl, CONH(C₁-C₆)alkyl, (C₁-C₆)alkylheterocycloalkyl, SO₂(C₃-C₆)cycloalkyl and (C₁-C₆)alkenyl.

10. The compound of claim 1, or a pharmaceutically acceptable salt thereof, wherein Y is a straight (C₁-C₅)alkyl, wherein one or more —CH₂— groups in Y are independently replaced with NR⁸C(O), wherein each occurrence of R⁸ is independently selected from the group consisting of H, (C₁-C₆)alkyl, (C₃-C₆)cycloalkyl, (C₁-C₆)alkylOH, (C₁-C₆)haloalkyl, (C₁-C₆)alkylcycloalkyl, (C₁-C₆)alkylO(C₁-C₆)alkyl, (C₁-C₆)alkylC(O)O(C₁-C₆)alkyl, SO₂(C₁-C₆)alkyl, SO₂(C₁-C₆)haloalkyl, CONH(C₁-C₆)alkyl, (C₁-C₆)alkylheterocycloalkyl, SO₂(C₃-C₆)cycloalkyl and (C₁-C₆)alkenyl.

11. The compound of claim 1, or a pharmaceutically acceptable salt thereof, wherein Y is a straight (C₁-C₅)alkyl, wherein one or more —CH₂— groups in Y are independently replaced with OC(O)N(R⁸)₂, wherein each occurrence of R⁸ is independently selected from the group consisting of H, (C₁-C₆)alkyl, (C₃-C₆)cycloalkyl, (C₁-C₆)alkylOH, (C₁-C₆)haloalkyl (C₁-C₆)alkylcycloalkyl, (C₁-C₆)alkylO(C₁-C₆)alkyl, (C₁-C₆)alkylC(O)O(C₁-C₆)alkyl, SO₂(C₁-C₆)alkyl, SO₂(C₁-C₆)haloalkyl, CONH(C₁-C₆)alkyl, (C₁-C₆)alkylheterocycloalkyl, SO₂(C₃-C₆)cycloalkyl and (C₁-C₆)alkenyl.

12. The compound of claim 1, or a pharmaceutically acceptable salt thereof, wherein Y is a straight (C₁-C₅)alkyl, wherein one or more —CH₂— groups in Y are independently replaced with a nitrogen-containing heterocycloalkyl, wherein each occurrence of R⁸ is independently selected from the group consisting of H, (C₁-C₆)alkyl, (C₃-C₆)cycloalkyl, (C₁-C₆)alkylOH, (C₁-C₆)haloalkyl (C₁-C₆)alkylcycloalkyl, (C₁-C₆)alkylO(C₁-C₆)alkyl, (C₁-C₆)alkylC(O)O(C₁-C₆)alkyl, SO₂(C₁-C₆)alkyl, SO₂(C₁-C₆)haloalkyl, CONH(C₁-C₆)alkyl, (C₁-C₆)alkylheterocycloalkyl, SO₂(C₃-C₆)cycloalkyl and (C₁-C₆)alkenyl.

13. The compound of claim 1, or a pharmaceutically acceptable salt thereof, wherein Y is a branched (C₁-C₅)alkyl, wherein one or more —CH₂— groups in Y are independently replaced with N(R⁸)₂, wherein R⁸ is independently selected from the group consisting of H, (C₁-C₆)alkyl, (C₃-C₆)cycloalkyl, (C₁-C₆)alkylOH, (C₁-C₆)haloalkyl (C₁-C₆)alkylcycloalkyl, (C₁-C₆)alkyIO(C₁-C₆)alkyl, (C₁-C₆)alkyIC(O)O(C₁-C₆)alkyl, SO₂(C₁-C₆)alkyl, SO₂(C₁-C₆)haloalkyl, CONH(C₁-C₆)alkyl, (C₁-C₆)alkylheterocycloalkyl, SO₂(C₃-C₆)cycloalkyl and (C₁-C₆)alkenyl.

14. The compound of claim 1, or a pharmaceutically acceptable salt thereof, wherein Y is 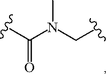 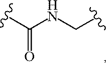 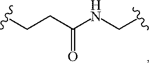 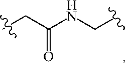 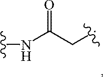 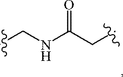 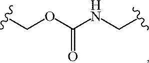 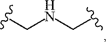 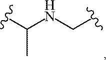 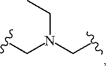 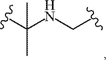 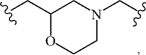 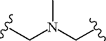 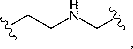 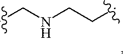 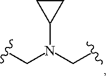 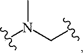 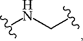 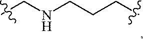 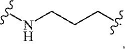 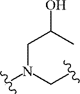 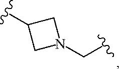 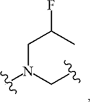 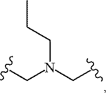 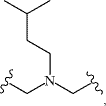 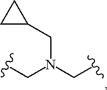 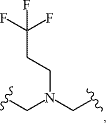 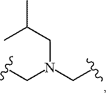 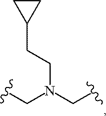 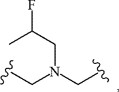 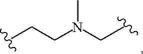 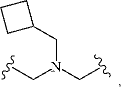 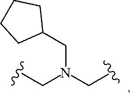 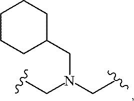 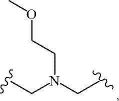 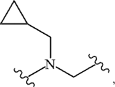 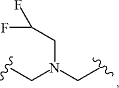 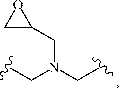 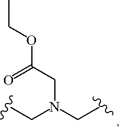 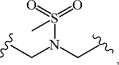 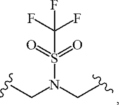 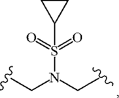 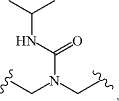 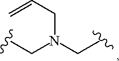 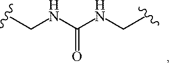 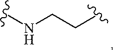 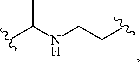 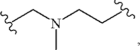 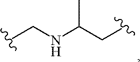 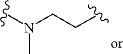 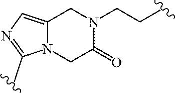
.

15. The compound of claim 1 or a pharmaceutically acceptable salt thereof, wherein R⁵ is phenyl and the phenyl is optionally substituted with one to three substituents selected from the group consisting of halogen, (C₁-C₆)alkyl, (C₁-C₆)haloalkyl, O(C₁-C₆)alkyl, O(C₁-C₆)haloalkyl, (C₃-C₆)cycloalkyl, O(C₃-C₆)cycloalkyl, (C₁-C₆)haloalkylOH, (C₁–C₆)alkylOH, SO₂NH₂, S(F)₅, haloaryl, halo(C₃-C₆)cycloalkyl, haloheteroaryl, heterocycloalkyl, NHC(O)(C₁-C₆)alkyl, heteroaryl, tert-butoxycarbonyl, and (C₁-C₆)alkylNH₂.

16. The compound of claim 1, or a pharmaceutically acceptable salt thereof, wherein R⁵ is an unsubstituted or substituted (C₃-C₆)cycloalkyl, wherein the unsubstituted or substituted (C₃-C₆)cycloalkyl is cyclopropyl or 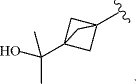
.

17. The compound of claim 1, or a pharmaceutically acceptable salt thereof, wherein R⁵ is an unsubstituted or substituted heteroaryl, wherein the unsubstituted or substituted heteroaryl is 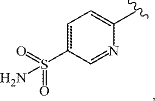 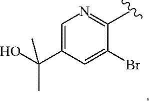 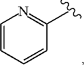 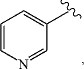 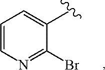 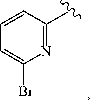 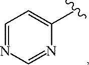 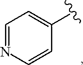 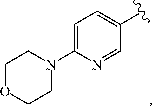 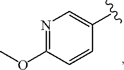 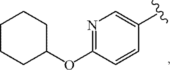 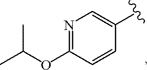 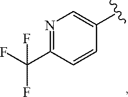 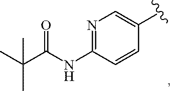 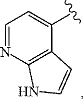 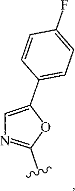 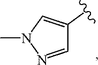 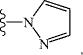 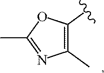 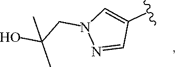 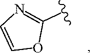 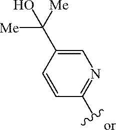 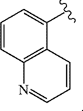
.

18. The compound of claim 1, or a pharmaceutically acceptable salt thereof, wherein R⁵ is an unsubstituted or substituted heterocycloalkyl, wherein the unsubstituted or substituted heterocycloalkyl is 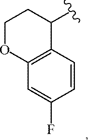 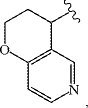 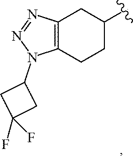 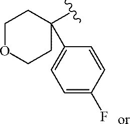 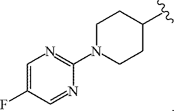
.

19. The compound of claim 1 or a pharmaceutically acceptable salt thereof, wherein R⁵ is methyl, ethyl, trifluoromethyl, OH, 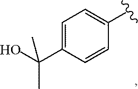 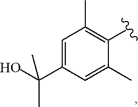 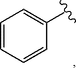 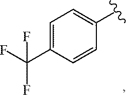 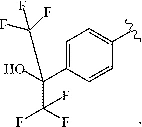 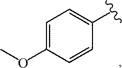 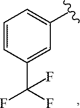 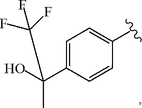 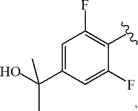 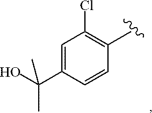 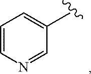 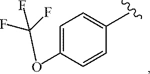 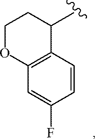 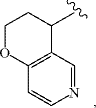 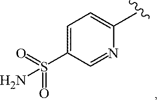 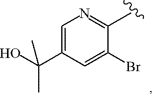 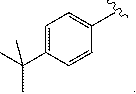 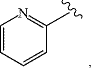 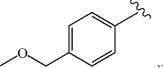 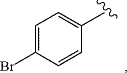 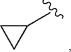 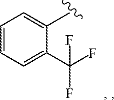 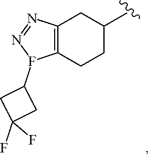 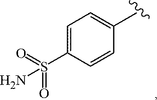 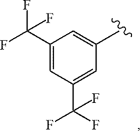 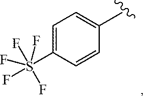 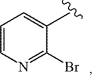 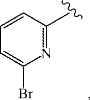 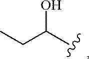 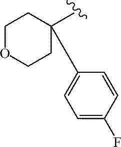 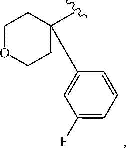 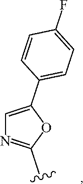 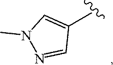 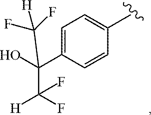 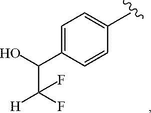 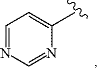 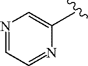 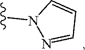 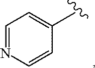 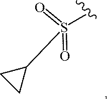 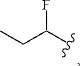 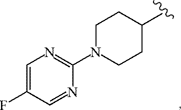 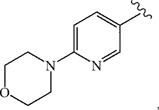 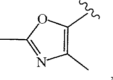 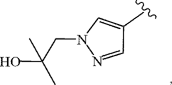 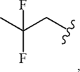 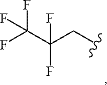 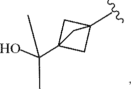 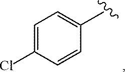 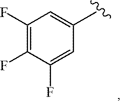 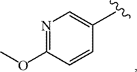 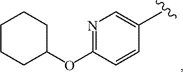 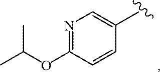 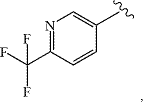 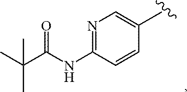 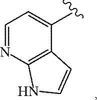 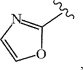 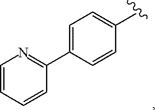 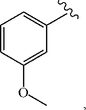 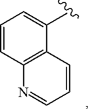 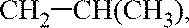 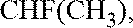 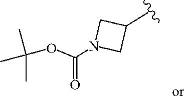 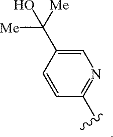
.

20. The compound of claim 1, or a pharmaceutically acceptable salt thereof, wherein R⁵ is SO₂R⁶, wherein R⁶ is cyclopropyl.

21. A compound, or pharmaceutically acceptable salt thereof, having the following structure:

| | |
|---|---|
| 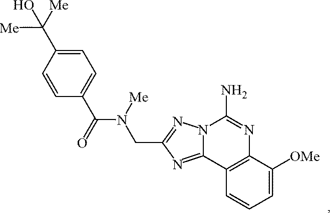 | 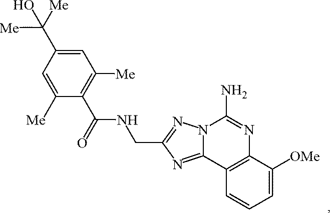 |
| 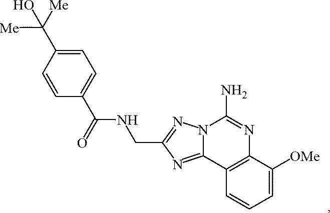 | 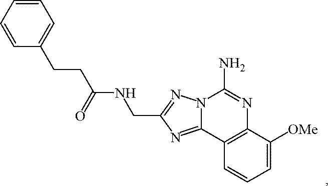 |
| 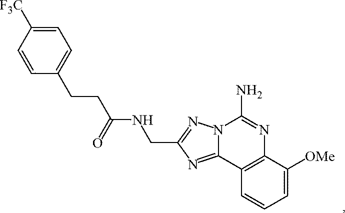 | 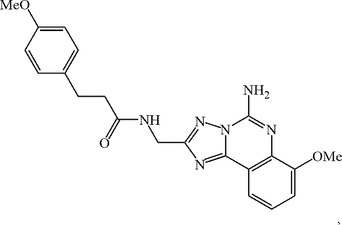 |
| 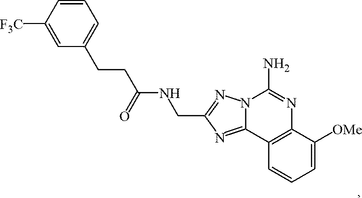 | 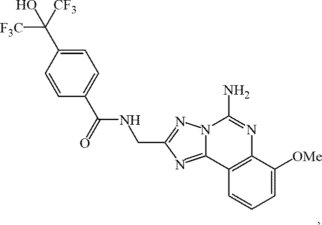 |
| 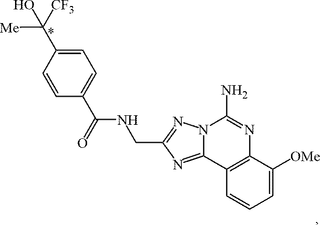 | 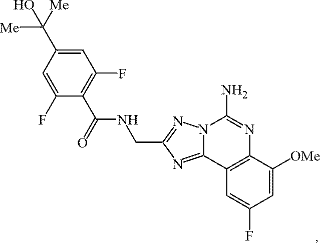 |
| 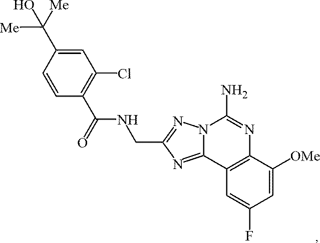 | 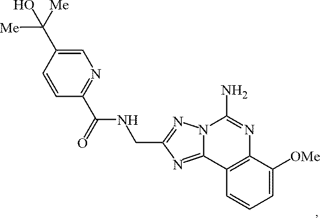 |
| 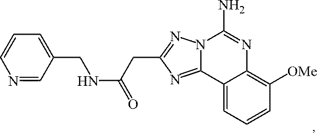 | 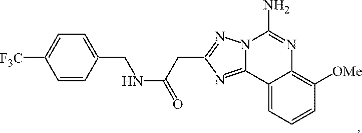 |
| 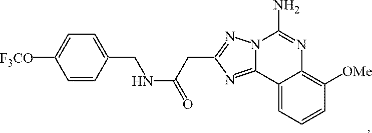 | 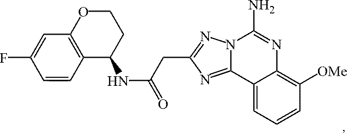 |
| 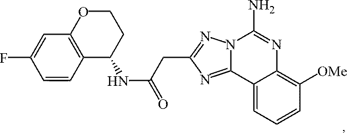 | 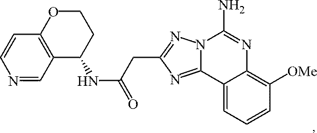 |
| 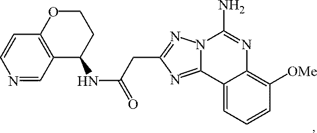 | 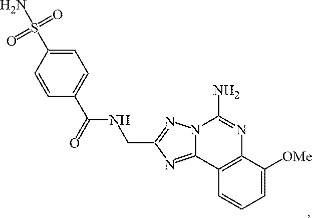 |
| 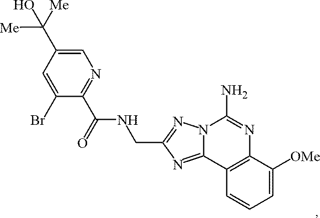 | 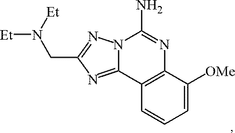 |
| 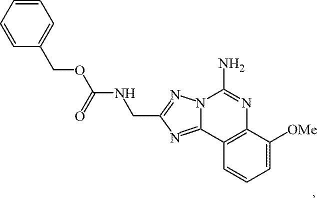 | 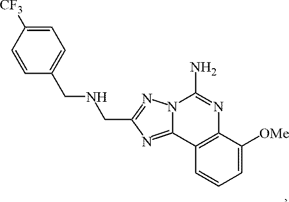 |
| 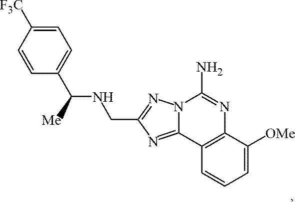 | 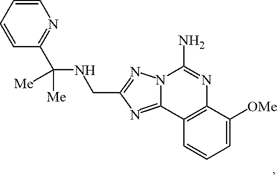 |
| 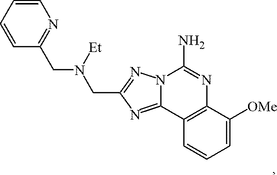 | 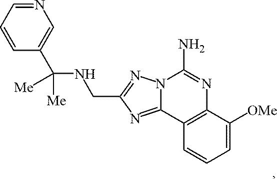 |
| 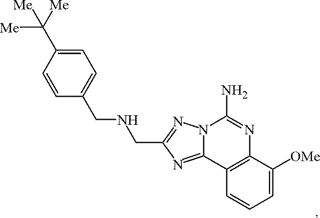 | 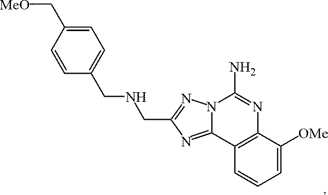 |
| 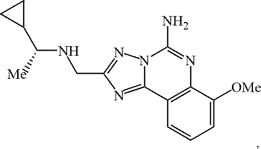 | 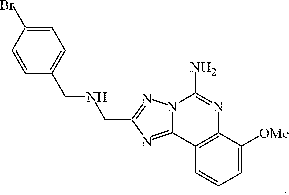 |
| 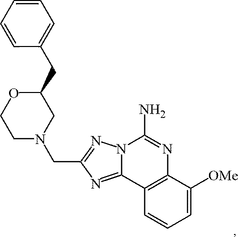 | 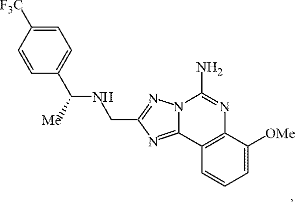 |
| 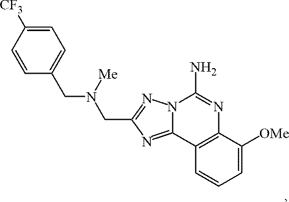 | 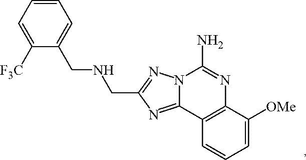 |
| 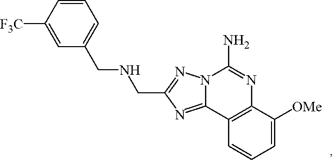 | 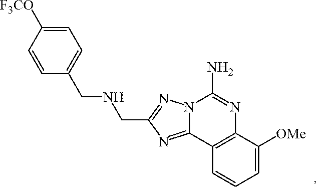 |
| 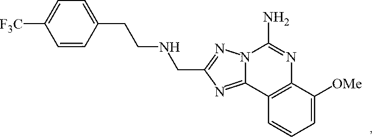 | 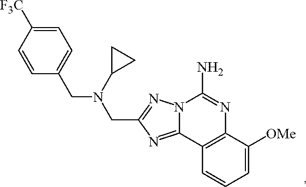 |
| 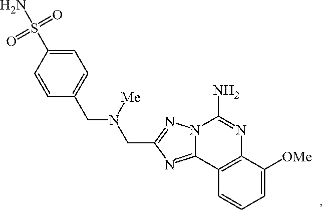 | 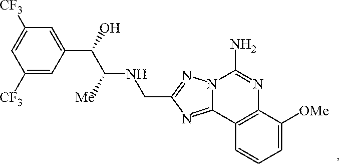 |
| 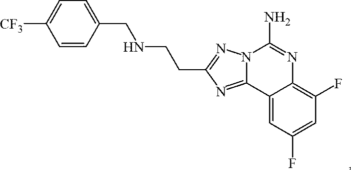 | 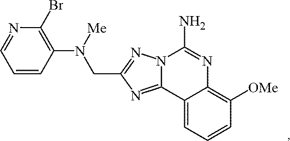 |
| 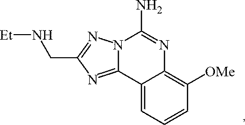 | 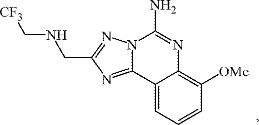 |
| 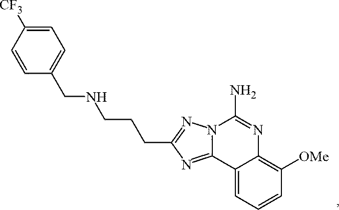 | 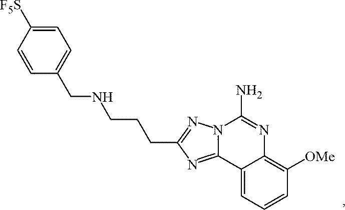 |
| 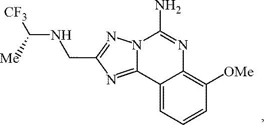 | 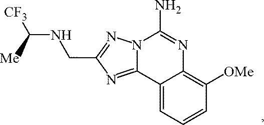 |
| 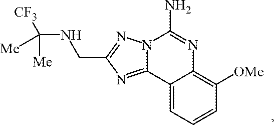 | 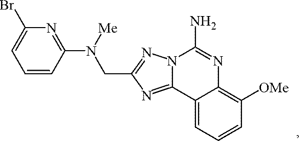 |
| 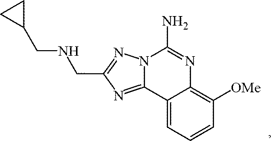 | 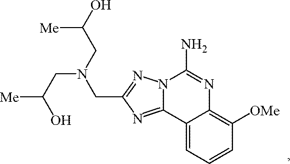 |
| 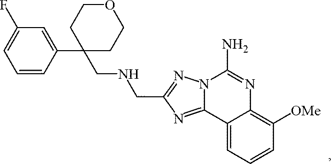 | 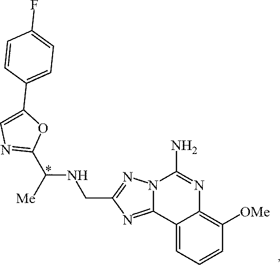 |
| 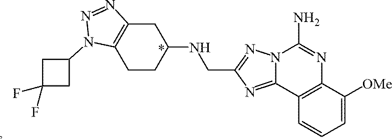 | 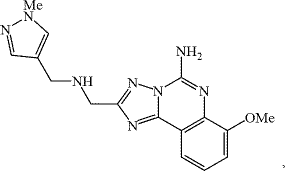 |
| 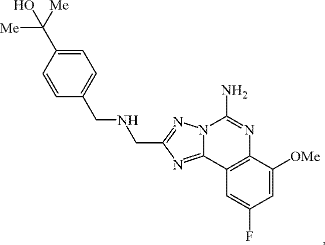 | 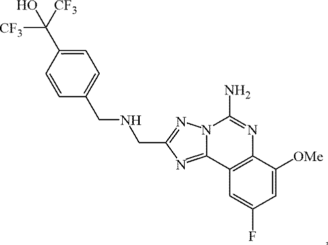 |
| 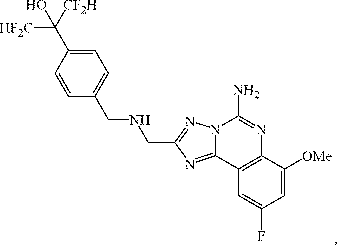 | 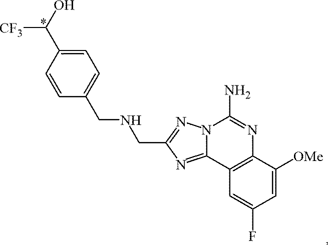 |
| 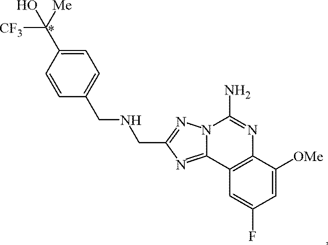 | 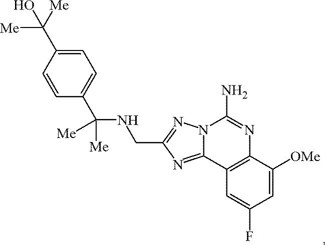 |
| 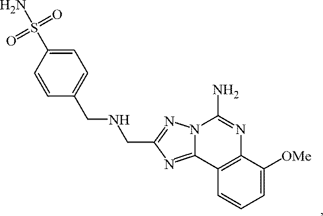 | 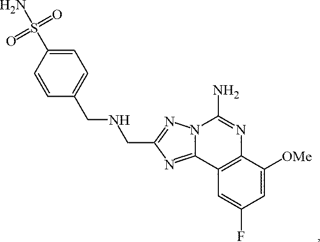 |
| 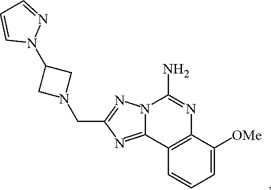 | 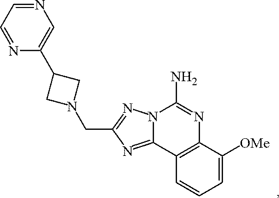 |
| 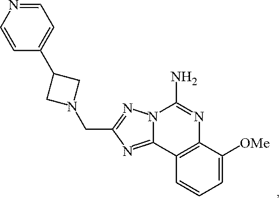 | 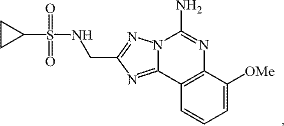 |
| 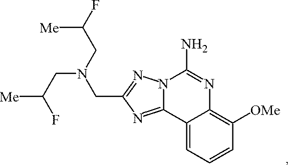 | 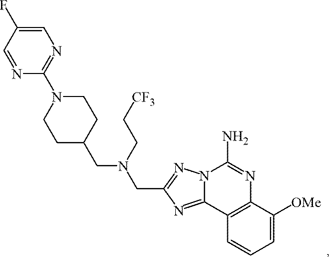 |
| 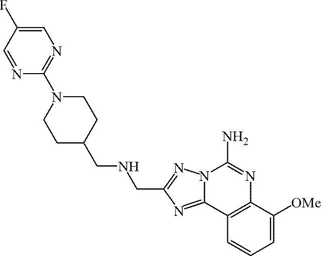 [SMILES: COc1cccc2c1nc(N)n1nc(CNCC3CCN(c4ncc(F)cn4)CC3)nc21] | 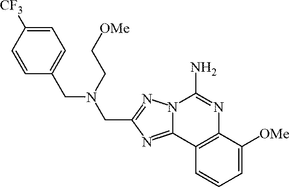 [SMILES: COCCN(Cc1ccc(C(F)(F)F)cc1)Cc1nc2c3cccc(OC)c3nc(N)n2n1] |
| 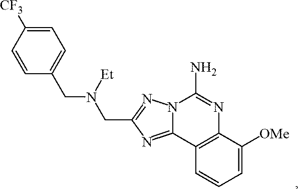 | 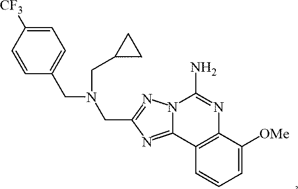 |
| 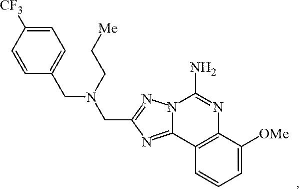 | 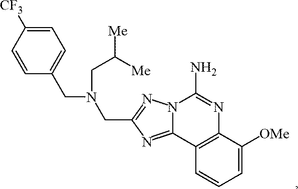 |
| 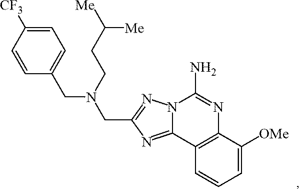 | 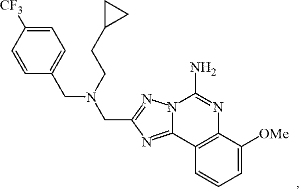 |
| 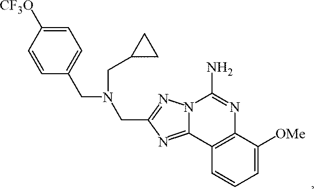 | 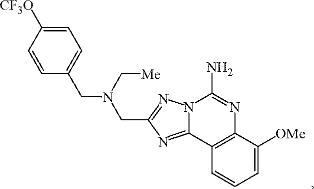 |
| 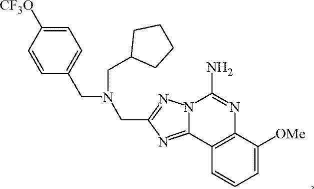 | 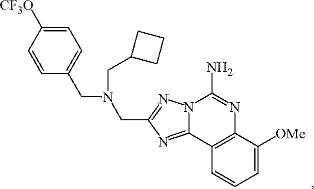 |
| 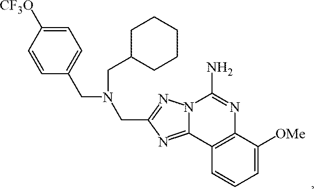 | 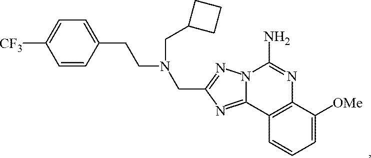 |
| 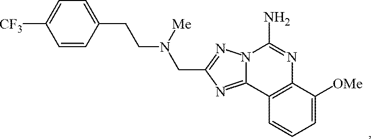 | 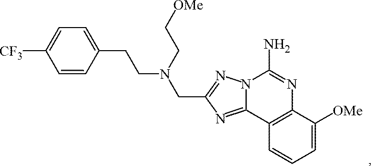 |
| 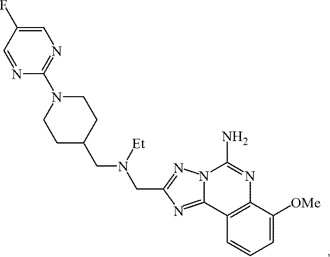 | 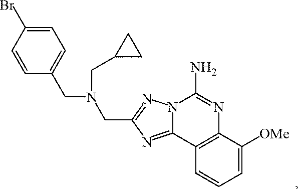 |
| 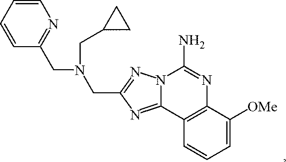 | 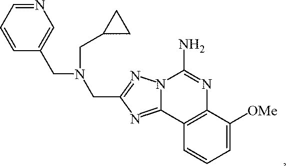 |
| 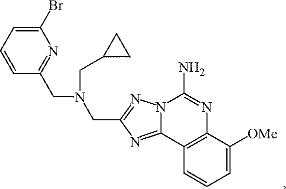 | 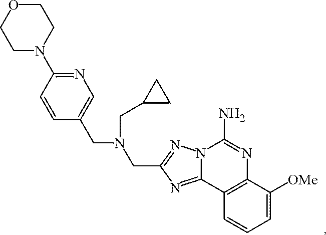 |
| 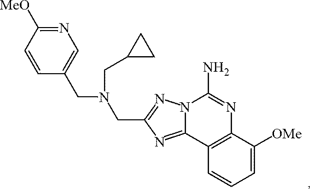 | 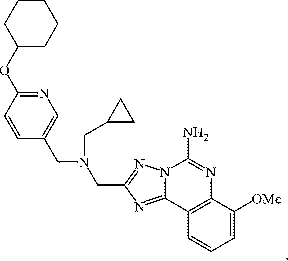 |
| 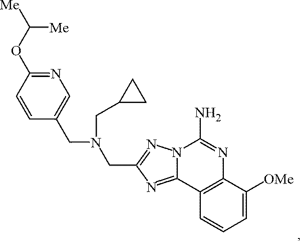 | 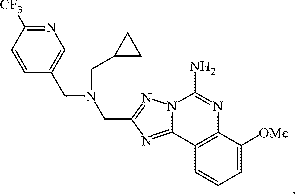 |
| 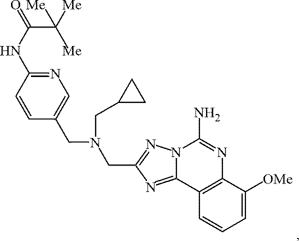 | 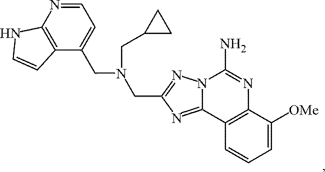 |
| 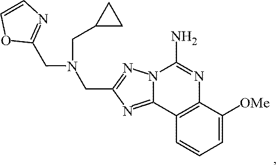 | 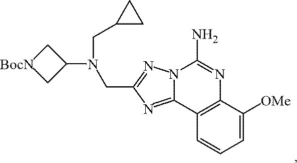 |
| 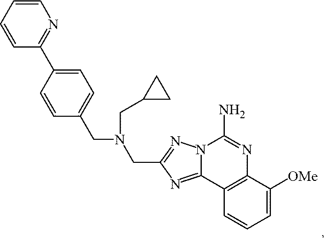 | 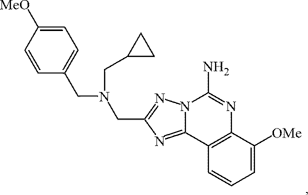 |
| 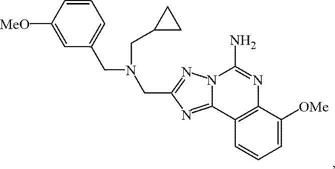 | 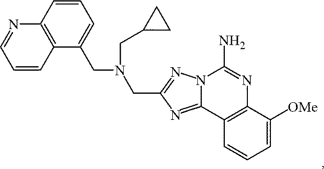 |
| 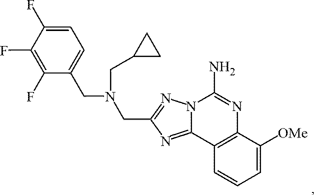 | 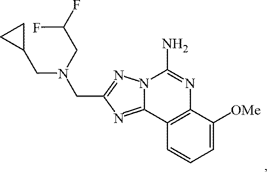 |
| 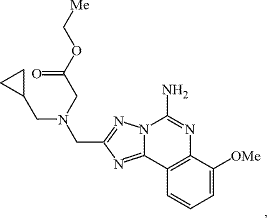 | 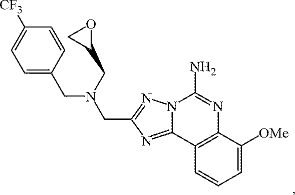 |
| 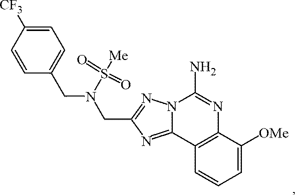 | 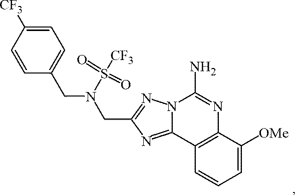 |
| 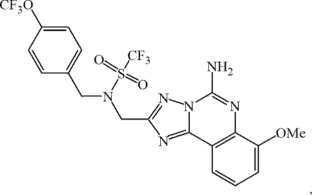 | 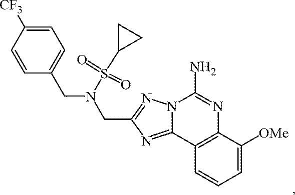 |
| 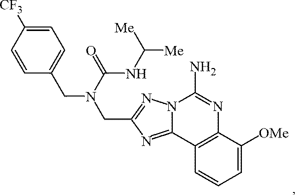 | 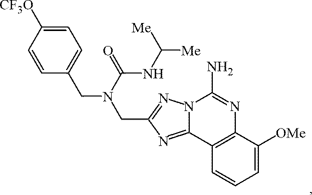 |
| 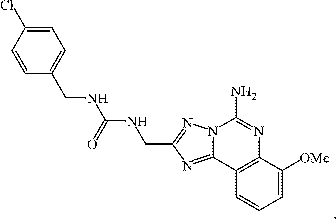 | 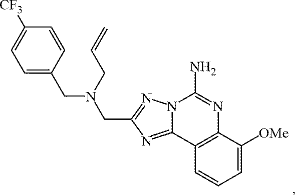 |
| 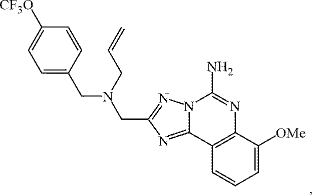 | 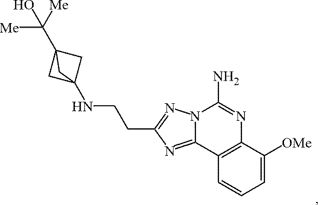 |
| 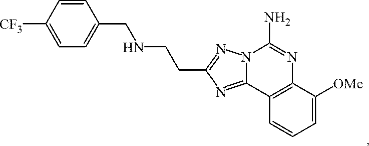 | 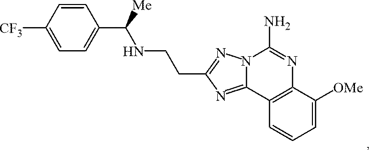 |
| 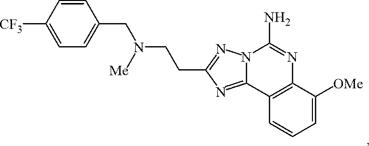 | 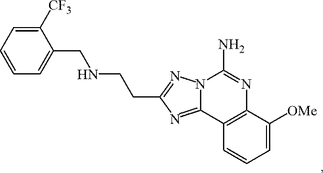 |
| 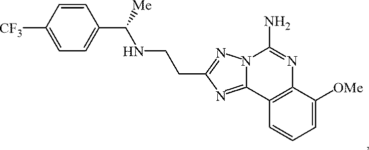 | 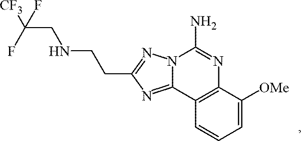 |
| 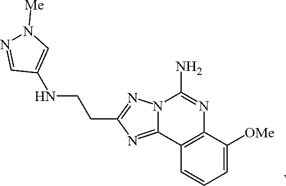 | 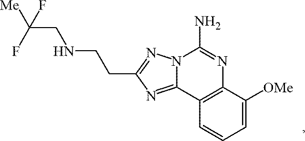 |
| 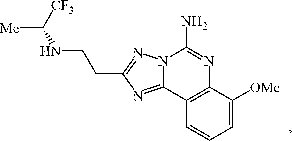 | 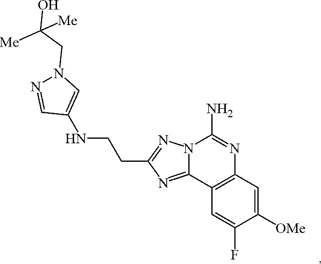 |
| 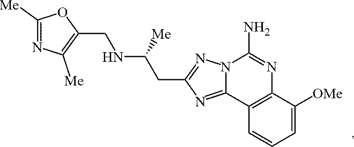 | 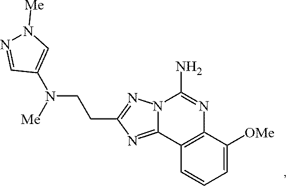 |
| 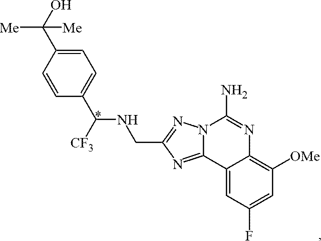 | 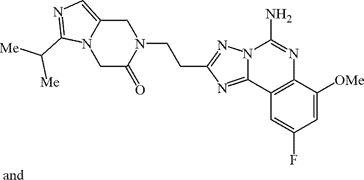 |
| 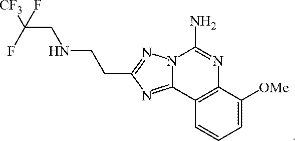 | |

.

22. A pharmaceutical composition comprising a compound of claim 1, or a pharmaceutically acceptable salt thereof, and a pharmaceutically acceptable carrier.

23. A method of treating cancer comprising administering an effective amount of a compound of claim 1, or a pharmaceutically acceptable salt thereof, to a person in need thereof.

24. The method of claim 23, wherein said cancer is selected from the group consisting of melanoma, head and neck cancer, classical Hodgkin lymphoma, urothelial carcinoma, gastric cancer, cervical cancer, primary mediastinal large-B-cell lymphoma, microsatellite instability-high cancer, non-small cell lung cancer, hepatocellular carcinoma, clear cell kidney cancer, colorectal cancer, breast cancer, squamous cell lung cancer, basal carcinoma, sarcoma, bladder cancer, endometrial cancer, pancreatic cancer, liver cancer, gastrointestinal cancer, multiple myeloma, renal cancer, mesothelioma, ovarian cancer, anal cancer, biliary tract cancer, esophageal cancer, salivary cancer, and prostate cancer, and metastatic castration resistant prostate cancer.

25. The method of claim 24, wherein said compound, or a pharmaceutically acceptable salt thereof, is administered in combination with an additional therapeutic agent.

26. The method of claim 25, wherein said additional therapeutic agent is a PD-1 antagonist.

27. The method of claim 26, wherein said additional therapeutic agent is selected from pembrolizumab, nivolumab, atezolizumab, durvalumab, and avelumab.

28. The method of claim 27, wherein said additional therapeutic agent is pembrolizumab.
